SEQUENCE LISTING (179 sequences)

SEQ ID NO: 1 - protein, 126 aa, synthetic construct
QVQLQESGGGLVQAGGSLKLSCAASGRAFIDNVMGWFRRAPGKEREFVAGLSRTGANTMYQDSVKGRFTISRDDAKNTLYLQMNSLKPEDTAVYYCAARSQGATVVITTTGGYDYWGQGTQVTVSS

SEQ ID NO: 2 - protein, 113 aa, synthetic construct
QVQLQESGGGLVQAGGSLRLSCAASGRTISNAFMGWYHQAPGEQREFVATISTTGTTNYRNSVKGRFTISRDNAKNTVYLQMNNLKPEDMGTYYCRHFGYDVWGQGTQVTVSS

SEQ ID NO: 3 - protein, 121 aa, synthetic construct
QVQLQESGGGLVQAGGSLRLSCAGSGRTFSLYSMGWFRQSAGKAREFVASINWNGEVTEYADSVKGRFIISKANANKTMSLQMNSLKFEDTGVYYCAAAPRFESTWLADYWGQGTQVTVSS

SEQ ID NO: 4 - protein, 124 aa, synthetic construct
QVQLQQFGGGLVKTGDSLRLSCAASGRSFRSYAMGWFRQNPGKERRFVAGVSWSGDITSYADSVKGRFIISRDNDKSTVYLQMHSLKAEDTAIYYCGARLGGAISEVADPYDYWGQGTLVTVSS

SEQ ID NO: 5 - protein, 123 aa, synthetic construct
QVQLQESGGGLVQAGDSLKLSCAASGDTFSRYAMAWFRQAPGKEREFVAAVSWSGGITHYADSLKGRFTISRDSAKNTVYLQMNSLKPDDTAIYYCTQDTIPGGAAREFRGYWGQGTQVTVSS

SEQ ID NO: 6 - protein, 118 aa, synthetic construct
QVQLVESGGGLVQPGGSLRLSCAASGFTFSSYWMYWVRQAPGKGLEWVSAINTGGGSTYYADSVKGRFTISRDNAKNTLYLQMNSLKSEDTAVYYCAYGNGVEGMDYWGKGTQVTVSS

SEQ ID NO: 7 - protein, 121 aa, synthetic construct
QVQLVESGGGLVQPGGSLRLSCAASGFTFSSYWMYWVRQAPGKGLEWVSAINTGGGSTYYADSVKGRFTISRDNAKNTLYLQMNSLKPEDTALYYCATRASVGTLEMYDNWGQGTQVTVSS

SEQ ID NO: 8 - protein, 123 aa, synthetic construct
QVQLVESGGGLMQPGGSLRLSCAASGFTFRSYDMSWVRQVPGKGPEWISSINSRGGSTYYTDPVKGRFTISRDNAKNTLYLEMNSLKPEDSAIYYCAKGRYGASWMFPPYDYWGQGTQVTVSS

SEQ ID NO: 9 - protein, 115 aa, synthetic construct
QVQLVESGGGLVQPGGSLRLSCAASGFTFSDSWIYWVRQAPGKGLEWVSAINTDGSNTYYADSVKGRFTISRDNAKNTLYLHMDNLKSEDTAVYYCAKGGLTGSWGQGTQVTVSS

SEQ ID NO: 10 - protein, 122 aa, synthetic construct
QVQLVESGGGLVQPGGSLRVSCAASGFTFSSYAMTWVRQAPGKGLEWVSAINGGGSTYYADSVKGRFTISRDNAKNTLYLQMNSLKPEDTALYYCARVYGSYYASFYAMDYWGKGTQVTVSS

SEQ ID NO: 11 - protein, 121 aa, synthetic construct
QVQLQQFGGGLVQAGGSLRLSCAASGLAFDSHQMGWFRQGPGKEREFVASIRSAGSTYYTDSVKGRFTISRDNAKNTVSLQMNMLKLEDTAVYYCVADRTYFGREADYDYCGQGTQVTVSS

SEQ ID NO: 12 - protein, 123 aa, synthetic construct
QVQLQQSGGGLVQAGGSLRLSCAASGRTFSNYAIGWFRQAQGKEREFVAAISWGGGNTYYAGSVKGRFTISRDNAKNTVYLQMNSLKFEDTAVYYCAASEVAHSDYEEEYDYWGQGTQVTVSS

SEQ ID NO: 13 - protein, 120 aa, synthetic construct
QVQLVESGGGLVQPGGSLRLSCAASGFTFSSYFMAWFRQAPGKEREFVAAVGWNGSLTSYADSVKGRFTISRDNAKSTLYLQMNSLKPEDTAVYYCAKESDTGWGEYDYWGQGTQVTVSS

SEQ ID NO: 14 - protein, 123 aa, synthetic construct
QVQLQQFGGGLVQAGGSLRLSCAASGRIFSTSAMAWFRQAPGKEREFVAEIGWTDESTRYADSVKGRFTISRDNAKTTIYLQMNMLKPEDTATYYCAARRFSNPPTIEAYDYWGQGTQVTVSS

SEQ ID NO: 15 - protein, 128 aa, synthetic construct
QVQLQESGGGLVQPGGSLRLSCAASGFTFSNYWMYWVRQAPGKGLEWVSGIDHGGDSTYYADSVKGRFTISRDGSKKMVYLQMDSLKPEDTAVYFCYVQAAAWWSLVGGPPPPPSDYWGQGTLVTVSS

SEQ ID NO: 16 - protein, 130 aa, synthetic construct
QVQLQESGGGLVQPGGSLRLSCAASGFTFSSYWMYWVRQAPGKGLEWVSAINTEGNTYYTDSVKGRFTISRDNARNTMYLQMDNLKSEDTGVYYCAKDAKILIARMRSQGRLSRSDFGSWGQGTQVTVSS

SEQ ID NO: 17 - protein, 128 aa, synthetic construct
QVQLQASGGGLVQPGGSLRLSCAGSGFTFSTYAMGWFRQAPGKEREFVAAISWGGSGTYYSDSAKGRFTISRDNAKNTVYLQMNSLKPEDTAVYYCAADKLRPNGTGFLARGTMIETWGQGTQVTVSS

SEQ ID NO: 18 - protein, 128 aa, synthetic construct
QVQLQQFGGGLVQPGDSLRLSCAASGRAISIYAMAWFRQAPGKEREFVATISWGGATTSYADSVKGRFTISRDNANAKSTMYLQMNDLKPEDTAVYICAAGPTDYRRNDPPAARYTYWGQGTQVTVSS

SEQ ID NO: 19 - protein, 126 aa, synthetic construct
QVQLQESGGGLVQAGGSLRLSCAASGRAFSTYVMGWFRQAPGKEREFVATINRAGGSTYYVDSVKDRFTISRDNAKNTVYLQMDSLKPEDTAVYSCAADTSSWGSNSVHESEYDYWGQGTQVTVSS

SEQ ID NO: 20 - DNA, 378 bp, synthetic construct
  source 1..378: mol_type = other DNA
caggtgcagctgcaggagagcggaggaggactggtgcaggcaggaggctctctgaagctgagctgcgcagcatccggaagggccttcatcgacaacgtgatgggctggtttaggagagcaccaggcaaggagagggagttcgtggcaggactgtccagaacaggcgccaataccatgtaccaggattctgtgaagggcaggtttacaatcagccgcgacgatgccaagaacaccctgtatctgcagatgaacagcctgaagcctgaggacacagccgtgtactattgtgcagcaagatctcagggagcaaccgtggtcatcaccacaaccggcggctacgattattggggccagggcacacaggtgaccgtgagctcc

SEQ ID NO: 21 - DNA, 339 bp, synthetic construct
  source 1..339: mol_type = other DNA
caggtgcagctgcaggagtccggaggaggactggtgcaggcaggaggctctctgaggctgagctgcgcagcatccggaaggaccatctccaacgccttcatgggctggtaccaccaggcaccaggagagcagagggagtttgtggccacaatctctaccacaggcaccacaaactaccggaacagcgtgaagggccggttcaccatcagcagagacaacgccaagaatacagtgtatctgcagatgaacaatctgaagcctgaggacatgggcacctactattgtcggcactttggctatgacgtgtggggccagggcacccaggtgacagtgagctcc

SEQ ID NO: 22 - DNA, 363 bp, synthetic construct
  source 1..363: mol_type = other DNA
caggtgcagctgcaggagtccggaggaggactggtgcaggcaggaggctccctgaggctgtcttgcgcaggcagcggacgcaccttctctctgtacagcatgggctggtttcggcagtctgccggcaaggccagagagttcgtggccagcatcaactggaatggcgaggtgacagagtatgccgacagcgtgaagggccggtttatcatctccaaggccaacgccaataagaccatgtccctgcagatgaactctctgaagttcgaggacacaggcgtgtactattgtgccgccgcccccaggtttgagtctacctggctggcagattactggggacagggaacccaggtgacagtgagctcc

SEQ ID NO: 23 - DNA, 372 bp, synthetic construct
  source 1..372: mol_type = other DNA
caggtgcagctgcagcagttcggaggaggactggtgaagaccggcgactccctgaggctgtcttgcgcagcatccggcaggtctttccgcagctacgccatgggctggtttcgccagaacccaggcaaggagcggagattcgtggcaggcgtgtcttggagcggcgacatcacaagctacgccgattccgtgaagggccggtttatcatctctagagacaatgataagagcaccgtgtatctgcagatgcactccctgaaggccgaggatacagccatctactattgtggagcaaggctgggaggagcaatctctgaggtggccgacccttacgattattggggccagggcaccctggtgacagtgagctcc

SEQ ID NO: 24 - DNA, 369 bp, synthetic construct
  source 1..369: mol_type = other DNA
caggtgcagctgcaggagtctggaggaggactggtgcaggcaggcgacagcctgaagctgtcctgcgcagcatctggcgataccttcagccggtacgcaatggcatggtttaggcaggcaccaggcaaggagagggagttcgtggcagccgtgtcttggagcggaggaatcacacactacgccgacagcctgaagggcaggtttaccatctcccgcgattctgccaagaacacagtgtatctgcagatgaacagcctgaagcccgacgataccgccatctactattgtacccaggacacaatccctggaggagcagcccgggagttcagaggctattggggccagggcacccaggtgacagtgagctcc

SEQ ID NO: 25 - DNA, 354 bp, synthetic construct
  source 1..354: mol_type = other DNA
caggtgcagctggtggagtccggaggaggactggtgcagccaggaggcagcctgaggctgtcctgcgccgcctctggcttcacctttagctcctactggatgtattgggtgcggcaggcacctggcaagggactggagtgggtgtctgccatcaacaccggcggcggcagcacatactatgccgactccgtgaagggccggttcaccatcagcagagataacgccaagaatacactgtacctgcagatgaactccctgaagtctgaggacacagccgtgtactattgtgcctacggcaatggcgtggagggcatggattattggggcaagggcacccaggtgacagtgtctagc

SEQ ID NO: 26 - DNA, 363 bp, synthetic construct
  source 1..363: mol_type = other DNA
caggtgcagctggtggagtctggaggaggactggtgcagccaggaggctccctgaggctgtcttgcgccgccagcggcttcacctttagctcctactggatgtattgggtgcgccaggcacctggcaagggactggagtgggtgagcgccatcaacaccggcggaggctccacatactatgccgactctgtgaagggccggttcaccatcagcagagataacgccaagaatacactgtacctgcagatgaactccctgaagcccgaggacacagccctgtactattgtgcaacccgggcctccgtgggcacactggagatgtatgataattggggccagggcacccaggtgacagtgtctagc

SEQ ID NO: 27 - DNA, 369 bp, synthetic construct
  source 1..369: mol_type = other DNA
caggtgcagctggtggagtccggaggaggactgatgcagccaggaggctccctgaggctgtcttgcgcagcaagcggcttcacctttaggtcctacgacatgtcttgggtgcgccaggtgccaggcaagggaccagagtggatcagctccatcaacagcaggggcggctccacctactatacagaccctgtgaagggccggttcaccatctccagagataacgccaagaatacactgtacctggagatgaacagcctgaagccagaggacagcgccatctactattgtgccaagggccggtatggcgcctcttggatgtttcccccttacgattattggggccagggcacccaggtgacagtgtctagc

SEQ ID NO: 28 - DNA, 345 bp, synthetic construct
  source 1..345: mol_type = other DNA
caggtgcagctggtggagagcggaggaggactggtgcagccaggaggctccctgaggctgtcttgcgccgccagcggcttcaccttttctgacagctggatctactgggtgcggcaggcacctggcaagggactggagtgggtgtccgccatcaacaccgacggctctaatacatactatgccgatagcgtgaagggccggttcaccatctccagagataacgccaagaatacactgtatctgcacatggacaacctgaagtccgaggataccgccgtgtactattgtgcaaagggaggactgacaggctcttggggacagggcacccaggtgacagtgagctcc

SEQ ID NO: 29 - DNA, 366 bp, synthetic construct
  source 1..366: mol_type = other DNA
caggtgcagctggtggagtccggaggaggactggtgcagccaggaggctccctgagggtgtcttgcgccgccagcggcttcacctttagctcctacgcaatgacatgggtgcgccaggcacctggcaagggactggagtgggtgtctgccatcaacggcggcggcagcacctactatgccgactccgtgaagggccggttcaccatctctagagataacgccaagaatacactgtacctgcagatgaatagcctgaagcccgaggacacagccctgtactattgtgcccgggtgtatggctcctactatgcctctttttacgccatggattattggggcaagggcacccaggtgacagtgtctagc

SEQ ID NO: 30 - DNA, 363 bp, synthetic construct
  source 1..363: mol_type = other DNA
caggtgcagctgcagcagttcggaggaggactggtgcaggcaggaggctctctgagactgagctgcgcagcatccggactggccttcgactcccaccagatgggatggtttaggcagggaccaggcaaggagagagagttcgtggcctctatcaggagcgccggctccacctactatacagactctgtgaagggccggtttaccatcagcagagataacgccaagaatacagtgtctctgcagatgaacatgctgaagctggaggacaccgccgtgtactattgcgtggccgataggacatactttggccgcgaggccgactacgattattgtggccagggcacccaggtgacagtgagctcc

SEQ ID NO: 31 - DNA, 369 bp, synthetic construct
  source 1..369: mol_type = other DNA
caggtgcagctgcagcagtctggaggaggactggtgcaggcaggaggctctctgcggctgagctgcgcagcatccggaaggaccttcagcaactacgccatcggctggtttaggcaggcacagggcaaggagagggagttcgtggcagcaatctcctggggaggaggaaatacatactatgccggctccgtgaagggccggttcaccatctctagagacaacgccaagaatacagtgtacctgcagatgaacagcctgaagtttgaggataccgccgtgtactattgtgccgcctctgaggtggcccacagcgactatgaggaggagtacgattattggggccagggcacccaggtgacagtgagctcc

SEQ ID NO: 32 - DNA, 360 bp, synthetic construct
  source 1..360: mol_type = other DNA
caggtgcagctggtggaaagcggcggcggactggtccagcctggaggatctctgagactgagctgcgccgcttctggcttcaccttcagcagctacttcatggcctggttcagacaggcccctggcaaggaacgggaattcgtggccgccgtgggctggaacggcagcctgaccagctacgccgacagcgtgaagggcagattcaccatcagccgggacaacgccaagtcaacactgtacctgcagatgaacagcctgaagcccgaggacaccgccgtgtactactgcgccaaagagagtgataccggctggggcgagtacgactactggggccagggcacacaggtgaccgtgtccagc

SEQ ID NO: 33 - DNA, 369 bp, synthetic construct
  source 1..369: mol_type = other DNA
caggtgcagctgcagcagttcggaggaggactggtgcaggcaggaggctccctgaggctgtcttgcgccgccagcggccgcatcttctctaccagcgccatggcatggtttaggcaggcaccaggcaaggagagagagttcgtggccgagatcggctggaccgacgagtccacacggtacgccgattctgtgaagggcaggtttaccatcagccgcgacaacgccaagaccacaatctatctgcagatgaatatgctgaagcccgaggataccgccacatactattgtgccgcccggagattttccaacccccctacaatcgaggcctacgactattggggccagggcacccaggtgacagtgagctcc

SEQ ID NO: 34 - DNA, 384 bp, synthetic construct
  source 1..384: mol_type = other DNA
caggtgcagctgcaggagtccggaggaggactggtgcagccaggaggctctctgaggctgagctgcgcagcatccggcttcaccttttctaactactggatgtattgggtgcggcaggcaccaggcaagggactggagtgggtgagcggaatcgaccacggaggcgattccacctactatgccgactctgtgaagggccggttcacaatctctagagatggcagcaagaagatggtgtacctgcagatggactctctgaagcctgaggatacagccgtgtacttttgttatgtgcaggcagcagcatggtggagcctggtgggaggcccccctccacccccttccgactattggggccagggcaccctggtgacagtgagctcc

SEQ ID NO: 35 - DNA, 390 bp, synthetic construct
  source 1..390: mol_type = other DNA
caggtgcagctgcaggagtccggaggaggactggtgcagccaggaggcagcctgaggctgtcctgcgccgcctctggcttcacctttagctcctactggatgtattgggtgcgccaggcacctggcaagggactggagtgggtgtccgccatcaacacagagggcaatacctactatacagacagcgtgaagggccggttcaccatctcccgggataacgccagaaatacaatgtacctgcagatggacaacctgaagtctgaggataccggcgtgtactattgtgccaaggacgccaagatcctgatcgcccggatgagatctcagggcaggctgtcccgctctgattttggcagctggggacagggaacccaggtgacagtgtctagc

SEQ ID NO: 36 - DNA, 384 bp, synthetic construct
  source 1..384: mol_type = other DNA
caggtgcagctgcaggcctctggaggaggactggtgcagccaggaggctccctgaggctgtcttgcgcaggcagcggcttcacctttagcacatacgcaatgggatggttcaggcaggcaccaggcaaggagagggagtttgtggcagcaatctcctggggaggctctggaacctactattctgacagcgccaagggccggttcaccatcagcagagataacgccaagaatacagtgtatctgcagatgaactccctgaagcccgaggacaccgccgtgtactattgtgccgccgataagctgcggcctaatggcacaggctttctggccaggggcaccatgatcgagacatggggccagggcacccaggtgacagtgagctcc

SEQ ID NO: 37 - DNA, 384 bp, synthetic construct
  source 1..384: mol_type = other DNA
caggtgcagctgcagcagttcggaggaggactggtgcagcctggcgactctctgaggctgagctgcgcagcatccggaagggccatcagcatctacgcaatggcatggttcaggcaggcaccaggcaaggagagagagtttgtggcaaccatctcctggggaggagcaaccacatcttacgcagacagcgtgaagggcaggtttaccatctcccgcgataacgccaatgccaagtctacaatgtatctgcagatgaacgacctgaagccagaggataccgccgtgtacatctgtgcagcaggaccaacagactatcggagaaatgatccccctgccgcccggtacacatattggggccagggcacccaggtgacagtgagctcc

SEQ ID NO: 38 - DNA, 378 bp, synthetic construct
  source 1..378: mol_type = other DNA
caggtgcagctgcaggagtccggaggaggactggtgcaggcaggaggcagcctgcggctgtcctgcgcagcatctggaagggccttctctacctacgtgatgggctggtttaggcaggcaccaggcaaggagagggagttcgtggcaaccatcaacagggcaggaggcagcacatactatgtggactccgtgaaggatcggtttaccatctccagagacaacgccaagaatacagtgtacctgcagatggactctctgaagcctgaggataccgccgtgtatagctgtgccgccgacacaagctcctggggctccaattctgtgcacgagagcgagtacgattattggggccagggcacccaggtgacagtgtctagc

SEQ ID NO: 39 - protein, 8 aa, synthetic construct
GRAFIDNV

SEQ ID NO: 40 - protein, 10 aa, synthetic construct
GLSRTGANTM

SEQ ID NO: 41 - protein, 19 aa, synthetic construct
AARSQGATVVITTTGGYDY

SEQ ID NO: 42 - protein, 8 aa, synthetic construct
GRTISNAF

SEQ ID NO: 43 - protein, 9 aa, synthetic construct
TISTTGTTN

SEQ ID NO: 44 - protein, 7 aa, synthetic construct
RHFGYDV

SEQ ID NO: 45 - protein, 8 aa, synthetic construct
GRTFSLYS

SEQ ID NO: 46 - protein, 9 aa, synthetic construct
INWNGEVTE

SEQ ID NO: 47 - protein, 14 aa, synthetic construct
AAAPRFESTWLADY

SEQ ID NO: 48 - protein, 8 aa, synthetic construct
GRSFRSYA

SEQ ID NO: 49 - protein, 9 aa, synthetic construct
VSWSGDITS

SEQ ID NO: 50 - protein, 17 aa, synthetic construct
GARLGGAISEVADPYDY

SEQ ID NO: 51 - protein, 8 aa, synthetic construct
GDTFSRYA

SEQ ID NO: 52 - protein, 9 aa, synthetic construct
VSWSGGITH

SEQ ID NO: 53 - protein, 16 aa, synthetic construct
TQDTIPGGAAREFRGY

SEQ ID NO: 54 - protein, 8 aa, synthetic construct
GFTFSSYW

SEQ ID NO: 55 - protein, 8 aa, synthetic construct
INTGGGST

SEQ ID NO: 56 - protein, 11 aa, synthetic construct
AYGNGVEGMDY

SEQ ID NO: 57 - protein, 14 aa, synthetic construct
ATRASVGTLEMYDN

SEQ ID NO: 58 - protein, 8 aa, synthetic construct
GFTFRSYD

SEQ ID NO: 59 - protein, 8 aa, synthetic construct
INSRGGST

SEQ ID NO: 60 - protein, 16 aa, synthetic construct
AKGRYGASWMFPPYDY

SEQ ID NO: 61 - protein, 8 aa, synthetic construct
GFTFSDSW

SEQ ID NO: 62 - protein, 8 aa, synthetic construct
INTDGSNT

SEQ ID NO: 63 - protein, 8 aa, synthetic construct
AKGGLTGS

SEQ ID NO: 64 - protein, 8 aa, synthetic construct
GFTFSSYA

SEQ ID NO: 65 - protein, 7 aa, synthetic construct
INGGGST

SEQ ID NO: 66 - protein, 16 aa, synthetic construct
ARVYGSYYASFYAMDY

SEQ ID NO: 67 - protein, 8 aa, synthetic construct
GLAFDSHQ

SEQ ID NO: 68 - protein, 7 aa, synthetic construct
IRSAGST

SEQ ID NO: 69 - protein, 15 aa, synthetic construct
VADRTYFGREADYDY

SEQ ID NO: 70 - protein, 8 aa, synthetic construct
GRTFSNYA

SEQ ID NO: 71 - protein, 8 aa, synthetic construct
ISWGGGNT

SEQ ID NO: 72 - protein, 16 aa, synthetic construct
AASEVAHSDYEEEYDY

SEQ ID NO: 73 - protein, 8 aa, synthetic construct
GFTFSSYF

SEQ ID NO: 74 - protein, 9 aa, synthetic construct
VGWNGSLTS

SEQ ID NO: 75 - protein, 13 aa, synthetic construct
AKESDTGWGEYDY

SEQ ID NO: 76 - protein, 8 aa, synthetic construct
GRIFSTSA

SEQ ID NO: 77 - protein, 9 aa, synthetic construct
IGWTDESTR

SEQ ID NO: 78 - protein, 16 aa, synthetic construct
AARRFSNPPTIEAYDY

SEQ ID NO: 79 - protein, 8 aa, synthetic construct
GFTFSNYW

SEQ ID NO: 80 - protein, 8 aa, synthetic construct
IDHGGDST

SEQ ID NO: 81 - protein, 21 aa, synthetic construct
YVQAAAWWSLVGGPPPPPSDY

SEQ ID NO: 82 - protein, 7 aa, synthetic construct
INTEGNT

SEQ ID NO: 83 - protein, 24 aa, synthetic construct
AKDAKILIARMRSQGRLSRSDFGS

SEQ ID NO: 84 - protein, 8 aa, synthetic construct
GFTFSTYA

SEQ ID NO: 85 - protein, 8 aa, synthetic construct
ISWGGSGT

SEQ ID NO: 86 - protein, 21 aa, synthetic construct
AADKLRPNGTGFLARGTMIET

SEQ ID NO: 87 - protein, 8 aa, synthetic construct
GRAISIYA

SEQ ID NO: 88 - protein, 9 aa, synthetic construct
ISWGGATTS

SEQ ID NO: 89 - protein, 19 aa, synthetic construct
AAGPTDYRRNDPPAARYTY

SEQ ID NO: 90 - protein, 8 aa, synthetic construct
GRAFSTYV

SEQ ID NO: 91 - protein, 8 aa, synthetic construct
INRAGGST

SEQ ID NO: 92 - protein, 19 aa, synthetic construct
AADTSSWGSNSVHESEYDY

SEQ ID NO: 93 - DNA, 375 bp, synthetic construct
  source 1..375: mol_type = other DNA
caggtgcagctggtggagtccggaggaggactggtgcagccaggaggcagcctgaggctgtcctgcgccgcctctggcttcacctttagctcctacgccatgagctgggtgcgccaggcaccaggcaagggacctgagtgggtgagcgccatcaactccggaggaggctccacatcttacgccgactctgtgaagggccggttcaccatcagcagagataacgccaagaatacactgtatctgcagatgaacagcctgaagccagaggacaccgccgtgtactattgtgccaagtcttggacagtgcggatcggccagatctaccaccaccccaccgattattggggccagggcacccaggtgacagtgtctagc

SEQ ID NO: 94 - protein, 125 aa, synthetic construct
QVQLVESGGGLVQPGGSLRLSCAASGFTFSSYAMSWVRQAPGKGPEWVSAINSGGGSTSYADSVKGRFTISRDNAKNTLYLQMNSLKPEDTAVYYCAKSWTVRIGQIYHHPTDYWGQGTQVTVSS

SEQ ID NO: 95 - DNA, 21 bp, synthetic construct
  source 1..21: mol_type = other DNA
ggcttcacctttagctcctac

SEQ ID NO: 96 - DNA, 21 bp, synthetic construct
  source 1..21: mol_type = other DNA
atcaactccggaggaggctcc

SEQ ID NO: 97 - DNA, 54 bp, synthetic construct
  source 1..54: mol_type = other DNA
gccaagtcttggacagtgcggatcggccagatctaccaccaccccaccgattat

SEQ ID NO: 98 - DNA, 24 bp, synthetic construct
  source 1..24: mol_type = other DNA
ggcttcacctttagctcctactgg

SEQ ID NO: 99 - DNA, 24 bp, synthetic construct
  source 1..24: mol_type = other DNA
atcaacaccggcggcggcagcaca

SEQ ID NO: 100 - DNA, 33 bp, synthetic construct
  source 1..33: mol_type = other DNA
gcctacggcaatggcgtggagggcatggattat

SEQ ID NO: 101 - DNA, 24 bp, synthetic construct
  source 1..24: mol_type = other DNA
atcaacaccggcggaggctccaca

SEQ ID NO: 102 - DNA, 42 bp, synthetic construct
  source 1..42: mol_type = other DNA
gcaacccgggcctccgtgggcacactggagatgtatgataat

SEQ ID NO: 103 - DNA, 24 bp, synthetic construct
  source 1..24: mol_type = other DNA
ggcttcacctttaggtcctacgac

SEQ ID NO: 104 - DNA, 21 bp, synthetic construct
  source 1..21: mol_type = other DNA
atcaacagcaggggcggctcc

SEQ ID NO: 105 - DNA, 48 bp, synthetic construct
  source 1..48: mol_type = other DNA
gccaagggccggtatggcgcctcttggatgtttcccccttacgattat

SEQ ID NO: 106 - DNA, 27 bp, synthetic construct
  source 1..27: mol_type = other DNA
ggcttcaccttttctgacagctggatc

SEQ ID NO: 107 - DNA, 24 bp, synthetic construct
  source 1..24: mol_type = other DNA
atcaacaccgacggctctaataca

SEQ ID NO: 108 - DNA, 24 bp, synthetic construct
  source 1..24: mol_type = other DNA
gcaaagggaggactgacaggctct

SEQ ID NO: 109 - DNA, 24 bp, synthetic construct
  source 1..24: mol_type = other DNA
ggaagggccttcatcgacaacgtg

SEQ ID NO: 110 - DNA, 30 bp, synthetic construct
  source 1..30: mol_type = other DNA
ggactgtccagaacaggcgccaataccatg

SEQ ID NO: 111 - DNA, 51 bp, synthetic construct
  source 1..51: mol_type = other DNA
gcagcaagatctcagggagcaaccgtggtcatcaccacaaccggcggctac

SEQ ID NO: 112 - DNA, 24 bp, synthetic construct
  source 1..24: mol_type = other DNA
ggaaggaccatctccaacgccttc

SEQ ID NO: 113 - DNA, 24 bp, synthetic construct
  source 1..24: mol_type = other DNA
acaatctctaccacaggcaccaca

SEQ ID NO: 114 - DNA, 21 bp, synthetic construct
  source 1..21: mol_type = other DNA
cggcactttggctatgacgtg

SEQ ID NO: 115 - DNA, 24 bp, synthetic construct
  source 1..24: mol_type = other DNA
ggacgcaccttctctctgtacagc

SEQ ID NO: 116 - DNA, 27 bp, synthetic construct
  source 1..27: mol_type = other DNA
atcaactggaatggcgaggtgacagag

SEQ ID NO: 117 - DNA, 42 bp, synthetic construct
  source 1..42: mol_type = other DNA
gccgccgcccccaggtttgagtctacctggctggcagattac

SEQ ID NO: 118 - DNA, 24 bp, synthetic construct
  source 1..24: mol_type = other DNA
ggcaggtctttccgcagctacgcc

SEQ ID NO: 119 - DNA, 27 bp, synthetic construct
  source 1..27: mol_type = other DNA
gtgtcttggagcggcgacatcacaagc

SEQ ID NO: 120 - DNA, 50 bp, synthetic construct
  source 1..50: mol_type = other DNA
ggagcaaggctggaggagcaatctctgaggtggccgacccttacgattat

SEQ ID NO: 121 - DNA, 24 bp, synthetic construct
  source 1..24: mol_type = other DNA
ggcgataccttcagccggtacgca

SEQ ID NO: 122 - DNA, 27 bp, synthetic construct
  source 1..27: mol_type = other DNA
gtgtcttggagcggaggaatcacacac

SEQ ID NO: 123 - DNA, 48 bp, synthetic construct
  source 1..48: mol_type = other DNA
acccaggacacaatccctggaggagcagcccgggagttcagaggctat

SEQ ID NO: 124 - DNA, 24 bp, synthetic construct
  source 1..24: mol_type = other DNA
ggcttcacctttagctcctacgca

SEQ ID NO: 125 - DNA, 21 bp, synthetic construct
  source 1..21: mol_type = other DNA
atcaacggcggcggcagcacc

SEQ ID NO: 126 - DNA, 48 bp, synthetic construct
  source 1..48: mol_type = other DNA
gcccgggtgtatggctcctactatgcctctttttacgccatggattat

SEQ ID NO: 127 - DNA, 24 bp, synthetic construct
  source 1..24: mol_type = other DNA
ggactggccttcgactcccaccag

SEQ ID NO: 128 - DNA, 21 bp, synthetic construct
  source 1..21: mol_type = other DNA
atcaggagcgccggctccacc

SEQ ID NO: 129 - DNA, 45 bp, synthetic construct
  source 1..45: mol_type = other DNA
gtggccgataggacatactttggccgcgaggccgactacgattat

SEQ ID NO: 130 - DNA, 24 bp, synthetic construct
  source 1..24: mol_type = other DNA
ggaaggaccttcagcaactacgcc

SEQ ID NO: 131 - DNA, 24 bp, synthetic construct
  source 1..24: mol_type = other DNA
atctcctggggaggaggaaataca

SEQ ID NO: 132 - DNA, 48 bp, synthetic construct
  source 1..48: mol_type = other DNA
gccgcctctgaggtggcccacagcgactatgaggaggagtacgattat

SEQ ID NO: 133 - DNA, 24 bp, synthetic construct
  source 1..24: mol_type = other DNA
ggcttcaccttcagcagctacttc

SEQ ID NO: 134 - DNA, 27 bp, synthetic construct
  source 1..27: mol_type = other DNA
gtgggctggaacggcagcctgaccagc

SEQ ID NO: 135 - DNA, 39 bp, synthetic construct
  source 1..39: mol_type = other DNA
gccaaagagagtgataccggctggggcgagtacgactac

SEQ ID NO: 136 - DNA, 24 bp, synthetic construct
  source 1..24: mol_type = other DNA
ggccgcatcttctctaccagcgcc

SEQ ID NO: 137 - DNA, 21 bp, synthetic construct
  source 1..21: mol_type = other DNA
atcggctggaccgacgagtcc

SEQ ID NO: 138 - DNA, 48 bp, synthetic construct
  source 1..48: mol_type = other DNA
gccgcccggagattttccaacccccctacaatcgaggcctacgactat

SEQ ID NO: 139 - DNA, 24 bp, synthetic construct
  source 1..24: mol_type = other DNA
ggcttcaccttttctaactactgg

SEQ ID NO: 140 - DNA, 18 bp, synthetic construct
  source 1..18: mol_type = other DNA
atcgaccacggaggcgat

SEQ ID NO: 141 - DNA, 63 bp, synthetic construct
  source 1..63: mol_type = other DNA
tatgtgcaggcagcagcatggtggagcctggtgggaggcccccctccacccccttccgactat

SEQ ID NO: 142 - DNA, 21 bp, synthetic construct
  source 1..21: mol_type = other DNA
atcaacacagagggcaatacc

SEQ ID NO: 143 - DNA, 72 bp, synthetic construct
  source 1..72: mol_type = other DNA
gccaaggacgccaagatcctgatcgcccggatgagatctcagggcaggctgtcccgctctgattttggcagc

SEQ ID NO: 144 - DNA, 21 bp, synthetic construct
  source 1..21: mol_type = other DNA
ggcttcacctttagcacatac

SEQ ID NO: 145 - DNA, 24 bp, synthetic construct
  source 1..24: mol_type = other DNA
atctcctggggaggctctggaacc

SEQ ID NO: 146 - DNA, 63 bp, synthetic construct
  source 1..63: mol_type = other DNA
gccgccgataagctgcggcctaatggcacaggctttctggccaggggcaccatgatcgagaca

SEQ ID NO: 147 - DNA, 24 bp, synthetic construct
  source 1..24: mol_type = other DNA
ggaagggccatcagcatctacgca

SEQ ID NO: 148 - DNA, 27 bp, synthetic construct
  source 1..27: mol_type = other DNA
atctcctggggaggagcaaccacatct

SEQ ID NO: 149 - DNA, 57 bp, synthetic construct
  source 1..57: mol_type = other DNA
gcagcaggaccaacagactatcggagaaatgatccccctgccgcccggtacacatat

SEQ ID NO: 150 - DNA, 24 bp, synthetic construct
  source 1..24: mol_type = other DNA
ggaagggccttctctacctacgtg

SEQ ID NO: 151 - DNA, 24 bp, synthetic construct
  source 1..24: mol_type = other DNA
atcaacagggcaggaggcagcaca

SEQ ID NO: 152 - DNA, 57 bp, synthetic construct
  source 1..57: mol_type = other DNA
gccgccgacacaagctcctggggctccaattctgtgcacgagagcgagtacgattat

SEQ ID NO: 153 - protein, 9 aa, synthetic construct
INSGGGSTS

SEQ ID NO: 154 - protein, 18 aa, synthetic construct
AKSWTVRIGQIYHHPTDY

SEQ ID NO: 155 - DNA, 685 bp, synthetic construct
  source 1..685: mol_type = other DNA
cgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 156 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - DNA, 699 bp, synthetic construct
  source 1..699: mol_type = other DNA
cccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 158 - protein, 232 aa, synthetic construct
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 159 - DNA, 1065 bp, synthetic construct
  source 1..1065: mol_type = other DNA
caggtgcagctggtggagtccggaggaggactggtgcagccaggaggcagcctgaggctgtcctgcgccgcctctggcttcacctttagctcctacgccatgagctgggtgcgccaggcaccaggcaagggacctgagtgggtgagcgccatcaactccggaggaggctccacatcttacgccgactctgtgaagggccggttcaccatcagcagagataacgccaagaatacactgtatctgcagatgaacagcctgaagccagaggacaccgccgtgtactattgtgccaagtcttggacagtgcggatcggccagatctaccaccaccccaccgattattggggccagggcacccaggtgacagtgtctagcgaattcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 160 - protein, 352 aa, synthetic construct
QVQLVESGGGLVQPGGSLRLSCAASGFTFSSYAMSWVRQAPGKGPEWVSAINSGGGSTSYADSVKGRFTISRDNAKNTLYLQMNSLKPEDTAVYYCAKSWTVRIGQIYHHPTDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 10 aa, synthetic construct
GGGGSGGGGS

SEQ ID NO: 162 - protein, 5 aa, synthetic construct
GGGGS

SEQ ID NO: 163 - protein, 15 aa, synthetic construct
GGGGSGGGGSGGGGS

SEQ ID NO: 164 - protein, 20 aa, synthetic construct
GGGGSGGGGSGGGGSGGGGS

SEQ ID NO: 165 - protein, 25 aa, synthetic construct
GGGGSGGGGSGGGGSGGGGSGGGGS

SEQ ID NO: 166 - protein, 30 aa, synthetic construct
GGGGSGGGGSGGGGSGGGGSGGGGSGGGGS

SEQ ID NO: 167 - protein, 35 aa, synthetic construct
GGGGSGGGGSGGGGSGGGGSGGGGSGGGGSGGGGS

SEQ ID NO: 168 - protein, 20 aa, synthetic construct
  VARIANT 3..20: note = GS repeats may be deleted
GSGSGSGSGSGSGSGSGSGS

SEQ ID NO: 169 - protein, 30 aa, synthetic construct
  VARIANT 4..30: note = GGS repeats may be deleted
GGSGGSGGSGGSGGSGGSGGSGGSGGSGGS

SEQ ID NO: 170 - protein, 40 aa, synthetic construct
  VARIANT 5..40: note = GGGS repeats may be deleted
GGGSGGGSGGGSGGGSGGGSGGGSGGGSGGGSGGGSGGGS

SEQ ID NO: 171 - protein, 40 aa, synthetic construct
  VARIANT 5..40: note = GGSG repeats may be deleted
GGSGGGSGGGSGGGSGGGSGGGSGGGSGGGSGGGSGGGSG

SEQ ID NO: 172 - protein, 50 aa, synthetic construct
  VARIANT 6..50: note = GGSGG repeats may be deleted
GGSGGGGSGGGGSGGGGSGGGGSGGGGSGGGGSGGGGSGGGGSGGGGSGG

SEQ ID NO: 173 - protein, 50 aa, synthetic construct
  VARIANT 6..50: note = GGGGS repeats may be deleted
GGGGSGGGGSGGGGSGGGGSGGGGSGGGGSGGGGSGGGGSGGGGSGGGGS

SEQ ID NO: 174 - protein, 4 aa, Homo sapiens
CPSC

SEQ ID NO: 175 - protein, 4 aa, Homo sapiens
CPPC

SEQ ID NO: 176 - protein, 20 aa, synthetic construct
METDTLLLWVLLLWVPGSTG

SEQ ID NO: 177 - protein, 209 aa, Homo sapiens
PEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLE

SEQ ID NO: 178 - protein, 18 aa, synthetic construct
AKSGTVGIGQIYHHPTDY

SEQ ID NO: 179 - protein, 18 aa, synthetic construct
AKSPTVEIGQIYHHPTDY

(19) United States
(12) Patent Application Publication
(10) Pub. No.: US 2025/0051755 A1
(43) Pub. Date: Feb. 13, 2025

(54) PLATFORM FOR ANTIBODY DISCOVERY

(71) Applicant: Dana-Farber Cancer Institute, Inc., Boston, MA (US)

(72) Inventors: Barbel Schrofelbauer, Somerville, MA (US); Patrick Kimes, San Francisco, CA (US); William Hahn, Newton, MA (US)

(21) Appl. No.: 18/719,186

(22) PCT Filed: Dec. 19, 2022
(86) PCT No.: PCT/US2022/053378

§ 371 Date: Jun. 12, 2024

Related U.S. Application Data
(60) Provisional application No. 63/290,827, filed on Dec. 17, 2021.

(51) Int. Cl. C12N 15/10; G01N 33/68

(52) U.S. Cl. CPC C12N 15/1037; G01N 33/6848; G01N 2333/70578

(57) ABSTRACT

The invention is directed to a high throughput platform for the simultaneous discovery of therapeutic antibodies and associated targets based on their phenotypic binding profiles, and monoclonal antibodies discovered therewith.

This application is the § 371 U.S. National Stage of International Application No. PCT/US2022/053378, filed 19 Dec. 2022, which claims priority to U.S. Provisional Application No. 63/290,827, filed 17 Dec. 2021, the disclosures of which are incorporated by reference herein in their entireties.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

This application contains a Sequence Listing electronically submitted to the United States Patent and Trademark Office via Patent Center as an XML file entitled “0680002943US01” having a size of 173,028 bytes and created on Jun. 12, 2024. The information contained in the Sequence Listing is incorporated by reference herein.

### FIELD OF THE INVENTION

Aspects of the invention are drawn to a high throughput platform for the simultaneous discovery of therapeutic antibodies and associated targets based on their phenotypic binding profiles, and monoclonal antibodies discovered therewith.

### BACKGROUND OF THE INVENTION

Therapeutic antibodies are approved for the treatment of specific cancers. Although molecularly targeted antibody therapies have been used successfully in treatment of cancer, the identification of cancer specific targets has remained a bottleneck in development of new therapeutics.

### SUMMARY OF THE INVENTION

Aspects of the invention are drawn towards methods of identifying one or more antibodies. In embodiments, the the method comprises subjecting an input library to affinity selection to produce an output library, wherein affinity selection comprises at least one panning step with a sample negative for a biomarker and/or at least one panning step with a sample positive for a biomarker; and analyzing the output library to identify one or more antibodies.

Embodiments can further comprise obtaining the input library.

Still further, embodiments can comprise isolating the one or more antibodies from the output library.

Further, embodiments can comprise producing the one or more antibodies. For example, producing the antibodies can comprise cloning or synthesizing, reformatting, and expressing the antibody.

For example, the one or more antibodies comprise a full-length antibody, a fusion protein, or an antibody fragment. For example, the antibody fragment comprises IgG, VH, Fab, scFv-Fc, diabody, scFv-CH3, scFab, scFv-zipper, scFv, or VHH.

Embodiments can further comprise an amplification step.

Embodiments can comprise validating the binding specificity of the one or more antibodies. For example, validating comprises an immunoassay, a live cell binding assay, high throughput cell line multiplexing through fluorescent barcoding, plate based binding assays, high content analysis, or any combination thereof. For example, the immunoassay comprises flow cytometry, enzyme-linked immunosorbent assay (ELISA), plate based fluorescence binding assays, immunohistochemistry/fluorescent imaging, western blotting. For example, flow cytometry comprises fluorescence-activated cell sorting (FACS).

Further, embodiments can comprise identifying the target of at least one antibody. For example, the target comprises HER2, EPHA2, ITGA3, ITGA6, BCAM, ICAM1, CADM1, MME, ANPEP, or ENG. For example, the identifying comprises immunoprecipitation, affinity purification, protein microarray, or genetic approaches. For example, immunoprecipitation or affinity purification comprises linking an antibody with a label to produce a labeled antibody; incubating the labeled antibody with a population of cells, wherein the labeled antibody binds to a target on the surface of the cells to produce an antibody-target conjugate; isolating the antibody-target conjugate from the population of cells; and identifying the target. For example, the antibody is linked to the label with a cleavable linker. For example, the immunoprecipitation or affinity purification comprises antibody crosslinking. For example, the antibody is labeled with a trifunctional crosslinker comprising biotin, a sulfhydryl group and an aldehyde-reactive aminooxy group linked by LC-SPDP or PEG4-SPDP, HRP, or a trifunctional crosslinker (TriCEPS). For example, immunoprecipitation or affinity purification comprises isolation of biotinylated proteins using streptavidin beads.

In embodiments, isolating the antibody-target conjugate comprises cell lysis.

In embodiments, identifying the target comprises mass spectrometry analysis. For example, mass spectrometry analysis comprises LC-MS/MS, MALDI-TOF MS, ESI, or label free analysis based on MS signal intensity. In embodiments, the labeled antibody is incubated with a population of cells.

In embodiments, identifying the target of the antibody comprises biotin transfer.

In embodiments, the input library comprises phage display, mammalian display, yeast display, bacterial display, ribosome display, or B-cells. For example, the phage display can be VHH phage display.

In embodiments, the input library comprises a naïve library, a synthetic library, a library generated after immunization of animals, or a combination thereof.

In embodiments, the sample negative for a biomarker and/or the sample positive for a biomarker comprises a cell or population of cells, a tissue, an organoid, or a combination thereof. For example, the cell comprises a live cell. For example, the cell, population of cells, tissue, organoid, or combination thereof comprises living material or fixed material. For example, the sample negative for a biomarker comprises a cell. For example, the cell comprises a primary peripheral blood mononuclear cell (PBMC) or a fibroblast. In embodiments, the population of cells comprise a cell line. For example, the cell line comprises KURAMOCHI, OVSAHO, OV8, ES2, OC314, RMUGS, or SKOV3.

In embodiments, the sample negative for a biomarker and/or the sample positive for a biomarker comprises a diseased state, a non-diseased state, and/or a combination thereof. For example, the disease comprises cancer. For example, the cancer comprises a solid cancer or a blood cancer. For example, the solid cancer comprises ovarian cancer. For example, the ovarian cancer comprises serous carcinoma, clear-cell carcinoma, mucinous ovarian cancer, or endometrial cancer.

In embodiments, the at least one panning step comprises subjecting the input library to panning with a sample negative for a biomarker to produce a first output library; and subjecting the first output library to panning with a sample positive for a biomarker to produce a second output library.

In embodiments, the at least one panning step comprises subjecting the input library to panning with a sample positive for a biomarker to produce a first output library; and subjecting the first output library to panning with a sample negative for a biomarker to produce a second output library.

In embodiments, analyzing comprises sequencing, computational pre-processing, and computational guided selection. For example, sequencing comprises next generation sequencing. For example, computational pre-processing comprises sequence filtering, sequence alignment, and sequence clustering. For example, computational guided selection comprises differential analysis, phage enrichment analysis, selection based on binding profiles, or any combination thereof.

Aspects of the invention are also drawn towards methods for generating a cancer cell surface map.

In embodiments, the method comprises producing the cancer cell surface map.

Aspects of the invention are further drawn towards a method for identifying a therapeutic target. For example, the method comprises subjecting an input display library to affinity selection to produce an output library, wherein affinity selection comprises live cell panning; analyzing the output library to identify one or more antibodies; and identifying the target of the one or more antibodies, thereby identifying a therapeutic target and therapeutic antibody.

Aspects of the invention are directed to a therapeutic target identified by methods as described herein.

Aspects of the invention are directed to a kit comprising reagents as described herein.

An aspect of the invention is directed to an isolated monoclonal antibody or fragment thereof, wherein the monoclonal antibody comprises a heavy chain variable region (VH) comprising an amino acid sequence about 90% identical to

a.
(SEQ ID NO: 1)
QVQLQESGGGLVQAGGSLKLSCAASGRAFIDNVMGWFRRAPGKEREF
VAGLSRTGANTMYQDSVKGRFTISRDDAKNTLYLQMNSLKPEDTAVY
YCAARSQGATVVITTTGGYDYWGQGTQVTVSS a-Her2 (1A11);
b.
(SEQ ID NO: 2)
QVQLQESGGGLVQAGGSLRLSCAASGRTISNAFMGWYHQAPGEQREF
VATISTTGTTNYRNSVKGRFTISRDNAKNTVYLQMNNLKPEDMGTYY
CRHFGYDVWGQGTQVTVSS a-Her2 (1A12);
c.
(SEQ ID NO: 3)
QVQLQESGGGLVQAGGSLRLSCAGSGRTFSLYSMGWFRQSAGKAREF
VASINWNGEVTEYADSVKGRFIISKANANKTMSLQMNSLKFEDTGVY
YCAAAPRFESTWLADYWGQGTQVTVSS a-Her2 (1A51);
d.
(SEQ ID NO: 4)
QVQLQQFGGGLVKTGDSLRLSCAASGRSFRSYAMGWFRQNPGKERR
FVAGVSWSGDITSYADSVKGRFIISRDNDKSTVYLQMHSLKAEDTAIY
YCGARLGGAISEVADPYDYWGQGTLVTVSS a-Her2 (1A68);
e.
(SEQ ID NO: 5)
QVQLQESGGGLVQAGDSLKLSCAASGDTFSRYAMAWFRQAPGKERE
FVAAVSWSGGITHYADSLKGRFTISRDSAKNTVYLQMNSLKPDDTAIY
YCTQDTIPGGAAREFRGYWGQGTQVTVSS a-Her2 (1A100);
f.
(SEQ ID NO: 6)
QVQLVESGGGLVQPGGSLRLSCAASGFTFSSYWMYWVRQAPGKGLE
WVSAINTGGGSTYYADSVKGRFTISRDNAKNTLYLQMNSLKSEDTAV
YYCAYGNGVEGMDYWGKGTQVTVSS a-CADM1 VHH (6N2_38);
g.
(SEQ ID NO: 7)
QVQLVESGGGLVQPGGSLRLSCAASGFTFSSYWMYWVRQAPGKGLE
WVSAINTGGGSTYYADSVKGRFTISRDNAKNTLYLQMNSLKPEDTAL
YYCATRASVGTLEMYDNWGQGTQVTVSS a-CADM1 VHH
(6N2_41);
h.
(SEQ ID NO 8)
QVQLVESGGGLMQPGGSLRLSCAASGFTFRSYDMSWVRQVPGKGPE
WISSINSRGGSTYYTDPVKGRFTISRDNAKNTLYLEMNSLKPEDSAIYY
CAKGRYGASWMFPPYDYWGQGTQVTVSS a-CADM1 VHH
(6N2_54);
i.
(SEQ ID NO: 9)
QVQLVESGGGLVQPGGSLRLSCAASGFTFSDSWIYWVRQAPGKGLEW
VSAINTDGSNTYYADSVKGRFTISRDNAKNTLYLHMDNLKSEDTAVY
YCAKGGLTGSWGQGTQVTVSS a-CADM1 VHH (6N2_56);
j.
(SEQ ID NO: 10)
QVQLVESGGGLVQPGGSLRVSCAASGFTFSSYAMTWVRQAPGKGLE
WVSAINGGGSTYYADSVKGRFTISRDNAKNTLYLQMNSLKPEDTALY
YCARVYGSYYASFYAMDYWGKGTQVTVSS a-EPHA2 (4N2_3);
k.
(SEQ ID NO: 11)
QVQLQQFGGGLVQAGGSLRLSCAASGLAFDSHQMGWFRQGPGKERE
FVASIRSAGSTYYTDSVKGRFTISRDNAKNTVSLQMNMLKLEDTAVY
YCVADRTYFGREADYDYCGQGTQVTVSS a-ITGA3/B1 (1A6);
l.
(SEQ ID NO: 12)
QVQLQQSGGGLVQAGGSLRLSCAASGRTFSNYAIGWFRQAQGKEREF
VAAISWGGGNTYYAGSVKGRFTISRDNAKNTVYLQMNSLKFEDTAV
YYCAASEVAHSDYEEEYDYWGQGTQVTVSS a-ITGA3/B1 (1A10);
m.
(SEQ ID NO: 13)
QVQLVESGGGLVQPGGSLRLSCAASGFTFSSYFMAWFRQAPGKEREF
VAAVGWNGSLTSYADSVKGRFTISRDNAKSTLYLQMNSLKPEDTAVY
YCAKESDTGWGEYDYWGQGTQVTVSS a-ITGA3/B1 (S14);
n.
(SEQ ID NO: 14)
QVQLQQFGGGLVQAGGSLRLSCAASGRIFSTSAMAWFRQAPGKEREF
VAEIGWTDESTRYADSVKGRFTISRDNAKTTIYLQMNMLKPEDTATY
YCAARRFSNPPTIEAYDYWGQGTQVTVSS a-ITGA6/B4 (1A61);
o.
(SEQ ID NO: 15)
QVQLQESGGGLVQPGGSLRLSCAASGFTFSNYWMYWVRQAPGKGLE
WVSGIDHGGDSTYYADSVKGRFTISRDGSKKMVYLQMDSLKPEDTA
VYFCYVQAAAWWSLVGGPPPPPSDYWGQGTLVTVSS a-ICAM1
(1A101);
p.
(SEQ ID NO: 16)
QVQLQESGGGLVQPGGSLRLSCAASGFTFSSYWMYWVRQAPGKGLE
WVSAINTEGNTYYTDSVKGRFTISRDNARNTMYLQMDNLKSEDTGVY
YCAKDAKILIARMRSQGRLSRSDFGSWGQGTQVTVSS a-MME
(1A102);
q.
(SEQ ID NO: 17)
QVQLQASGGGLVQPGGSLRLSCAGSGFTFSTYAMGWFRQAPGKEREF
VAAISWGGSGTYYSDSAKGRFTISRDNAKNTVYLQMNSLKPEDTAVY
YCAADKLRPNGTGFLARGTMIETWGQGTQVTVSS a-ANPEP
(1A105);
r.
(SEQ ID NO: 18)
QVQLQQFGGGLVQPGDSLRLSCAASGRAISIYAMAWFRQAPGKEREF
VATISWGGATTSYADSVKGRFTISRDNANAKSTMYLQMNDLKPEDTA
VYICAAGPTDYRRNDPPAARYTYWGQGTQVTVSS a-ANPEP
(1A108);
or
s.
(SEQ ID NO: 19)
QVQLQESGGGLVQAGGSLRLSCAASGRAFSTYVMGWFRQAPGKERE
FVATINRAGGSTYYVDSVKDRFTISRDNAKNTVYLQMDSLKPEDTAV
YSCAADTSSWGSNSVHESEYDYWGQGTQVTVSS a-ENG (1A107).

In another embodiment, the monoclonal antibody comprises a VH encoded by a nucleic acid having a nucleotide sequence at least 90% identical to:

a.
(SEQ ID NO: 20)
CAGGTGCAGCTGCAGGAGAGCGGAGGAGGACTGGTGCAGGCAGGAGGCT
CTCTGAAGCTGAGCTGCGCAGCATCCGGAAGGGCCTTCATCGACAACGTG
ATGGGCTGGTTTAGGAGAGCACCAGGCAAGGAGAGGGAGTTCGTGGCAG
GACTGTCCAGAACAGGCGCCAATACCATGTACCAGGATTCTGTGAAGGGC
AGGTTTACAATCAGCCGCGACGATGCCAAGAACACCCTGTATCTGCAGAT
GAACAGCCTGAAGCCTGAGGACACAGCCGTGTACTATTGTGCAGCAAGAT
CTCAGGGAGCAACCGTGGTCATCACCACAACCGGCGGCTACGATTATTGG
GGCCAGGGCACACAGGTGACCGTGAGCTCC a-Her2
(1A11)];
b.
(SEQ ID NO: 21)
CAGGTGCAGCTGCAGGAGTCCGGAGGAGGACTGGTGCAGGCAGGAGGCT
CTCTGAGGCTGAGCTGCGCAGCATCCGGAAGGACCATCTCCAACGCCTTC
ATGGGCTGGTACCACCAGGCACCAGGAGAGCAGAGGGAGTTTGTGGCCA
CAATCTCTACCACAGGCACCACAAACTACCGGAACAGCGTGAAGGGCCG
GTTCACCATCAGCAGAGACAACGCCAAGAATACAGTGTATCTGCAGATGA
ACAATCTGAAGCCTGAGGACATGGGCACCTACTATTGTCGGCACTTTGGC
TATGACGTGTGGGGCCAGGGCACCCAGGTGACAGTGAGCTCC
a-Her2 (1A12)];
c.
(SEQ ID NO: 22)
CAGGTGCAGCTGCAGGAGTCCGGAGGAGGACTGGTGCAGGCAGGAGGCT
CCCTGAGGCTGTCTTGCGCAGGCAGCGGACGCACCTTCTCTCTGTACAGC
ATGGGCTGGTTTCGGCAGTCTGCCGGCAAGGCCAGAGAGTTCGTGGCCAG
CATCAACTGGAATGGCGAGGTGACAGAGTATGCCGACAGCGTGAAGGGC
CGGTTTATCATCTCCAAGGCCAACGCCAATAAGACCATGTCCCTGCAGAT
GAACTCTCTGAAGTTCGAGGACACAGGCGTGTACTATTGTGCCGCCGCCC
CCAGGTTTGAGTCTACCTGGCTGGCAGATTACTGGGGACAGGGAACCCAG
GTGACAGTGAGCTCC a-Her2 (1A51)];
d.
(SEQ ID NO: 23)
CAGGTGCAGCTGCAGCAGTTCGGAGGAGGACTGGTGAAGACCGGCGACT
CCCTGAGGCTGTCTTGCGCAGCATCCGGCAGGTCTTTCCGCAGCTACGCC
ATGGGCTGGTTTCGCCAGAACCCAGGCAAGGAGCGGAGATTCGTGGCAGG
CGTGTCTTGGAGCGGCGACATCACAAGCTACGCCGATTCCGTGAAGGGCC
GGTTTATCATCTCTAGAGACAATGATAAGAGCACCGTGTATCTGCAGATG
CACTCCCTGAAGGCCGAGGATACAGCCATCTACTATTGTGGAGCAAGGCT
GGGAGGAGCAATCTCTGAGGTGGCCGACCCTTACGATTATTGGGGCCAGG
GCACCCTGGTGACAGTGAGCTCC a-Her2 (1A68)];
e.
(SEQ ID NO: 24)
CAGGTGCAGCTGCAGGAGTCTGGAGGAGGACTGGTGCAGGCAGGCGACA
GCCTGAAGCTGTCCTGCGCAGCATCTGGCGATACCTTCAGCCGGTACGCA
ATGGCATGGTTTAGGCAGGCACCAGGCAAGGAGAGGGAGTTCGTGGCAG
CCGTGTCTTGGAGCGGAGGAATCACACACTACGCCGACAGCCTGAAGGGC
AGGTTTACCATCTCCCGCGATTCTGCCAAGAACACAGTGTATCTGCAGAT
GAACAGCCTGAAGCCCGACGATACCGCCATCTACTATTGTACCCAGGACA
CAATCCCTGGAGGAGCAGCCCGGGAGTTCAGAGGCTATTGGGGCCAGGGC
ACCCAGGTGACAGTGAGCTCC a-Her2 (1A100)];
f.
(SEQ ID NO: 25)
CAGGTGCAGCTGGTGGAGTCCGGAGGAGGACTGGTGCAGCCAGGAGGCA
GCCTGAGGCTGTCCTGCGCCGCCTCTGGCTTCACCTTTAGCTCCTACTGG
ATGTATTGGGTGCGGCAGGCACCTGGCAAGGGACTGGAGTGGGTGTCTGC
CATCAACACCGGCGGCGGCAGCACATACTATGCCGACTCCGTGAAGGGCC
GGTTCACCATCAGCAGAGATAACGCCAAGAATACACTGTACCTGCAGATG
AACTCCCTGAAGTCTGAGGACACAGCCGTGTACTATTGTGCCTACGGCAA
TGGCGTGGAGGGCATGGATTATTGGGGCAAGGGCACCCAGGTGACAGTGT
CTAGC a-CADM1 VHH (6N2_38);
g.
(SEQ ID NO: 26)
CAGGTGCAGCTGGTGGAGTCTGGAGGAGGACTGGTGCAGCCAGGAGGCT
CCCTGAGGCTGTCTTGCGCCGCCAGCGGCTTCACCTTTAGCTCCTACTGG
ATGTATTGGGTGCGCCAGGCACCTGGCAAGGGACTGGAGTGGGTGAGCGC
CATCAACACCGGCGGAGGCTCCACATACTATGCCGACTCTGTGAAGGGCC
GGTTCACCATCAGCAGAGATAACGCCAAGAATACACTGTACCTGCAGATG
AACTCCCTGAAGCCCGAGGACACAGCCCTGTACTATTGTGCAACCCGGGC
CTCCGTGGGCACACTGGAGATGTATGATAATTGGGGCCAGGGCACCCAGG
TGACAGTGTCTAGC a-CADM1 VHH (6N2_41);
h.
(SEQ ID NO: 27)
CAGGTGCAGCTGGTGGAGTCCGGAGGAGGACTGATGCAGCCAGGAGGCT
CCCTGAGGCTGTCTTGCGCAGCAAGCGGCTTCACCTTTAGGTCCTACGAC
ATGTCTTGGGTGCGCCAGGTGCCAGGCAAGGGACCAGAGTGGATCAGCTC
CATCAACAGCAGGGGCGGCTCCACCTACTATACAGACCCTGTGAAGGGCC
GGTTCACCATCTCCAGAGATAACGCCAAGAATACACTGTACCTGGAGATG
AACAGCCTGAAGCCAGAGGACAGCGCCATCTACTATTGTGCCAAGGGCCG
GTATGGCGCCTCTTGGATGTTTCCCCCTTACGATTATTGGGGCCAGGGCA
CCCAGGTGACAGTGTCTAGC a-CADM1 VHH (6N2_54)];
i.
(SEQ ID NO: 28)
CAGGTGCAGCTGGTGGAGAGCGGAGGAGGACTGGTGCAGCCAGGAGGCT
CCCTGAGGCTGTCTTGCGCCGCCAGCGGCTTCACCTTTTCTGACAGCTGG
ATCTACTGGGTGCGGCAGGCACCTGGCAAGGGACTGGAGTGGGTGTCCGC
CATCAACACCGACGGCTCTAATACATACTATGCCGATAGCGTGAAGGGCC
GGTTCACCATCTCCAGAGATAACGCCAAGAATACACTGTATCTGCACATG
GACAACCTGAAGTCCGAGGATACCGCCGTGTACTATTGTGCAAAGGGAGG
ACTGACAGGCTCTTGGGGACAGGGCACCCAGGTGACAGTGAGCTCC
a-CADM1 VHH (6N2_56)];
j.
(SEQ ID NO: 29)
CAGGTGCAGCTGGTGGAGTCCGGAGGAGGACTGGTGCAGCCAGGAGGCT
CCCTGAGGGTGTCTTGCGCCGCCAGCGGCTTCACCTTTAGCTCCTACGCA
ATGACATGGGTGCGCCAGGCACCTGGCAAGGGACTGGAGTGGGTGTCTGC
CATCAACGGCGGCGGCAGCACCTACTATGCCGACTCCGTGAAGGGCCGGT
TCACCATCTCTAGAGATAACGCCAAGAATACACTGTACCTGCAGATGAAT
AGCCTGAAGCCCGAGGACACAGCCCTGTACTATTGTGCCCGGGTGTATGG
CTCCTACTATGCCTCTTTTTACGCCATGGATTATTGGGGCAAGGGCACCC
AGGTGACAGTGTCTAGC a-EPHA2 (4N2_3)];
k.
(SEQ ID NO: 30)
CAGGTGCAGCTGCAGCAGTTCGGAGGAGGACTGGTGCAGGCAGGAGGCT
CTCTGAGACTGAGCTGCGCAGCATCCGGACTGGCCTTCGACTCCCACCAG
ATGGGATGGTTTAGGCAGGGACCAGGCAAGGAGAGAGAGTTCGTGGCCT
CTATCAGGAGCGCCGGCTCCACCTACTATACAGACTCTGTGAAGGGCCGG
TTTACCATCAGCAGAGATAACGCCAAGAATACAGTGTCTCTGCAGATGAA
CATGCTGAAGCTGGAGGACACCGCCGTGTACTATTGCGTGGCCGATAGGA
CATACTTTGGCCGCGAGGCCGACTACGATTATTGTGGCCAGGGCACCCAG
GTGACAGTGAGCTCC a-ITGA3/B1 (1A6)];
l.
(SEQ ID NO: 31)
CAGGTGCAGCTGCAGCAGTCTGGAGGAGGACTGGTGCAGGCAGGAGGCT
CTCTGCGGCTGAGCTGCGCAGCATCCGGAAGGACCTTCAGCAACTACGCC
ATCGGCTGGTTTAGGCAGGCACAGGGCAAGGAGAGGGAGTTCGTGGCAG
CAATCTCCTGGGGAGGAGGAAATACATACTATGCCGGCTCCGTGAAGGGC
CGGTTCACCATCTCTAGAGACAACGCCAAGAATACAGTGTACCTGCAGAT
GAACAGCCTGAAGTTTGAGGATACCGCCGTGTACTATTGTGCCGCCTCTG
AGGTGGCCCACAGCGACTATGAGGAGGAGTACGATTATTGGGGCCAGGG
CACCCAGGTGACAGTGAGCTCC a-ITGA3/B1 (1A10)];
m.
(SEQ ID NO: 32)
CAGGTGCAGCTGGTGGAAAGCGGCGGCGGACTGGTCCAGCCTGGAGGAT
CTCTGAGACTGAGCTGCGCCGCTTCTGGCTTCACCTTCAGCAGCTACTTC
ATGGCCTGGTTCAGACAGGCCCCTGGCAAGGAACGGGAATTCGTGGCCGC
CGTGGGCTGGAACGGCAGCCTGACCAGCTACGCCGACAGCGTGAAGGGCA
GATTCACCATCAGCCGGGACAACGCCAAGTCAACACTGTACCTGCAGATG
AACAGCCTGAAGCCCGAGGACACCGCCGTGTACTACTGCGCCAAAGAGA
GTGATACCGGCTGGGGCGAGTACGACTACTGGGGCCAGGGCACACAGGT
GACCGTGTCCAGC a-ITGA3/B1 (S14)];
n.
(SEQ ID NO: 33)
CAGGTGCAGCTGCAGCAGTTCGGAGGAGGACTGGTGCAGGCAGGAGGCT
CCCTGAGGCTGTCTTGCGCCGCCAGCGGCCGCATCTTCTCTACCAGCGCC
ATGGCATGGTTTAGGCAGGCACCAGGCAAGGAGAGAGAGTTCGTGGCCGA
GATCGGCTGGACCGACGAGTCCACACGGTACGCCGATTCTGTGAAGGGCA
GGTTTACCATCAGCCGCGACAACGCCAAGACCACAATCTATCTGCAGATG
AATATGCTGAAGCCCGAGGATACCGCCACATACTATTGTGCCGCCCGGAG
ATTTTCCAACCCCCCTACAATCGAGGCCTACGACTATTGGGGCCAGGGCA
CCCAGGTGACAGTGAGCTCC a-ITGA6/B4 (1A61)];
o.
(SEQ ID NO: 34)
CAGGTGCAGCTGCAGGAGTCCGGAGGAGGACTGGTGCAGCCAGGAGGCT
CTCTGAGGCTGAGCTGCGCAGCATCCGGCTTCACCTTTTCTAACTACTGG
ATGTATTGGGTGCGGCAGGCACCAGGCAAGGGACTGGAGTGGGTGAGCGG
AATCGACCACGGAGGCGATTCCACCTACTATGCCGACTCTGTGAAGGGCC
GGTTCACAATCTCTAGAGATGGCAGCAAGAAGATGGTGTACCTGCAGATG
GACTCTCTGAAGCCTGAGGATACAGCCGTGTACTTTTGTTATGTGCAGGC
AGCAGCATGGTGGAGCCTGGTGGGAGGCCCCCCTCCACCCCCTTCCGACT
ATTGGGGCCAGGGCACCCTGGTGACAGTGAGCTCC a-ICAM1
(1A101)];
p.
(SEQ ID NO: 35)
CAGGTGCAGCTGCAGGAGTCCGGAGGAGGACTGGTGCAGCCAGGAGGCA
GCCTGAGGCTGTCCTGCGCCGCCTCTGGCTTCACCTTTAGCTCCTACTGG
ATGTATTGGGTGCGCCAGGCACCTGGCAAGGGACTGGAGTGGGTGTCCGC
CATCAACACAGAGGGCAATACCTACTATACAGACAGCGTGAAGGGCCGGT
TCACCATCTCCCGGGATAACGCCAGAAATACAATGTACCTGCAGATGGAC
AACCTGAAGTCTGAGGATACCGGCGTGTACTATTGTGCCAAGGACGCCAA
GATCCTGATCGCCCGGATGAGATCTCAGGGCAGGCTGTCCCGCTCTGATT
TTGGCAGCTGGGGACAGGGAACCCAGGTGACAGTGTCTAGC a-MME
(1A102)];
q.
(SEQ ID NO: 36)
CAGGTGCAGCTGCAGGCCTCTGGAGGAGGACTGGTGCAGCCAGGAGGCTC
CCTGAGGCTGTCTTGCGCAGGCAGCGGCTTCACCTTTAGCACATACGCAA
TGGGATGGTTCAGGCAGGCACCAGGCAAGGAGAGGGAGTTTGTGGCAGCA
ATCTCCTGGGGAGGCTCTGGAACCTACTATTCTGACAGCGCCAAGGGCCG
GTTCACCATCAGCAGAGATAACGCCAAGAATACAGTGTATCTGCAGATGA
ACTCCCTGAAGCCCGAGGACACCGCCGTGTACTATTGTGCCGCCGATAAG
CTGCGGCCTAATGGCACAGGCTTTCTGGCCAGGGGCACCATGATCGAGAC
ATGGGGCCAGGGCACCCAGGTGACAGTGAGCTCC a-ANPEP
(1A105)];
r.
(SEQ ID NO: 37)
CAGGTGCAGCTGCAGCAGTTCGGAGGAGGACTGGTGCAGCCTGGCGACTC
TCTGAGGCTGAGCTGCGCAGCATCCGGAAGGGCCATCAGCATCTACGCAA
TGGCATGGTTCAGGCAGGCACCAGGCAAGGAGAGAGAGTTTGTGGCAAC
CATCTCCTGGGGAGGAGCAACCACATCTTACGCAGACAGCGTGAAGGGCA
GGTTTACCATCTCCCGCGATAACGCCAATGCCAAGTCTACAATGTATCTG
CAGATGAACGACCTGAAGCCAGAGGATACCGCCGTGTACATCTGTGCAGC
AGGACCAACAGACTATCGGAGAAATGATCCCCCTGCCGCCCGGTACACAT
ATTGGGGCCAGGGCACCCAGGTGACAGTGAGCTCC a-ANPEP
(1A108);
or
s.
(SEQ ID NO: 38)
CAGGTGCAGCTGCAGGAGTCCGGAGGAGGACTGGTGCAGGCAGGAGGCA
GCCTGCGGCTGTCCTGCGCAGCATCTGGAAGGGCCTTCTCTACCTACGTG
ATGGGCTGGTTTAGGCAGGCACCAGGCAAGGAGAGGGAGTTCGTGGCAAC
CATCAACAGGGCAGGAGGCAGCACATACTATGTGGACTCCGTGAAGGATC
GGTTTACCATCTCCAGAGACAACGCCAAGAATACAGTGTACCTGCAGATG
GACTCTCTGAAGCCTGAGGATACCGCCGTGTATAGCTGTGCCGCCGACAC
AAGCTCCTGGGGCTCCAATTCTGTGCACGAGAGCGAGTACGATTATTGGG
GCCAGGGCACCCAGGTGACAGTGTCTAGC a-ENG (1A107)].

In embodiments, the antibody is fully human or humanized. In another embodiment, the antibody is an IgG. In other embodiments, the antibody is a single chain fragment. In further embodiments, the antibody comprises a pharmaceutical composition.

An aspect of the invention is directed to an isolated monoclonal antibody or fragment thereof, wherein the monoclonal antibody comprises a heavy chain variable region (VH) comprising three complementarity determining regions (CDRs), wherein
a) CDR1 comprises the amino acid sequence GRAFIDNV (SEQ ID NO: 39), wherein CDR2 comprises the amino acid sequence GLSRTGANTM (SEQ ID NO: 40), and wherein CDR3 comprises the amino acid sequence AARSQGATVVITTTGGYDY (SEQ ID NO: 41) [Ab α-Her2 (1A11)];
b) CDR1 comprises the amino acid sequence GRTISNAF (SEQ ID NO: 42), wherein CDR2 comprises the amino acid sequence TISTTGTTN (SEQ ID NO: 43), and wherein CDR3 comprises the amino acid sequence RHFGYDV (SEQ ID NO: 44) [Ab α-Her2 (1A12)];
c) CDR1 comprises the amino acid sequence GRTFSLYS (SEQ ID NO: 45), wherein CDR2 comprises the amino acid sequence INWNGEVTE (SEQ ID NO: 46), and wherein CDR3 comprises the amino acid sequence AAAPRFESTWLADY (SEQ ID NO: 47) [Ab α-Her2 (1A51)];
d) CDR1 comprises the amino acid sequence GRSFRSYA (SEQ ID NO: 48), wherein CDR2 comprises the amino acid sequence VSWSGDITS (SEQ ID NO: 49), and wherein CDR3 comprises the amino acid sequence GARLGGAISEVADPYDY (SEQ ID NO: 50) [Ab α-Her2 (1A68)];
e) CDR1 comprises the amino acid sequence GDTFSRYA (SEQ ID NO: 51), wherein CDR2 comprises the amino acid sequence VSWSGGITH (SEQ ID NO: 52), and wherein CDR3 comprises the amino acid sequence TQDTIPGGAAREFRGY (SEQ ID NO: 53) [Ab α-Her2 (1A100)];
f) CDR1 comprises the amino acid sequence GFTFSSYW (SEQ ID NO: 54), wherein CDR2 comprises the amino acid sequence INTGGGST (SEQ ID NO: 55), and wherein CDR3 comprises the amino acid sequence AYGNGVEGMDY (SEQ ID NO: 56) [Ab a-CADM1 VHH (6N2_38)];
g) CDR1 comprises the amino acid sequence GFTFSSYW (SEQ ID NO: 54), wherein CDR2 comprises the amino acid sequence INTGGGST (SEQ ID NO: 55), and wherein CDR3 comprises the amino acid sequence ATRASVGTLEMYDN (SEQ ID NO: 57) [Ab a-CADM1 VHH (6N2_41)];
h) CDR1 comprises the amino acid sequence GFTFRSYD (SEQ ID NO: 58), wherein CDR2 comprises the amino acid sequence INSRGGST (SEQ ID NO: 59), and wherein CDR3 comprises the amino acid sequence AKGRYGASWMFPPYDY (SEQ ID NO: 60) [Ab a-CADM1 VHH (6N2_54)];
i) CDR1 comprises the amino acid sequence GFTFSDSW (SEQ ID NO: 61), wherein CDR2 comprises the amino acid sequence INTDGSNT (SEQ ID NO: 62), and wherein CDR3 comprises the amino acid sequence AKGGLTGS (SEQ ID NO: 63) [Ab a-CADM1 VHH (6N2_56)];
j) CDR1 comprises the amino acid sequence GFTFSSYA (SEQ ID NO: 64), wherein CDR2 comprises the amino acid sequence INGGGST (SEQ ID NO: 65), and wherein CDR3 comprises the amino acid sequence ARVYGSYYASFYAMDY (SEQ ID NO: 66) [Ab a-EPHA2 (4N2_3)];
k) CDR1 comprises the amino acid sequence GLAFDSHQ (SEQ ID NO: 67), wherein CDR2 comprises the amino acid sequence IRSAGST (SEQ ID NO: 68), and wherein CDR3 comprises the amino acid sequence VADRTYFGREADYDY (SEQ ID NO: 69) [Ab a-ITGA3/B1 (1A6)];
l) CDR1 comprises the amino acid sequence GRTFSNYA (SEQ ID NO: 70), wherein CDR2 comprises the amino acid sequence ISWGGGNT (SEQ ID NO: 71), and wherein CDR3 comprises the amino acid sequence AASEVAHSDYEEEYDY (SEQ ID NO: 72) [Ab a-ITGA3/B1 (1A10)];
m) CDR1 comprises the amino acid sequence GFTFSSYF (SEQ ID NO: 73), wherein CDR2 comprises the amino acid sequence VGWNGSLTS (SEQ ID NO: 74), and wherein CDR3 comprises the amino acid sequence AKESDTGWGEYDY (SEQ ID NO: 75) [Ab a-ITGA3/B1 (S14)];
n) CDR1 comprises the amino acid sequence GRIFSTSA (SEQ ID NO: 76), wherein CDR2 comprises the amino acid sequence IGWTDESTR (SEQ ID NO: 77), and wherein CDR3 comprises the amino acid sequence AARRFSNPPTIEAYDY (SEQ ID NO: 78) [Ab a-ITGA6/B4 (1A61)];
o) CDR1 comprises the amino acid sequence GFTFSNYW (SEQ ID NO: 79), wherein CDR2 comprises the amino acid sequence IDHGGDST (SEQ ID NO: 80), and wherein CDR3 comprises the amino acid sequence YVQAAAWWSLVGGPPPPPSDY (SEQ ID NO: 81) [Ab a-ICAM1 (1A101)];
p) CDR1 comprises the amino acid sequence GFTFSSYW (SEQ ID NO: 54), wherein CDR2 comprises the amino acid sequence INTEGNT (SEQ ID NO: 82), and wherein CDR3 comprises the amino acid sequence AKDAKILIARMRSQGRLSRSDFGS (SEQ ID NO: 83) [Ab a-MME (1A102)];
q) CDR1 comprises the amino acid sequence GFTFSTYA (SEQ ID NO: 84), wherein CDR2 comprises the amino acid sequence ISWGGSGT (SEQ ID NO: 85), and wherein CDR3 comprises the amino acid sequence AADKLRPNGTGFLARGTMIET (SEQ ID NO: 86) [Ab a-ANPEP (1A105)],
r) CDR1 comprises the amino acid sequence GRAISIYA (SEQ ID NO: 87), wherein CDR2 comprises the amino acid sequence ISWGGATTS (SEQ ID NO: 88), and wherein CDR3 comprises the amino acid sequence AAGPTDYRRNDPPAARYTY (SEQ ID NO: 89) [Ab a-ANPEP (1A108)];
s) CDR1 comprises the amino acid sequence GRAFSTYV (SEQ ID NO: 90), wherein CDR2 comprises the amino acid sequence INRAGGST (SEQ ID NO: 91), and wherein CDR3 comprises the amino acid sequence AADTSSWGSNSVHESEYDY (SEQ ID NO: 92) [Ab a-ENG (1A107)], or amino acid sequences that are 90% identical thereto.

In embodiments, the antibody is fully human or humanized. In another embodiment, the antibody is an IgG. In other embodiments, the antibody is a single chain fragment. In further embodiments, the antibody comprises a pharmaceutical composition.

Aspects of the invention are also drawn towards methods for the sequential selection and identification of an anti-cancer antibody and its target. For example, the method comprises subjecting a phage display input library to affinity selection to produce an output library, wherein affinity selection comprises at least one panning step with a live cell sample positive for a biomarker and at least one panning step with a live cell sample negative for a biomarker; selecting from the output library at least one antibody candidate based on sequence analysis, binding profiles, or both; synthesizing, manufacturing, or isolating the selected antibody candidate; optionally, validating the antibody candidate by flow cytometry; and identifying the antibody candidate's target by mass spectrometry.

Embodiments can further comprise obtaining the input library.

Further, embodiments can comprise producing the one or more antibodies. For example, producing the antibodies can comprise cloning or synthesizing, reformatting, and expressing the antibody.

Embodiments can further comprise analyzing the output library to identify the antibody candidate.

Embodiments can further comprise an amplification step.

In embodiments, the input library comprises phage display, mammalian display, yeast display, bacterial display, ribosome display, or B-cells. For example, the phage display can be VHH phage display or a fully human scFv phage display.

In embodiments, the input library comprises a naïve library, a synthetic library, a library generated after immunization of animals, or a combination thereof.

In embodiments, the sample negative for a biomarker and/or the sample positive for a biomarker comprises a cell or population of cells, a tissue, an organoid, or a combination thereof. For example, the cell comprises a live cell. For example, the cell, population of cells, tissue, organoid, or combination thereof comprises living material or fixed material. For example, the sample negative for a biomarker comprises a cell. For example, the cell comprises a primary peripheral blood mononuclear cell (PBMC) or a fibroblast. In embodiments, the population of cells comprise a cell line. For example, the cell line comprises KURAMOCHI, OVSAHO, OV8, ES2, OC314, RMUGS, or SKOV3.

In embodiments, the sample negative for a biomarker and/or the sample positive for a biomarker comprises a diseased state, a non-diseased state, and/or a combination thereof. For example, the disease comprises cancer. For example, the cancer comprises a solid cancer or a blood cancer. For example, the solid cancer comprises ovarian cancer. For example, the ovarian cancer comprises serous carcinoma, clear-cell carcinoma, mucinous ovarian cancer, or endometrial cancer.

In embodiments, analyzing comprises sequencing, computational pre-processing, and computational guided selection. For example, sequencing comprises next generation sequencing. For example, computational pre-processing comprises sequence filtering, sequence alignment, and sequence clustering. For example, computational guided selection comprises differential analysis, phage enrichment analysis, selection based on binding profiles, or any combination thereof.

For example, the antibody fragment comprises a full-length antibody, a fusion protein, or an antibody fragment. For example, the antibody fragment comprises IgG, VH, Fab, scFv-Fc, diabody, scFv-CH3, scFab, scFv-zipper, scFv, or VHH.

Embodiments can comprise validating the binding specificity of the one or more antibodies. For example, validating comprises an immunoassay, a live cell binding assay, high throughput cell line multiplexing through fluorescent barcoding, plate based binding assays, high content analysis, or any combination thereof. For example, the immunoassay comprises flow cytometry, enzyme-linked immunosorbent assay (ELISA), plate based fluorescence binding assays, immunohistochemistry/fluorescent imaging, western blotting. For example, flow cytometry comprises fluorescence-activated cell sorting (FACS).

Further, embodiments can comprise identifying the target of at least one antibody. For example, the target comprises HER2, EPHA2, ITGA3, ITGA6, BCAM, ICAM1, CADM1, MME, ANPEP, or ENG. For example, the identifying comprises immunoprecipitation, affinity purification, protein microarray, or genetic approaches. For example, immunoprecipitation or affinity purification comprises linking an antibody with a label to produce a labeled antibody; incubating the labeled antibody with a population of cells, wherein the labeled antibody binds to a target on the surface of the cells to produce an antibody-target conjugate; isolating the antibody-target conjugate from the population of cells; and identifying the target. For example, the antibody is linked to the label with a cleavable linker. For example, the immunoprecipitation or affinity purification comprises antibody crosslinking. For example, the antibody is labeled with a trifunctional crosslinker comprising biotin, a sulfhydryl group and an aldehyde-reactive aminooxy group linked by LC-SPDP or PEG4-SPDP, HRP, or a trifunctional crosslinker (TriCEPS). For example, immunoprecipitation or affinity purification comprises isolation of biotinylated proteins using streptavidin beads.

In embodiments, isolating the antibody-target conjugate comprises cell lysis.

In embodiments, identifying the target comprises mass spectrometry analysis. For example, mass spectrometry analysis comprises LC-MS/MS, MALDI-TOF MS, ESI, or label free analysis based on MS signal intensity. In embodiments, the labeled antibody is incubated with a population of cells.

In embodiments, identifying the target of the antibody comprises biotin transfer.

In embodiments, the sample negative for a biomarker and/or the sample positive for a biomarker comprises a cell or population of cells, a tissue, an organoid, or a combination thereof. For example, the cell comprises a live cell. For example, the cell, population of cells, tissue, organoid, or combination thereof comprises living material or fixed material. For example, the sample negative for a biomarker comprises a cell. For example, the cell comprises a primary peripheral blood mononuclear cell (PBMC) or a fibroblast. In embodiments, the population of cells comprise a cell line. For example, the cell line comprises KURAMOCHI, OVSAHO, OV8, ES2, OC314, RMUGS, or SKOV3.

In embodiments, the at least one panning step comprises subjecting the input library to panning with a sample negative for a biomarker to produce a first output library; and subjecting the first output library to panning with a sample positive for a biomarker to produce a second output library.

In embodiments, the at least one panning step comprises subjecting the input library to panning with a sample positive for a biomarker to produce a first output library; and subjecting the first output library to panning with a sample negative for a biomarker to produce a second output library.

In embodiments, analyzing comprises sequencing, computational pre-processing, and computational guided selection. For example, sequencing comprises next generation sequencing. For example, computational pre-processing comprises sequence filtering, sequence alignment, and sequence clustering. For example, computational guided selection comprises differential analysis, phage enrichment analysis, selection based on binding profiles, or any combination thereof.

Aspects of the invention are also drawn towards methods for generating a cancer cell surface map.

In embodiments, the method comprises producing the cancer cell surface map.

Aspects of the invention are further drawn towards a method for identifying a therapeutic target. For example, the method comprises subjecting an input display library to affinity selection to produce an output library, wherein affinity selection comprises live cell panning; analyzing the output library to identify one or more antibodies; and identifying the target of the one or more antibodies, thereby identifying a therapeutic target and therapeutic antibody.

Aspects of the invention are directed to a therapeutic target identified by methods as described herein.

Aspects of the invention are directed to a kit comprising reagents as described herein. Other objects and advantages of this invention will become readily apparent from the ensuing description.

### BRIEF DESCRIPTION OF THE FIGURES

FIG. 1 provides an overview of the antibody discovery platform (i.e., the PhASTdiscovery platform). Enrichment of the input library for binders to ovarian cancer cell lines is performed by 1 round of live cell biopanning with lymphocytes for negative selection and a pool of ovarian cell lines for positive selection. The non-binders from an additional round of negative selection are than subjected to biopanning against each positive and negative cell line individually. The rescued output libraries are characterized by NGS and sequences are selected based on differential analysis. Candidates are then reformatted to VHH-hIgG-Fc antibodies, produced in a mammalian expression system and binding specificity is characterized in a live cell multiplex FACS binding assay. Targets of antibodies with binding specificities are identified using an antibody directed crosslinking and biotin transfer-based protocol on live cells followed by proteomic analysis.

FIG. 2 shows antibody selection strategy and overview of results. (Panel A) Schematic overview of the NGS analysis and antibody selection pipeline. Each output library is characterized by paired-end MiSeq. Following fragment stitching and several quality control steps full length VHH sequences are clustered based on CDR3 sequence homology and clusters subjected to differential analysis to identify sequences enriched in ovarian cancer cell lines over negative control lines. (Panel B) Schematic outline of key steps and associated numbers. (Panel C) Heatmap showing the flow cytometry binding pattern of validated antibodies tested in a set of ovarian cell lines that was used for selection, lymphocytes and fibroblasts as negative cell lines, and a set of additional non-ovarian cancer cell lines. Hierarchical clustering was performed based on antibody binding patterns. Color coding is based on % antibody binding over negative controls. gray <65%, dark blue 100%, dark grey not analyzed. Only antibodies with >65% positive binding to at least one ovarian cell line are shown.

FIG. 3 shows identification of a set of representative antibodies specific for Her2. (Panel A) FACS binding profile of clone 1A12. Flow cytometry staining was performed with 1A12-hIgG1-Fc followed by a-human-APC secondary antibody in a multiplexed format. (Panel B) Alignment of the CDR3 sequences of a set of 5 antibodies with similar FACS binding profiles. FIG. 3 (Panel B) discloses SEQ ID NOS 47, 50, 53, 41 and 44, respectively, in order of appearance. (Panel C) Target identification of ASB crosslinked antibodies. Representative mass spectrometry results for 1A68 are plotted against hIgG1 negative control. The total number of peptides/protein is shown. (Panel D) FACS binding profile of CRISPRa induced Her2 expressing OVCAR8 cells (blue) and the parental cell line (gray) stained with 1A12-hIgG1-Fc and a-human-APC antibody (left panel). In parallel cells were lyzed and Her2 expression analyzed by western blotting (right panel). (Panel E) FACS staining with 1A12-hIgG1-Fc of SKOV3 cells with (blue) or without (green) Her2 siRNA transfection (left panel). Her2 Knockdown efficiency was verified by western blotting (right panel). (Panel F) Immunoprecipitation with indicated antibodies from SKOV3 whole cell extracts. Her2 precipitation was analyzed by western blotting with an α-Her2 specific antibody. (Panel G) Western blot of indicated cell lines probed with α-Her2 antibody. Equal loading was verified by probing with a-aTubulin. (H) Binding of serial dilutions of the indicated antibodies to SKOV3 cells was analyzed by flow cytometry. APC mean fluorescent intensity was plotted against antibody dilutions in a non-linear three parameter fit (left panel) and EC50 values calculated (right panel) using PRISM software. (Panel I) Antigen dependent cytotoxicity was evaluated by quantifying antibody induced apoptosis induced by PBMCS in SKOV3 target cells by Annexin V-488 staining and subsequent flow cytometry analysis. To distinguish effector and target cells, SKOV3 cells were labeled with CellTrace-violet prior to the assay. Results represent % of annexin V positive SKOV3 cells relative to a no antibody control. Results are representative of at least 2 repetitions. Results shown in panels A, C, D and E are representative of all 5 identified antibodies. (Panel J) Internalization of indicated antibodies was measured on SKOV3 cells. Antibodies were labelled with pHrodo iFL Red and incubated with cells O/N before fluorescence analysis by flow cytometry. Internalization was quantified as mean fluorescent intensity of PE.

FIG. 4 shows the discovery of antibodies with binding specificity to high grade ovarian cell lines. (Panel A) FACS binding profile of 6N2_41 representative of cluster B antibodies. Staining was performed with 6N2_41 antibody followed by incubation with APC conjugated a-human Fc secondary antibody. HGSOC lines are underlined. (Panel B) Mass spectrometry identification of CADM1 representative for cluster B antibodies. Total number of peptides obtained with 6N2_41 vs hIgG control antibody are plotted. (Panel C) FACS binding of 6N2_38 to Kuramochi cells transfected with siRNA targeting CADM1 (blue), control transfected Kuramochi cells (green), and secondary only antibody (gray). (Panel D) FACS binding profile of clone 6N2_22. Flow cytometry staining was performed with 6N2_22-hIgG1-Fc followed by a-human-APC secondary antibody in a multiplexed format. High grad ovarian cell lines are underlined. (Panel E) Target ID of 6N2_22-hIgG1-F-ASB antibody. Mass spectrometry results for 6N2_22 are plotted against hIgG1 negative control. The total number of peptides/protein is shown. (Panel F) FACS staining with 6N2_22-hIgG1-Fc of Kuramochi cells with (blue) or without (green) BCAM transient transfection in 293T cells (left panel). BCAM overexpression was verified by western blotting (right panel). (Panel G) FACS staining with 6N2_22-hIgG1-Fc of OVSAHO cells with (blue) or without (green) BCAM siRNA transfection (left panel). BCAM knockdown efficiency was verified by western blotting (right panel). (Panel H) ELISA binding of 6N2_22-hIgG1-Fc dilutions to untreated (blue) or PNGase treated (red) recombinant human BCAM.

FIG. 5 shows graphs and schematics which indicate that 6N2 22 binds within BCAMs domains and BCAM D310/312 are essential for binding. For example, FIG. 5 shows expemplary internalization properties and induction of ADCC were tested. Data for 6N2 22 critical binding domains/residues on BCAM was collected. Panel A provides a schematic showing chimeras between BCAM and MCAM, a closely related protein with similar Ig-like domain architecture that 6N2_22 does not bind to. FIG. 5 (Panel A) discloses SEQ ID NO: 177. (Panel B, lower panel) To further map the amino acids involved in binding, mutagenesis within this region was performed, replacing structure predicted surface exposed charged residues with alanine. Expression of all constructs were comparable. (Panel B, upper panel) Although most mutations had no effect on binding, mutation of aspartic acids 310 and 312 to alanine both abolished 6N2_22 binding to BCAM, indicating that these residues are an essential part of the binding interface. (Panel C) 6N2_22 potently induced ADCC of Kuramochi cells in a dose dependent manner. The activity was dependent on BCAM expression as the antibody lost its ability to induce ADCC of BCAM KO cells.

FIG. 6 shows (Panel A) Western blot analysis of expression of BCAM in indicated ovarian cell lines. Equal loading was verified with probing for a-Tubulin. (Panel B) Box plot comparing BCAM gene expression between HGSOC (blue) and other ovarian CCLE cell lines (gray). Kuramochi and OVSAHO (6N2_22 binders) are highlighted in red. The statistical significance was tested using a Kruskal-Wallis test p=6.7×10-5. (Panel C) Representative facs histogram of ovarian cancer derived organoids stained for BCAM (blue) or IgG control (gray) (left panel). BCAM expression is shown as % positive cells compared to IgG control (right panel) (Panel D) Representative images of IHC staining of ovarian tumor tissue microarrays. Microarrays were stained with a-BCAM and fluorescently labelled a-rabbit secondary antibody (green) and counterstained with DAPI (blue). Top 2 panels represent cores from HGSOC, bottom left Mucinous adenocarcinoma, bottom right Endometrioid adenocarcinoma. (Panel E) Quantification of BCAM expression from tissue microarray of 36 HGSOV and 33 other ovarian subtypes. Statistical significance was tested using unpaired t-test p<0.0001.

FIG. 7 shows the discovery of an antibody with binding specificity to high grade ovarian cell lines leads to the identification of BCAM as therapeutic target against high grade serous ovarian cancers (HGSOC). (Panel A) FACS binding profile of clone 6N2_22. Flow cytometry staining was performed with 6N2_22-hIgG1-Fc followed by a-human-APC secondary antibody in a multiplexed format. High grad ovarian cell lines are underlined. (Panel B) Target ID of 6N2_22-hIgG1-F-ASB antibody. Mass spectrometry results for 6N2_22 are plotted against hIgG1 negative control. The total number of peptides/protein is shown. (Panel C) FACS staining with 6N2_22-hIgG1-Fc of Kuramochi cells with (blue) or without (green) BCAM transient transfection in 293T cells (left panel). BCAM overexpression was verified by western blotting (right panel). (Panel D) FACS staining with 6N2_22-hIgG1-Fc of OVSAHO cells with (blue) or without (green) BCAM siRNA transfection (left panel). BCAM knockdown efficiency was verified by western blotting (right panel). (Panel E) Western blot analysis of expression of BCAM in indicated ovarian cell lines. Equal loading was verified with probing for a-Tubulin. (Panel F) Box plot comparing BCAM gene expression between HGSOC (blue) and other ovarian CCLE cell lines (gray). Kuramochi and OVSAHO (6N2_22 binders) are highlighted in red. The statistical significance was tested using a Kruskal-Wallis test p=6.7×10-5. (Panel G) ELISA binding of 6N2_22-hIgG1-Fc dilutions to extend discussion on other targets-such as CADM1 as highly HGSOC specific target. ANPEP another protease of interest. Another class that was surprising was Integrins, in particular ITGA3/B1. Interestingly we identified different anti-ITGA3/B1 antibodies, with distinct binding profiles. Expression analysis indicates high expression across all ovarian lines, our data indicates that ITGA3/B1 can be in different states in different ovarian cell lines (different conformation, or different modification); while a commercially available antibody didn't show any cell line specificity, it bound to recombinant ITGA3/B1, while neither of our ITGA/B3 antibody showed much binding in ELISAs despite clear specificity in cell based knockdown and overexpression experiments. Without wishing to be bound by theory, previously unrecognized forms of ITGA3/B1 could be attractive therapeutic targets. This is supported by the observation that several ovarian cancer cell lines are dependent on ITGA3/B1. Highlights the suitability of our approach to identify new therapeutic targets.

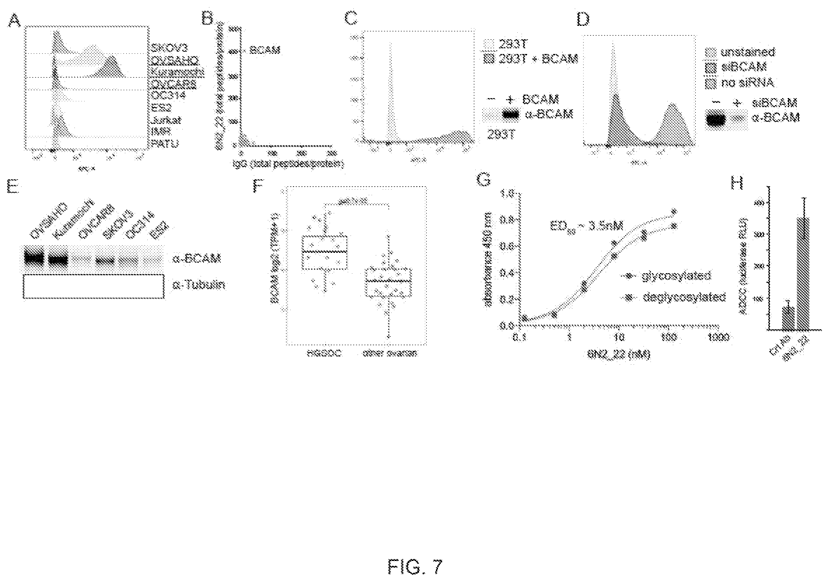

FIG. 8 shows (Panel A) Diagram showing Erbb2 gene expression plotted against Erbb2 dependency across all CCLE cell lines. Ovarian cell lines are highlighted in blue. (Panel B) Erbb2 gene expression in tumors based on TCGA data. (Panel C) Schematic illustrating the epitope binning assay. (Panel D) Epitope binning of Trastuzumab and Pertuzumab with indicated Her2-VHH antibodies.

FIG. 9 shows (Panel A) anti-BCAM 6N2_22 binding curve on Kuramochi live cells with a EC50 of 7.2 nM (Panel B) Coomassie stain of recombinant BCAM with or without PNGase treatment for deglycosylation.

FIG. 10 shows (Panel A) 6N2_22 triggered BCAM internalization was tested on Kuramochi (left panel) and OVSAHO (right panel) by comparing FACS binding upon incubation of antibody for 3h on ice versus 37° C. and subsequent staining with a-human-APC secondary antibody. (Panel B) Adhesion of Kuramochi cells with or without 6N2_22 treatment was tested by cell titer glow after a 4h incubation. Mean luminescence signal of quadruplicates is shown.

FIG. 11 shows binding of 6N2_22 to BCAM polymorphisms. (Panel A) Indicated BCAM constructs were transiently expressed in 293T cells followed by flow cytometry analysis using 6N2_22 antibody followed by a-human APC secondary antibody. Expression of the constructs was validated by western blotting using a a-BCAM and a-goat secondary antibody. Representative results from one out of three independent experiments are shown. (Panel B) Western blot of BCAM and GFP transfected cells with a-BCAM intracellular domain antibody constructs and a-GFP transfection control.

FIG. 12 shows (Panel A) BCAM expression of TCGA Pan-Cancer atlas (Panel B) BCAM expression across healthy tissues (consensus data set from Protein Atlas) (Panel C) representative images of Kidney and Thyroid stained for BCAM. Images were counterstained with DAPI and where indicated color enhanced to visualize weak BCAM staining. (Panel D) Spearman correlation between BCAM and LAMA5 expression of HGSOC tissue microarray cores of epithelial BCAM positivity with epithelial LAMA5 (left) and stromal LAMA5 (right).

FIG. 13 shows (Panel A) red blood cells were stained with 6N2_22, CD235 and Cd47 respectively and analyzed by flow cytometry. (Panel B) As control for 6N2_22 staining Kuramochi cells were analyzed in parallel. (Panel C) Expression of BCAM, Tubulin and Band3 was analyzed by western blottng of indicated cells.

FIG. 14 shows non-limiting examples of biologics used in cancer treatment.

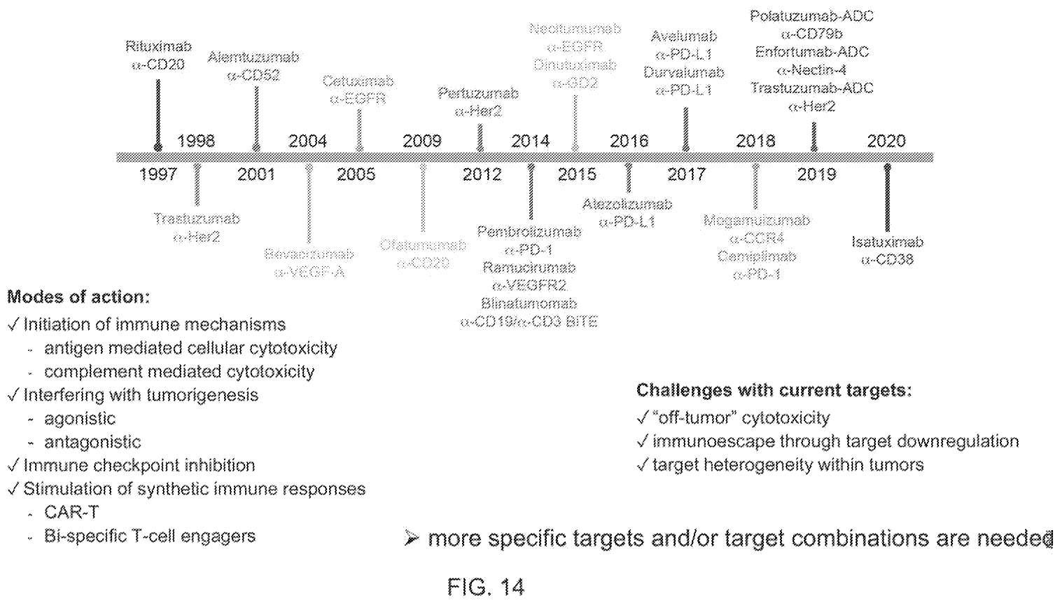

FIG. 15 shows common target ID approaches and their shortcomings

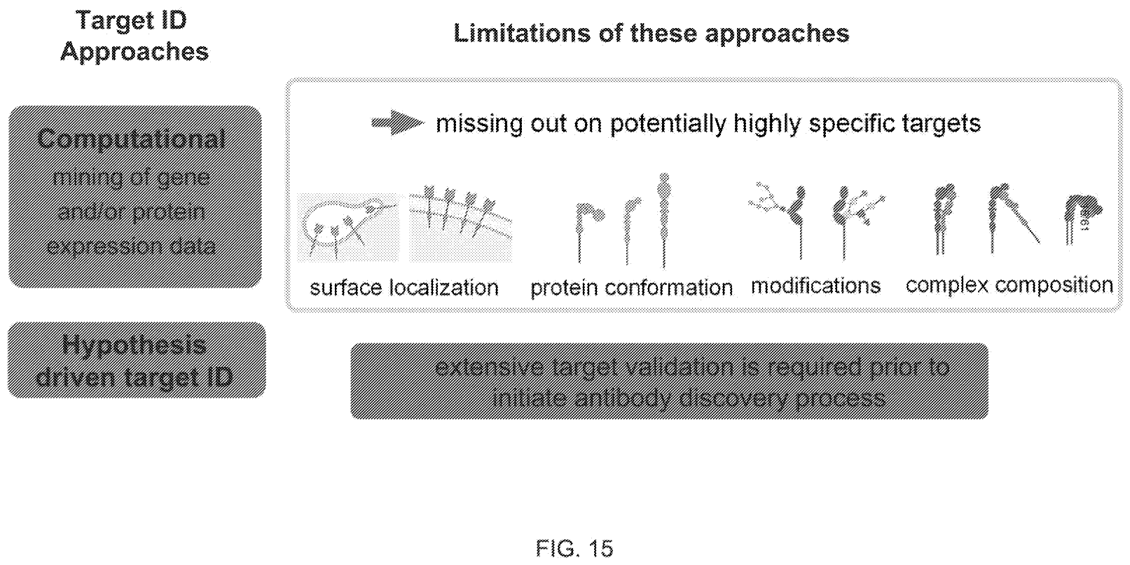

FIG. 16 shows conventional target focused antibody discovery workflow.

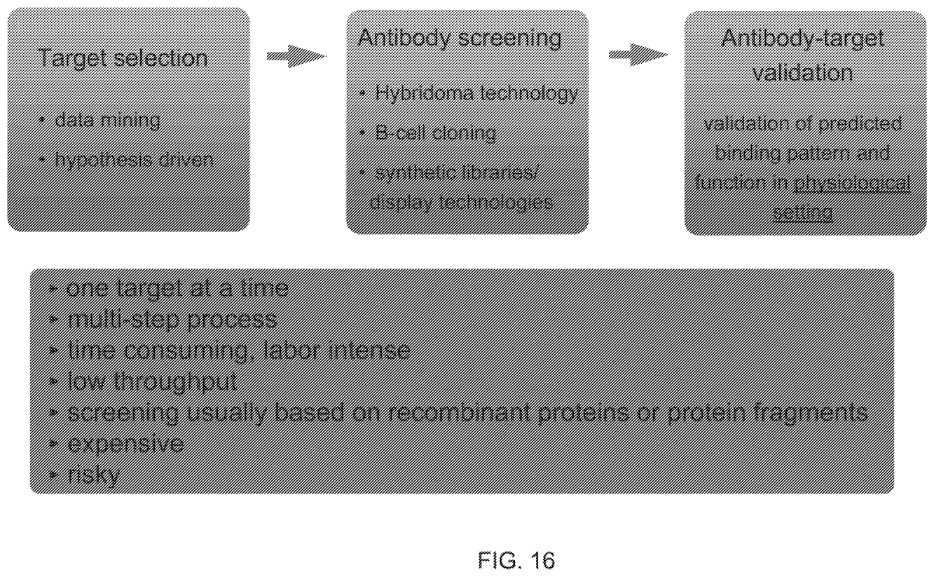

FIG. 17 shows an embodiment of the invention-simultaneous discovery of therapeutic antibodies and their cancer specific targets based on desired binding specificity.

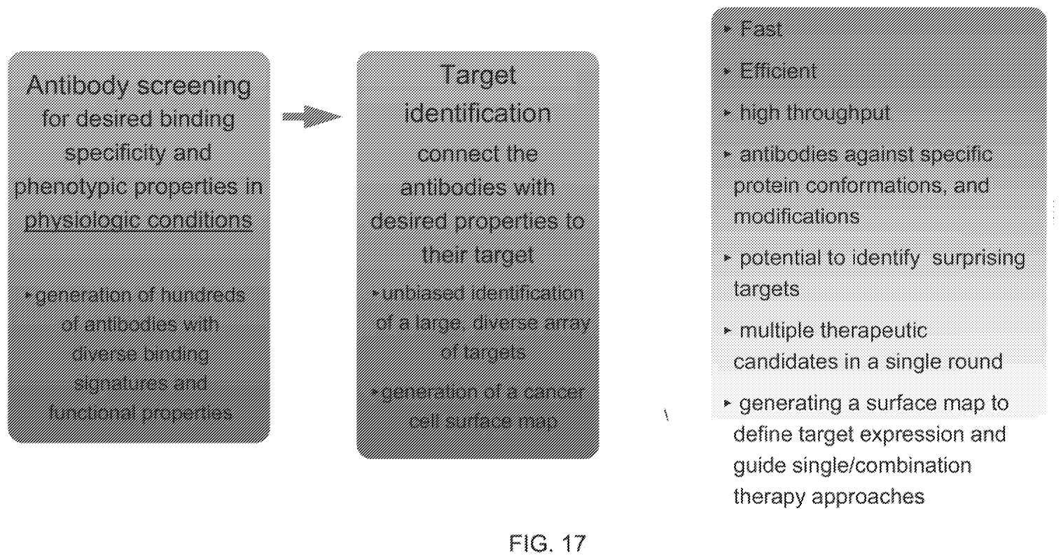

FIG. 18 shows an embodiment of the invention-simultaneous discovery of therapeutic antibodies and their cancer specific targets based on desired binding specificity.

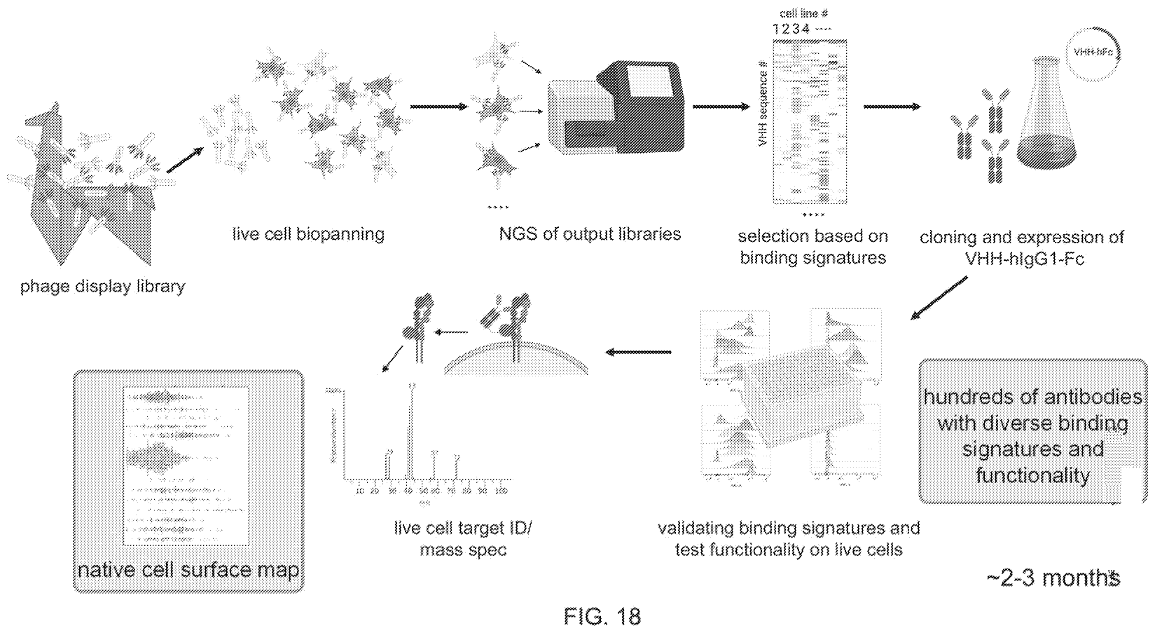

FIG. 19 shows non-limiting examples of antibody formats that can be used in embodiments described herein. For example, the antibody format can be a heavy chain only antibody (VHH/nanobody) based system.

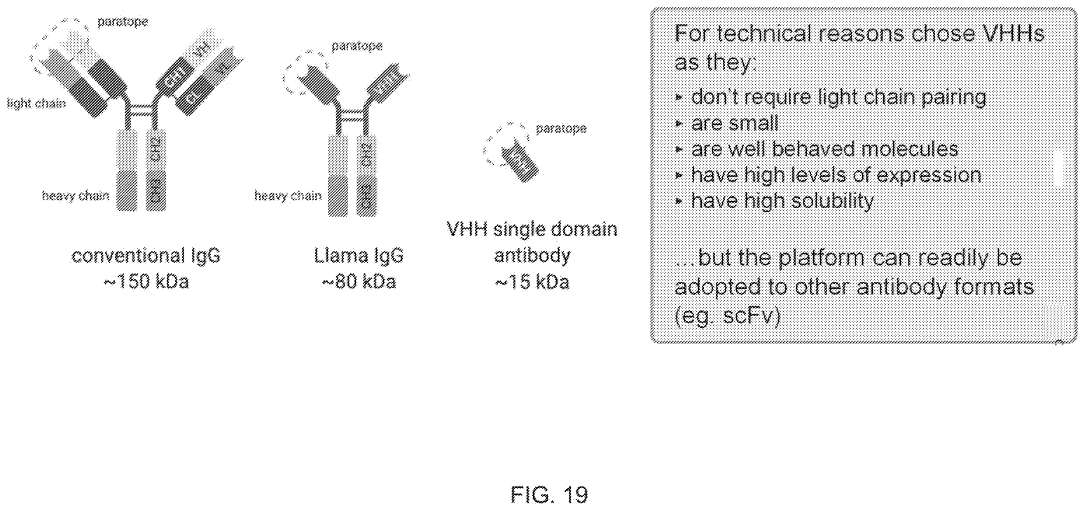

FIG. 20 shows a non-limiting example of display technology/type of library that can be used in embodiments described herein. For example, the display technology can be VHH-phage display.

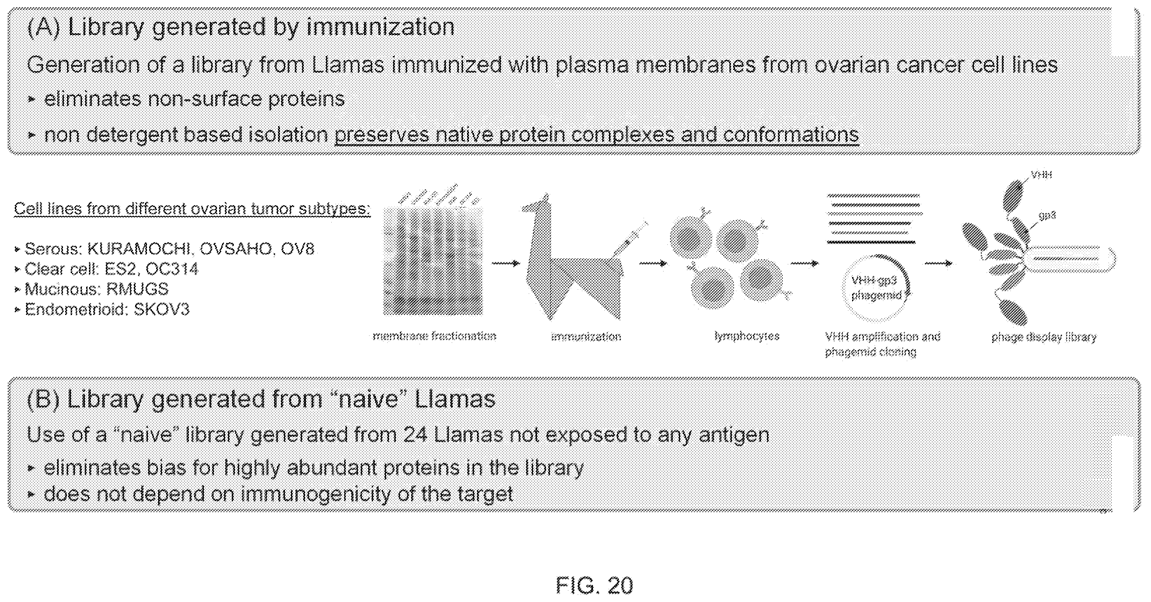

FIG. 21 shows a non-limiting example of a selection strategy that can be used in embodiments described herein. For example, the selection strategy can be biopanning and NGS for candidate selection.

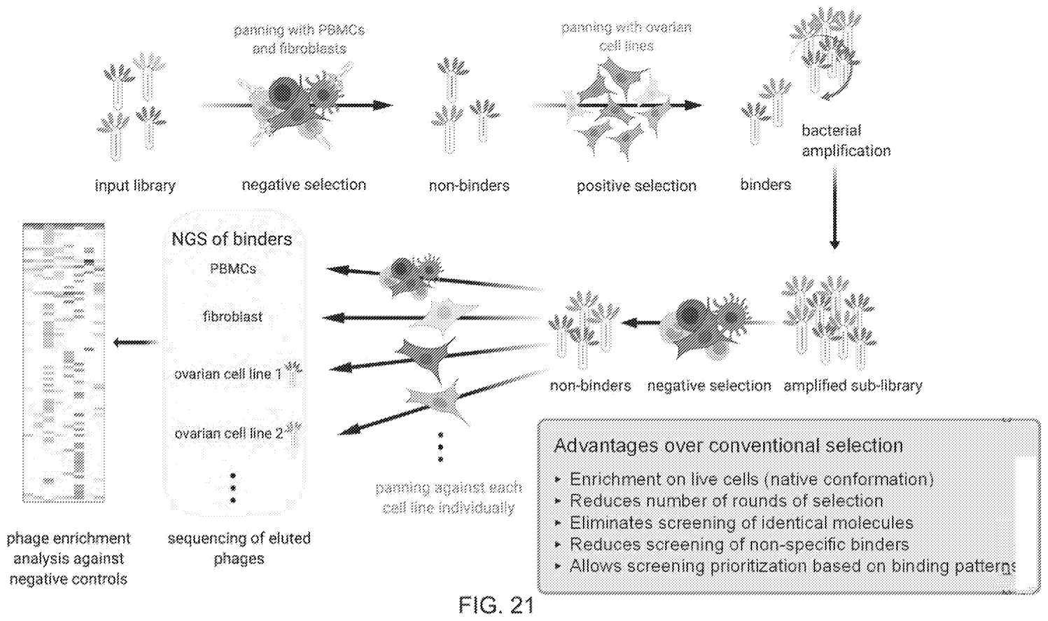

FIG. 22 shows candidate selection by Next Generation Sequencing (NGS).

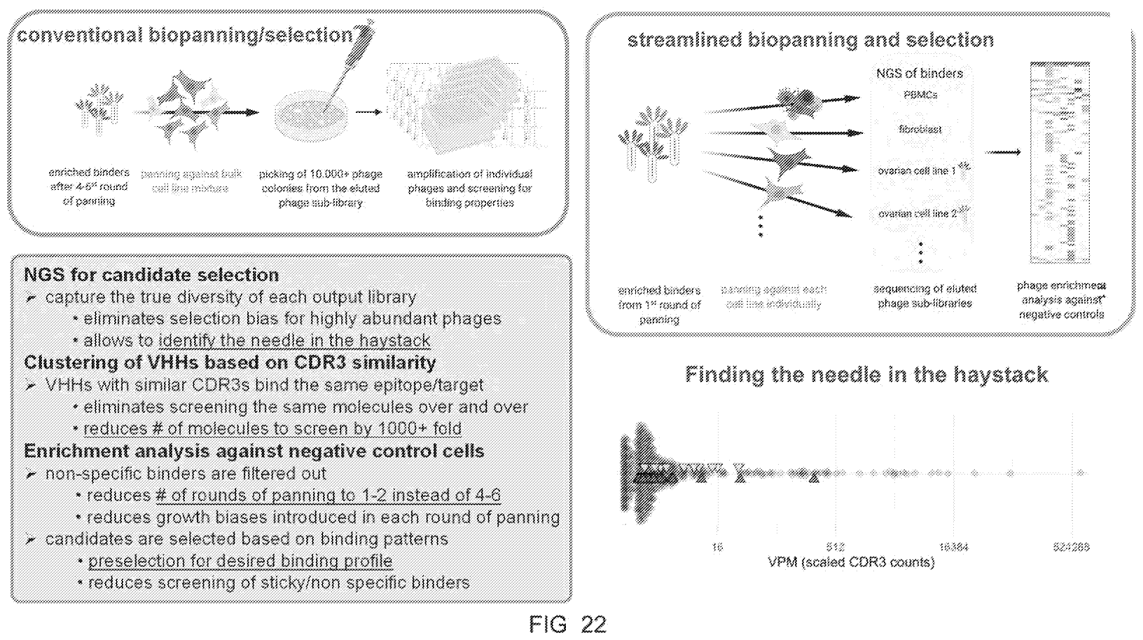

FIG. 23 shows candidate selection and expression in an embodiment of the invention.

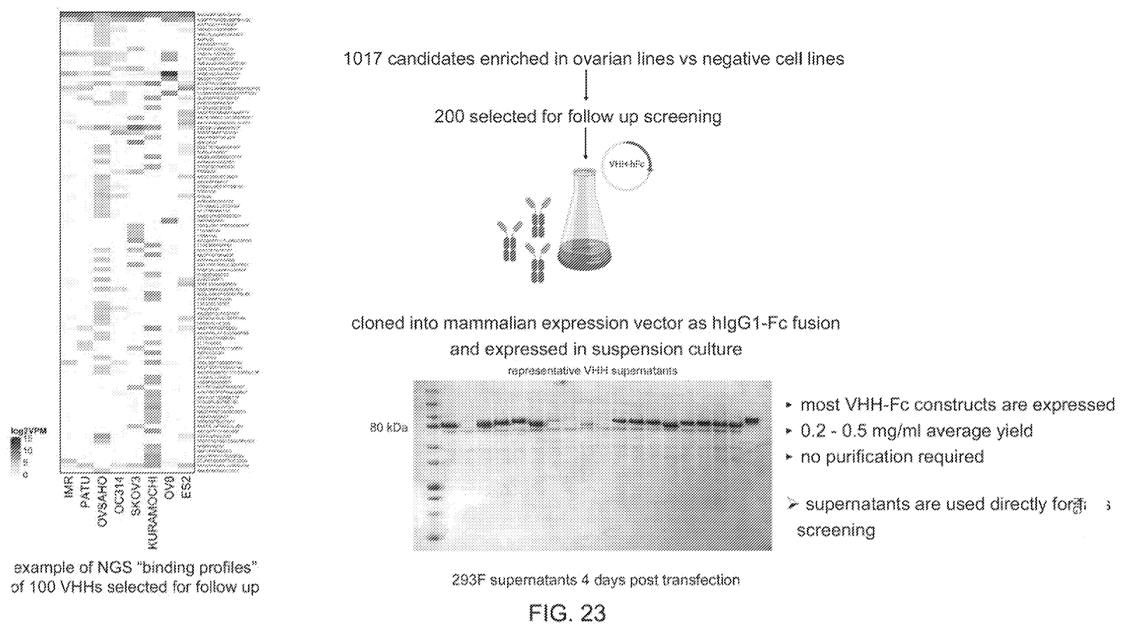

FIG. 24 shows screening technology of an embodiment of the invention. For example, the screening technology can be a multiplexed facs binding assay.

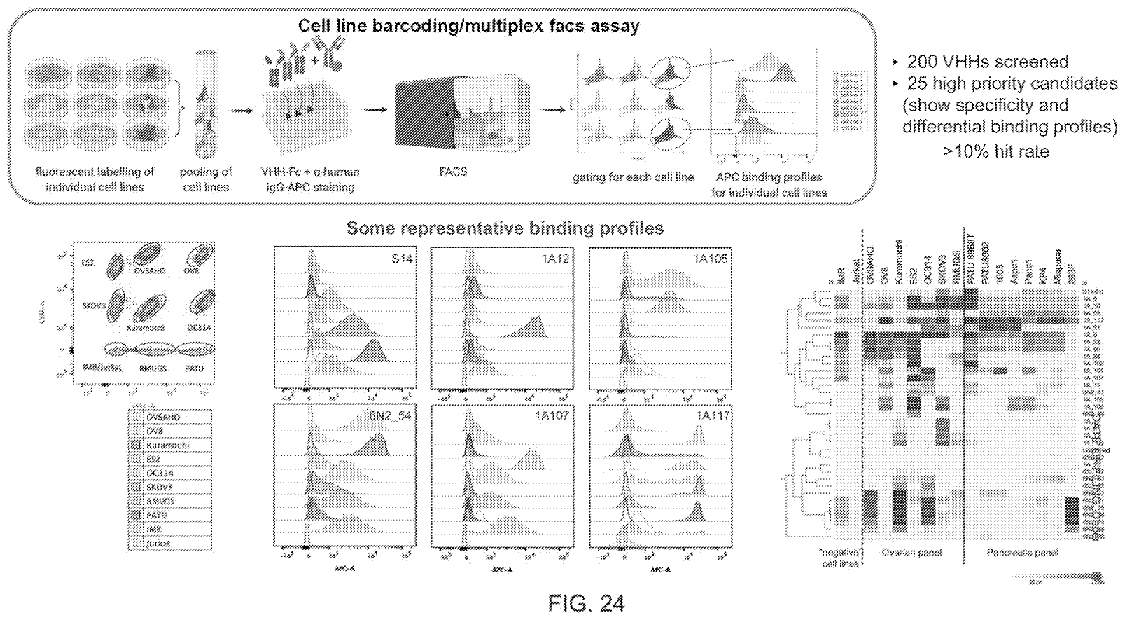

FIG. 25 shows target identification in an embodiment of the invention. For example, target identification can be by live cell target ID by biotn transfer.

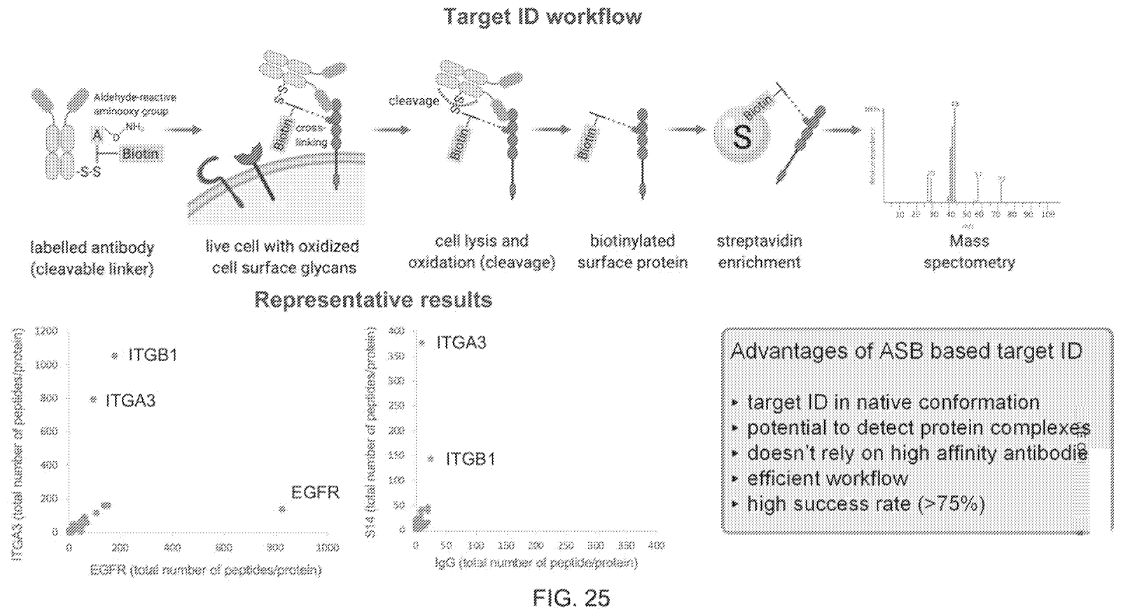

FIG. 26 shows a summary of an embodiment of the invention, including the workflow and timeline.

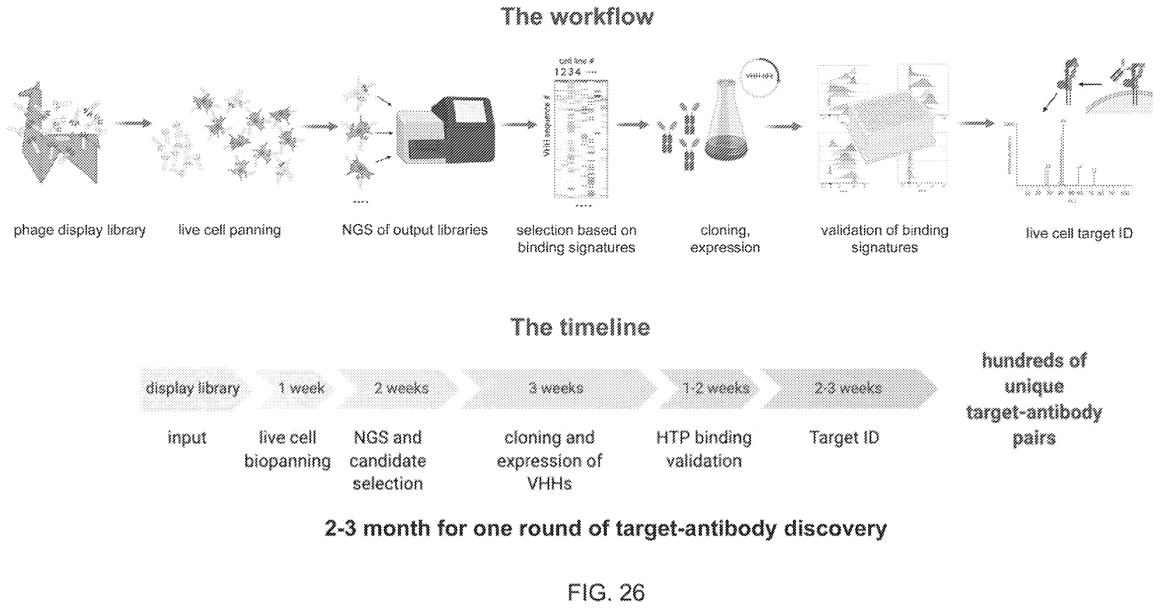

FIG. 27 shows results from a study utilizing an embodiment of the invention.

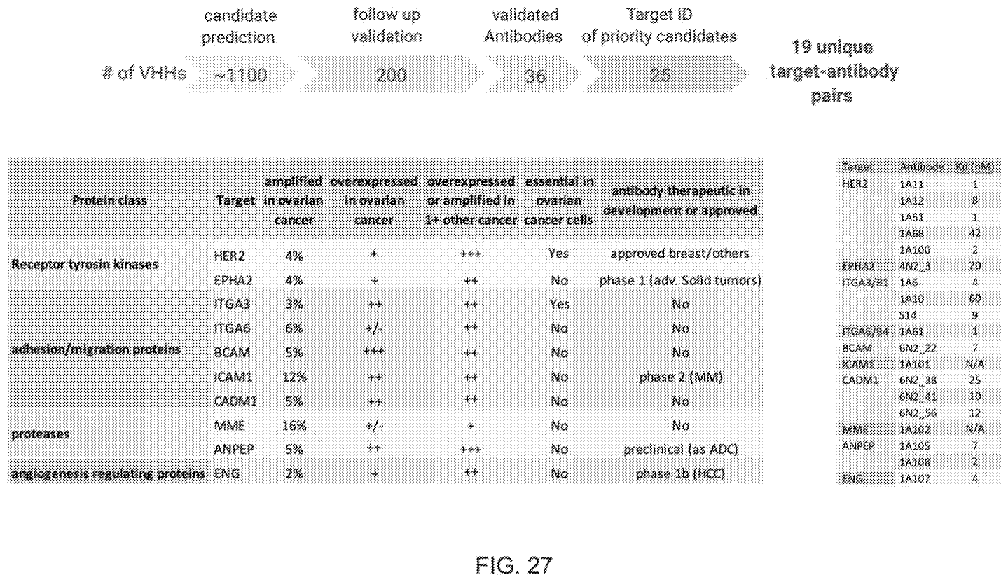

FIG. 28 shows the identification of VHHs targeting Her2.

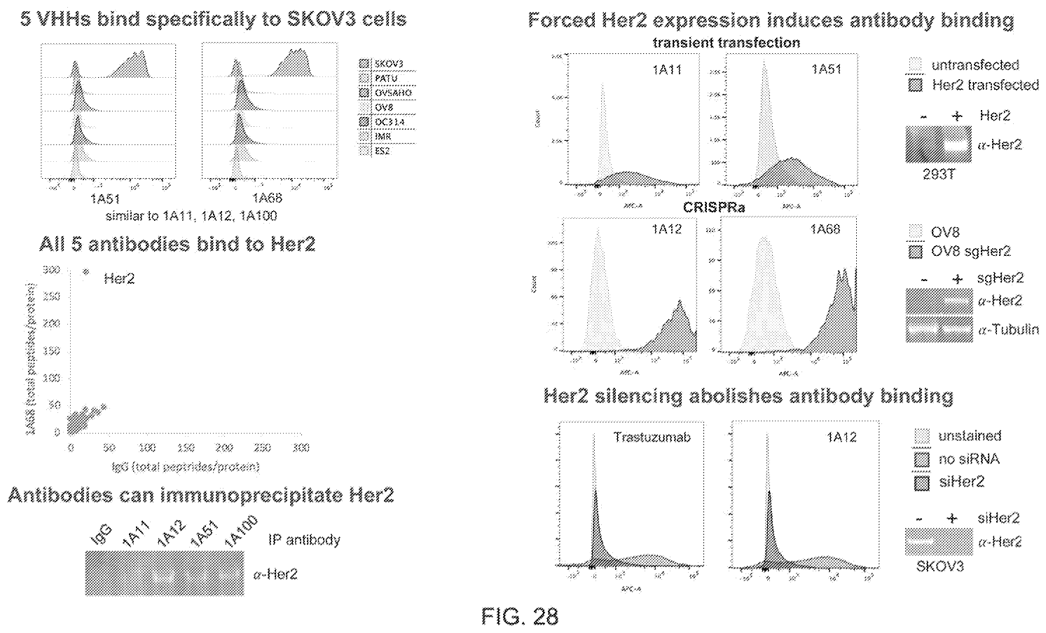

FIG. 29 shows graphs and a Western blot of Her2 expression in SKOV3 cells. Her2 is highly expressed in SKOV3 cells and ovarian cancers.

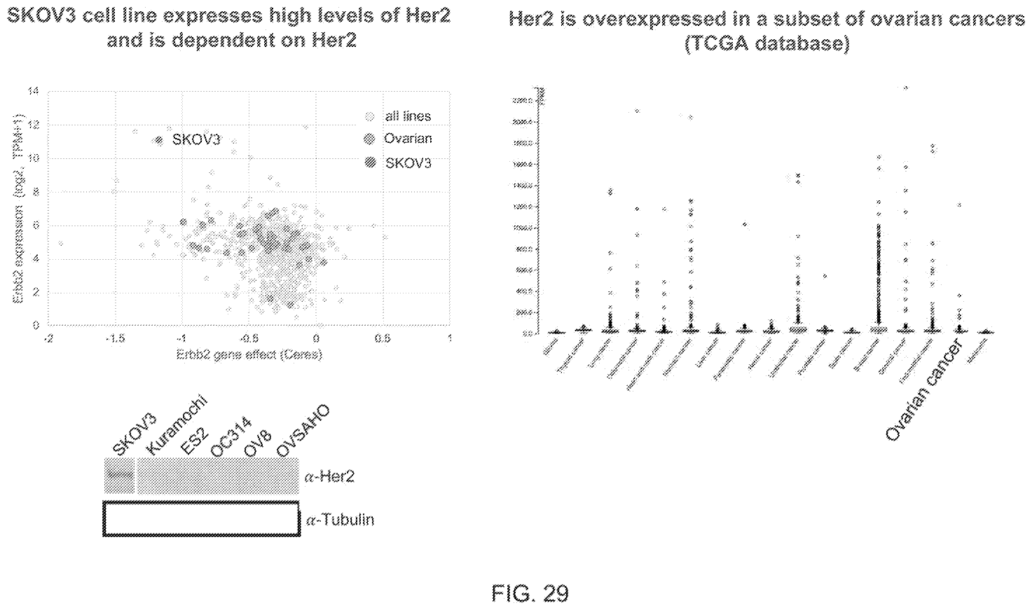

FIG. 30 shows schematics and binding data for anti-Her2 VHHs and anti-Her2 mAbs. Anti-Her2 VHHs bind different epitopes than FDA approved anti-HER2 mAbs. FIG. 30 discloses SEQ ID NOS 3-5 and 1-2, respectively, in order of appearance.

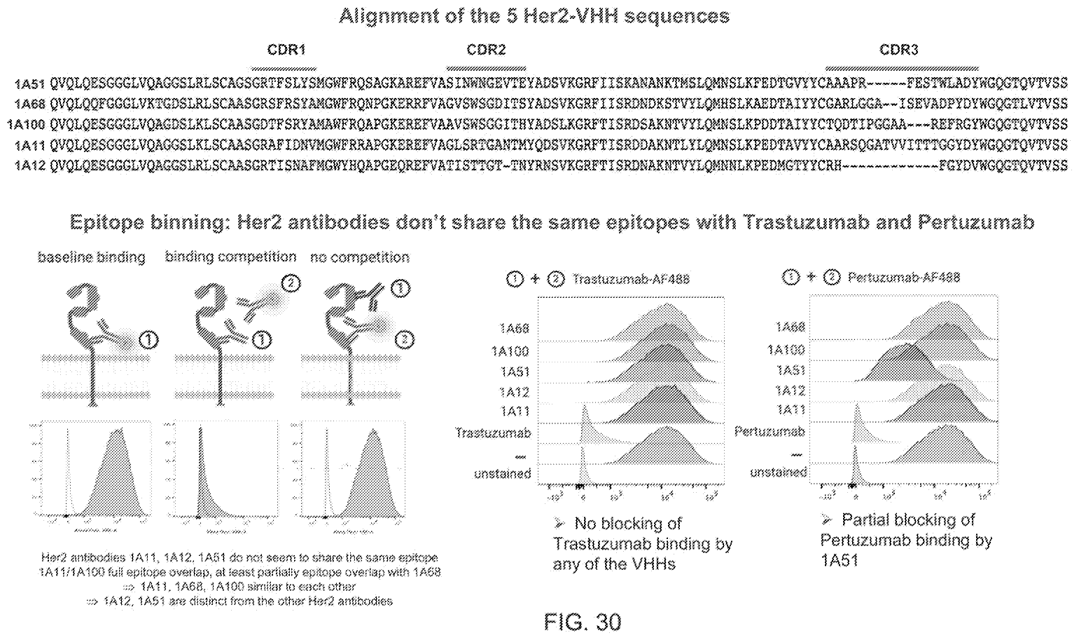

FIG. 31 shows results for anti-Her2 VHHs and anti-Her2 mAbs affinity and ADCC activity. Anti-Her2 VHHs have comparable affinity and ADCC activity to FDA approved anti-Her2 mAbs. For example, FIG. 26 shows chimeric single domain antibody affinity and ADCC activity compared to α-Her2 mAbs. For example, chimeric single domain antibody show comparable affinity and ADCC activity as FDA approved anti-Her2 mAbs.

FIG. 32 shows binding data for α-BCAM VHH against high grade serous ovarian cancers (HGSOC). For example, identification of anti-BCAM VHH as a therapeutic antibody against HGSOC is shown. For example, FIG. 27 shows identification of targets in HGSOC: BCAM. For example, panels show binding and mass spectrometry data of identification of tarets in HGSOC: BCAM.

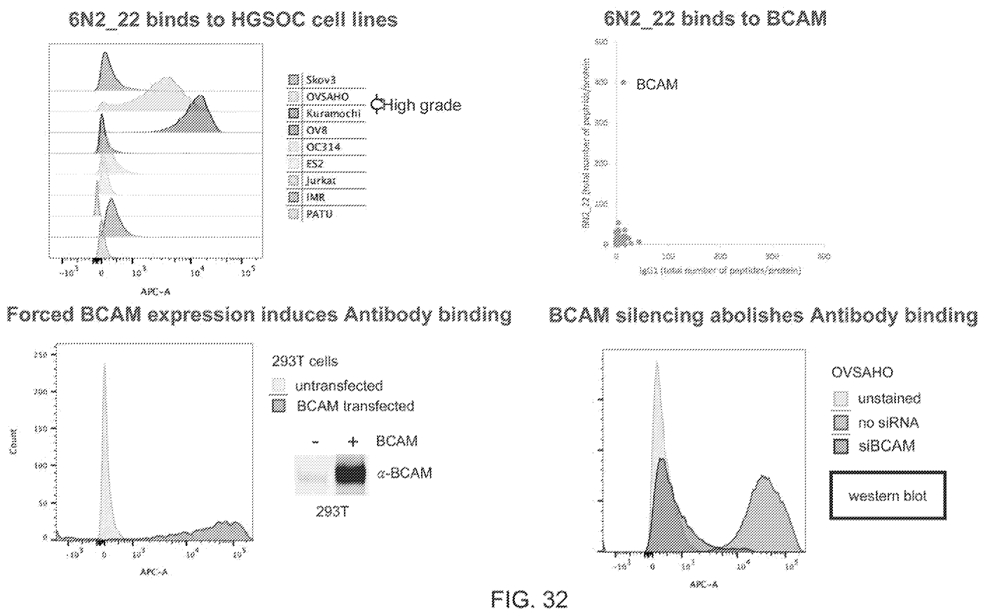

FIG. 33 shows BCAM expression is high in HGSOC cell lines.

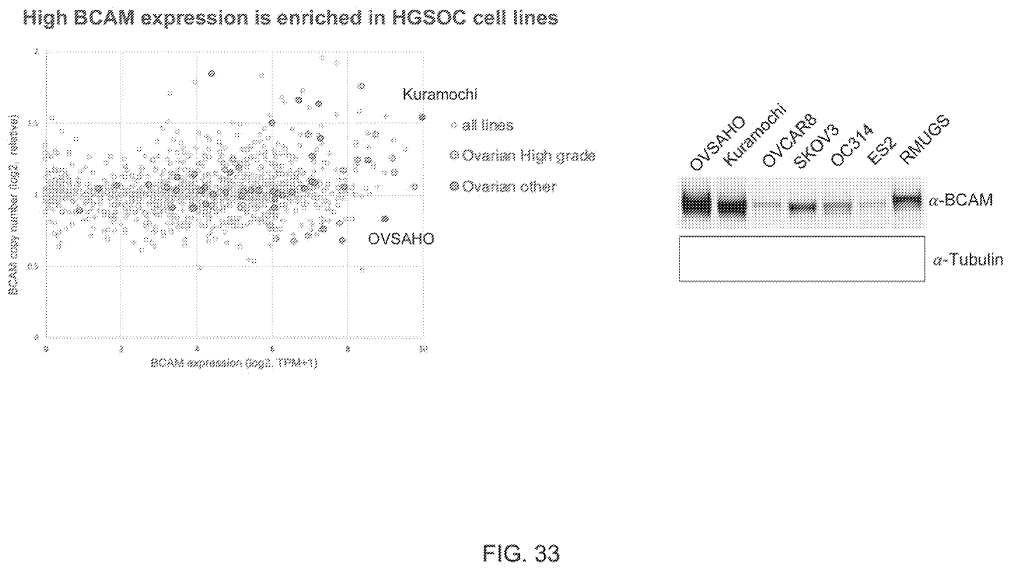

FIG. 34 shows anti-BCAM binding data. Anti-BCAM VHH binds to BCAM with low nM affinity.

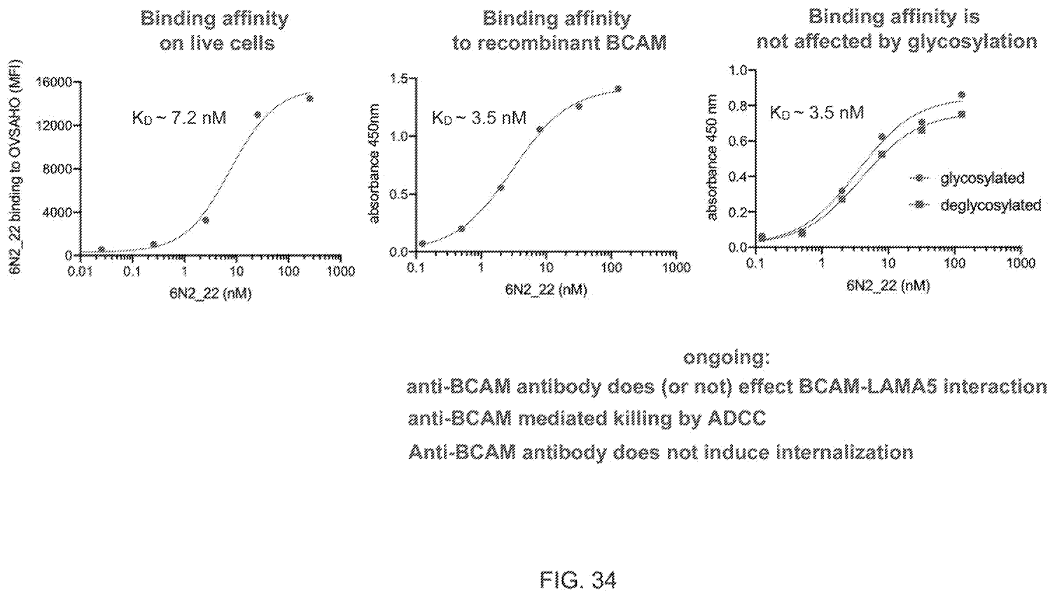

FIG. 35 shows graphs and histology of BCAM expression in HGSOC. BCAM is highly expressed in HGSOC. See also, for example, Määttä et al., J Histochem Cytochem, 53 (10), 2005; and Garinchesa, P. et al., IntJOnc, 5 (6), 1994.

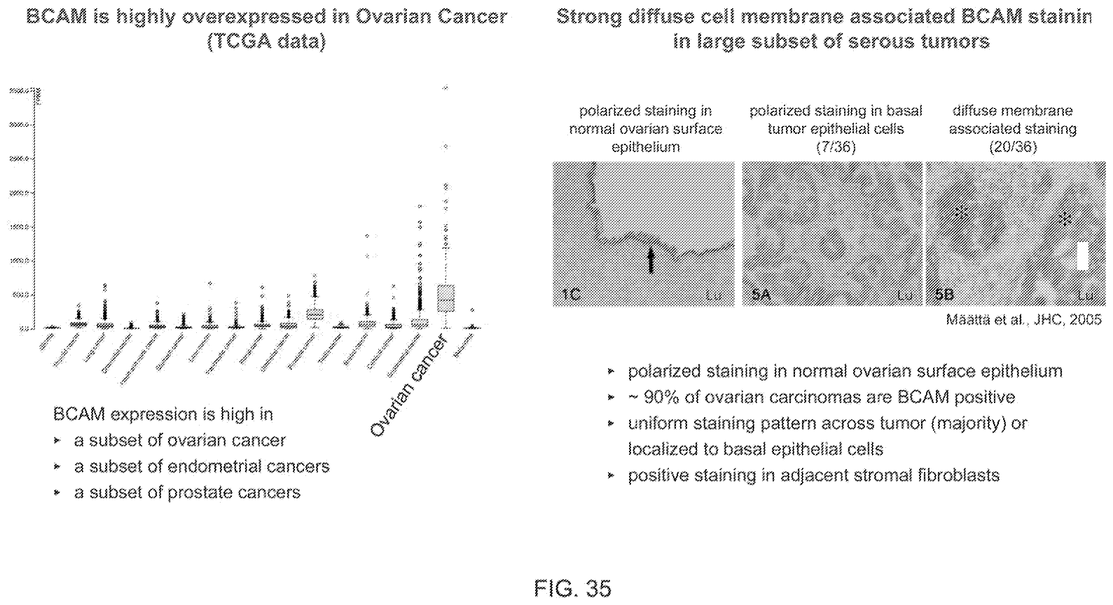

FIG. 36 shows graphs and histology of BCAM expression. For example, BCAM can be a target in colon and endometrial cancers (see also, for example, Bertolini et al., Clin Clinical Research, 22 (19), 2016).

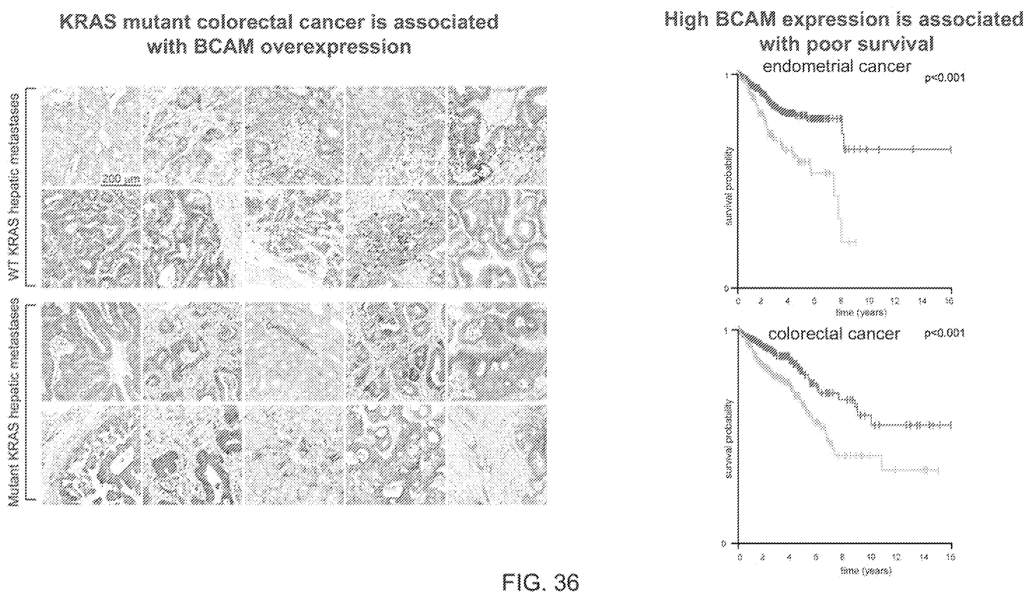

FIG. 37 shows a schematic of timeline and versatility of the FASTdisocovery platform.

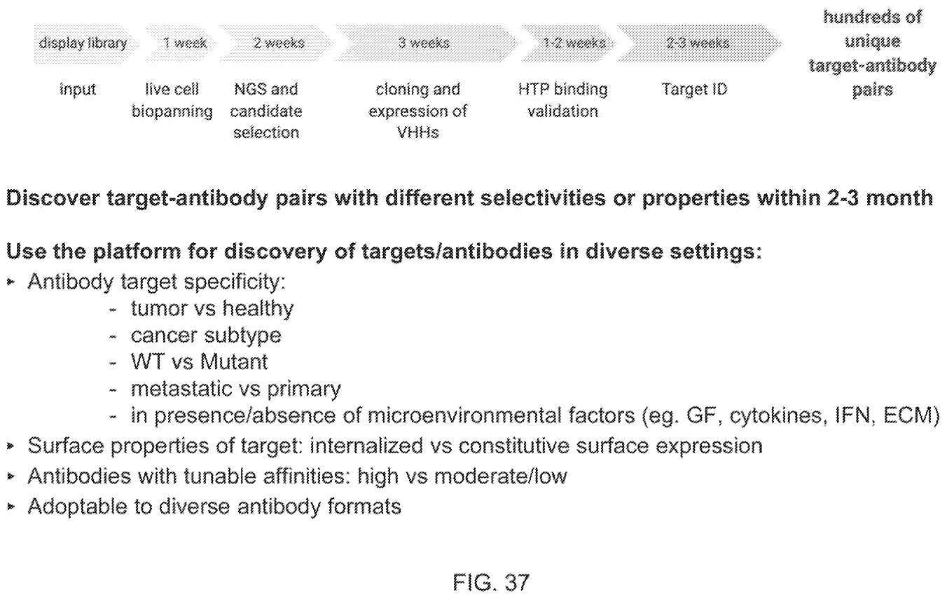

FIG. 38 shows antibody binding profiles. For example, antibodies with diverse binding profiles target Integrin A3/B1.

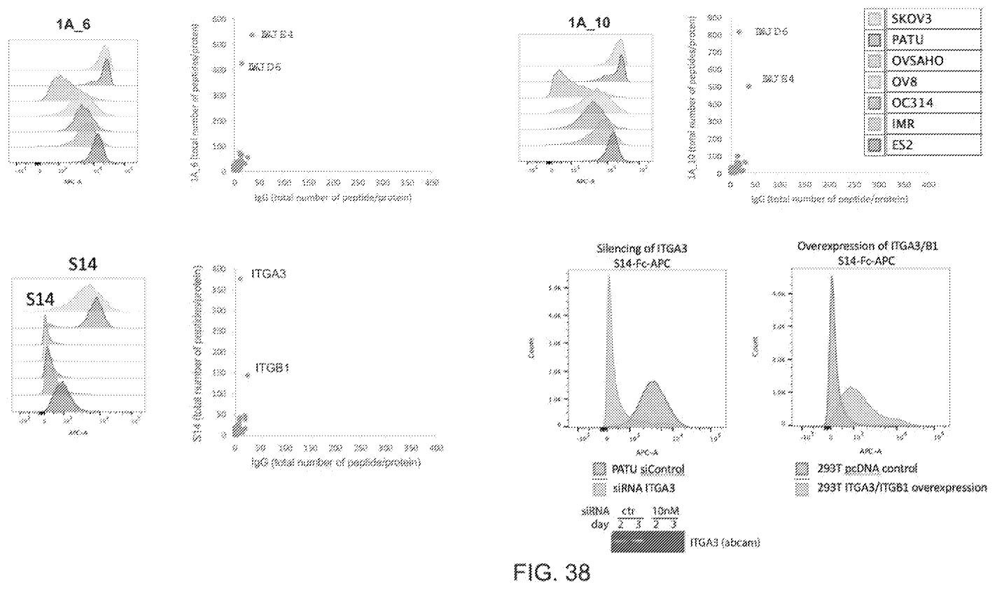

FIG. 39 shows binding profiles of ITGA3/B1. For example, ITGA3/B1 antibodies can bind to different conformations.

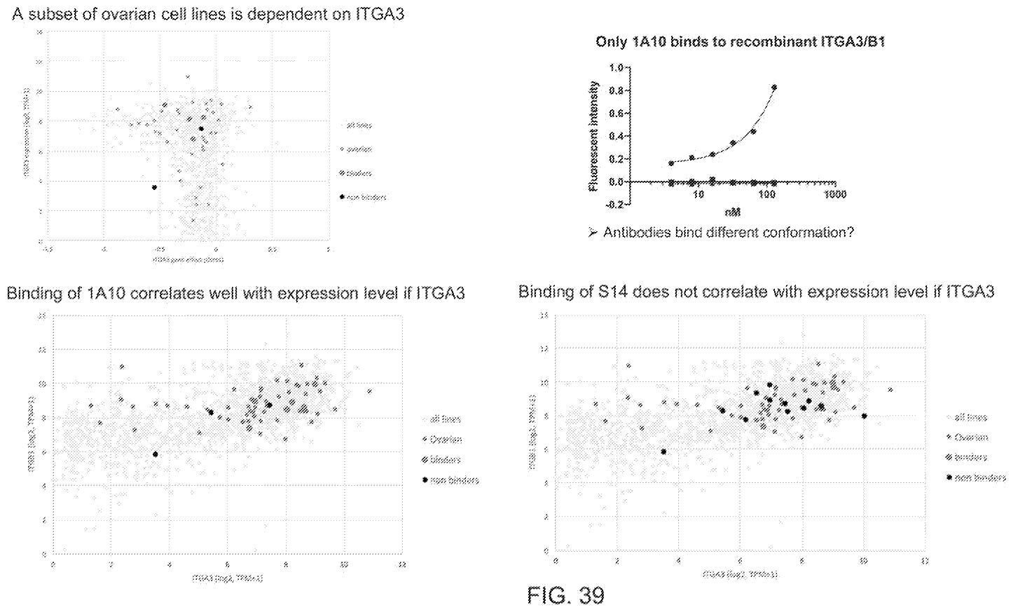

FIG. 40 shows limitations of conventional target-focused antibody discovery.

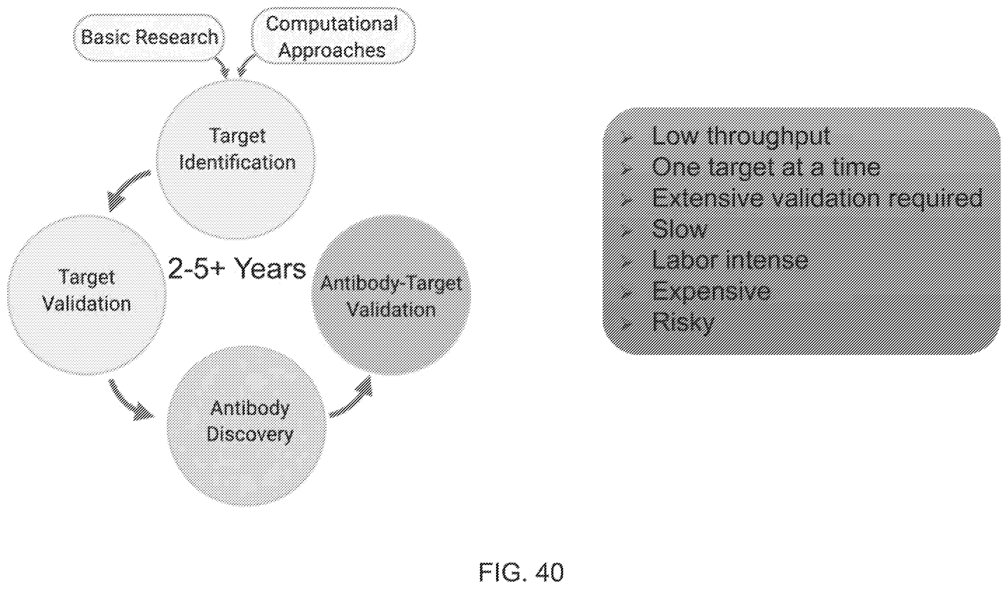

FIG. 41 shows a schematic of simultaneous discovery of therapeutic antibodies and their cancer specific targets based on desired binding specificity as described herein. In an embodiment, turnaround time can be about 2 to 3 months.

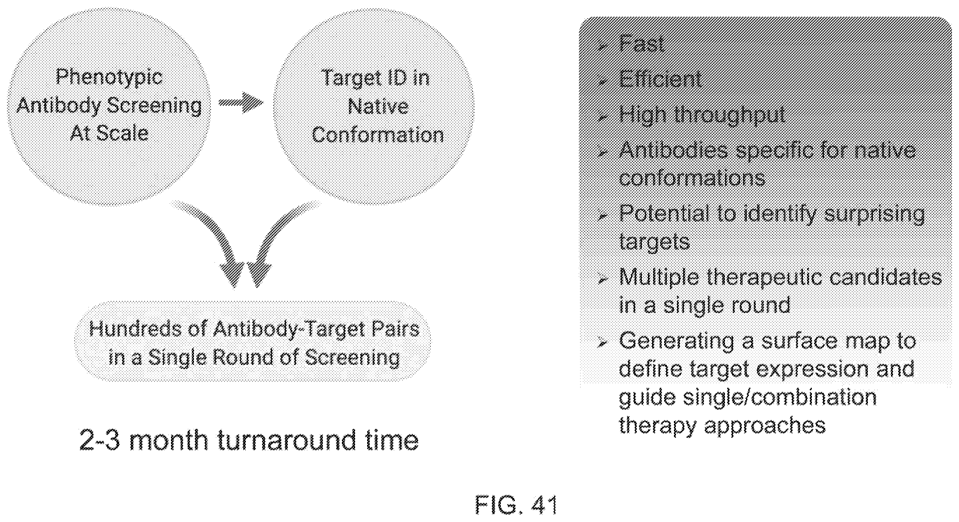

FIG. 42 shows a schematic of the PhASTdiscovery Platform workflow and timeline. In am embodiment, target-antibody discovery can take about 2 to 3 months.

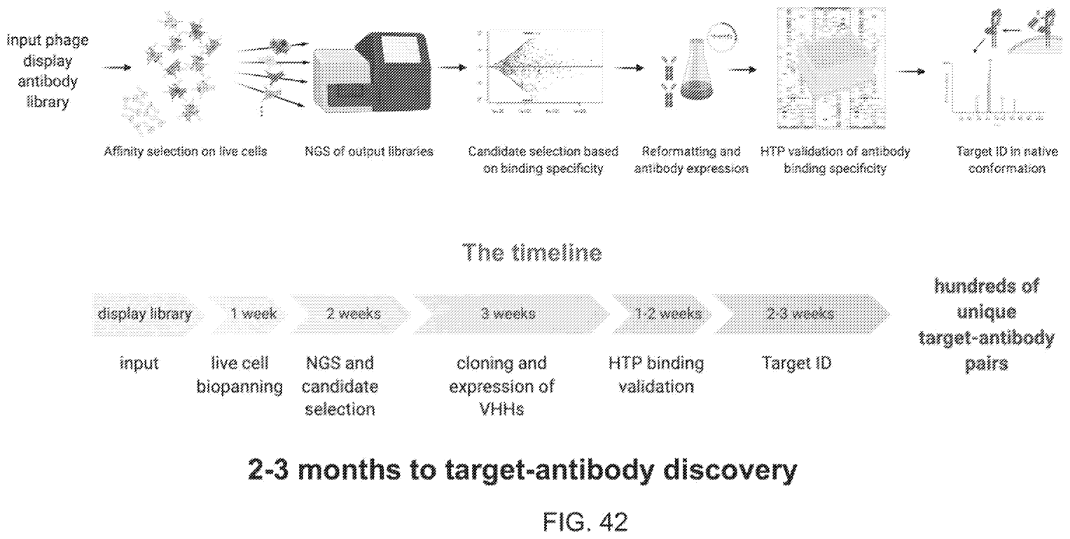

FIG. 43 shows the discovery of ovarian specific antibody target pairs in a single round of screening.

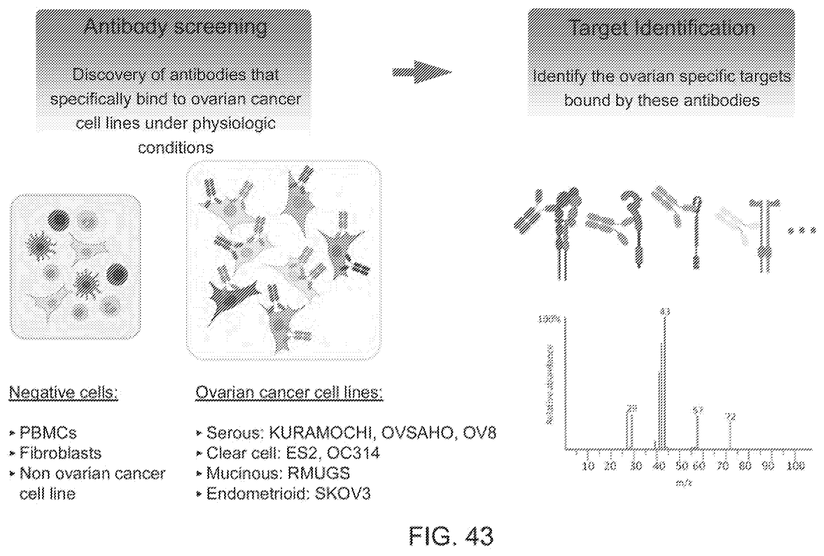

FIG. 44 shows identification of ovarian specific antibody-target pairs.

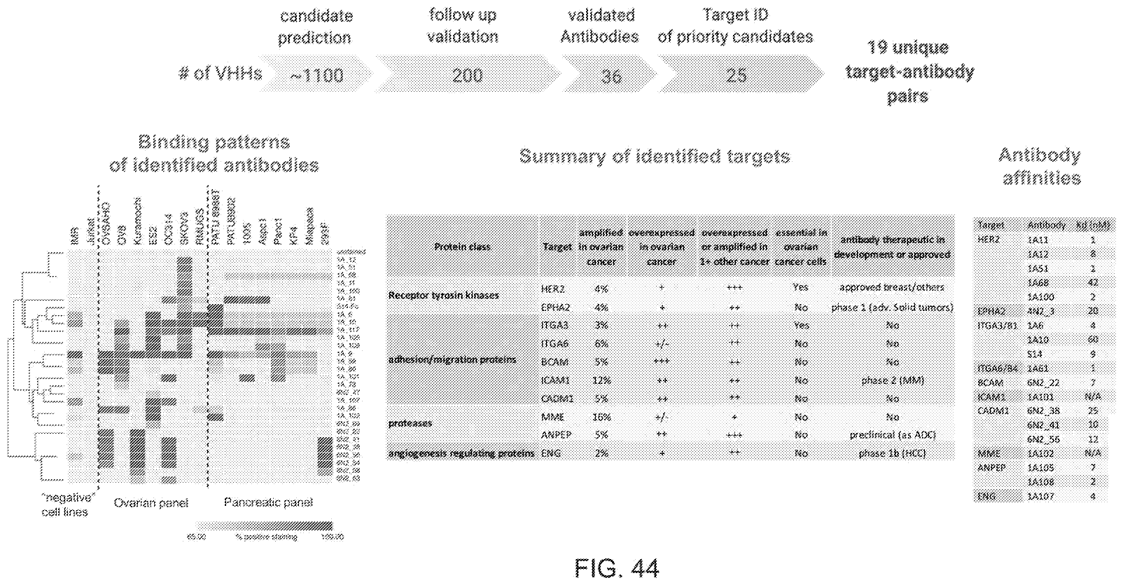

FIG. 45 shows identification and characterization of anti-Her2 chimeric antibodies. FIG. 45 discloses SEQ ID NOS 47, 50, 53, 41 and 44, respectively, in order of appearance.

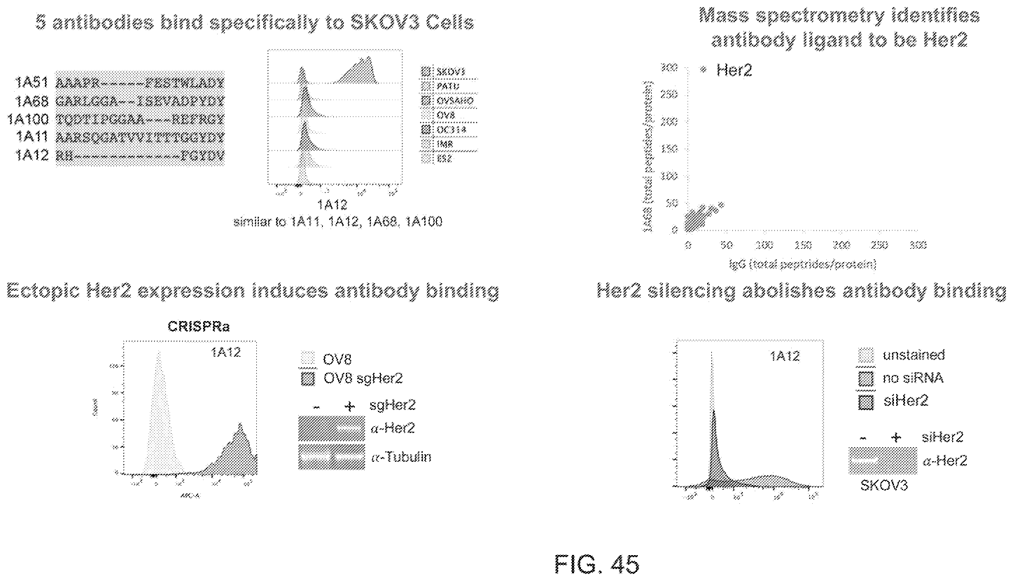

FIG. 46 shows schematics and results of anti-Her2 single domain antibody binding to distinct eptiopes from anti-Her2 IgGs. FIG. 46 discloses SEQ ID NOS 3-5 and 1-2, respectively, in order of appearance.

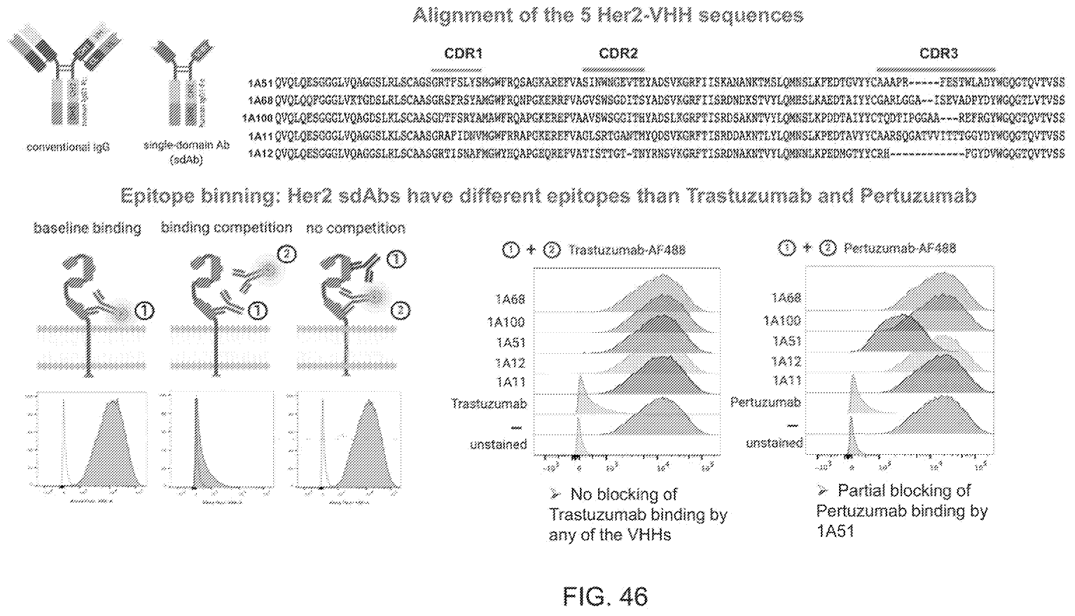

FIG. 47 shows BCAM expression data. For example, BCAM is highly expressed in HGSOC cell lines.

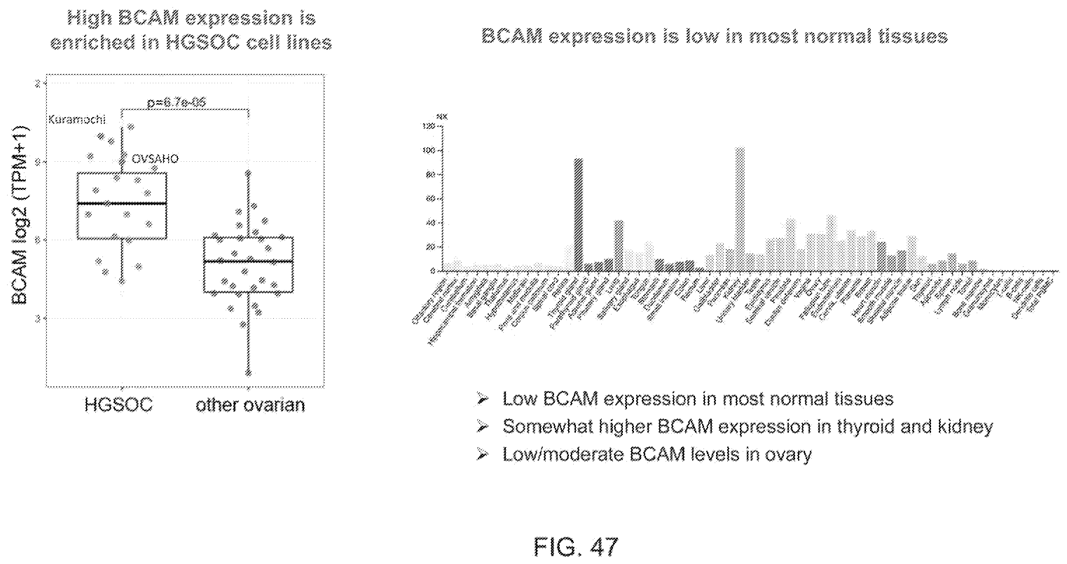

FIG. 48 shows BCAM is highly overexpressed in HGSOC tumors and can be associated with poor survival. See also, for example,, Määttä et al., J Histochem Cytochem, 53 (10), 2005; Garinchesa, P. et al., IntJOnc, 5 (6), 1994; and Bertolini et al., Clin Clinical Research, 22 (19), 2016).

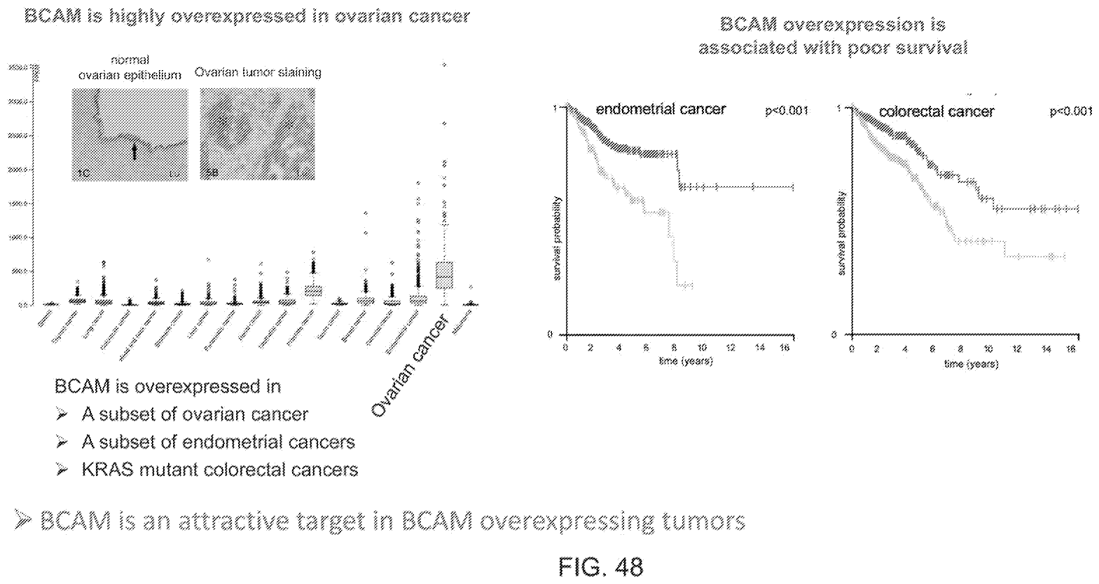

FIG. 49 shows binding data for anti-BCAM chimeric single domain antibody. For example, anti-BCAM chimeric single domain antibody has nM affinity for BCAM and induces ADCC in high BCAM expressing cells.

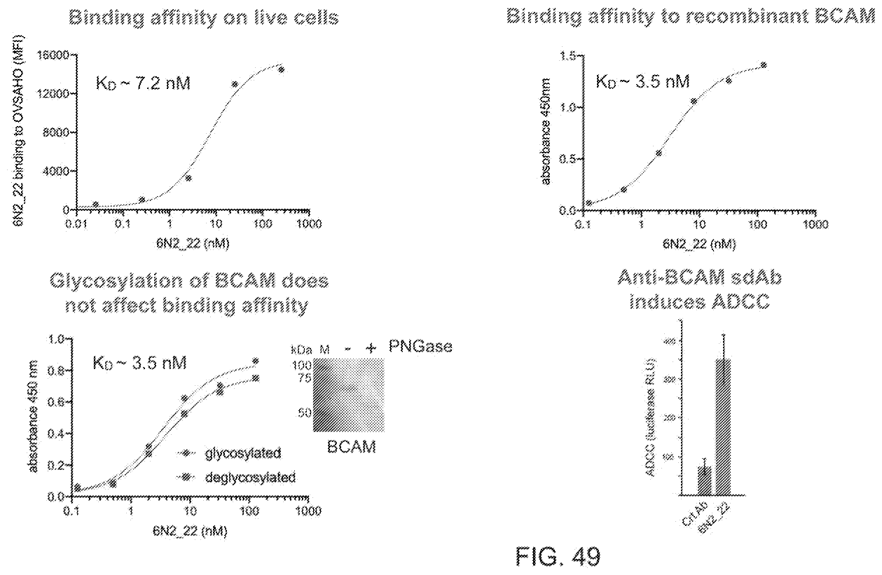

FIG. 50 shows a schematic displaying an embodiment of PhASTdiscovery phenotypic candidate selection strategy.

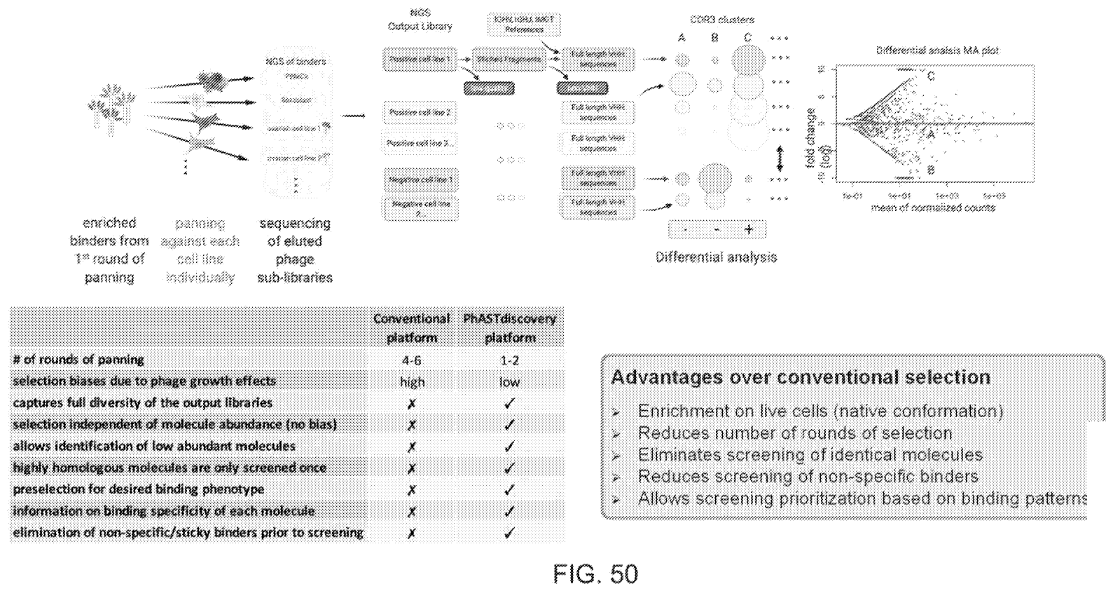

FIG. 51 shows Her2 expression in SKOV3 cells and a subset of ovarian cancers. Her2 is highly expressed in SKOV3 cells and a subset of ovarian cancers.

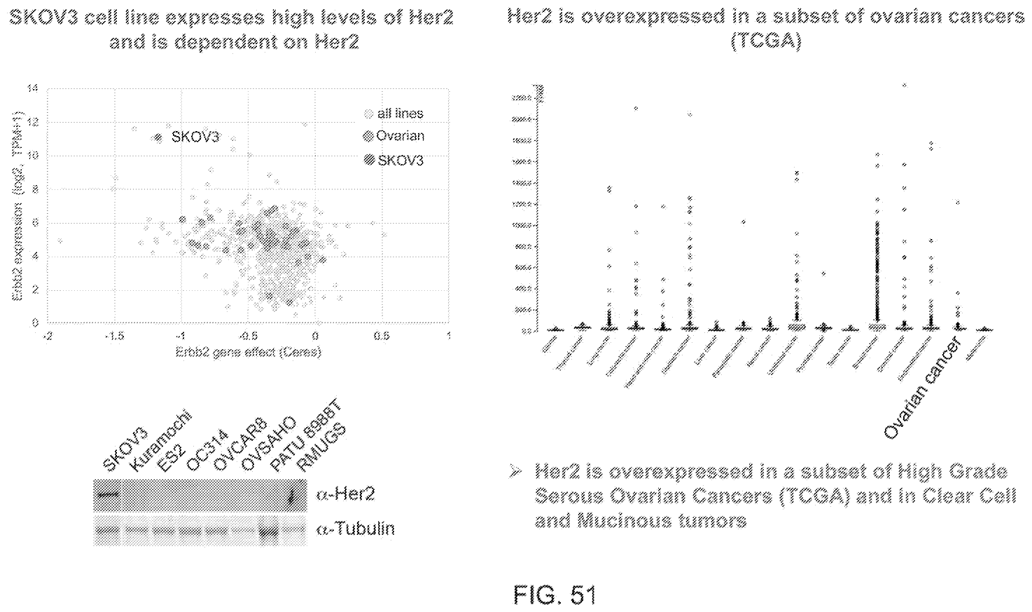

FIG. 52 shows (Panel A) FACS binding profile of 6N2_41 representative of cluster B antibodies. Staining was performed with 6N2_41 antibody followed by incubation with APC conjugated a-human Fc secondary antibody. HGSOC lines are underlined. (Panel B) Mass spectrometry identification of CADM1 representative for cluster B antibodies. Total number of peptides obtained with 6N2_41 vs hIgG control antibody are plotted. (Panel C) FACS binding of 6N2_38 to Kuramochi cells transfected with siRNA targeting CADM1 (blue), control transfected Kuramochi cells (green), and secondary only antibody (gray). (Panel D) FACS binding profile of clone 6N2_22. Flow cytometry staining was performed with 6N2_22-hIgG1-Fc followed by a-human-APC secondary antibody in a multiplexed format. High grad ovarian cell lines are underlined. (Panel E) Target ID of 6N2_22-hIgG1-F-ASB antibody. Mass spectrometry results for 6N2_22 are plotted against hIgG1 negative control. The total number of peptides/protein is shown. (Panel F) FACS staining with 6N2_22-hIgG1-Fc of Kuramochi cells with (blue) or without (green) BCAM transient transfection in 293T cells (left panel). BCAM overexpression was verified by western blotting (right panel). (Panel G) FACS staining with 6N2_22-hIgG1-Fc of OVSAHO cells with (blue) or without (green) BCAM siRNA transfection (left panel). BCAM knockdown efficiency was verified by western blotting (right panel). (Panel H) ELISA binding of 6N2_22-hIgG1-Fc dilutions to untreated (blue) or PNGase treated (red) recombinant human BCAM.

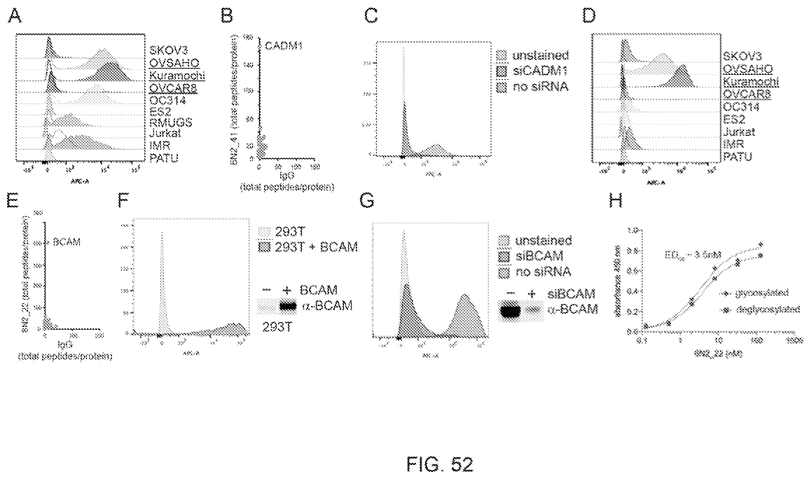

FIG. 53 shows (Panel A) Epitope mapping of 6N2_22 was performed in 293T cells transiently transfected with indicated BCAM/MCAM chimeras followed by flow cytometry. Binding is quantified as % APC positive cells compared to secondary only antibody staining. FIG. 53 (Panel A) discloses SEQ ID NO: 177. (Panel B) 6N2_22 antibody binding to indicated Myc-tagged BCAM point mutants was tested by flow cytometry of transfected 293T cells (top panel). Expression was verified by western blotting using a-Myc antibody. (Panel C) Antigen dependent cytotoxicity was evaluated by quantifying antibody induced apoptosis mediated by PBMCs in CellTrace Violet labelled parental or BCAM ko Kuramochi target cells by Annexin V-488 staining and subsequent flow cytometry analysis. Results represent % of Annexin V/Violet positive cells relative to a no antibody control.

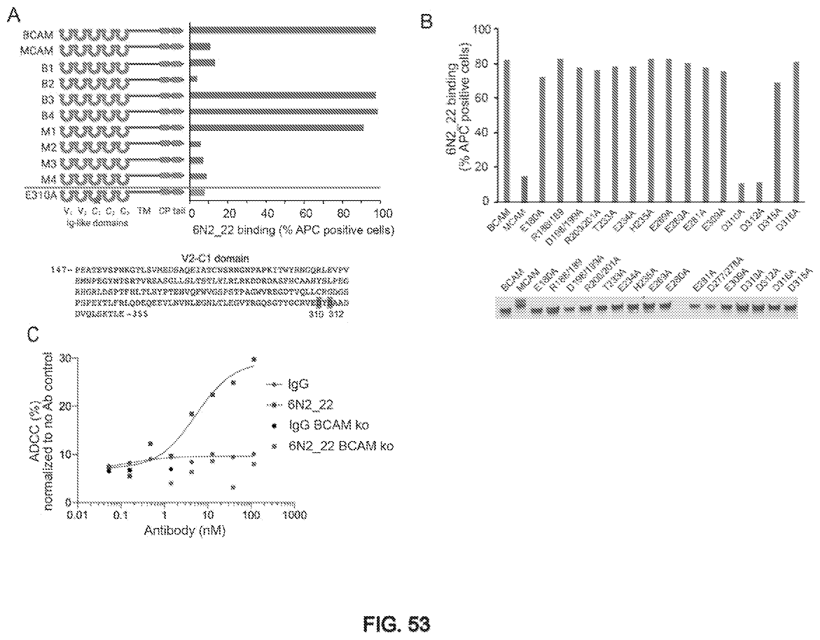

FIG. 54 shows (Panel A) Western blot analysis of expression of BCAM in indicated ovarian cell lines. Equal loading was verified with probing for a-Tubulin. (Panel B) Box plot comparing BCAM gene expression between HGSOC (blue) and other ovarian CCLE cell lines (gray). Kuramochi and OVSAHO (6N2_22 binders) are highlighted in red. The statistical significance was tested using a Kruskal-Wallis test p=6.7×10⁻⁵. (Panel C) Representative facs histogram of ovarian cancer derived organoids stained for BCAM (blue) or IgG control (gray) (left panel). BCAM expression is shown as % positive cells compared to IgG control (right panel) (Panel D) Representative images of IHC staining of ovarian tumor tissue microarrays. Microarrays were stained with a-BCAM and fluorescently labelled a-rabbit secondary antibody (green) and counterstained with DAPI (blue). Top 2 panels represent cores from HGSOC, bottom left Mucinous adenocarcinoma, bottom right Endometrioid adenocarcinoma. (Panel E) Quantification of BCAM expression from tissue microarray of 36 HGSOV and 33 other ovarian subtypes. Statistical significance was tested using unpaired t-test p<0.0001.

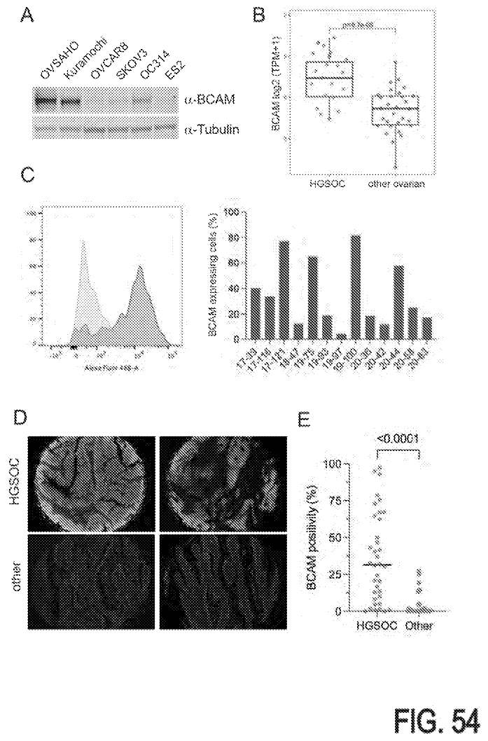

FIG. 55 shows (Panel A) Diagram showing Erbb2 gene expression plotted against Erbb2 dependency across all CCLE cell lines. Ovarian cell lines are highlighted in blue. (Panel B) Erbb2 gene expression in tumors based on TCGA data. (Panel C) Schematic illustrating the epitope binning assay. (Panel D) Epitope binning of Trastuzumab and Pertuzumab with indicated Her2-VHH antibodies.

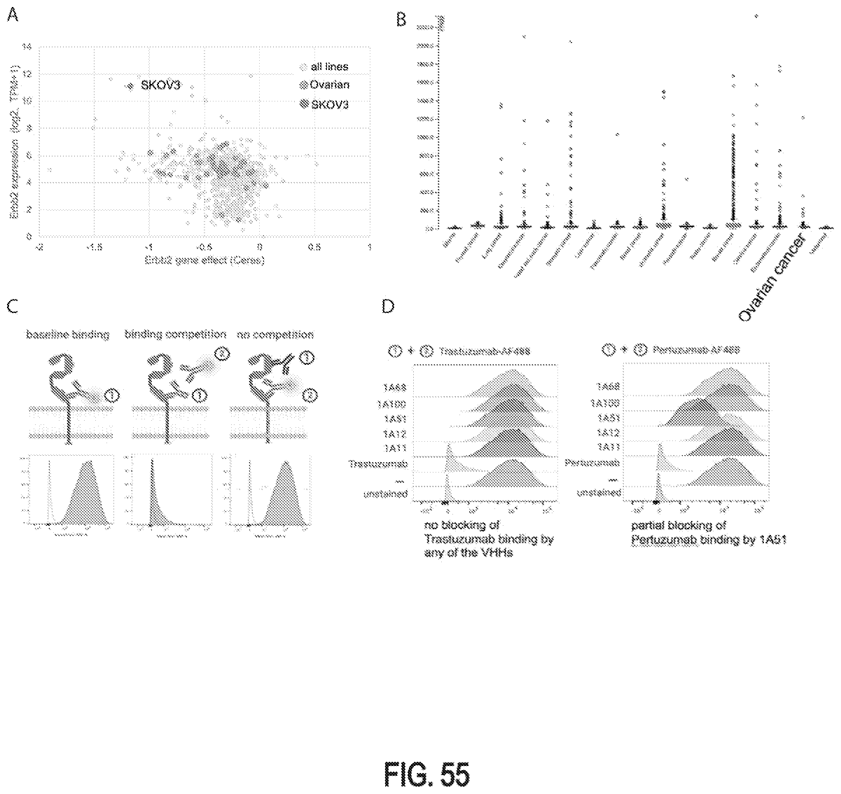

FIG. 56 shows (Panel A) anti-BCAM 6N2_22 binding curve on Kuramochi live cells with a EC50 of 7.2 nM (Panel B) Coomassie stain of recombinant BCAM with or without PNGase treatment for deglycosylation.

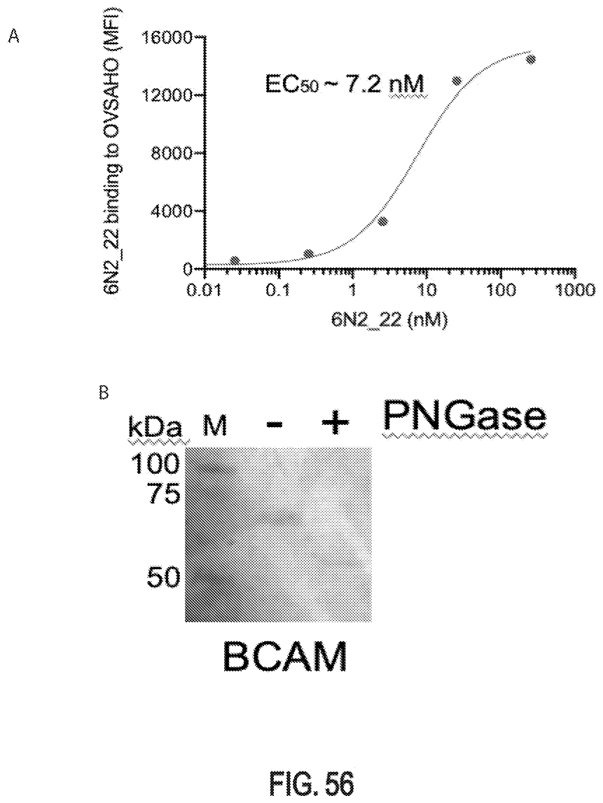

FIG. 57 shows (Panel A) 6N2_22 triggered BCAM internalization was tested on Kuramochi (left panel) and OVSAHO (right panel) by comparing FACS binding upon incubation of antibody for 3h on ice versus 37° C. and subsequent staining with a-human-APC secondary antibody. (Panel B) Adhesion of Kuramochi cells with or without 6N2_22 treatment was tested by cell titer glow after a 4h incubation. Mean luminescence signal of quadruplicates is shown.

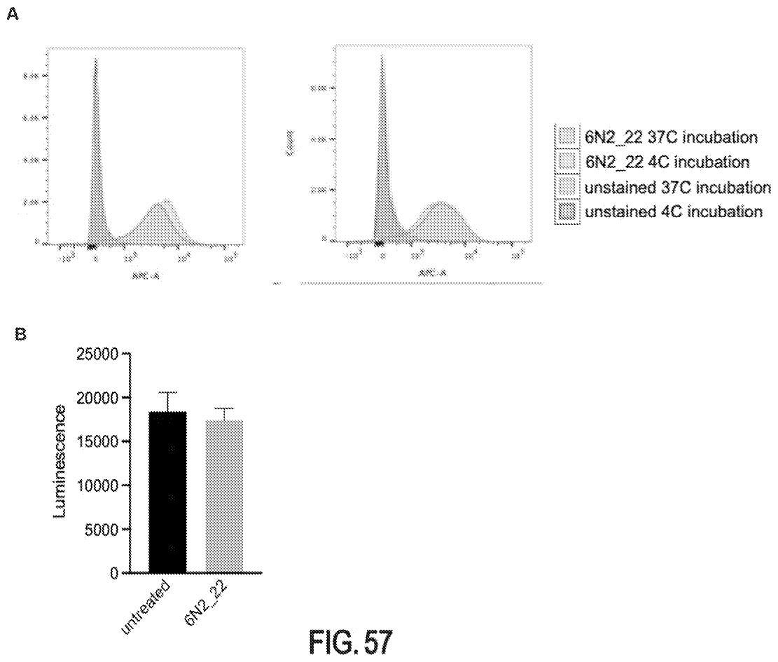

FIG. 58 shows Panel A) BCAM expression of TCGA Pan-Cancer atlas (Panel B) BCAM expression across healthy tissues (consensus data set from Protein Atlas) (Panel C) representative images of Kidney and Thyroid stained for BCAM. Images were counterstained with DAPI and where indicated color enhanced to visualize weak BCAM staining. (Panel D) Spearman correlation between BCAM and LAMA5 expression of HGSOC tissue microarray cores of epithelial BCAM positivity with epithelial LAMA5 (left) and stromal LAMA5 (right).

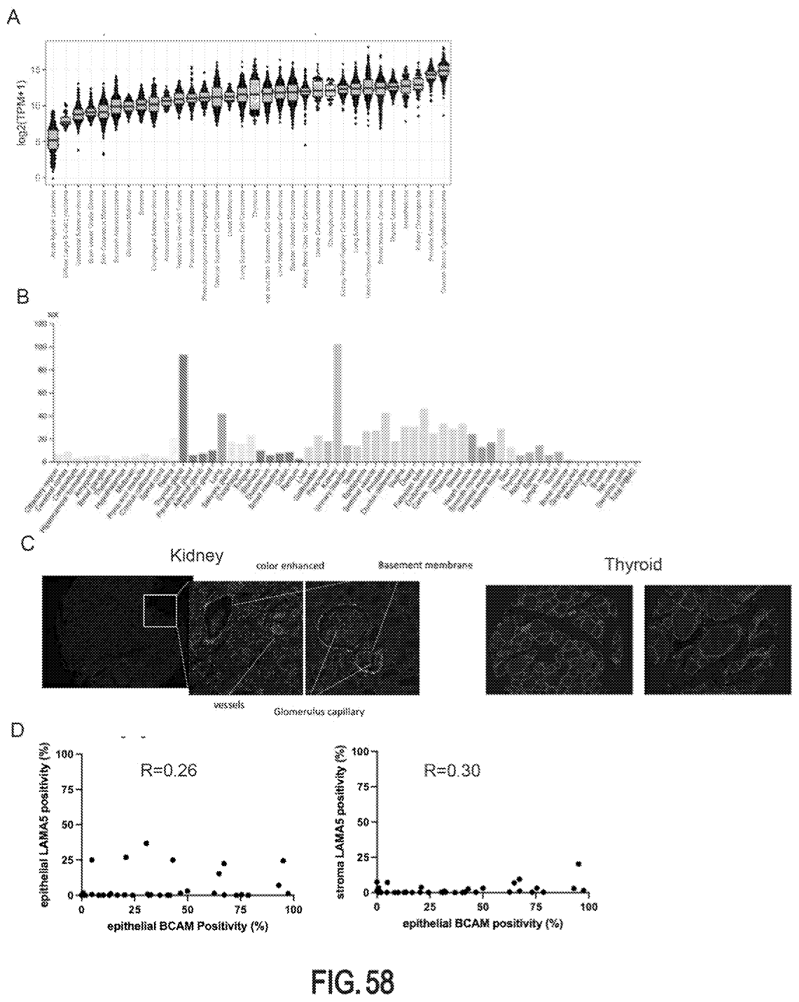

FIG. 59 shows graphs and schematics which indicate that 6N2_22 binds within BCAMs domains and BCAM D310/312 are essential for binding. For example, FIG. 54 shows expemplary internalization properties and induction of ADCC were tested. Data for 6N2_22 critical binding domains/residues on BCAM was collected. FIG. 59 discloses SEQ ID NO: 177.

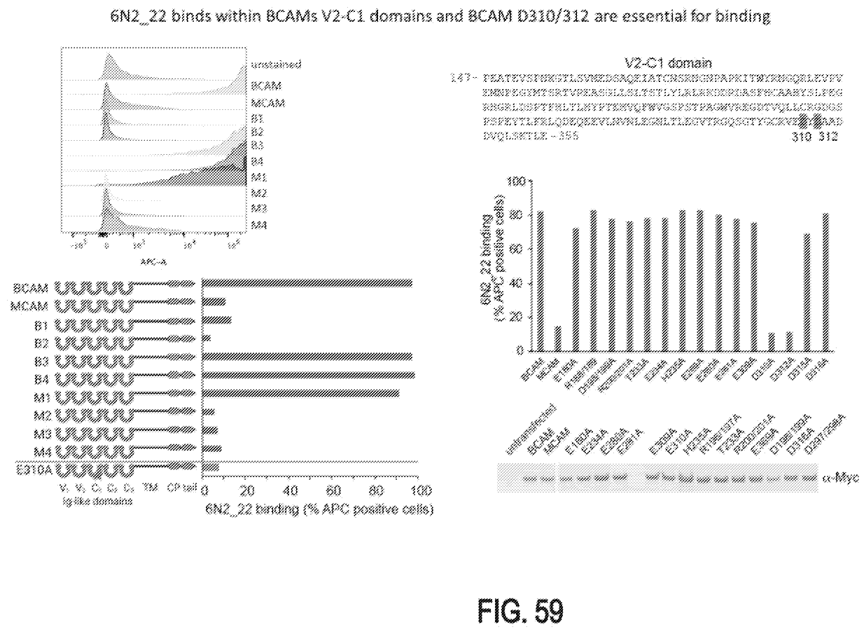

FIG. 60 shows graphs and schematics which indicate W4 and R7 within the CDR3 are essential for 6N2_22 BCAM binding. The 6N2_22 CDR3 was also mutated to identify key residues. FIG. 60 discloses SEQ ID NOS 154 and 178-179, respectively, in order of appearance.

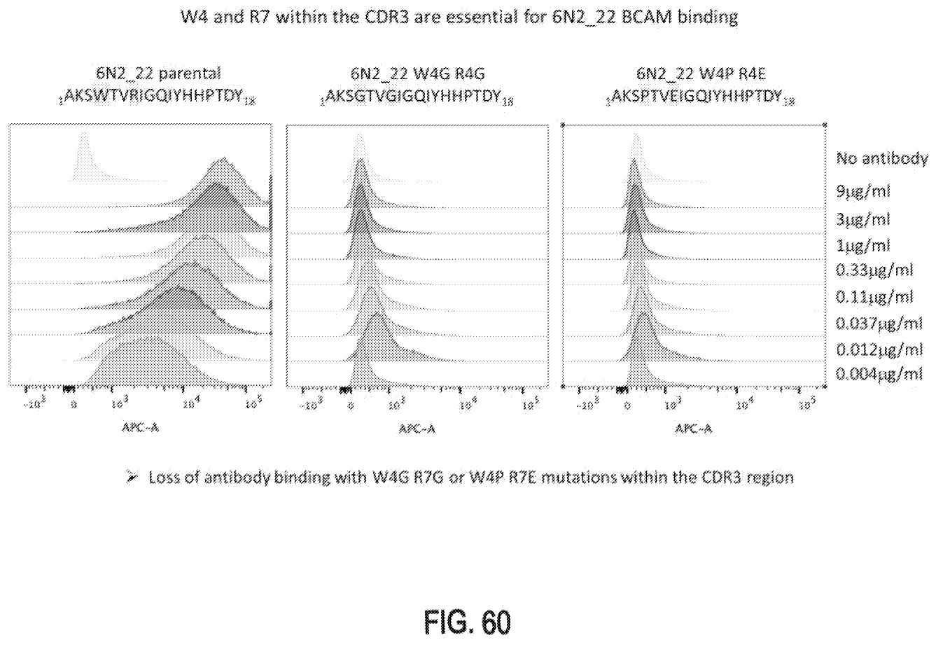

FIG. 61 shows BCAM is a therapeutic target in HGSOC. (Panel A) Western blot analysis of expression of BCAM in indicated ovarian cell lines. Equal loading was verified with probing for anti-Tubulin. (Panel B) Box plot comparing BCAM gene expression between HGSOC (blue) and other ovarian CCLE cell lines (gray). Kuramochi and OVSAHO (6N2_22 binders) are highlighted in red. The statistical significance was tested using a Kruskal-Wallis test p=6.7×10-5. (Panel C) Representative images of IHC staining of ovarian tumor tissue microarrays. Microarrays were stained with a-BCAM and fluorescently labelled a-rabbit secondary antibody (green) and mouse a-LAMA5 antibodies (purple) and counterstained with DAPI (blue). (Panel D) Epitope mapping of 6N2_22 was performed in 293T cells transient transfected with indicated BCAM/MCAM chimeras followed by flow cytometry. Binding is quantified as % cells APC positive compared to secondary only antibody staining. (Panel E) Antigen dependent cytotoxicity was evaluated by quantifying antibody induced apoptosis mediated by PBMCs in CellTrace Violet labelled parental or BCAM ko Kuramochi target cells by Annexin V-488 staining and subsequent flow cytometry analysis. Results represent % of Annexin V/Violet positive cells relative to a no antibody control.

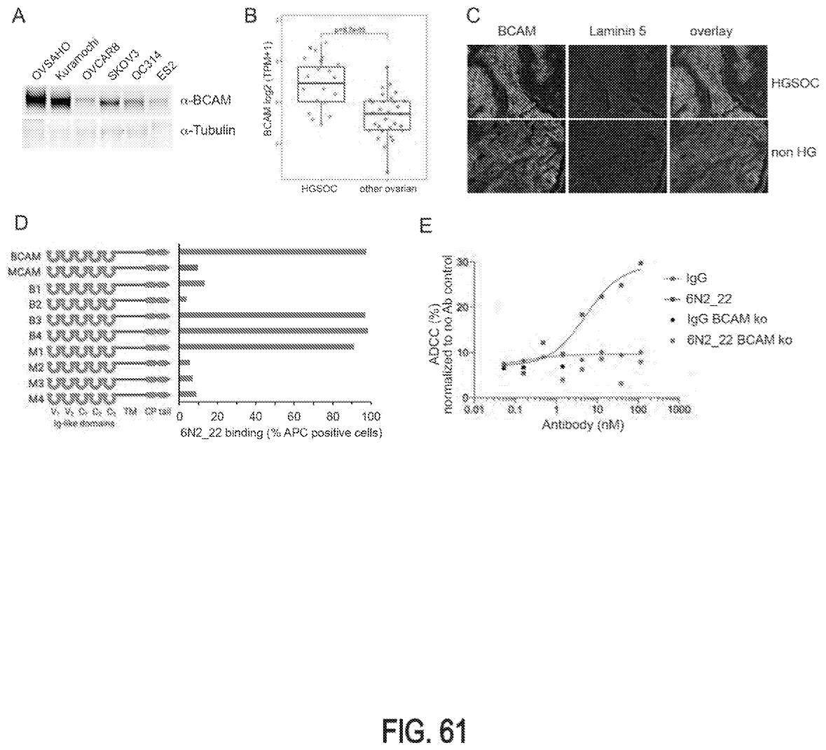

### DETAILED DESCRIPTION OF THE INVENTION

Aspects of the invention are drawn to a compositions and methods to identify one or more antibodies and the antibodies identified thereby. For example, embodiments comprise compositions and methods that identify one or more antibodies based on cell surface binding patterns on cells. Embodiments of the invention can further comprise compositions and methods for identification of antibody targets. Thus, compositions and methods described herein can provide the simultaneous discovery target-antibody pairs specific to the native or cancer specific state in a single round of screening. As a representative example, compositions and methods described herein were applied to ovarian cancer, thereby identifying Her2 specific VHH antibodies (e.g., nanobodies). Thus, representative examples described herein demonstrate that the platform can identify highly potent antibodies against cancer targets, such as targets in high grade ovarian cancer.

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower).

### Antibodies

Aspects of the invention are drawn to isolated monoclonal antibodies, antibody fusions, or fragments thereof.

The term “isolated” as used herein with respect to cells, nucleic acids, such as DNA or RNA, or polypeptides can refer to molecules separated from other cells, DNAs or RNAs, or polypeptides, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and would not be found in the natural state. “Isolated” can also refer to cells or polypeptides which are isolated from other cellular proteins or tissues. Isolated polypeptides can include both purified and recombinant polypeptides.

Unique recombinant monoclonal antibodies are described herein. These include, for example, 6N2_22, 6N2_38, 6N2_41, 6N2_54, 6N2_56, 1A11, 1A12, 1A51, 1A68, 1A100, 4N2_3, 1A6, 1A10, S14, 1A61, 1A101, 1A102, 1A105, 1A108, or 1A107, and antibodies that compete with the binding of 6N2_22, 6N2_38, 6N2_41, 6N2_54, 6N2_56, 1A11, 1A12, 1A51, 1A68, 1A100, 4N2_3, 1A6, 1A10, S14, 1A61, 1A101, 1A102, 1A105, 1A108, or 1A107. “Recombinant” as it pertains to polypeptides (such as antibodies) or polynucleotides can refer to a form of the polypeptide or polynucleotide that does not exist naturally, a non-limiting example of which can be created by combining polynucleotides or polypeptides that would not normally occur together.

The nucleic acid and amino acid sequence of the monoclonal antibodies are provided below. The amino acid sequences of the heavy chain complementary determining regions (CDRs) are underlined (CDR1), underlined and bolded (CDR2), or underlined, italicized, and bolded (CDR3) below:

TABLE A1
Ab a-BCAM VHH (6N2_22) Variable Region nucleic
acid sequences V_{H} chain (SEQ ID NO: 93)

CAGGTGCAGCTGGTGGAGTCCGGAGGAGGACTGGTGCAGCCAGGAGGCAG
CCTGAGGCTGTCCTGCGCCGCCTCTGGCTTCACCTTTAGCTCCTACGCCA
TGAGCTGGGTGCGCCAGGCACCAGGCAAGGGACCTGAGTGGGTGAGCGCC
ATCAACTCCGGAGGAGGCTCCACATCTTACGCCGACTCTGTGAAGGGCCG
GTTCACCATCAGCAGAGATAACGCCAAGAATACACTGTATCTGCAGATGA
ACAGCCTGAAGCCAGAGGACACCGCCGTGTACTATTGTGCCAAGTCTTGG
ACAGTGCGGATCGGCCAGATCTACCACCACCCCACCGATTATTGGGGCCA
GGGCACCCAGGTGACAGTGTCTAGC

TABLE A2
Ab a-BCAM VHH Variable Region amino acid sequences
V_{H} chain (SEQ ID NO: 94)

QVQLVESGGGLVQPGGSLRLSCAASGFTFSSYAMSWVRQAPGKGPEWVSA
INSGGGSTSYADSVKGRFTISRDNAKNTLYLQMNSLKPEDTAVYYCAKSW
TVRIGQIYHHPTDYWGQGTQVTVSS

TABLE B1
Ab a-CADM1 VHH (6N2_38) Variable Region nucleic
acid sequences V_{H} chain (SEQ ID NO: 25)

CAGGTGCAGCTGGTGGAGTCCGGAGGAGGACTGGTGCAGCCAGGAGGCAG
CCTGAGGCTGTCCTGCGCCGCCTCTGGCTTCACCTTTAGCTCCTACTGGA
TGTATTGGGTGCGGCAGGCACCTGGCAAGGGACTGGAGTGGGTGTCTGCC
ATCAACACCGGCGGCGGCAGCACATACTATGCCGACTCCGTGAAGGGCCG
GTTCACCATCAGCAGAGATAACGCCAAGAATACACTGTACCTGCAGATGA
ACTCCCTGAAGTCTGAGGACACAGCCGTGTACTATTGTGCCTACGGCAAT
GGCGTGGAGGGCATGGATTATTGGGGCAAGGGCACCCAGGTGACAGTGTC
TAGC

TABLE B2
Ab a-CADM1 VHH (6N2_38) Variable Region amino acid sequences
V_{H} chain (SEQ ID NO: 6)

QVQLVESGGGLVQPGGSLRLSCAASGFTFSSYWMYWVRQAPGKGLEWVSAINTGGG
STYYADSVKGRFTISRDNAKNTLYLQMNSLKSEDTAVYYCAYGNGVEGMDYWGKGT
QVTVSS

TABLE C1
Ab a-CADM1 VHH (6N2_41) Variable Region nucleic acid sequences
V_{H} chain (SEQ ID NO: 26)

CAGGTGCAGCTGGTGGAGTCTGGAGGAGGACTGGTGCAGCCAGGAGGCTCCCTGAGGCTGTCTT
GCGCCGCCAGCGGCTTCACCTTTAGCTCCTACTGGATGTATTGGGTGCGCCAGGCACCTGGCAA
GGGACTGGAGTGGGTGAGCGCCATCAACACCGGCGGAGGCTCCACATACTATGCCGACTCTGTG
AAGGGCCGGTTCACCATCAGCAGAGATAACGCCAAGAATACACTGTACCTGCAGATGAACTCCC
TGAAGCCCGAGGACACAGCCCTGTACTATTGTGCAACCCGGGCCTCCGTGGGCACACTGGAGAT
GTATGATAATTGGGGCCAGGGCACCCAGGTGACAGTGTCTAGC

TABLE C2
Ab a-CADM1 VHH (6N2_41) Variable Region amino acid sequences
V_{H} chain (SEQ ID NO: 7)

QVQLVESGGGLVQPGGSLRLSCAASGFTFSSYWMYWVRQAPGKGLEWVSAINTGGG
STYYADSVKGRFTISRDNAKNTLYLQMNSLKPEDTALYYCATRASVGTLEMYDNWGQ
GTQVTVSS

TABLE D1
Ab a-CAMD1 VHH (6N2_54) Variable Region nucleic acid sequences
V_{H} chain (SEQ ID NO: 27)

CAGGTGCAGCTGGTGGAGTCCGGAGGAGGACTGATGCAGCCAGGAGGCTCCCTGAGGCTGTCTT
GCGCAGCAAGCGGCTTCACCTTTAGGTCCTACGACATGTCTTGGGTGCGCCAGGTGCCAGGCAA
GGGACCAGAGTGGATCAGCTCCATCAACAGCAGGGGCGGCTCCACCTACTATACAGACCCTGTG
AAGGGCCGGTTCACCATCTCCAGAGATAACGCCAAGAATACACTGTACCTGGAGATGAACAGCC
TGAAGCCAGAGGACAGCGCCATCTACTATTGTGCCAAGGGCCGGTATGGCGCCTCTTGGATGTT
TCCCCCTTACGATTATTGGGGCCAGGGCACCCAGGTGACAGTGTCTAGC

TABLE D2
Ab a-CAMD1 VHH (6N2_54) Variable Region amino acid sequences
V_{H} chain (SEQ ID NO: 8)

QVQLVESGGGLMQPGGSLRLSCAASGFTFRSYDMSWVRQVPGKGPEWISSINSRGGS
TYYTDPVKGRFTISRDNAKNTLYLEMNSLKPEDSAIYYCAKGRYGASWMFPPYDYWG
QGTQVTVSS

TABLE E1
Ab a-CADM1 VHH (6N2_56) Variable Region nucleic acid sequences
V_{H} chain (SEQ ID NO: 28)

CAGGTGCAGCTGGTGGAGAGCGGAGGAGGACTGGTGCAGCCAGGAGGCTCCCTGAGGCTGTCTT
GCGCCGCCAGCGGCTTCACCTTTTCTGACAGCTGGATCTACTGGGTGCGGCAGGCACCTGGCAA
GGGACTGGAGTGGGTGTCCGCCATCAACACCGACGGCTCTAATACATACTATGCCGATAGCGTG
AAGGGCCGGTTCACCATCTCCAGAGATAACGCCAAGAATACACTGTATCTGCACATGGACAACC
TGAAGTCCGAGGATACCGCCGTGTACTATTGTGCAAAGGGAGGACTGACAGGCTCTTGGGGACA
GGGCACCCAGGTGACAGTGAGCTCC

TABLE E2
Ab a-CADM1 VHH (6N2_56) Variable Region amino acid sequences
V_{H} chain (SEQ ID NO: 9)

QVQLVESGGGLVQPGGSLRLSCAASGFTFSDSWIYWVRQAPGKGLEWVSAINTDGSN
TYYADSVKGRFTISRDNAKNTLYLHMDNLKSEDTAVYYCAKGGLTGSWGQGTQVTV
SS

TABLE F1
Ab a-Her2 (1A11) Variable Region nucleic acid sequences
V_{H} chain (SEQ ID NO: 20)

CAGGTGCAGCTGCAGGAGAGCGGAGGAGGACTGGTGCAGGCAGGAGGCTCTCTGAAGCTGAGCT
GCGCAGCATCCGGAAGGGCCTTCATCGACAACGTGATGGGCTGGTTTAGGAGAGCACCAGGCAA
GGAGAGGGAGTTCGTGGCAGGACTGTCCAGAACAGGCGCCAATACCATGTACCAGGATTCTGTG
AAGGGCAGGTTTACAATCAGCCGCGACGATGCCAAGAACACCCTGTATCTGCAGATGAACAGCC
TGAAGCCTGAGGACACAGCCGTGTACTATTGTGCAGCAAGATCTCAGGGAGCAACCGTGGTCAT
CACCACAACCGGCGGCTACGATTATTGGGGCCAGGGCACACAGGTGACCGTGAGCTCC

TABLE F2
Ab a-Her2 (1A11) Variable Region amino acid sequences
V_{H} chain (SEQ ID NO: 1)

QVQLQESGGGLVQAGGSLKLSCAASGRAFIDNVMGWFRRAPGKEREFVAGLSRTGA
NTMYQDSVKGRFTISRDDAKNTLYLQMNSLKPEDTAVYYCAARSQGATVVITTTGGY
DYWGQGTQVTVSS

TABLE G1
Ab a-Her2 (1A12) Variable Region nucleic acid sequences
V_{H} chain (SEQ ID NO: 21)

CAGGTGCAGCTGCAGGAGTCCGGAGGAGGACTGGTGCAGGCAGGAGGCTCTCTGAGGCTGAGCT
GCGCAGCATCCGGAAGGACCATCTCCAACGCCTTCATGGGCTGGTACCACCAGGCACCAGGAGA
GCAGAGGGAGTTTGTGGCCACAATCTCTACCACAGGCACCACAAACTACCGGAACAGCGTGAAG
GGCCGGTTCACCATCAGCAGAGACAACGCCAAGAATACAGTGTATCTGCAGATGAACAATCTGA
AGCCTGAGGACATGGGCACCTACTATTGTCGGCACTTTGGCTATGACGTGTGGGGCCAGGGCAC
CCAGGTGACAGTGAGCTCC

TABLE G2
Ab a-Her2 (1A12) Variable Region amino acid sequences
V_{H} chain (SEQ ID NO: 2)

QVQLQESGGGLVQAGGSLRLSCAASGRTISNAFMGWYHQAPGEQREFVATISTTGTT
NYRNSVKGRFTISRDNAKNTVYLQMNNLKPEDMGTYYCRHFGYDVWGQGTQVTVSS

TABLE H1
Ab a-Her2 (1A51) Variable Region nucleic acid sequences
V_{H} chain (SEQ ID NO: 22)

CAGGTGCAGCTGCAGGAGTCCGGAGGAGGACTGGTGCAGGCAGGAGGCTCCCTGAGGCTGTCTT
GCGCAGGCAGCGGACGCACCTTCTCTCTGTACAGCATGGGCTGGTTTCGGCAGTCTGCCGGCAA
GGCCAGAGAGTTCGTGGCCAGCATCAACTGGAATGGCGAGGTGACAGAGTATGCCGACAGCGTG
AAGGGCCGGTTTATCATCTCCAAGGCCAACGCCAATAAGACCATGTCCCTGCAGATGAACTCTC
TGAAGTTCGAGGACACAGGCGTGTACTATTGTGCCGCCGCCCCCAGGTTTGAGTCTACCTGGCT
GGCAGATTACTGGGGACAGGGAACCCAGGTGACAGTGAGCTCC

TABLE H2
Ab a-Her2 (1A51) Variable Region amino acid sequences
V_{H} chain (SEQ ID NO: 3)

QVQLQESGGGLVQAGGSLRLSCAGSGRTFSLYSMGWFRQSAGKAREFVASINWNGE
VTEYADSVKGRFIISKANANKTMSLQMNSLKFEDTGVYYCAAAPRFESTWLADYWGQ
GTQVTVSS

TABLE I1
Ab a-Her2 (1A68) Variable Region nucleic acid sequences
V_{H} chain (SEQ ID NO: 23)

CAGGTGCAGCTGCAGCAGTTCGGAGGAGGACTGGTGAAGACCGGCGACTCCCTGAGGCTGTCTT
GCGCAGCATCCGGCAGGTCTTTCCGCAGCTACGCCATGGGCTGGTTTCGCCAGAACCCAGGCAA
GGAGCGGAGATTCGTGGCAGGCGTGTCTTGGAGCGGCGACATCACAAGCTACGCCGATTCCGTG
AAGGGCCGGTTTATCATCTCTAGAGACAATGATAAGAGCACCGTGTATCTGCAGATGCACTCCC
TGAAGGCCGAGGATACAGCCATCTACTATTGTGGAGCAAGGCTGGGAGGAGCAATCTCTGAGG
TGGCCGACCCTTACGATTATTGGGGCCAGGGCACCCTGGTGACAGTGAGCTCC

TABLE I2
Ab a-Her2 (1A68) Variable Region amino acid sequences
V_{H} chain (SEQ ID NO: 4)

QVQLQQFGGGLVKTGDSLRLSCAASGRSFRSYAMGWFRQNPGKERRFVAGVSWSGD
ITSYADSVKGRFIISRDNDKSTVYLQMHSLKAEDTAIYYCGARLGGAISEVADPYDYWG
QGTLVTVSS

TABLE J1
Ab a-Her2 (1A100) Variable Region nucleic acid sequences
V_{H} chain (SEQ ID NO: 24)

CAGGTGCAGCTGCAGGAGTCTGGAGGAGGACTGGTGCAGGCAGGCGACAGCCTGAAGCTGTCCT
GCGCAGCATCTGGCGATACCTTCAGCCGGTACGCAATGGCATGGTTTAGGCAGGCACCAGGCAA
GGAGAGGGAGTTCGTGGCAGCCGTGTCTTGGAGCGGAGGAATCACACACTACGCCGACAGCCTG
AAGGGCAGGTTTACCATCTCCCGCGATTCTGCCAAGAACACAGTGTATCTGCAGATGAACAGCC
TGAAGCCCGACGATACCGCCATCTACTATTGTACCCAGGACACAATCCCTGGAGGAGCAGCCCG
GGAGTTCAGAGGCTATTGGGGCCAGGGCACCCAGGTGACAGTGAGCTCC

TABLE J2
Ab a-Her2 (1A100) Variable Region amino acid sequences
V_{H} chain (SEQ ID NO: 5)

QVQLQESGGGLVQAGDSLKLSCAASGDTFSRYAMAWFRQAPGKEREFVAAVSWSGG
ITHYADSLKGRFTISRDSAKNTVYLQMNSLKPDDTAIYYCTQDTIPGGAAREFRGYWG
QGTQVTVSS

TABLE K1
Ab a-EPHA2 (4N2_3) Variable Region nucleic acid sequences
V_{H} chain (SEQ ID NO: 29)

CAGGTGCAGCTGGTGGAGTCCGGAGGAGGACTGGTGCAGCCAGGAGGCTCCCTGAGGGTGTCTT
GCGCCGCCAGCGGCTTCACCTTTAGCTCCTACGCAATGACATGGGTGCGCCAGGCACCTGGCAA
GGGACTGGAGTGGGTGTCTGCCATCAACGGCGGCGGCAGCACCTACTATGCCGACTCCGTGAAG
GGCCGGTTCACCATCTCTAGAGATAACGCCAAGAATACACTGTACCTGCAGATGAATAGCCTGA
AGCCCGAGGACACAGCCCTGTACTATTGTGCCCGGGTGTATGGCTCCTACTATGCCTCTTTTTA
CGCCATGGATTATTGGGGCAAGGGCACCCAGGTGACAGTGTCTAGC

TABLE K2
Ab a-EPHA2 (4N2_3) Variable Region amino acid sequences
V_{H} chain (SEQ ID NO: 10)

QVQLVESGGGLVQPGGSLRVSCAASGFTFSSYAMTWVRQAPGKGLEWVSAINGGGS
TYYADSVKGRFTISRDNAKNTLYLQMNSLKPEDTALYYCARVYGSYYASFYAMDYWG
KGTQVTVSS

TABLE L1
Ab a-ITGA3/B1 (1A6) Variable Region nucleic acid sequences
V_{H} chain (SEQ ID NO: 30)

CAGGTGCAGCTGCAGCAGTTCGGAGGAGGACTGGTGCAGGCAGGAGGCTCTCTGAGACTGAGCT
GCGCAGCATCCGGACTGGCCTTCGACTCCCACCAGATGGGATGGTTTAGGCAGGGACCAGGCAA
GGAGAGAGAGTTCGTGGCCTCTATCAGGAGCGCCGGCTCCACCTACTATACAGACTCTGTGAAG
GGCCGGTTTACCATCAGCAGAGATAACGCCAAGAATACAGTGTCTCTGCAGATGAACATGCTGA
AGCTGGAGGACACCGCCGTGTACTATTGCGTGGCCGATAGGACATACTTTGGCCGCGAGGCCGA

TABLE L2
Ab a-ITGA3/B1 (1A6) Variable Region amino acid sequences
V_{H} chain (SEQ ID NO: 11)

QVQLQQFGGGLVQAGGSLRLSCAASGLAFDSHQMGWFRQGPGKEREFVASIRSAGST
YYTDSVKGRFTISRDNAKNTVSLQMNMLKLEDTAVYYCVADRTYFGREADYDYCGQ
GTQVTVSS

TABLE M1
Ab a-ITGA3/B1 (1A10) Variable Region nucleic acid sequences
V_{H} chain (SEQ ID NO: 31)

CAGGTGCAGCTGCAGCAGTCTGGAGGAGGACTGGTGCAGGCAGGAGGCTCTCTGCGGCTGAGCT
GCGCAGCATCCGGAAGGACCTTCAGCAACTACGCCATCGGCTGGTTTAGGCAGGCACAGGGCAA
GGAGAGGGAGTTCGTGGCAGCAATCTCCTGGGGAGGAGGAAATACATACTATGCCGGCTCCGTG
AAGGGCCGGTTCACCATCTCTAGAGACAACGCCAAGAATACAGTGTACCTGCAGATGAACAGCC
TGAAGTTTGAGGATACCGCCGTGTACTATTGTGCCGCCTCTGAGGTGGCCCACAGCGACTATGA
GGAGGAGTACGATTATTGGGGCCAGGGCACCCAGGTGACAGTGAGCTCC

TABLE M2
Ab a-ITGA3/B1 (1A10) Variable Region amino acid sequences
V_{H} chain (SEQ ID NO: 12)

QVQLQQSGGGLVQAGGSLRLSCAASGRTFSNYAIGWFRQAQGKEREFVAAISWGGG
NTYYAGSVKGRFTISRDNAKNTVYLQMNSLKFEDTAVYYCAASEVAHSDYEEEYDYW
GQGTQVTVSS

TABLE N1
Ab a-ITGA3/B1 (S14) Variable Region nucleic acid sequences
V_{H} chain (SEQ ID NO: 32)

CAGGTGCAGCTGGTGGAAAGCGGCGGCGGACTGGTCCAGCCTGGAGGATCTCTGAGACTGAGCT
GCGCCGCTTCTGGCTTCACCTTCAGCAGCTACTTCATGGCCTGGTTCAGACAGGCCCCTGGCAAG
GAACGGGAATTCGTGGCCGCCGTGGGCTGGAACGGCAGCCTGACCAGCTACGCCGACAGCGTGA
AGGGCAGATTCACCATCAGCCGGGACAACGCCAAGTCAACACTGTACCTGCAGATGAACAGCCT
GAAGCCCGAGGACACCGCCGTGTACTACTGCGCCAAAGAGAGTGATACCGGCTGGGGCGAGTAC
GACTACTGGGGCCAGGGCACACAGGTGACCGTGTCCAGC

TABLE N2
Ab a-ITGA3/B1 (S14) Variable Region amino acid sequences
V_{H} chain (SEQ ID NO: 13)

QVQLVESGGGLVQPGGSLRLSCAASGFTFSSYFMAWFRQAPGKEREFVAAVGWNGSL
TSYADSVKGRFTISRDNAKSTLYLQMNSLKPEDTAVYYCAKESDTGWGEYDYWGQGT
QVTVSS

TABLE O1
Ab a-ITGA6/B4 (1A61) Variable Region nucleic acid sequences
V_{H} chain (SEQ ID NO: 33)

CAGGTGCAGCTGCAGCAGTTCGGAGGAGGACTGGTGCAGGCAGGAGGCTCCCTGAGGCTGTCTT
GCGCCGCCAGCGGCCGCATCTTCTCTACCAGCGCCATGGCATGGTTTAGGCAGGCACCAGGCAA
GGAGAGAGAGTTCGTGGCCGAGATCGGCTGGACCGACGAGTCCACACGGTACGCCGATTCTGTG
AAGGGCAGGTTTACCATCAGCCGCGACAACGCCAAGACCACAATCTATCTGCAGATGAATATGC
TGAAGCCCGAGGATACCGCCACATACTATTGTGCCGCCCGGAGATTTTCCAACCCCCCTACAAT
CGAGGCCTACGACTATTGGGGCCAGGGCACCCAGGTGACAGTGAGCTCC

TABLE O2
Ab a-ITGA6/B4 (1A61) Variable Region amino acid sequences
V_{H} chain (SEQ ID NO: 14)

QVQLQQFGGGLVQAGGSLRLSCAASGRIFSTSAMAWFRQAPGKEREFVAEIGWTDES
TRYADSVKGRFTISRDNAKTTIYLQMNMLKPEDTATYYCAARRESNPPTIEAYDYWGQ
GTQVTVSS

TABLE P1
Ab a-ICAM1 (1A101) Variable Region nucleic acid sequences
V_{H} chain (SEQ ID NO: 34)

CAGGTGCAGCTGCAGGAGTCCGGAGGAGGACTGGTGCAGCCAGGAGGCTCTCTGAGGCTGAGCT
GCGCAGCATCCGGCTTCACCTTTTCTAACTACTGGATGTATTGGGTGCGGCAGGCACCAGGCAA
GGGACTGGAGTGGGTGAGCGGAATCGACCACGGAGGCGATTCCACCTACTATGCCGACTCTGTG
AAGGGCCGGTTCACAATCTCTAGAGATGGCAGCAAGAAGATGGTGTACCTGCAGATGGACTCTC
TGAAGCCTGAGGATACAGCCGTGTACTTTTGTTATGTGCAGGCAGCAGCATGGTGGAGCCTGGT
GGGAGGCCCCCCTCCACCCCCTTCCGACTATTGGGGCCAGGGCACCCTGGTGACAGTGAGCTCC

TABLE P2
Ab a-ICAM1 (1A101) Variable Region amino acid sequences
V_{H} chain (SEQ ID NO: 15)

QVQLQESGGGLVQPGGSLRLSCAASGFTFSNYWMYWVRQAPGKGLEWVSGIDHGGD
STYYADSVKGRFTISRDGSKKMVYLQMDSLKPEDTAVYFCYVQAAAWWSLVGGPPPP
PSDYWGQGTLVTVSS

TABLE Q1
Ab a-MME (1A102) Variable Region nucleic acid sequences
V_{H} chain (SEQ ID NO: 35)

CAGGTGCAGCTGCAGGAGTCCGGAGGAGGACTGGTGCAGCCAGGAGGCAGCCTGAGGCTGTCCT
GCGCCGCCTCTGGCTTCACCTTTAGCTCCTACTGGATGTATTGGGTGCGCCAGGCACCTGGCAA
GGGACTGGAGTGGGTGTCCGCCATCAACACAGAGGGCAATACCTACTATACAGACAGCGTGAAG
GGCCGGTTCACCATCTCCCGGGATAACGCCAGAAATACAATGTACCTGCAGATGGACAACCTGA
AGTCTGAGGATACCGGCGTGTACTATTGTGCCAAGGACGCCAAGATCCTGATCGCCCGGATGAG
ATCTCAGGGCAGGCTGTCCCGCTCTGATTTTGGCAGCTGGGGACAGGGAACCCAGGTGACAGTG
TCTAGC

TABLE Q2
Ab a-MME (1A102) Variable Region amino acid sequences
V_{H} chain (SEQ ID NO: 16)

QVQLQESGGGLVQPGGSLRLSCAASGFTFSSYWMYWVRQAPGKGLEWVSAINTEGN
TYYTDSVKGRFTISRDNARNTMYLQMDNLKSEDTGVYYCAKDAKILIARMRSQGRLS
RSDFGSWGQGTQVTVSS

TABLE R1
Ab a-ANPEP (1A105) Variable Region nucleic acid sequences
V_{H} chain (SEQ ID NO: 36)

CAGGTGCAGCTGCAGGCCTCTGGAGGAGGACTGGTGCAGCCAGGAGGCTCCCTGAGGCTGTCTT
GCGCAGGCAGCGGCTTCACCTTTAGCACATACGCAATGGGATGGTTCAGGCAGGCACCAGGCAA
GGAGAGGGAGTTTGTGGCAGCAATCTCCTGGGGAGGCTCTGGAACCTACTATTCTGACAGCGCC
AAGGGCCGGTTCACCATCAGCAGAGATAACGCCAAGAATACAGTGTATCTGCAGATGAACTCCC
TGAAGCCCGAGGACACCGCCGTGTACTATTGTGCCGCCGATAAGCTGCGGCCTAATGGCACAGG
CTTTCTGGCCAGGGGCACCATGATCGAGACATGGGGCCAGGGCACCCAGGTGACAGTGAGCTCC

TABLE R2
Ab a-ANPEP (1A105) Variable Region amino acid sequences
V_{H} chain (SEQ ID NO: 17)

QVQLQASGGGLVQPGGSLRLSCAGSGFTFSTYAMGWFRQAPGKEREFVAAISWGGSG
TYYSDSAKGRFTISRDNAKNTVYLQMNSLKPEDTAVYYCAADKLRPNGTGFLARGTM
IETWGQGTQVTVSS

TABLE S1
Ab a-ANPEP (1A108) Variable Region nucleic acid sequences
V_{H} chain (SEQ ID NO: 37)

CAGGTGCAGCTGCAGCAGTTCGGAGGAGGACTGGTGCAGCCTGGCGACTCTCTGAGGCTGAGCT
GCGCAGCATCCGGAAGGGCCATCAGCATCTACGCAATGGCATGGTTCAGGCAGGCACCAGGCAA
GGAGAGAGAGTTTGTGGCAACCATCTCCTGGGGAGGAGCAACCACATCTTACGCAGACAGCGTG
AAGGGCAGGTTTACCATCTCCCGCGATAACGCCAATGCCAAGTCTACAATGTATCTGCAGATGA
ACGACCTGAAGCCAGAGGATACCGCCGTGTACATCTGTGCAGCAGGACCAACAGACTATCGGAG
AAATGATCCCCCTGCCGCCCGGTACACATATTGGGGCCAGGGCACCCAGGTGACAGTGAGCTCC

TABLE S2
Ab a-ANPEP (1A108) Variable Region amino acid sequences
V_{H} chain (SEQ ID NO: 18)

QVQLQQFGGGLVQPGDSLRLSCAASGRAISIYAMAWFRQAPGKEREFVATISWGGAT
TSYADSVKGRFTISRDNANAKSTMYLQMNDLKPEDTAVYICAAGPTDYRRNDPPAAR
YTYWGQGTQVTVSS

TABLE T1
Ab a-ENG (1A107) [] Variable Region nucleic acid sequences
V_{H} chain (SEQ ID NO: 38)

CAGGTGCAGCTGCAGGAGTCCGGAGGAGGACTGGTGCAGGCAGGAGGCAGCCTGCGGCTGTCCT
GCGCAGCATCTGGAAGGGCCTTCTCTACCTACGTGATGGGCTGGTTTAGGCAGGCACCAGGCAA
GGAGAGGGAGTTCGTGGCAACCATCAACAGGGCAGGAGGCAGCACATACTATGTGGACTCCGTG
AAGGATCGGTTTACCATCTCCAGAGACAACGCCAAGAATACAGTGTACCTGCAGATGGACTCTC
TGAAGCCTGAGGATACCGCCGTGTATAGCTGTGCCGCCGACACAAGCTCCTGGGGCTCCAATTC
TGTGCACGAGAGCGAGTACGATTATTGGGGCCAGGGCACCCAGGTGACAGTGTCTAGC

TABLE T2
Ab a-ENG (1A107) Variable Region amino acid sequences
V_{H} chain (SEQ ID NO: 19)

QVQLQESGGGLVQAGGSLRLSCAASGRAFSTYVMGWFRQAPGKEREFVATINRAGGS
TYYVDSVKDRFTISRDNAKNTVYLQMDSLKPEDTAVYSCAADTSSWGSNSVHESEYD
YWGQGTQVTVSS

The amino acid sequences of the heavy chain complementary determining regions of the monoclonal antibodies are shown below:

TABLE A3

| a-BCAM | | |
|---|---|---|
| VHH | | |
| (6N2_22) | | |
| CDR-VH | Nucleotide sequence | Protein Sequence |
| VH CDR-1 | GGC TTC ACC TTT AGC TCC TAC | GFTFSSYA |
| | (SEQ ID NO: 95) | (SEQ ID NO: 64) |
| VH CDR-2 | ATC AAC TCC GGA GGA GGC TCC | INSGGGSTS |
| | (SEQ ID NO: 96) | (SEQ ID NO: 153) |
| VH CDR-3 | GCC AAG TCT TGGACA GTG CGG | AKSWTVRIGQIYHHPT |
| | ATC GGC CAG ATC TAC CAC CAC | DY |
| | CCC ACC GAT TAT | (SEQ ID NO: 154) |
| | (SEQ ID NO: 97) | |

TABLE B3

| a-CADM1 | | |
|---|---|---|
| VHH | | |
| (6N2_38) | | |
| CDR-VH | Nucleotide sequence | Protein Sequence |
| VH CDR-1 | GGC TTC ACC TTT AGC TCC TAC | GFTFSSYW |
| | TGG (SEQ ID NO: 98) | (SEQ ID NO: 54) |
| VH CDR-2 | ATC AAC ACC GGC GGC GGC | INTGGGST |
| | AGC ACA (SEQ ID NO: 99) | (SEQ ID NO: 55) |
| VH CDR-3 | GCC TAC GGC AATGGC GTG GAG | AYGNGVEGMDY |
| | GGC ATG GAT TAT | (SEQ ID NO: 56) |
| | (SEQ ID NO: 100) | |

TABLE C3

| a-CADM1 | | |
|---|---|---|
| VHH | | |
| (6N2_41) | | |
| CDR-VH | Nucleotide sequence | Protein Sequence |
| VH CDR-1 | GGC TTC ACC TTT AGC TCC TAC | GFTFSSYW |
| | TGG (SEQ ID NO: 98) | (SEQ ID NO: 54) |
| VH CDR-2 | ATC AAC ACC GGC GGA GGC | INTGGGST |
| | TCC ACA (SEQ ID NO: 101) | (SEQ ID NO: 55) |
| VH CDR-3 | GCA ACC CGG GCC TCC GTG GGC | ATRASVGTLEMYDN |
| | ACA CTG GAG ATG TAT GAT AAT | (SEQ ID NO: 57) |
| | (SEQ ID NO: 102) | |

TABLE D3

| a-CAMD1 | | |
|---|---|---|
| VHH | | |
| (6N2_54) | | |
| CDR-VH | Nucleotide sequence | Protein Sequence |
| VH CDR-1 | GGC TTC ACC TTT AGG TCC TAC | GFTFRSYD |
| | GAC | (SEQ ID NO: 58) |
| | (SEQ ID NO: 103) | |
| VH CDR-2 | ATC AAC AGC AGG GGC GGC | INSRGGST |
| | TCC (SEQ ID NO: 104) | (SEQ ID NO: 59) |
| VH CDR-3 | GCC AAG GGC CGGTAT GGC GCC | AKGRYGASWMFPPYD |
| | TCT TGG ATG TTT CCC CCT TAC | Y |
| | GAT TAT (SEQ ID NO: 105) | (SEQ ID NO: 60) |

TABLE E3

| a-CADM1 | | |
|---|---|---|
| VHH | | |
| (6N2_56) | | |
| CDR-VH | Nucleotide sequence | Protein Sequence |
| VH CDR-1 | GGC TTC ACC TTT TCT GAC AGC | GFTFSDSW |
| | TGG ATC (SEQ ID NO: 106) | (SEQ ID NO: 61) |
| VH CDR-2 | ATC AAC ACC GAC GGC TCT AAT | INTDGSNT |
| | ACA (SEQ ID NO: 107) | (SEQ ID NO: 62) |
| VH CDR-3 | GCA AAG GGA GGA CTG ACA | AKGGLTGS |
| | GGC TCT (SEQ ID NO: 108) | (SEQ ID NO: 63) |

TABLE F3

| a-Her2 | | |
|---|---|---|
| (1A11) | | |
| CDR-VH | Nucleotide sequence | Protein Sequence |
| VH CDR-1 | GGA AGG GCC TTC ATC GAC | GRAFIDNV |
| | AAC GTG (SEQ ID NO: 109) | (SEQ ID NO: 39) |
| VH CDR-2 | GGA CTG TCC AGA ACA GGC | GLSRTGANTM |
| | GCC AAT ACC ATG | (SEQ ID NO: 40) |
| | (SEQ ID NO: 110) | |
| VH CDR-3 | GCA GCA AGA TCT CAG GGA | AARSQGATVVITTTGG |
| | GCA ACC GTG GTC ATC ACC ACA | YDY |
| | ACC GGC GGC TAC | (SEQ ID NO: 41) |
| | (SEQ ID NO: 111) | |

TABLE G3

| a-Her2 | | |
|---|---|---|
| (1A12) | | |
| CDR-VH | Nucleotide sequence | Protein Sequence |
| VH CDR-1 | GGA AGG ACC ATC TCC AAC | GRTISNAF |
| | GCC TTC (SEQ ID NO: 112) | (SEQ ID NO: 42) |
| VH CDR-2 | ACAATC TCT ACC ACA GGC ACC | TISTTGTTN |
| | ACA (SEQ ID NO: 113) | (SEQ ID NO: 43) |
| VH CDR-3 | CGG CAC TTT GGC TATGAC GTG | RHFGYDV |
| | (SEQ ID NO: 114) | (SEQ ID NO: 44) |

TABLE H3

| a-Her2 | | |
|---|---|---|
| (1A51) | | |
| CDR-VH | Nucleotide sequence | Protein Sequence |
| VH CDR-1 | GGA CGC ACC TTC TCT CTG TAC | GRTFSLYS |
| | AGC (SEQ ID NO: 115) | (SEQ ID NO: 45) |
| VH CDR-2 | ATC AAC TGG AAT GGC GAG | INWNGEVTE |
| | GTG ACA GAG (SEQ ID NO: 116) | (SEQ ID NO: 46) |
| VH CDR-3 | GCC GCC GCC CCCAGG TTT GAG | AAAPRFESTWLADY |
| | TCT ACC TGG CTG GCA GAT | (SEQ ID NO: 47) |
| | TAC (SEQ ID NO: 117) | |

TABLE I3

| a-Her2 | | |
|---|---|---|
| (1A68) | | Protein |
| CDR-VH | Nucleotide sequence | Sequence |
| VH | GGC AGG TCT TTC CGC AGC TAC | GRSFRSYA |
| CDR-1 | GCC (SEQ ID NO: 118) | (SEQ ID |
| | | NO: 48) |
| VH | GTG TCT TGG AGC GGC GAC ATC | VSWSGDITS |
| CDR-2 | ACA AGC (SEQ ID NO: 119) | (SEQ ID |
| | | NO: 49) |
| VH | GGA GCA AGG CTG GA GGA GCA | GARLGGAIS |
| CDR-3 | ATC TCT GAG GTG GCC GAC CCT | YEVADPYD |
| | TAC GAT TAT (SEQ ID NO: | (SEQ ID |
| | 120) | NO: 50) |

TABLE J3

| a-Her2 | | |
|---|---|---|
| (1A100) | | Protein |
| CDR-VH | Nucleotide sequence | Sequence |
| VH | GGC GAT ACC TTC AGC CGG TAC | GDTFSRYA |
| CDR-1 | GCA (SEQ ID NO: 121) | (SEQ ID |
| | | NO: 51) |
| VH | GTG TCT TGG AGC GGA GGA ATC | VSWSGGITH |
| CDR-2 | ACA CAC (SEQ ID NO: 122) | (SEQ ID |
| | | NO: 52) |
| VH | ACC CAG GAC ACAATC CCT GGA | TQDTIPGGA |
| CDR-3 | GGA GCA GCC CGG GAG TTC | AREFRGY |
| | AGA GGC TAT (SEQ ID NO: | (SEQ ID |
| | 123) | NO: 53) |

TABLE K3

| a-EPHA2 | | |
|---|---|---|
| (4N2 3) | | Protein |
| CDR-VH | Nucleotide sequence | Sequence |
| VH | GGC TTC ACC TTT AGC TCC TAC | GFTFSSYA |
| CDR-1 | GCA (SEQ ID NO: 124) | (SEQ ID |
| | | NO: 64) |
| VH | ATC AAC GGC GGC GGC AGC | INGGGST |
| CDR-2 | ACC (SEQ ID NO: 125) | (SEQ ID |
| | | NO: 65) |
| VH | GCC CGG GTG TAT GGCTCC TAC | ARVYGSYY |
| CDR-3 | TAT GCC TCT TTT TAC GCC ATG | ASFYAMDY |
| | GAT TAT (SEQ ID NO: 126) | (SEQ ID |
| | | NO: 66) |

TABLE L3

| a- | | |
|---|---|---|
| ITGA3/ | | |
| B1 | | |
| (1A6) | | Protein |
| CDR-VH | Nucleotide sequence | Sequence |
| VH | GGA CTG GCC TTC GAC TCC CAC | GLAFDSHQ |
| CDR-1 | CAG (SEQ ID NO: 127) | (SEQ ID |
| | | NO: 67) |
| VH | ATC AGG AGC GCC GGC TCC | IRSAGST |
| CDR-2 | ACC (SEQ ID NO: 128) | (SEQ ID |
| | | NO: 68) |
| VH | GTG GCC GAT AGG ACA TAC TTT | VADRTYFG |
| CDR-3 | GGC CGC GAG GCC GAC TAC | READYDY |
| | GATTAT (SEQ ID NO: 129) | (SEQ ID |
| | | NO: 69) |

TABLE M3

| a- | | |
|---|---|---|
| ITGA3/ | | |
| B1 | | |
| (1A10) | | Protein |
| CDR-VH | Nucleotide sequence | Sequence |
| VH | GGA AGG ACC TTC AGC AAC | GRTFSNYA |
| CDR-1 | TAC GCC (SEQ ID NO: 130) | (SEQ ID |
| | | NO: 70) |
| VH | ATC TCC TGG GGA GGA GGA | ISWGGGNT |
| CDR-2 | AAT ACA (SEQ ID NO: 131) | (SEQ ID |
| | | NO: 71) |
| VH | GCC GCC TCT GAG GTG GCC | AASEVAHS |
| CDR-3 | CAC AGC GAC TAT GAG GAG | DYEEEYDY |
| | GAG TAC GAT TAT (SEQ ID | (SEQ ID |
| | NO: 132) | NO: 72) |

TABLE N3

| a- | | |
|---|---|---|
| ITGA3/ | | |
| B1 | | |
| (S14) | | Protein |
| CDR-VH | Nucleotide sequence | Sequence |
| VH | GGC TTC ACC TTC AGC AGC | GFTFSSYF |
| CDR-1 | TAC TTC (SEQ ID NO: 133) | (SEQ ID |
| | | NO: 73) |
| VH | GTG GGC TGG AAC GGC AGC | VGWNGSLTS |
| CDR-2 | CTG ACC AGC (SEQ ID NO: | (SEQ ID |
| | 134) | NO: 74) |
| VH | GCC AAA GAG AGT GAT ACC | AKESDTGWG |
| CDR-3 | GGC TGG GGC GAG TAC GAC | EYDY |
| | TAC (SEQ ID NO: 135) | (SEQ ID |
| | | NO: 75) |

TABLE O3

| a- | | |
|---|---|---|
| ITGA6/ | | |
| B4 | | |
| (1A61) | | Protein |
| CDR-VH | Nucleotide sequence | Sequence |
| VH | GGC CGC ATC TTC TCT ACC AGC | GRIFSTSA |
| CDR-1 | GCC (SEQ ID NO: 136) | (SEQ ID |
| | | NO: 76) |
| VH | ATC GGC TGG ACC GAC GAG TCC | IGWTDESTR |
| CDR-2 | (SEQ ID NO: 137) | (SEQ ID |
| | | NO: 77) |
| VH | GCC GCC CGG AGA TTT TCC AAC | AARRFSNPP |
| CDR-3 | CCC CCT ACA ATC GAG GCC TAC | TIEAYDY |
| | GAC TAT (SEQ ID NO: 138) | (SEQ ID |
| | | NO: 78) |

TABLE P3

| a-ICAM1 | | |
|---|---|---|
| (1A101) | | Protein |
| CDR-VH | Nucleotide sequence | Sequence |
| VH | GGC TTC ACC TTT TCT AAC | GFTFSNYW |
| CDR-1 | TAC TGG (SEQ ID NO: 139) | (SEQ ID |
| | | NO: 79) |
| VH | ATC GAC CAC GGA GGC GAT | IDHGGDST |
| CDR-2 | ] (SEQ ID NO: 140) | (SEQ ID |
| | | NO: 80) |
| VH | TAT GTG CAG GCA GCA GCA | YVQAAAWW |
| CDR-3 | TGG TGG AGC CTG GTG GGA | SLVGGPPP |
| | GGC CCC CCT CCA CCC CCT | PPSDY |
| | TCC GAC TAT (SEQ ID NO: | (SEQ ID |
| | 141) | NO: 81) |

TABLE Q3

| a-MME | | |
|---|---|---|
| (1A102) | | Protein |
| CDR-VH | Nucleotide sequence | Sequence |
| VH | GGC TTC ACC TTT AGC TCC TAC | GFTFSSYW |
| CDR-1 | TGG (SEQ ID NO: 98) | (SEQ ID |
| | | NO: 54) |
| VH | ATC AAC ACA GAG GGC AAT | INTEGNT |
| CDR-2 | ACC (SEQ ID NO: 142) | (SEQ ID |
| | | NO: 82) |
| VH | GCC AAG GAC GCC AAG ATC CTG | AKDAKILI |
| CDR-3 | ATC GCC CGG ATG AGA TCT CAG | ARMRSQGR |
| | GGC AGG CTG TCC CGC TCT GAT | LSRSDFGS |
| | TTT GGC AGC (SEQ ID NO: | (SEQ ID |
| | 143) | NO: 83) |

TABLE R3

| a-ANPEP | | |
|---|---|---|
| (1A105) | | Protein |
| CDR-VH | Nucleotide sequence | Sequence |
| VH | GGC TTC ACC TTT AGC ACA | GFTFSTYA |
| CDR-1 | TAC (SEQ ID NO: 144) | (SEQ ID |
| | | NO: 84) |
| VH | ATC TCC TGG GGA GGC TCT | ISWGGSGT |
| CDR-2 | GGA ACC (SEQ ID NO: 145) | (SEQ ID |
| | | NO: 85) |
| VH | GCC GCC GAT AAG CTG CGG | AADKLRPN |
| CDR-3 | CCT AAT GGC ACA GGC TTT | GTGFLARG |
| | CTG GCC AGG GGC ACC ATG | TMIET |
| | ATC GAG ACA (SEQ ID NO: | (SEQ ID |
| | 146) | NO: 86) |

TABLE S3

| a-ANPEP | | |
|---|---|---|
| (1A108) | | Protein |
| CDR-VH | Nucleotide sequence | Sequence |
| VH | GGA AGG GCC ATC AGC ATC | GRAISIYA |
| CDR-1 | TAC GCA (SEQ ID NO: | (SEQ ID |
| | 147) | NO: 87) |
| VH | ATC TCC TGG GGA GGA GCA | ISWGGATTS |
| CDR-2 | ACC ACA TCT (SEQ ID NO: | (SEQ ID |
| | 148) | NO: 88) |
| VH | GCA GCA GGA CCA ACA GAC | AAGPTDYRR |
| CDR-3 | TAT CGG AGA AAT GAT CCC | NDPPAARYT |
| | CCT GCC GCC CGG TAC ACA | Y (SEQ ID |
| | TAT (SEQ ID NO: 149) | NO: 89) |

TABLE T3

| a-ENG | | |
|---|---|---|
| (1A107) | | Protein |
| CDR-VH | Nucleotide sequence | Sequence |
| VH | GGA AGG GCC TTC TCT ACC | GRAFSTYV |
| CDR-1 | TAC GTG (SEQ ID NO: 150) | (SEQ ID |
| | | NO: 90) |
| VH | ATC AAC AGG GCA GGA GGC | INRAGGST |
| CDR-2 | AGC ACA (SEQ ID NO: 151) | (SEQ ID |
| | | NO: 91) |
| VH | GCC GCC GAC ACA AGC TCC | AADTSSWG |
| CDR-3 | TGG GGC TCC AAT TCT GTG | SNSVHESE |
| | CAC GAG AGC GAG TAC GAT | YDY |
| | TAT (SEQ ID NO: 152) | (SEQ ID |
| | | NO: 92) |

Embodiments also feature antibodies that have a specified percentage identity or similarity to the amino acid or nucleotide sequences of the antibodies described herein. For example, “homology” or “identity” or “similarity” can refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the antibodies described herein. Sequence identity or similarity to the nucleic acids and proteins of the present invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e. BLAST or BLAST 2.0), manual alignment or visual inspection can be utilized to determine percent sequence identity or similarity for the nucleic acids and proteins of the present invention.

“Polypeptide” as used herein can encompass a singular “polypeptide” as well as plural “polypeptides,” and can refer to a molecule composed of monomers (amino acids) linearly linked by amide bonds (also known as peptide bonds). The term “polypeptide” can refer to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It can be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In embodiments the alteration results in the substitution of an amino acid with a chemically similar amino acid. Conservative substitution tables providing functionally similar amino acids are well known in the art.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide can be replaced with another amino acid residue from the same side chain family. In another embodiment, a string of amino acids can be replaced with a structurally similar string that differs in order and/or composition of side chain family members.

As used herein, the terms “antibody” or “antigen-binding polypeptide” can refer to a polypeptide or a polypeptide complex that specifically recognizes and binds to an antigen. “Specifically binds” or “immunoreacts with” can refer to the interaction of the antibody with one or more epitopes (e.g., antigenic determinant) of an antigen, but interacts weakly with or does not interact with other polypeptides.

An antibody or antigen-binding polypeptide can include any protein- or peptide-containing molecule that comprises at least a portion of an immunoglobulin molecule having biological activity of binding to the antigen. Non-limiting examples of such immunoglobulin portions comprise one or more complementarity determining regions (CDR) of a heavy chain or light chain or a ligand binding portion thereof, a heavy chain or light chain variable region, a heavy chain or light chain constant region, a framework (FR) region, or any portion thereof, or at least one portion of a binding protein.

In embodiments, the antibody or antigen-binding fragment can comprise an immunoglobulin molecule, for example, a molecule that contains an immunologically active portion (e.g., an antigen binding site) that specifically binds (immunoreacts with) an antigen.

In embodiments, the antibody can be a whole antibody, an antibody fusion, or an antibody fragment.

The term “whole antibody” can refer to an immunoglobulin molecule comprising two “heavy chains” and two “light chains”, each of which comprises a variable and constant region.

For example, the antibody can be a mammalian antibody, such as derived from a human, mouse, rabbit, or other mammal. In embodiments, the antibody can be an IgG antibody (i.e., IgG1, IgG2, IgG3, or IgG4). In embodiments, the antibody can be a fully human antibody or a humanized antibody.

Antibody molecules obtained from humans fall into five classes of immunoglubulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (y, u, a, 8, ¿) with some subclasses among them (e.g., γ1-γ4). Certain classes have subclasses as well, such as IgG1, IgG2, IgG3 and IgG4 and others. The immunoglobulin subclasses (isotypes) e.g., IgG1, IgG2, IgG3, IgG4, IgG5, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as either kappa or lambda (K, A). Each heavy chain class can be bound with either a kappa or lambda light chain. The light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions can adopt a β-sheet conformation and the CDRs form loops which connect, and in some cases form part of, the β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” can refer to the numbering system set forth by Kabat et al., U.S. Dept. of Health and Human Services, “Sequence of Proteins of Immunological Interest” (1983).

The term “antibody fragment” can refer to a molecule other than the complete antibody, such as a molecule that comprises a portion of the complete antibody that binds to an antigen to which the complete antibody binds. Examples of antibody fragments include, but are not limited to, scFv, Fv, Fab, Fab′, Fab′-SH, F (ab′) 2, F (ab) 2, diabodies, triabodies, tetrabodies, cross-Fab fragments; linear antibodies; single chain antibody molecules (e.g., scFv); multispecific antibodies and single domain antibodies formed from antibody fragments. For a review of antibody fragments, see Hudson et al., Nat Med 9, 129-134 (2003). For review of the scFv fragment, see, for example, Pluckthun, in The Pharmacology of Monoclonal Antibodies, vol. 113, Rosenburg and Moore eds., Springer-Verlag, New York, pp. 269-315 (1994); WO 93/16185; and U.S. Pat. Nos. 5,571,894 and 5,587,458. For a discussion of Fab fragments containing structural receptor binding epitope residues and increased in vivo half life, see U.S. Pat. No. 5,869,046. A diabody is an antibody fragment having two antigen-binding sites that can be bivalent or bispecific. For example, EP 0404097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat. Med. 9, 129-134 (2003). A single-domain antibody is an antibody fragment comprising part or all of the heavy chain variable domain of the antibody or a portion or all of the light chain variable domain. In some embodiments, the single-domain antibody is a human single-domain antibody (see Domantis, Inc., Waltham, MA; see, for example, U.S. Pat. No. 6,248,516 B1). In addition, the antibody fragment can be designed to have a characteristic of the VH domain, that is, to be assembled with the VL domain, or to have the characteristics of the VL domain, i.e. to be assembled with the VH domain. Antibody fragments can be produced by various techniques such as, but not limited to, proteolytic cleavage of whole antibodies, as described in the present invention, as well as production by recombinant host cells (e.g., Escherichia coli or phage).

Regardless of structure, an antibody fragment can bind with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F (ab′) 2, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” can refer to a fusion protein of the variable regions of the heavy (VH) and light chains (VL) of immunoglobulins. A single chain Fv (“scFv”) polypeptide molecule is a covalently linked VH: VL heterodimer, which can be expressed from a gene fusion including VH- and VL-encoding genes linked by a peptide-encoding linker. (See Huston et al. (1988) Proc Nat Acad Sci USA 85 (16): 5879-5883). In embodiments the regions are connected with a short linker peptide, such as a short linker peptide of about ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Very large naive human scFv libraries have been and can be created to offer a large source of rearranged antibody genes against a plethora of target molecules. Smaller libraries can be constructed from individuals with infectious diseases in order to isolate disease-specific antibodies. (See Barbas et al., Proc. Natl. Acad. Sci. USA 89:9339-43 (1992); Zebedee et al, Proc. Natl. Acad. Sci. USA 89:3 175-79 (1992)).

Embodiments can also comprise scFv-Fc fragments. “scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., VH-VL or VL-VH). Any suitable Fc domain known in the art or described herein may be used. In some cases, the Fc domain comprises an IgG4 Fc domain.

In embodiments, the antibody can be a single domain antibody. The term “single domain antibody” can refer to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245. Single domain antibodies are also known as sdAbs or nanobodies.

In embodiments, the antibody fusion can be an Fc-fusion antibody (e.g., α-BCAM-VHH-IgG fusion). For example, embodiments can comprise:

Nucleic acid sequence of hIgG1-Fc
(SEQ ID NO: 155)
CGACAAAACTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTACGTGGACGG
CGTGGAGGTGCATAATGCCAAGACAAAGCCGCGGGAGGAGCAGTACAACAGCACGTACCGT
GTGGTCAGCGTCCTCACCGTCCTGCACCAGGACTGGCTGAATGGCAAGGAGTACAAGTGCA
AGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGAGGAGATGACCAAGAACCAG
GTCAGCCTGACCTGCCTGGTCAAAGGCTTCTATCCCAGCGACATCGCCGTGGAGTGGGAGA
GCAATGGGCAGCCGGAGAACAACTACAAGACCACGCCTCCCGTGCTGGACTCCGACGGCTC
CTTCTTCCTCTACAGCAAGCTCACCGTGGACAAGAGCAGGTGGCAGCAGGGGAACGTCTTC
TCATGCTCCGTGATGCACGAGGCTCTGCACAACCACTACACGCAGAAGAGCCTCTCCCTGT
CTCCGGGTAAATGA
Amino acid sequence of hIgG1-Fc (* indicates a stop;
LALA mutations are highlighted in bold (LL is
changed to AA))
(SEQ ID NO: 156)
D K T H T C P P C P A P E L L G G P S V F L F P P K P K D T L
M I S R T P E V T C V V V D V S H E D P E V K F N W Y V D G V
E V H N A K T K P R E E Q Y N S T Y R V V S V L T V L H Q D W
L N G K E Y K C K V S N K A L P A P I E K T I S K A K G Q P R
E P Q V Y T L P P S R E E M T K N Q V S L T C L V K G F Y P S
D I A V E W E S N G Q P E N N Y K T T P P V L D S D G S F F L
Y S K L T V D K S R W Q Q G N V F S C S V M H E A L H N H Y T
Q K S L S L S P G K *

One of skill in the art understands that any other human/mouse (or other species) IgF Fcs can be used based on desired effector function. In one embodiment, an IgG1-Fc fusion was cloned.

Nucleic acid sequence of mIgG2a-Fc
(SEQ ID NO: 157)
CCCAGAGGGCCCACAATCAAGCCCTGTCCTCCATGCAAATGCCCAGCACCTAACCTCTTGG
GTGGACCATCCGTCTTCATCTTCCCTCCAAAGATCAAGGATGTACTCATGATCTCCCTGAG
CCCCATAGTCACATGTGTGGTGGTGGATGTGAGCGAGGATGACCCAGATGTCCAGATCAGC
TGGTTTGTGAACAACGTGGAAGTACACACAGCTCAGACACAAACCCATAGAGAGGATTACA
ACAGTACTCTCCGGGTGGTCAGTGCCCTCCCCATCCAGCACCAGGACTGGATGAGTGGCAA
GGAGTTCAAATGCAAGGTCAACAACAAAGACCTCCCAGCGCCCATCGAGAGAACCATCTCA
AAACCCAAAGGGTCAGTAAGAGCTCCACAGGTATATGTCTTGCCTCCACCAGAAGAAGAGA
TGACTAAGAAACAGGTCACTCTGACCTGCATGGTCACAGACTTCATGCCTGAAGACATTTA
CGTGGAGTGGACCAACAACGGGAAAACAGAGCTAAACTACAAGAACACTGAACCAGTCCTG
GACTCTGATGGTTCTTACTTCATGTACAGCAAGCTGAGAGTGGAAAAGAAGAACTGGGTGG
AAAGAAATAGCTACTCCTGTTCAGTGGTCCACGAGGGTCTGCACAATCACCACACGACTAA
GAGCTTCTCCCGGACTCCGGGTAAATGA
Amino acid sequence of mIgG2a-Fc (* indicates a stop)
(SEQ ID NO: 158)
P R G P T I K P C P P C K C P A P N L L G G P S V F I F P P K
I K D V L M I S L S P I V T C V V V D V S E D D P D V Q I S W
F V N N V E V H T A Q T Q T H R E D Y N S T L R V V S A L P I
Q H Q D W M S G K E F K C K V N N K D L P A P I E R T I S K P
K G S V R A P Q V Y V L P P P E E E M T K K Q V T L T C M V T
D F M P E D I Y V E W T N N G K T E L N Y K N T E P V L D S D
G S Y F M Y S K L R V E K K N W V E R N S Y S C S V V H E G L
H N H H T T K S F S R T P G K *
Nucleic acid sequence of full a-BCAM VHH-hIgG1-Fc
fusion construct
(SEQ ID NO: 159)
CAGGTGCAGCTGGTGGAGTCCGGAGGAGGACTGGTGCAGCCAGGAGGCAGCCTGAGGCTGT
CCTGCGCCGCCTCTGGCTTCACCTTTAGCTCCTACGCCATGAGCTGGGTGCGCCAGGCACC
AGGCAAGGGACCTGAGTGGGTGAGCGCCATCAACTCCGGAGGAGGCTCCACATCTTACGCC
GACTCTGTGAAGGGCCGGTTCACCATCAGCAGAGATAACGCCAAGAATACACTGTATCTGC
AGATGAACAGCCTGAAGCCAGAGGACACCGCCGTGTACTATTGTGCCAAGTCTTGGACAGT
GCGGATCGGCCAGATCTACCACCACCCCACCGATTATTGGGGCCAGGGCACCCAGGTGACA
GTGTCTAGCgaattcGACAAAACTCACACATGCCCACCGTGCCCAGCACCTGAACTCCTGG
GGGGACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACA
ACAGCACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGAGGAGA
TGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTG
GACTCCGACGGCTCCTTCTTCCTCTACAGCAAGCTCACCGTGGACAAGAGCAGGTGGCAGC
AGGGGAACGTCTTCTCATGCTCCGTGATGCACGAGGCTCTGCACAACCACTACACGCAGAA
GAGCCTCTCCCTGTCTCCGGGTAAATGA
Amino acid sequence of full a-BCAM VHH-hIgG1-Fc
fusion construct. The amino acid sequences of
the heavy chain complementary determining regions
(CDRs) are underlined (CDR1), underlined and
bolded (CDR2), or underlined, italicized, and
bolded (CDR3). (* indicates a stop)
(SEQ ID NO: 160)
QVQLVESGGGLVQPGGSLRLSCAASGFTFSSYAMSWVRQAPGKGPEWVSAINSGGGSTSYA
DSVKGRFTISRDNAKNTLYLOMNSLKPEDTAVYYCAKSWTVRIGQIYHHPTDYWGQGTQVT
VSS D K T H T C P P C P A P E L L G G P S V F L F P P K P K D
T L M I S R T P E V T C V V V D V S H E D P E V K F N W Y V D
G V E V H N A K T K P R E E Q Y N S T Y R V V S V L T V L H Q
D W L N G K E Y K C K V S N K A L P A P I E K T I S K A K G Q
P R E P Q V Y T L P P S R E E M T K N Q V S L T C L V K G F Y
P S D I A V E W E S N G Q P E N N Y K T T P P V L D S D G S F
F L Y S K L T V D K S R W Q Q G N V F S C S V M H E A L H N H
Y T Q K S L S L S P G K *

The term “Fc-fusion” can refer to a fusion protein including the Fc region of an immunoglobulin. The Fc-fusion may include an Fc comprising least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a naturally occurring Fc. In embodiments, the Fc is a mouse IgG Fc. In embodiments, the Fc is a mouse IgG2A Fc. In embodiments, the Fc is a human IgG Fc. In embodiments, the Fc is a human IgG₁ Fc. In embodiments, the Fc is an engineered Fc. For example, the Fc can be engineered to have enhanced effector fuction. For example, the Fc can be engineered to have dimished effector function. For example, the Fc can be engineered to contain glycosylation sites. For example, the Fc can be engineered to contain mutations which affect protein half-life. For example, the Fc can contain a LALA mutation to abolish ADCC activity.

In embodiments, the antibody can be monospecific or multispecific.

A “monospecific antibody” is an antibody that comprises one or more binding sites that specifically bind to a single epitope. An example of a monospecific ABP is a naturally occurring IgG molecule which, while divalent (i.e., having two antigen-binding domains), recognizes the same epitope at each of the two antigen-binding domains. The binding specificity can be present in any suitable valency.

A “multispecific antibody” is an antibody that comprises two or more different antigen-binding domains that collectively specifically bind two or more different epitopes. The two or more different epitopes can be epitopes on the same antigen (e.g., a single molecule expressed by a cell) or on different antigens (e.g., different molecules expressed by the same cell). In aspects, a multi-specific antibody binds two different epitopes (i.e., a “bispecific antibody”). In some aspects, a multi-specific ABP binds three different epitopes (i.e., a “trispecific antibody”). In some aspects, a multi-specific ABP binds four different epitopes (i.e., a “quadspecific antibody”). In some aspects, a multi-specific ABP binds 5, 6, 7, 8, or more different epitopes. Each binding specificity can be present in any suitable valency.

In embodiments, the invention provides for multispecific antibodies, such as bi-specific antibodies that recognize a first antigen and a second antigen. For example, the first antigen and/or the second antigen can be a tumor antigen. As a tumor antigen targeting molecule, an antibody or antigen-binding fragment can be combined with a second antigen-binding fragment specific to an immune cell to generate a bispecific antibody. In embodiments, the immune cell is selected from the group consisting of a T cell, a B cell, a monocyte, a macrophage, a neutrophil, a dendritic cell, a phagocyte, a natural killer cell, an eosinophil, a basophil, and a mast cell. Molecules on the immune cell which can be targeted include, but not limited to, for example, CD3, CD16, CD19, CD28, and CD64. Other non-limiting examples include PD-1, CTLA-4, LAG-3 (also known as CD223), CD28, CD122, 4-1BB (also known as CD137), TIM3, OX-40 or OX40L, CD40 or CD40L, LIGHT, ICOS/ICOSL, GITR/GITRL, TIGIT, CD27, VISTA, B7H3, B7H4, HEVM or BTLA (also known as CD272), killer-cell immunoglobulin-like receptors (KIRs), and CD47. Exemplary second antigens include tumor associated antigens (e.g., LINGO1, EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, aVB3, a5B1, ERBB2, ERBB3, MET, IGFIR, EPHA3, TRAILR1, TRAILR2, RANKL, FAP and Tenascin), cytokines (e.g., IL-2, IL-3, IL-4, IL-5, IL-6, IL-7, IL-10, IL-12, IL-13, IL-15, GM-CSF, TNF-α, CD40L, OX40L, CD27L, CD30L, 4-1BBL, LIGHT and GITRL), and cell surface receptors. Different formats of bispecific antibodies are also provided herein. In embodiments, each of the first antibody fragment and the second antibody fragment is each independently selected from a Fab fragment, a single-chain variable fragment (scFv), or a single-domain antibody. In embodiments, the bispecific antibody further includes a Fc fragment. A bi-specific antibody of the invention comprises a heavy chain and a light chain combination or scFv of the antibodies disclosed herein.

Multispecific antibodies (e.g., bispecific antibodies and trispecific antibodies) of the invention can be constructed using methods known art. In some embodiments, the bi-specific antibody is a single polypeptide wherein the two scFv fragments are joined by a long linker polypeptide, of sufficient length to allow intramolecular association between the two scFv units to form an antibody. In other embodiments, the bi-specific antibody is more than one polypeptide linked by covalent or non-covalent bonds. In some embodiments, the amino acid linker (GGGGSGGGGS; “(G4S) 2” (SEQ ID NO: 161)) that can be used with scFv fusion constructs described herein can be generated with a longer G4S linker (SEQ ID NO: 162) to improve flexibility. For example, the linker can also be

“(G4S)3”

(SEQ ID NO: 163)

(e.g., GGGGSGGGGSGGGGS);
“(G4S)4”

(SEQ ID NO: 164)

(e.g., GGGGSGGGGGGGGSGGGGS);
“(G4S)5”

(SEQ ID NO: 165)

(e.g., GGGGSGGGGSGGGGSGGGGSGGGGS);
“(G4S)6”

(SEQ ID NO: 166)

(e.g., GGGGSGGGGSGGGGSGGGGSGGGGSGGGGS);
“(G4S)7”

(SEQ ID NO: 167)

(e.g., GGGGSGGGGSGGGGSGGGGSGGGGSGGGGSGGGGS);

and the like. For example, use of the (G4S) 5 linker (SEQ ID NO: 165) can provide more flexibility and can improve expression. In some embodiments, the linker can also be (GS) n (SEQ ID NO: 168), (GGS)n (SEQ ID NO: 169), (GGGS)n (SEQ ID NO: 170), (GGSG)n (SEQ ID NO: 171), (GGSGG)n (SEQ ID NO: 172), or (GGGGS)n (SEQ ID NO: 173), wherein n is 1, 2, 3, 4, 5, 6, 7, 8, 9, or 10. Non-limiting examples of linkers known to those skilled in the art that can be used are described in U.S. Pat. No. 9,708,412; U.S. Patent Application Publication Nos. US20180134789 and US20200148771; and PCT Publication No. WO2019051122 (each of which are incorporated by reference in their entireties).

In embodiments, the multispecific antibodies (e.g., bispecific antibodies and trispecific antibodies) can be constructed using the “knob into hole” method (Ridgway et al, Protein Eng 7:617-621 (1996)). In this method, the Ig heavy chains of the two different variable domains are reduced to selectively break the heavy chain pairing while retaining the heavy-light chain pairing. The two heavy-light chain heterodimers that recognize two different antigens are mixed to promote heteroligation pairing, which is mediated through the engineered “knob into holes” of the CH3 domains.

In embodiments, multispecific antibodies (e.g., bispecific antibodies and trispecific antibodies) can be constructed through exchange of heavy-light chain dimers from two or more different antibodies to generate a hybrid antibody where the first heavy-light chain dimer recognizes a first antigen and the second heavy-light chain dimer recognizes a second antigen. In some embodiments, the bi-specific antibody can be constructed through exchange of heavy-light chain dimers from two or more different antibodies to generate a hybrid antibody where the first heavy-light chain dimer recognizes a second antigen and the second heavy-light chain dimer recognizes the first antigen. The mechanism for heavy-light chain dimer is similar to the formation of human IgG4, which also functions as a bispecific molecule. Dimerization of IgG heavy chains is driven by intramolecular force, such as the pairing the CH3 domain of each heavy chain and disulfide bridges. Presence of a specific amino acid in the CH3 domain (R409) has been shown to promote dimer exchange and construction of the IgG4 molecules. Heavy chain pairing is also stabilized further by interheavy chain disulfide bridges in the hinge region of the antibody. For example, in IgG4, the hinge region contains the amino acid sequence Cys-Pro-Ser-Cys (SEQ ID NO: 174) (in comparison to the stable IgG1 hinge region which contains the sequence Cys-Pro-Pro-Cys (SEQ ID NO: 175)) at amino acids 226-230. This sequence difference of Serine at position 229 has been linked to the tendency of IgG4 to form intrachain disulfides in the hinge region (Van der Neut Kolfschoten, M. et al, 2007, Science 317:1554-1557 and Labrijn, A. F. et al, 2011, Journal of Immunol 187:3238-3246).

Therefore, bi-specific antibodies of the invention can be created through introduction of the R409 residue in the CH3 domain and the Cys-Pro-Ser-Cys sequence (SEQ ID NO: 174) in the hinge region of antibodies that recognize a first antigen or a second antigen, so that the heavy-light chain dimers exchange to produce an antibody molecule with one heavy-light chain dimer recognizing a first antigen and the second heavy-light chain dimer recognizing a second antigen, wherein the second antigen is any antigen disclosed herein. Known IgG4 molecules can also be altered such that the heavy and light chains recognize a first antigen or a second antigen, as disclosed herein. Use of this method for constructing the bi-specific antibodies of the invention can be beneficial due to the intrinsic characteristic of IgG4 molecules wherein the Fc region differs from other IgG subtypes in that it interacts poorly with effector systems of the immune response, such as complement and Fc receptors expressed by certain white blood cells. This specific property makes these IgG4-based bi-specific antibodies attractive for therapeutic applications, in which the antibody is required to bind the target(s) and functionally alter the signaling pathways associated with the target(s), however not trigger effector activities.

In embodiments, mutations are introduced to the constant regions of the bsAb such that the antibody dependent cell-mediated cytotoxicity (ADCC) activity of the bsAb is altered. For example, the mutation is a LALA mutation in the CH2 domain. In one aspect, the bsAb contains mutations on one scFv unit of the heterodimeric bsAb, which reduces the ADCC activity. In another aspect, the bsAb contains mutations on both chains of the heterodimeric bsAb, which completely ablates the ADCC activity. For example, the mutations introduced one or both scFv units of the bsAb are LALA mutations in the CH2 domain. These bsAbs with variable ADCC activity can be optimized such that the bsAbs exhibits maximal selective killing towards cells that express one antigen that is recognized by the bsAb, however exhibits minimal killing towards the second antigen that is recognized by the bsAb.

The bi-specific antibodies disclosed herein can be useful in treatment of medical conditions, for example cancer.

As used herein, the term “epitope” can include any protein determinant capable of specific binding to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants can consist of chemically active surface groupings of molecules such as amino acids or sugar side chains and can have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e. CDR-H1, CDR-H2, CDR-H3, CDR-L1, CDR-L2 and CDR-L3).

In embodiments, an antibody described herein can be a “therapeutic candidate” or a “diagnostic candidate”. A candidate antibody, for example, can refer to an antibody which can or has the potential to provide an effect, such as a therapeutic effect of a diagnostic effect.

The terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ allows the cancellation of all parameters not related to affinity, and is equal to the dissociation constant K_{d}. (See, generally, Davies et al. (1990) Annual Rev Biochem 59:439-473).

An antibody of the invention can specifically bind to a target epitope when the equilibrium binding constant K_{d} is less than about 100 nM. For example, the Kais less than about 90 nM, less than about 80 nM, less than about 70 nM, less than about 60 nM, less than about 50 nM, less than about 40 nM, less than about 30 nM, less than about 20 nM, less than about 10 nM, or less than about 5 nM. In embodiments, the Kais about 10-25 nM, about 25-50 nM, about 50-75 nM, or about 75-100 nM. In embodiments, the K_{d} is about 1 nM, about 10 nM, about 20 nM, about 30 nM, about 40 nM, about 50 nM, about 60 nM, about 70 nM, about 80 nM, about 90 nM, about 100 nM, or greater than about 100 nM. Functionally, the binding affinity of the of the target antibody is from about 1 nM to about 50 nM.

Those skilled in the art will recognize that one can determine, without undue experimentation, if a human monoclonal antibody has the same specificity as a human monoclonal antibody of the invention by ascertaining whether the former prevents the latter from binding to a target epitope. For example, if the human monoclonal antibody being tested competes with the human monoclonal antibody of the invention, as shown by a decrease in binding by the human monoclonal antibody of the invention, then the two monoclonal antibodies can bind to the same, or to a closely related, epitope.

Another way to determine whether a human monoclonal antibody has the specificity of a human monoclonal antibody of the invention is to pre-incubate the human monoclonal antibody of the invention with the target protein, with which it is normally reactive, and then add the human monoclonal antibody being tested to determine if the human monoclonal antibody being tested is inhibited in its ability to bind to the target. If the human monoclonal antibody being tested is inhibited then, it can have the same, or functionally equivalent, epitopic specificity as the monoclonal antibody of the invention. Screening of human monoclonal antibodies of the invention can be also carried out by utilizing the target and determining whether the test monoclonal antibody is able to neutralize the target.

Various procedures known within the art can be used for the production of polyclonal or monoclonal antibodies directed against a protein of the invention, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen, which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “mAb” or “Mab” or “monoclonal antibody composition” can refer to a population of antibody molecules that contain only one molecular species of antibody molecule consisting of a unique light chain gene product and a unique heavy chain gene product. For example, the complementarity determining regions (CDRs) of the monoclonal antibody are identical in all the molecules of the population. MAbs contain an antigen binding site that can immunoreact with an epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is immunized with an immunizing agent to elicit lymphocytes that produce or can produce antibodies that will specifically bind to the immunizing agent. Alternatively, the lymphocytes can be immunized in vitro.

The immunizing agent can include the protein antigen, a fragment thereof or a fusion protein thereof. For example, peripheral blood lymphocytes can be used if cells of human origin are desired, or spleen cells or lymph node cells can be used if non-human mammalian sources are desired. The lymphocytes are then fused with an immortalized cell line using a suitable fusing agent, such as polyethylene glycol, to form a hybridoma cell (See Goding, Monoclonal Antibodies: Principles and Practice, Academic Press, (1986) pp. 59-103). Immortalized cell lines can be transformed mammalian cells, particularly myeloma cells of rodent, bovine and human origin. For example, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas will include hypoxanthine, aminopterin, and thymidine (“HAT medium”), which substances prevent the growth of HGPRT-deficient cells.

Immortalized cell lines that are useful are those that fuse efficiently, support stable high-level expression of antibody by the selected antibody-producing cells, and are sensitive to a medium such as HAT medium. For example, immortalized cell lines can be murine myeloma lines, which can be obtained, for instance, from the Salk Institute Cell Distribution Center (San Diego, California) and the American Type Culture Collection (Manassas, Virginia). Human myeloma and mouse-human heteromyeloma cell lines also have been described for the production of human monoclonal antibodies. (See Kozbor, J. Immunol, 133:3001 (1984); Brodeur et al, Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. For example, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567 (incorporated herein by reference in its entirety). DNA encoding the monoclonal antibodies of the invention can be readily isolated and sequenced using conventional procedures (e.g., by using oligonucleotide probes that can bind specifically to genes encoding the heavy and light chains of murine antibodies). The hybridoma cells of the invention serve as a source of such DNA. Once isolated, the DNA can be placed into expression vectors, which are then transfected into host cells, for example simian

COS cells, Chinese hamster ovary (CHO) cells, or myeloma cells, that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (See U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

In embodiments, the antibody can be a fully human antibody or a humanized antibody. Fully human antibodies are antibody molecules in which the entire sequence of both the light chain and the heavy chain, including the CDRs, arise from human genes. Such antibodies can be referred to as “human antibodies” or “fully human antibodies”. Human monoclonal antibodies can be prepared by using trioma technique; the human Bcell hybridoma technique (see Kozbor, et al., 1983 Immunol Today 4:72); and the EBV hybridoma technique to produce human monoclonal antibodies (see Cole, et al., 1985 In: MONOCLONAL ANTIBODIES AND CANCER THERAPY, Alan R. Liss, Inc., pp. 7796). Human monoclonal antibodies can be utilized and can be produced by using human hybridomas (see Cote, et al., 1983. Proc Natl Acad Sci USA 80:20262030) or by transforming human Bcells with Epstein Barr Virus in vitro (see Cole, et al., 1985 In: MONOCLONAL ANTIBODIES AND CANCER THERAPY, Alan R. Liss, Inc., pp. 7796).

“Humanized antibodies” can be antibodies from non-human species (such as a mouse) whose light chain and heavy chain protein sequences have been modified to increase their similarity to antibody variants produced in humans. Humanized antibodies are antibody molecules derived from a non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, for example, improve, antigen-binding. These framework substitutions are identified by methods well known in the art, e.g., by modeling of the interactions of the CDR and framework residues to identify framework residues important for antigen-binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) For example, the non-human part of the antibody (such as the CDR(s) of a light chain and/or heavy chain) can bind to the target antigen.

Antibodies can be humanized using a variety of techniques known in the art including, for example, CDR-grafting (EP 239,400; PCT publication WO 91 09967; U.S. Pat. Nos. 5,225,539; 5,530,101; and 5,585,089), veneering or resurfacing (EP 592,106; EP 519,596; Padlan, Molecular Immunology 28 (4 5): 489-498 (1991); Studnicka et al., Protein Engineering 7 (6): 805-814 (1994); Roguska. et al., Proc. Natl. Sci. USA 91:969-973 (1994)), and chain shuffling (U.S. Pat. No. 5,565,332, which is incorporated by reference in its entirety). “Humanization” (also called Reshaping or CDR-grafting) is a well-established technique understood by the skilled artisan for reducing the immunogenicity of monoclonal antibodies (mAbs) from xenogeneic sources (commonly rodent) and for improving their activation of the human immune system (See, for example, Hou S, Li B, Wang L, Qian W, Zhang D, Hong X, Wang H, Guo Y (July 2008). “Humanization of an anti-CD34 monoclonal antibody by complementarity-determining region grafting based on computer-assisted molecular modeling”. J Biochem. 144 (1): 115-20). Antibodies can be humanized by methods known in the art, such as CDR-grafting. See also, Safdari et al., (2013) Biotechnol Genet Eng Rev.; 29:175-86. In addition, humanized antibodies can be produced in transgenic plants, as an inexpensive production alternative to existing mammalian systems. For example, the transgenic plant may be a tobacco plant, i.e., Nicotiania benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can be readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the invention further provides any cell or plant comprising a vector that encodes an antibody of the invention, or produces an antibody of the invention.

Human monoclonal antibodies, such as fully human and humanized antibodies, can be prepared by using trioma technique; the human B-cell hybridoma technique (see Kozbor, et al, 1983 Immunol Today 4:72); and the EBV hybridoma technique to produce human monoclonal antibodies (see Cole, et al, 1985 In: MONOCLONAL ANTIBODIES AND CANCER THERAPY, Alan R. Liss, Inc., pp. 77-96). Human monoclonal antibodies can be utilized and can be produced by using human hybridomas (see Cote, et al, 1983. Proc Natl Acad Sci USA 80:2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using other techniques, including phage display libraries. (See Hoogenboom and Winter, J. Mol. Biol, 227:381 (1991); Marks et al., J. Mol. Biol, 222:581 (1991)). Human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio Technology 10, 779-783 (1992); Lonberg et al, Nature 368, 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies can additionally be produced using transgenic nonhuman animals which are modified so as to produce fully human antibodies rather than the animal's endogenous antibodies in response to challenge by an antigen. (See, PCT publication no. WO94 02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. A non-limiting example of such a nonhuman animal is a mouse, and is termed the Xenomouse™ as disclosed in PCT publication nos. WO96 33735 and WO96 34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

Thus, using such a technique, therapeutically useful IgG, IgA, IgM and IgE antibodies can be produced. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publications WO 98 24893; WO 96 34096; WO 96 33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Creative BioLabs (Shirley, NY) can be engaged to provide human antibodies directed against a selected antigen using technology similar to that described herein.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody described herein, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, is disclosed in PCT publication No. WO99 53049.

In embodiments, the antibody can also be expressed by a vector containing a DNA segment encoding the single chain antibody described herein.

These vectors can include liposomes, naked DNA, adjuvant-assisted DNA, gene gun, catheters, etc. Vectors can further include chemical conjugates such as described in WO 93/64701, which has targeting moiety (e.g. a ligand to a cellular surface receptor), and a nucleic acid binding moiety (e.g. polylysine), viral vectors (e.g. a DNA or RNA viral vector), fusion proteins such as described in PCT/U.S. 95/02140 (WO 95/22618), which is a fusion protein containing a target moiety (e.g. an antibody specific for a target cell) and a nucleic acid binding moiety (e.g. a protamine), plasmids, phage, viral vectors, etc. The vectors can be chromosomal, non-chromosomal or synthetic. Retroviral vectors can also be used, and include moloney murine leukemia viruses.

DNA viral vectors can also be used, and include pox vectors such as orthopox or avipox vectors, herpesvirus vectors such as a herpes simplex I virus (HSV) vector (See Geller, A. I. et al, J. Neurochem, 64:487 (1995); Lim, F., et al, in DNA Cloning: Mammalian Systems, D. Glover, Ed. (Oxford Univ. Press, Oxford England) (1995); Geller, A. I. et al, Proc Natl. Acad. Sci.: U.S.A. 90:7603 (1993); Geller, A. I., et al, Proc Natl. Acad. Sci USA 87:1149 (1990), Adenovirus Vectors (see LeGal LaSalle et al, Science, 259:988 (1993); Davidson, et al, Nat. Genet 3:219 (1993); Yang, et al, J. Virol. 69:2004 (1995) and Adeno-associated Virus Vectors (see Kaplitt, M. G . . . et al, Nat. Genet. 8:148 (1994).

Pox viral vectors introduce the gene into the cell's cytoplasm. Avipox virus vectors result in only a short-term expression of the nucleic acid. Adenovirus vectors, adeno-associated virus vectors, and herpes simplex virus (HSV) vectors can be used for introducing the nucleic acid into neural cells. The adenovirus vector results in a shorter-term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaP04 precipitation, DEAE dextran, electroporation, protoplast fusion, lipofection, cell microinjection, and viral vectors.

The vector can be employed to target any desired target cell. For example, stereotaxic injection can be used to direct the vectors (e.g. adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and can be useful in delivering the vector to the target cell. (See Bobo et al, Proc. Natl. Acad. Sci. USA 91:2076-2080 (1994); Morrison et al, Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways, for example, to detect the presence of a target in a sample. In an embodiment, the antibodies of the invention are full-length antibodies, containing an Fc region similar to wild-type Fc regions that bind to Fc receptors. In other embodiments, the antibodies of the invention are antibody fragments, such as scFv antibodies.

Techniques can be adapted for the production of single-chain antibodies specific to an antigenic protein of the invention (See e.g., U.S. Pat. No. 4,946,778). In addition, methods can be adapted for the construction of Fab expression libraries (See e.g., Huse, et al, 1989 Science 246:1275-1281) to allow rapid and effective identification of monoclonal F_{ab} fragments with the desired specificity for a protein or derivatives, fragments, analogs or homologs thereof. Antibody fragments that contain the idiotypes to a protein antigen can be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Heteroconjugate antibodies are also within the scope of the present invention. Heteroconjugate antibodies are composed of two covalently joined antibodies. Such antibodies can, for example, target immune system cells to unwanted cells (see U.S. Pat. No. 4,676,980), and for treatment of infection (See PCT Publication Nos. WO91 00360; WO92 20373). The antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The antibody of the invention can be modified with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al, J. Exp Med., 176:1 191-1 195 (1992) and Shopes, J. Immunol., 148:2918-2922 (1992)). Alternatively, an antibody can be engineered that has dual Fc regions and can thereby have enhanced complement lysis and ADCC capabilities. (See Stevenson et al, Anti-Cancer Drug Design, 3:219-230 (1989)).

In certain embodiments, an antibody of the invention can comprise an Fc variant comprising an amino acid substitution which alters the antigen-independent effector functions of the antibody, in particular the circulating half-life of the antibody. Such antibodies exhibit either increased or decreased binding to FcRn when compared to antibodies lacking these substitutions, therefore, have an increased or decreased half-life in serum, respectively. Fc variants with improved affinity for FcRn can have longer serum half-lives, and such molecules have useful applications in methods of treating mammals where long half-life of the administered antibody is desired, e.g., to treat a chronic disease or disorder. In contrast, Fc variants with decreased FcRn binding affinity can have shorter halt-lives, and such molecules are also useful, for example, for administration to a mammal where a shortened circulation time can be advantageous, e.g., for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity can be desired include those applications in which localization to the brain, kidney, and/or liver is desired. In one embodiment, the Fc variant-containing antibodies can exhibit reduced transport across the epithelium of kidney glomeruli from the vasculature. In another embodiment, the Fc variant-containing antibodies can exhibit reduced transport across the blood brain barrier (BBB) from the brain, into the vascular space. In embodiments, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05 047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering).

In embodiments, mutations are introduced to the constant regions of the mAb such that the antibody dependent cell-mediated cytotoxicity (ADCC) activity of the mAb is altered. For example, the mutation is a LALA mutation in the CH2 domain. In one embodiment, the antibody (e.g., a human mAb, or a bispecific Ab) contains mutations on one scFv unit of the heterodimeric mAb, which reduces the ADCC activity. In another embodiment, the mAb contains mutations on both chains of the heterodimeric mAb, which completely ablates the ADCC activity. For example, the mutations introduced into one or both scFv units of the mAb are LALA mutations in the CH2 domain. These mAbs with variable ADCC activity can be optimized such that the mAbs exhibits maximal selective killing towards cells that express one antigen that is recognized by the mAb, however exhibits minimal killing towards the second antigen that is recognized by the mAb.

In embodiments, antibodies of the invention for use in the diagnostic and treatment methods described herein have a constant region, e.g., an IgG₁ or IgG₄ heavy chain constant region, which can be altered to reduce or eliminate glycosylation. For example, an antibody of the invention can also comprise an Fc variant comprising an amino acid substitution which alters the glycosylation of the antibody. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In one embodiment, the antibody comprises an Fc variant with an amino acid substitution at amino acid position 228 or 299 (EU numbering). In more particular embodiments, the antibody comprises an IgG1 or IgG4 constant region comprising an S228P and a T299A mutation (EU numbering).

Exemplary amino acid substitutions which confer reduced or altered glycosylation are described in PCT Publication No, WO05 018572, which is incorporated by reference herein in its entirety. In some embodiments, the antibodies of the invention, or fragments thereof, are modified to eliminate glycosylation. Such antibodies, or fragments thereof, can be referred to as “agly” antibodies, or fragments thereof, (e.g. “agly” antibodies). While not wishing to be bound by theory “agly” antibodies, or fragments thereof, can have an improved safety and stability profile in vivo. In yet other embodiments, antibodies of the invention, or fragments thereof, comprise an altered glycan. For example, the antibody can have a reduced number of fucose residues on an N-glycan at Asn297 of the Fc region, i.e., is afucosylated. In another embodiment, the antibody can have an altered number of sialic acid residues on the N-glycan at Asn297 of the Fc region.

The invention also is directed to immunoconjugates comprising an antibody conjugated to at least one additional active agent, such as a therapeutic agent, a labelling agent, or a radioactive isotope (i.e., a radioconjugate). In embodiments, the therapeutic agent comprises a cytotoxic agent such as a toxin (e.g., an enzymatically active toxin of bacterial, fungal, plant, or animal origin, or fragments thereof). In embodiments, the therapeutic agent comprises an siRNA, a radiolabel, a small molecule, cytokine, or the like. For example, the therapeutic agent can be an anti-cancer agent. As used herein, the term “anti-cancer agent” can refer to an agent effective in inhibiting, slowing or arresting the growth or metastasis of a cancerous cell or which exhibits a cytotoxic effect on a cancerous cell.

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Non-limiting examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See PCT Publication No. WO94 11026, and U.S. Pat. No. 5,736,137).

Those of ordinary skill in the art understand that a large variety of moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding, and complexation. In one embodiment, binding is, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al, Science 238:1098 (1987)). Non-limiting examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Non-limiting examples of useful linkers that can be used with the antibodies of the invention include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (-hydroxysulfo-succinimide: Pierce Chem. Co., Cat. #24510) conjugated to EDC.

The linkers described herein contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, for example, can be less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes.

Liposomes containing the antibody are prepared by methods known in the art, such as described in Epstein et al, Proc. Natl. Acad. Sci. USA, 82:3688 (1985); Hwang et al, Proc. Natl Acad. Sci. USA, 77:4030 (1980); and U.S. Pat. Nos. 4,485,045 and 4,544,545. Liposomes with enhanced circulation time are disclosed in U.S. Pat. No. 5,013,556.

Non-limiting examples of useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al, J. Biol. Chem., 257:286-288 (1982) via a disulfide-interchange reaction.

Aspects of the invention are also drawn to nucleic acids encoding antibodies, such as those described herein. The term “nucleic acid” can refer to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the reference sequence explicitly indicated.

In embodiments, the nucleic acid is a codon optimized nucleic acid. The phrase “codon optimized” can refer to changes in the codons of the polynucleotide encoding a protein to those used in a particular cell or organism such that the encoded protein is efficiently expressed in the cell or organism of interest.

### Pharmaceutical Compositions

Aspects of the invention further drawn to pharmaceutical compositions. Pharmaceutical combinations described herein can comprise one or more antibodies described herein, and/or those identified by screen methods described here.

A pharmaceutical composition of the invention can formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In embodiments, the composition is sterile and is fluid to the extent that easy syringeability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Embodiments can include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. For example, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

The compounds can also be prepared in the form of suppositories (e.g., with conventional suppository bases such as cocoa butter and other glycerides) or retention enemas for rectal delivery.

In embodiments, the pharmaceutical composition can comprise a pharmaceutically acceptable carrier, excipient, or diluent. The term “pharmaceutically acceptable carrier” can include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Non-limiting examples of such carriers or diluents include water, saline, ringer's solutions, dextrose solution, and 5% human serum albumin. Liposomes and non-aqueous vehicles such as fixed oils can also be used. The use of such media and agents for pharmaceutically active substances is well known in the art. Except insofar as any conventional media or agent is incompatible with the active compound, use thereof in the compositions can be useful. Supplementary active compounds can also be incorporated into the compositions.

In embodiments, carriers can protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form as used herein can refer to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

A specific dosage and treatment regimen for any particular patient will depend upon a variety of factors, such as the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

As used herein, the terms “effective amount” and “dose effective” can refer to an amount sufficient to achieve a result or effect on an undesired condition. For example, a “therapeutically effective amount” can refer to an amount sufficient to achieve a therapeutic result or effect on an undesirable condition, but insufficient to cause an adverse side effect. The specific therapeutically effective dose level for any particular patient will depend upon a variety of factors including the condition being treated and the severity of the condition; the specific ingredients used; the age, weight, general health, sex, and diet of the patient; the time of administration; the route of administration; the rate of excretion of the particular compound used; the duration of the treatment; drugs used in combination or concomitantly with the specific compound employed and similar factors well known in the medical arts. For example, one skilled in the art will start doses of the compound at levels below those required to achieve the desired therapeutic effect and gradually increase the dose until the desired effect is achieved. If desired, the effective daily dose can be divided into multiple doses for administration purposes. Thus, a single dose composition can contain such amounts or submultiples thereof to make up the daily dose. In the case of contraindications, the dosage may be adjusted by the individual physician. The dosage may vary, and may be administered once or multiple times daily for one or more days. Guidelines for appropriate dosing of a given class of pharmaceutical products can be found in the literature. In further various aspects, the formulation can be administered in a “prophylactically effective amount”; i.e., an amount effective to prevent a disease or disorder.

As noted herein, this can be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. The dosage administered to a subject (e.g., a patient) of the antibodies described herein can comprise about 0.1 mg/kg to about 100 mg/kg of the patient's body weight, between about 0.1 mg/kg and about 20 mg/kg of the patient's body weight, or about 1 mg/kg to about 10 mg/kg of the patient's body weight. Human antibodies have a longer half-life within the human body than antibodies from other species due to the immune response to the foreign polypeptides. Thus, lower dosages of human antibodies and less frequent administration can be useful. Further, the dosage and frequency of administration of antibodies of the disclosure may be reduced by enhancing uptake and tissue penetration (e.g., into the brain) of the antibodies by modifications such as, for example, lipidation. Common ranges for therapeutically effective dosing of an antibody or antibody fragment of the invention can be, by way of nonlimiting example, from about 0.1 mg/kg body weight to about 50 mg/kg body weight. Common dosing frequencies can range, for example, from twice daily to once a week.

Where antibody fragments are used, the smallest inhibitory fragment that specifically binds to the binding domain of the target protein can be useful. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90:7889-7893 (1993)). The formulation can also contain more than one active compound as necessary for the particular indication being treated, such as those with complementary activities that do not adversely affect each other. Alternatively, or in addition, the composition can comprise an agent that enhances its function, such as, for example, a cytotoxic agent, cytokine (e.g. IL-15), chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration can be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid allow release of molecules for over 100 days, certain hydrogels release proteins for shorter time periods.

Aspects of the invention are further drawn to combination compositions. For example, the phrase “combination composition” can refer to a composition which comprises a mixture of at least two different active compounds. For example, combination compositions can comprise one or more antibodies, such as an antibody described herein, and at least one additional active agent. The at least one additional active agent can be, for example, a toxin, a radiolabel, a siRNA, a small molecule, or a cytokine.

### Methods of Treatment

Aspects of the invention are also drawn towards methods of treating a subject afflicted with a disease or condition. In embodiments, the disease comprises cancer.

The terms “treat” or “treatment” can refer to both therapeutic treatment and prophylactic or preventative measures, wherein the object is to prevent or slow down (lessen) an undesired physiological change or disorder, such as the progression of cancer. Beneficial or desired clinical results can include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment include those already with the condition or disorder as well as those prone to have the condition or disorder or those in which the condition or disorder is to be prevented.

The invention provides for both prophylactic and therapeutic methods of treating a subject at risk of (or susceptible to) a cancer, or other cell proliferation-related diseases or disorders. Subjects at risk for cancer or cell proliferation-related diseases or disorders can include patients who have a family history of cancer or a subject exposed to a known or suspected cancer-causing agent. Administration of an anti-cancer agent can occur prior to the manifestation of cancer such that the disease is prevented or, alternatively, delayed in its progression.

In embodiments, the methods are used to treat, prevent or alleviate a symptom of cancer. In an embodiment, the methods are used to treat, prevent or alleviate a symptom of a solid tumor. Non-limiting examples of other tumors that can be treated by embodiments herein comprise lung cancer, ovarian cancer, prostate cancer, colon cancer, cervical cancer, brain cancer, thyroid cancer, skin cancer, liver cancer, pancreatic cancer or stomach cancer, neuroblastoma, rhabdomyosarcoma. Additionally, the methods of the invention can be used to treat hematologic cancers such as leukemia and lymphoma. Alternatively, the methods can be used to treat, prevent or alleviate a symptom of a cancer that has metastasized. For example, the cancer can be ovarian cancer or neuroblastoma.

In another aspect, tumor cell growth is inhibited by contacting a cell with an antibody of the invention. The cell can be any cell that expresses the target antigen.

In embodiments, the cancer expresses (or is characterized by the presence of) at least one biomarker. Non-limiting examples of such biomarkers comprise BCAM, CADM1, Her2, EPHA2, ITGA3, ITGA6, ICAM1, MME, ANPEP, or ENG.

The term “subject” or “patient” can refer to any organism to which aspects of the invention can be administered, e.g., for experimental, diagnostic, prophylactic, research and/or therapeutic purposes. For example, subjects to which compounds of the present disclosure can be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

In embodiments, a subject comprises a mammal, such as a human or vertebrate animal. Examples of such include but are not limited to a dog, cat, horse, cow, pig, sheep, goat, chicken, primate, e.g., monkey, fish (aquaculture species), e.g. salmon, rat, and mouse. A human comprises a preterm neonate, an infant, a child, an adolescent, an adult, or an elderly individual.

Although aspects of the invention as described herein relate to human cell proliferative disorders, aspects of the invention are also applicable to other nonhuman vertebrates. Aspects of the invention are applicable for veterinary use, such as with domestic animals. Aspects will vary according to the type of use and mode of administration, as well as the particularized requirements of individual subjects.

In embodiments, methods can comprise administering to a subject a therapeutically effective amount of a composition, such as a composition comprising a monoclonal antibody described herein or identified herein. The terms “administering” and “administration” can refer to any method of providing a pharmaceutical composition to a subject. Such methods are well known to those skilled in the art and include, but are not limited to, oral administration, transdermal administration, inhalation administration, nasal administration, topical administration, intravaginal administration, ocular administration, intra-aural administration, intracerebral administration, rectal administration, sublingual administration, buccal administration, intraurethral administration, and parenteral administration, including injectable, such as intravenous administration, intraarterial administration, intramuscular administration, and subcutaneous administration. Administration can be continuous or intermittent. In various aspects, the composition can be administered therapeutically; i.e., for treating an existing disease or disorder. In further various aspects, the composition can be administered prophylactically; i.e., for the prevention of a disease or disorder.

Aspects of the invention are also drawn to methods for diagnosing a subject with a condition or disease. The term “diagnosing” can refer to classifying a pathology (e.g., a disease, disorder, syndrome, medical condition and/or a symptom thereof), determining a severity of the pathology, monitoring the progression of a pathology, forecasting an outcome of the pathology and/or prospects of recovery (e.g., prognosis).

An antibody according to the invention can be used as an agent for detecting the presence of a biomarker (or a protein fragment thereof) in a biological sample. For example, an embodiment can comprise the the detection of cancer, cancer relapse or cancer recurrence. In embodiments, detection can comprise early detection, such as prior to radiographic scans. For example, the antibody can contain a detectable label. Antibodies can be polyclonal, monoclonal, or a fragment. An intact antibody, or a fragment thereof (e.g., Fab, scFv, or F (ab) 2) can be used. The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA includes Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations.

Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Antibodies described herein (or a fragment thereof) can be used in methods known within the art relating to the localization and/or quantitation of a biomarker (e.g., for use in measuring levels of the biomarker within appropriate physiological samples, for use in diagnostic methods, for use in imaging the protein, and the like). In a given embodiment, antibodies specific to a biomarker, or derivative, fragment, analog or homolog thereof, that contain the antibody derived antigen binding domain, are utilized as pharmacologically active compounds (referred to herein as “therapeutics”).

An antibody of the invention can be used to isolate an target-specific polypeptide by standard techniques, such as immunoaffinity, chromatography or immunoprecipitation. Antibodies described herein (or a fragment thereof) can be used diagnostically to monitor protein levels in tissue as part of a clinical testing procedure, e.g., to, for example, determine the efficacy of a given treatment regimen.

Detection can be facilitated by coupling (i.e., physically linking) the antibody to a detectable substance. Examples of detectable substances include, but are not limited to, various enzymes, prosthetic groups, fluorescent materials, luminescent materials, bioluminescent materials, and radioactive materials. Non-limiting examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S, 32P or 3H.

In embodiments, methods can comprise contacting a sample with a monoclonal antibody described herein or identified herein. The term “contacting” can refer to bringing a monoclonal antibody, sample, cells, target receptors, or other biological entities together such that the monoclonal antibodies can bind to a target (e.g., receptor, cell, etc.) For example, contacting the sample can determined with approaches known in the art, such as immunohistochemical approaches (e.g., immunoprecipitation, immunofluorescence, western blot, ELISA, and the like).

In embodiments, the sample can be obtained from or isolated from a subject. For example, the term “sample” can refer to a sample of fluid or tissue derived from a subject. Non-limiting examples of samples comprise whole blood, a blood component, a body fluid (e.g., pleural fluid, peritoneal fluid, CSF, or urine), a biopsy, a tissue (e.g., brain tissue or nervous system tissue), serum or one or more cells (including but not limited to those in an in vitro culture). As described herein, the sample can be a normal sample (such as a non-cancer sample), or the sample can be a non-normal sample (such as a cancerous sample)

The methods described herein can involve obtaining a biological sample from the subject. As used herein, the phrase “obtaining a biological sample” can refer to any process for directly or indirectly acquiring a biological sample from a subject. Methods of obtaining samples are known in the art. For example, a biological sample can be obtained (e.g., at a point-of-care facility, such as a physician's office, a hospital, laboratory facility) by procuring a tissue or fluid sample (e.g., blood draw, marrow sample, spinal tap) from a subject. Alternatively, a biological sample can be obtained by receiving the biological sample (e.g., at a laboratory facility) from one or more persons who procured the sample directly from the subject. The biological sample can be, for example, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

Embodiments can further comprise detecting the presence or absence of an antibody-antigen complex, wherein the presence of an antibody-antigen complex indicates the presence of cancer in the subject. The term “detecting” can refer to obtaining at least one item of information from a sample, such as the presence and/or concentration of a molecule in the sample.

The phrase “antibody-antigen complex” can refer to the complex formed by an antibody that is specifically bound to an epitope on an antigen.

Embodiments also comprise administering to a subject an anticancer agent, thereby treating cancer in the subject. For example, embodiments can comprise administering to a subject an anti-cancer agent if an antibody-antigen complex is detected.

Anti-cancer agents can include, but are not limited to, those described herein. In embodiments, the anti-cancer agent can be one or more antibodies as described herein or identified with methods described herein.

Compositions of the invention as described herein can also be administered in combination with a chemotherapeutic agent. Chemotherapeutic agents that can be administered with the compositions described herein include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In embodiments, the compositions of the invention as described herein can be administered in combination with cytokines. Cytokines that may be administered with the compositions include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions described herein can be administered in combination with other therapeutic or prophylactic regimens, such as, for example, radiation therapy.

In some embodiments, the compositions described herein can be administered in combination with other immunotherapeutic agents. Non-limiting examples of immunotherapeutic agents include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8.

### Diagnostic Assays

Antibodies as described herein can be used diagnostically to, for example, monitor the development or progression of a disease, such as cancer, as part of a clinical testing procedure to, e.g., determine the efficacy of a given treatment and/or prevention regimen.

In some aspects, for diagnostic purposes, the antibody of the invention is linked to a detectable moiety, for example, so as to provide a method for detecting a cancer cell in a subject at risk of or suffering from a cancer.

The detectable moieties can be conjugated directly to the antibodies or fragments, or indirectly by using, for example, a fluorescent secondary antibody. Direct conjugation can be accomplished by standard chemical coupling of, for example, a fluorophore to the antibody or antibody fragment, or through genetic engineering. Chimeras, or fusion proteins can be constructed which contain an antibody or antibody fragment coupled to a fluorescent or bioluminescent protein. For example, Casadei, et al, (Proc Natl Acad Sci USA. 1990 March; 87 (6): 2047-51) describe a method of making a vector construct that can express a fusion protein of aequorin and an antibody gene in mammalian cells.

As used herein, the term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject (such as a biopsy), as well as tissues, cells and fluids present within a subject. That is, the detection method of the invention can be used to detect cells that express a biomarker in a biological sample in vitro as well as in vivo. For example, in vitro techniques for detection of the biomarker include enzyme linked immunosorbent assays Western (ELISAs), blots, immunoprecipitations, and immunofluorescence. Furthermore, in vivo techniques for detection of the biomarker include introducing into a subject a labeled antibody as described herein. For example, the antibody can be labeled with a radioactive marker whose presence and location in a subject can be detected by standard imaging techniques.

In the case of “targeted” conjugates, that is, conjugates which contain a targeting moiety—a molecule or feature designed to localize the conjugate within a subject or animal at a particular site or sites, localization can refer to a state when an equilibrium between bound, “localized”, and unbound, “free” entities within a subject has been essentially achieved. The rate at which such equilibrium is achieved depends upon the route of administration. For example, a conjugate administered by intravenous injection can achieve localization within minutes of injection. On the other hand, a conjugate administered orally can take hours to achieve localization. Alternatively, localization can refer to the location of the entity within the subject or animal at selected time periods after the entity is administered. By way of another example, localization is achieved when an moiety becomes distributed following administration.

A reasonable estimate of the time to achieve localization can be made by one skilled in the art. Furthermore, the state of localization as a function of time can be followed by imaging the detectable moiety (e.g., a light-emitting conjugate) according to the methods of the invention, such as with a photodetector device. The “photodetector device” used should have a high enough sensitivity to allow for the imaging of faint light from within a mammal in a reasonable amount of time, and to use the signal from such a device to construct an image.

In cases where use of light-generating moieties which are extremely bright, and/or to detect light-generating fusion proteins localized near the surface of the subject or animal being imaged, a pair of “night-vision” goggles or a standard high-sensitivity video camera, such as a Silicon Intensified Tube (SIT) camera (e.g., from Hammamatsu Photonic Systems, Bridgewater, N.J.), can be used. However, a more sensitive method of light detection can be useful.

In extremely low light levels the photon flux per unit area becomes so low that the scene being imaged no longer appears continuous. Instead, it is represented by individual photons which are both temporally and spatially distinct form one another. Viewed on a monitor, such an image appears as scintillating points of light, each representing a single detected photon. By accumulating these detected photons in a digital image processor over time, an image can be acquired and constructed. In contrast to conventional cameras where the signal at each image point is assigned an intensity value, in photon counting imaging the amplitude of the signal carries no significance. The objective is to detect the presence of a signal (photon) and to count the occurrence of the signal with respect to its position over time.

At least two types of photodetector devices, described below, can detect individual photons and generate a signal which can be analyzed by an image processor. Reduced-Noise Photodetection devices achieve sensitivity by reducing the background noise in the photon detector, as opposed to amplifying the photon signal. Noise is reduced primarily by cooling the detector array. The devices include charge coupled device (CCD) cameras referred to as “backthinned”, cooled CCD cameras. In the more sensitive instruments, the cooling is achieved using, for example, liquid nitrogen, which brings the temperature of the CCD array to approximately −120° C. “Backthinned” refers to an ultra-thin backplate that reduces the path length that a photon follows to be detected, thereby increasing the quantum efficiency. A particularly sensitive backthinned cryogenic CCD camera is the “TECH 512”, a series 200 camera available from Photometries, Ltd. (Tucson, Ariz.).

“Photon amplification devices” amplify photons before they hit the detection screen. This class includes CCD cameras with intensifiers, such as microchannel intensifiers. A microchannel intensifier contains a metal array of channels perpendicular to and co-extensive with the detection screen of the camera. The microchannel array is placed between the sample, subject, or animal to be imaged, and the camera. Most of the photons entering the channels of the array contact a side of a channel before exiting. A voltage applied across the array results in the release of many electrons from each photon collision. The electrons from such a collision exit their channel of origin in a “shotgun” pattern, and are detected by the camera.

Even greater sensitivity can be achieved by placing intensifying microchannel arrays in series, so that electrons generated in the first stage in turn result in an amplified signal of electrons at the second stage. Increases in sensitivity, however, are achieved at the expense of spatial resolution, which decreases with each additional stage of amplification. An exemplary microchannel intensifier-based single-photon detection device is the C2400 series, available from Hamamatsu.

Image processors process signals generated by photodetector devices which count photons in order to construct an image which can be, for example, displayed on a monitor or printed on a video printer. Such image processors can be sold as part of systems which include the sensitive photon-counting cameras described above, and accordingly, are available from the same sources. The image processors can be connected to a personal computer, such as an IBM-compatible PC or an Apple Macintosh (Apple Computer, Cupertino, Calif), which may or may not be included as part of a purchased imaging system. Once the images are in the form of digital files, they can be manipulated by a variety of image processing programs (such as “ADOBE PHOTOSHOP”, Adobe Systems, Adobe Systems, Mt. View, Calif.) and printed.

In an embodiment, the biological sample contains protein molecules from the test subject. One exemplary biological sample is a peripheral blood leukocyte sample isolated by conventional means from a subject.

The invention also encompasses kits for detecting the presence of a biomarker or a cell expressing a biomarker in a biological sample. For example, the kit can comprise: a labeled compound or agent that can detect a cancer or tumor cell in a biological sample; means for determining the amount of a biomarker in the sample; and means for comparing the amount of a biomarker in the sample with a standard. The standard is, in some embodiments, a non-cancer cell or cell extract thereof. The compound or agent can be packaged in a suitable container. The kit can further comprise instructions for using the kit to detect cancer in a sample.

### Nucleic Acid-Based Expression Systems

Monoclonal antibodies of the present invention can be expressed from an expression vector. Recombinant techniques to generate such expression vectors are well known in the art.

The term “vector” can refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” can refer to any type of genetic construct comprising a nucleic acid coding for an RNA that can be transcribed. In cases, RNA molecules are then translated into a protein, polypeptide, or peptide. In other cases, these sequences are not translated, for example, in the production of antisense molecules or ribozymes. Expression vectors can contain a variety of “control sequences,” which can refer to nucleic acid sequences necessary for the transcription and translation of an operably linked coding sequence in a particular host cell. In addition to control sequences that govern transcription and translation, vectors and expression vectors may contain nucleic acid sequences that serve other functions as well and are described herein.

A “promoter” can refer to a control sequence that is a region of a nucleic acid sequence at which initiation and rate of transcription are controlled. It can contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter can comprise a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. These can be located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which can refer to a cis-acting regulatory sequence involved in the transcriptional activation of a nucleic acid sequence.

A promoter can be one naturally associated with a nucleic acid sequence, as may be obtained by isolating the 5 prime′ non-coding sequences located upstream of the coding segment and/or exon. Such a promoter can be referred to as “endogenous.” Similarly, an enhancer can be one naturally associated with a nucleic acid sequence, located either downstream or upstream of that sequence. Alternatively, advantages will be gained by positioning the coding nucleic acid segment under the control of a recombinant or heterologous promoter, which can refer to a promoter that is not normally associated with a nucleic acid sequence in its natural environment. A recombinant or heterologous enhancer can also refer to an enhancer not normally associated with a nucleic acid sequence in its natural environment. Such promoters or enhancers can include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR.TM., in connection with the compositions disclosed herein (see U.S. Pat. Nos. 4,683,202 and 5,928,906, each incorporated herein by reference). Furthermore, the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the organelle, cell type, tissue, organ, or organism chosen for expression. Those of skill in the art of molecular biology know the use of promoters, enhancers, and cell type combinations for protein expression, (see, for example Sambrook et al. 1989, incorporated herein by reference). The promoters employed can be constitutive, tissue-specific, inducible, and/or useful under the appropriate conditions to direct high level expression of the introduced DNA segment, such as is advantageous in the large-scale production of recombinant proteins and/or peptides. The promoter can be heterologous or endogenous.

Additionally, any promoter/enhancer combination could also be used to drive expression. Use of a T3, T7 or SP6 cytoplasmic expression system is another embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art.

A specific initiation signal also can be required for efficient translation of coding sequences. These signals include the ATG initiation codon or adjacent sequences. Exogenous translational control signals, including the ATG initiation codon, may need to be provided. One of ordinary skill in the art would readily be able to determine this and provide the necessary signals.

In embodiments of the invention, the use of internal ribosome entry sites (IRES) elements are used to create multigene, or polycistronic, messages, and these can be used in the invention.

Vectors can include a multiple cloning site (MCS), which is a nucleic acid region that contains multiple restriction enzyme sites, any of which can be used in conjunction with standard recombinant technology to digest the vector. “Restriction enzyme digestion” can refer to catalytic cleavage of a nucleic acid molecule with an enzyme that functions only at specific locations in a nucleic acid molecule. Many of these restriction enzymes are commercially available. Use of such enzymes is widely understood by those of skill in the art. A vector can be linearized or fragmented using a restriction enzyme that cuts within the MCS to allow for exogenous sequences to be ligated to the vector. “Ligation” can refer to the process of forming phosphodiester bonds between two nucleic acid fragments, which may or may not be contiguous with each other. Techniques involving restriction enzymes and ligation reactions are well known to those of skill in the art of recombinant technology.

Splicing sites, termination signals, origins of replication, and selectable markers can also be employed.

In embodiments, a plasmid vector can be used to transform a host cell. Plasmid vectors containing replicon and control sequences which are derived from species compatible with the host cell can be used in connection with these hosts. The vector ordinarily carries a replication site, as well as marking sequences which can provide phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM.TM. 11 can be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with galactosidase, ubiquitin, and the like.

Bacterial host cells, for example, E. coli, comprising the expression vector, are grown in any of a number of suitable media, for example, LB. The expression of the recombinant protein in certain vectors can be induced, as would be understood by those of skill in the art, by contacting a host cell with an agent specific for certain promoters, e.g., by adding IPTG to the media or by switching incubation to a higher temperature. After culturing the bacteria for a further period, for example, between 2 and 24 h, the cells are collected by centrifugation and washed to remove residual media.

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the invention can be a viral vector that encodes one or more monoclonal antibodies of the invention. Non-limiting examples of virus vectors that may be used to deliver a nucleic acid of the present invention are described herein.

A method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

The nucleic acid can be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the cells of the invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; Mclaughlin et al., 1988). Details describing the generation and use of rAAV vectors are described in U.S. Pat. Nos. 5,139,941 and 4,797,368, each incorporated herein by reference.

Retroviruses are useful as delivery vectors because of their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid (e.g., one encoding the desired sequence) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors can infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors can infect non-dividing cells and can be used for both in vivo and ex vivo gene transfer and expression of nucleic acid sequences. For example, recombinant lentivirus can infect a non-dividing cell wherein a suitable host cell is transfected with two or more vectors carrying the packaging functions, namely gag, pol and env, as well as rev and tat is described in U.S. Pat. No. 5,994,136, incorporated herein by reference. One can target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

Other viral vectors can be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus can be employed. They offer several attractive features for various mammalian cells (Friedmann, 1989; Ridgeway, 1988; Baichwal and Sugden, 1986; Coupar et al., 1988; Horwich et al., 1990).

In embodiments, a nucleic acid to be delivered can be housed within an infective virus that has been engineered to express a specific binding ligand. The virus particle will thus bind specifically to the cognate receptors of the target cell and deliver the contents to the cell. An approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

Methods for nucleic acid delivery for transfection or transformation of cells are known to one of ordinary skill in the art. Such methods include, but are not limited to, direct delivery of DNA such as by ex vivo transfection, by injection, and so forth. Through the application of techniques known in the art, cells may be stably or transiently transformed.

### Ex Vivo Transformation

Methods for transfecting eukaryotic cells and tissues removed from an organism in an ex vivo setting are known to those of skill in the art. Thus, cells or tissues can be removed and transfected ex vivo using nucleic acids of the invention. In aspects, the transplanted cells or tissues can be placed into an organism. In embodiments, a nucleic acid is expressed in the transplanted cells.

### Chimeric Antigen Receptor (CAR) T-Cell Therapies

Cellular therapies, such as chimeric antigen receptor (CAR) T-cell therapies, are also provided herein. CAR T-cell therapies redirect a patient's T-cells to kill tumor cells by the exogenous expression of a CAR on a T-cell, for example. A CAR can be a membrane spanning fusion protein that links the antigen recognition domain of an antibody to the intracellular signaling domains of the T-cell receptor and co-receptor. A suitable cell can be used, for example, that can secrete an antibody of the present invention (or alternatively engineered to express an antibody as described herein to be secreted). The antibody “payloads” to be secreted, can be, for example, minibodies, VHH, scFvs, IgG molecules, bispecific fusion molecules, and other antibody fragments as described herein. Upon contact or engineering, the cell described herein can then be introduced to a patient in need of a treatment by infusion therapies known to one of skill in the art. The patient may have a cancer, such as ovarian cancer. The cell (e.g., a T cell) can be, for instance, T lymphocyte, a CD4+ T cell, a CD8+ T cell, or the combination thereof, without limitation.

Exemplary CARs and CAR factories useful in aspects of the invention include those disclosed in, for example, PCT/US2015/067225 and PCT/US2019/022272, each of which are hereby incorporated by reference in their entireties. In one embodiment, the antibodies discussed herein can be used in the construction of the payload for a CAR-T cell. For example, in one embodiment, the antibodies discussed herein can be used for the targeting of the CARS (i.e., as the targeting moiety). In another embodiment, the antibodies discussed herein can be used as the targeting moiety, and a different antibody that targets a different epitope can be used as the payload. In another embodiment, the payload can be an immunomodulatory antibody payload.

### Methods to Identify Antibodies

Aspects of the invention are also directed towards compositions, methods, and kits to identify one or more antibody candidates.

In embodiments, the method comprises subjecting an input library to affinity selection to produce an output library.

The term “input library” can refer to can refer to a group of or mixture of molecules prior to undergoing one or more selection steps. For example, an “antibody library” can be a collection of various antibodies and/or antibody genes have different sequences.

In embodiments, the input library can be a “display library”. A “display library” can refer to a population of display vehicles, often, but not always, cells or viruses. The phrase “display library” includes a collection of nucletotide sequences within clones or a genetically diverse collection of polypeptides displayed on replicable display packages that can select or screen to provide an individual polypeptide or mixed population of polypeptides.

The “display vehicle” provides both the nucleic acid encoding a peptide as well as the peptide, such that the peptide is available for binding to a target molecule and further, provides a link between the peptide and the nucleic acid sequence that encodes the peptide. Various “display libraries” are known to those of skill in the art and include libraries such as phage, phagemids, yeast and other eukaryotic cells, bacterial display libraries, plasmid display libraries as well as in vitro libraries that do not require cells, for example ribosome display libraries or mRNA display libraries, where a physical linkage occurs between the mRNA or cDNA nucleic acid, and the protein encoded by the mRNA or cDNA. As used herein, the term “display” can refer to a biological entity, or “display host”, of which genetically engineered proteins are placed on the surface so that the properties of entities that bind to them can be analyzed. Non-limiting examples of an input library comprises phage display, mammalian display, yeast display, bacterial display, ribosome display, or B-cells.

In embodiments, the input library can be phage display. The phrase “phage display” can refer to exogenous proteins expressed on the surface of bacteriophages or phagemid particles. For example, a phage display can be used as a technique for to study protein-protein, protein-peptide, or protein-DNA interactions using bacteriophages to connect proteins with the genetic materials which encode them. For example, the phage display can be VHH phage display, which is a phage display host which displays a VHH.

In embodiments, the phage display can be VHH phage display. A VHH phage display library can refer to a display library of antibody fragments comprising single variable domain on a heavy chain (VHH), VHH antibodies can also be referred to as nanobodies.

Embodments can comprise obtaining an input library. For example, input libraries can be obtained from immunization of a donor or a naïve library. For example, the donor can comprise a human, a horse, a llama, a cow, a pig, a dog, a cat, a mouse, a rat, or a suitable animal.

The term “affinity selection” can refer to a technique which relies on interactions or bindings between a candidate, such as a candidate antibody, and targets, such as an antigen. In embodiments, affinity selection comprises biopanning. In embodiments, subjecting an input library to affinity selection produces an output library. The term “output library” can refer to the library, such as a library of displays, that are the product of an affinity selection process.

In embodiments, the affinity selection comprises at least one panning step. The term “panning” can refer to a process where the input library is exposed to and/or screened against proteins, cells, or other targets to detect interactions. In embodiments, a “target” can refer to an object or entity whose detection or modulation is desired. A target can be known at the time of panning, or unknown at the time of panning. For example, the target can refer to a therapeutic target. For example, a therapeutic target in ovarian cancer can comprise BCAM.

For example, the affinity selection can comprise one panning step, two panning steps, three panning steps, four panning steps, five panning steps, six panning steps, seven panning steps, eight panning steps, or more than eight panning steps.

In embodiments, the affinity selection can comprise a panning step with a sample positive for a biomarker and/or a sample negative for a biomarker. The term “biomarker” can refer to a measurable indicator of a biological state. For example, the biological state can be the presence or the absence of a disease or condition. For example, the biomarker can be objectively measured and can be a sign of a normal or abnormal process, or a condition or a disease.

For example, a sample negative for a biomarker and/or the sample positive for a biomarker comprise a diseased state, a non-diseased state, and/or a combination thereof. As used herein, the term “diseased” can refer to a subject or an object affected with or as if with a disease. As used herein, the term “diseased” can refer to a subject or object lacking health.

The terms “sample”, “test sample”, “biological sample” can refer to a fluid sample containing or suspected of containing one or more analytes of interest. The sample can be from any suitable source. In embodiments, the sample can include a liquid, a flowable particulate solid, or a fluid suspension of solid particles. In embodiments, the sample can be processed prior to analysis as described herein. For example, the sample can be separated or purified from its source prior to analysis (eg, a cell, cell line, population of cells, an organoid), but in embodiments, the raw sample containing the analyte can be assayed directly. The source of the analyte molecule can be synthetic (eg, produced in a laboratory), environment (eg, air, soil, fluid sample, eg, water supply), animal (eg, mammal), plant, or any combination thereof obtain. In certain instances, the source of the analyte is human body material (eg, body fluid, blood, serum, plasma, urine, saliva, sweat, sputum, semen, mucus, tears, lymph, amniotic fluid, interstitial fluid, lungs) Lavage, cerebrospinal fluid, feces, tissue, organ, or the like). Tissues can include, but are not limited to, skeletal muscle tissue, liver tissue, lung tissue, kidney tissue, myocardial tissue, brain tissue, bone marrow, cervical tissue, skin, and the like. The sample can be a liquid sample or a liquid extract of a solid sample. In certain cases, the source of the sample can be an organ or tissue (such as a biopsy sample), which can be solubilized by tissue disruption/cytolysis.

In embodiments, the sample can be a normal sample or a non-normal sample. The phrase “normal sample” can refer to a sample which does not contain a target and/or presents typically. For example, the target can comprise a disease biomarker. For example, the normal sample can comprise a healthy cell. The phrase “non-normal sample” can refer to to a sample which contains a target and/or presents atypically. For example, a non-normal sample can be a cancer sample. It will be clear to the skilled artisan that the cancer sample can comprise a sample of any cancer tissue or cells, including but not limited to a solid cancer or a liquid cancer (i.e., blood cancer).

The term “solid cancer” can refer to abnormal cellular growths in solid organs. Non-limiting examples of a solid cancer comprises ovarian cancer, breast cancer, brain cancer, prostate cancer, skin cancer, cervical cancer, gastric cancer, bladder cancer, liver cancer, lung cancer, kidney cancer, colon cancer, and oral cancer.

The phrase “ovarian cancer” can refer to a cancer that is located in and/or begins in the ovaries. Ovarian cancer comprises serous carcinoma, clear-cell carcinoma, mucinous ovarian cancer, or endometrial cancer.

In embodiments where the sample is a cell, cell line, or population of cells, the cell can be a mammalian cell. A mammalian cell can refer to any cell, cell line, or population thereof derived from any mammal (e.g. human, hamster, mouse, monkey, rat, pig, cow or rabbit). Non-limiting examples of mammalian cells include primary peripheral blood mononuclear cells (PBMC) and fibroblasts, for example. In embodiments, the cell can be a human cell.

In embodiments, the sample can comprise a cell line. The phrase “cell line” can refer to cells that are cultured in vitro, comprising primary cell lines, finite cells lines, continuous cell lines, and transformed cell lines. In embodiments, the cell line can be a cell culture selected for uniformity from a cell population which can be derived from a homogenous tissue source. Non-limiting examples of cell lines comprise KURAMOCHI, OVSAHO, OV8, ES2, OC314, RMUGS, or SKOV3, for example.

In embodiments, the sample can comprise one or more live cells, such as a population of live cells. The phrase “live cell” can refer to a cell in a state that the cell can proliferate and exhibits metabolic activity when it is cultured under culture conditions. The phrase “viable cell” can refer to a cell capable of living. As used herein, the terms “live cell” and “viable cell” can be used interchangeably.

For example, the affinity selection can comprise at least one panning step with a sample negative for a biomarker, at least one panning step with a sample positive for a biomarker, or both at least one panning step with a sample negative for a biomarker and at least one panning step with a sample positive for a biomarker.

As described herein, embodiments can comprise subjecting an input library to one or more affinity selection steps (i.e., panning steps) to produce an output library. In embodiments, the affinity selection steps can be completed sequentially. For example, the input library can be first subjected to panning with a sample negative for a biomarker, wherein the non-bound fraction from the first panning step is then subjected to a second panning with a sample positive for a biomarker, thereby producing an output library. In embodiments, the input library can be subjected to first panning with a sample negative for a biomarker, thereby producing a first output library, and then the first output library can be subjected to a second panning with a sample positive for a biomarker to produce a second output library. One or more additional panning steps (with either a positive sample and/or a negative sample) can further be performed, if necessary. The skilled artisan would recognize that the input library can alternatively be subjected to panning with a sample positive for a biomarker first, and the then first output library can be subjected to panning with a sample negative for a biomarker, thereby producing a second output library.

The output library can then be analyzed to identify one or more antibody candidates. The phrase “analyzing an output library” can refer to subjecting an output library to one or more analysis methods. Non-limiting examples of such analysis methods comprise sequencing (e.g., next generation sequencing), computational pre-processing (e.g., sequence fitting, sequence alignment, and sequence clustering), computational guided selection (e.g., differential analysis, phage enrichment analysis, selection based on predicted binding profiles), colony picking, and computational guided selection. In embodiments, phage enrichment analysis can comprise selection of clusters based on the number of positive samples with number of reads greater or less than a specified threshold, the number of negative samples with number of reads greater or less than a specified threshold, or any combination thereof. In embodiments, the selection based on binding profiles can comprise selection of clusters based on the exploratory analysis of positive samples and negative samples with number of reads greater or less than a specified threshold. In embodiments, the computational pre-processing can be performed using nucleic acid or amino acid sequences. In embodiments, the computation pre-processing can be performed using full length sequences or with shorter substances. For example, the the shorter substance can be CDRs. In embodiments, computational pre-processing can comprise sequence filtering, sequence alignment, and sequence clustering. In embodiments the sequence clustering can comprise gropuing sequences with equal length, similarity greater than a specified threshold, or any combination thereof. For example, the threshold can comprise 60% for sequences shorter than 10 amino acids and 70% for sequences 10 amino acids and longer.

In embodiments, sequencing can comprise sequencing nucleic acid sequences. In embodiments, the sequencing can further comprise read stitching prior to sequence alignment when paried-end sequencing is used. In embodiments, the sequencing can further comprise translating nucleic acid sequences to amino acid sequences before, after, or between any step. In embodiments, the sequencing can futher comprise extracting subunits of sequences to amino acid sequences before, after, or between any step. In embodiments, the subunits comprise antibody CDR1, CDR2, CDR3, FR1, FR2, FR3, FR4, or any combination thereof. In embodiments, the sequencing comprises sequence filtering and/or sequence pre-filtering. In embodiments, sequence pre-filtering comprises exclusion of sequences with low base calling quality. For example, sequence filtering can comprise exclusion of sequences with poor alignment to reference sequences, out-of-frame alignment to reference sequences, low similarity to reference sequences, missing conserved positions, or any combination thereof. In embodiments, similarity can be measured using subunits of sequences. For example, the subunits can comprise antibody CDR1, CDR2, CDR3, FR1, FR2, FR3, FR4, or any combination thereof. In embodiments, similarity can be measured using maximal position weight maxtrix (PWM) scoring. In embodiments, conserved positions can comprise cysteine at position 23, tryptophan at position 41, hydrophobic amino acid at position 89, and cysteine at position 104, or any combination thereof. In embodiments, the sequencing can futher comprise sequence trimming. For example, the sequence trimming can comprise adapter sequence clipping, low quality base trimming, fixed width cropping, or any combination thereof. In embodiments, sequence alignment can comprise alignment of sequences against reference sequences publically reported or internally validated sequences. In embodiments, the sequence alignment can be performed using subunits of sequences. For example, the subunits can comprise antibody CDR1, CDR2, CDR3, FR1, FR2, FR3, FR4, or any combination thereof.

One or more antibodies can be isolated from the output library. For example, the antibody can be one or more antibodies as described herein, such as a full-length antibody, a fusion protein, or an antibody fragment.

The phrase “isolate an antibody” can refer to any method which purifies an antibody or a group of antibodies based upon a specific characteristic. Non-limiting examples of methods that can isolate an antibody comprise physiochemical fractionation and antigen-specific purification. As used herein, the term “physiochemical fractionation” can refer to methods that separate antibodies based upon their size, charge, or chemical properties. For example, physiochemical fractionation can comprise size exclusion chromatography, ammonium sulfate precipitation, ion exchange chromatorgraphy, immobilized protein resins, and immobilized metal chelate chromatography. For example, the immobilized protein resin contains immobilized protein A. As used herein, the term “antigen-specifc purification” can refer to a method that uses antibody binding to a specific antigen to separate the from those which do not bind the antigen.

Embodiments can further comprise producing (i.e., synthesize, manufacture, isolate) the one or more antibody candidates. Steps to produce an antibody are known in the art, see for example Basic Methods in Antibody Production and Characterization, eds. Gary C. Howard and Delia R. Bethell, CRC Press, 2000, which include but are not limited to cloning and synthesizing, reformatting, and expressing the antibody.

Embodiments can also comprise one or more amplification steps. The phrase “amplification step” can refer to an exponential increase in a target nucleic acid. Non-limiting examples of methods of amplification include, but are not limited to PCR method (including RT-PCR method), NASBA (Nucleic Acid Sequence-Based Amplification) method, ICAN (Isothermal and Chimeric primer-initiated Amplification of Nucleic acids) method, LAMP (Loop-Mediated Isothermal Amplification) Method (including RT-LAMP method).

In embodiments, the binding specificity of the one or more antibody candidates can be validated. Non-limiting examples of such validation methods comprise an immunoassay, a live cell binding assay, high throughput cell line multiplexing through fluorescent barcoding, plate based binding assays, high content analysis, or any combination thereof.

For example, an “immunoassay” can refer to a method of detection of a specific antigen or a group of related or similar antigens through their ability to be recognized and bond by a specific antibody directed against them. Non-limiting examples of immunoassays comprise comprises flow cytometry (e.g., fluorescence-activated cell sorting (FACS)), enzyme-linked immunosorbent assay (ELISA), plate based fluorescence binding assays, high content analysis, immunohistochemistry/fluorescent imaging, western blotting.

Embodiments can further comprise identifying and/or validating the target of the antibody candidate. Methods of identification and/or validation will be known to the skilled artisan, non-limiting examples of which include antibody labeling, immunoprecipitation, antibody crosslinking, protein microarray, mass spectrometry (e.g., LC-MS/MS, MALDI-TOF MS, ESI, or label free analysis based on MS signal intensity), biotin transfer, or genetic approaches. For example, genetic approaches can comprise over expression library screens and genetic knockdown and/or knockout libraries. Without wishing to be bound by theory, the antibody can be expressed as a fusion protein to an enzyme that mediates labelling of proximity target proteins. For example, the proximity target proteins are secretases.

The term “antibody labeling” can refer to the attachment of an entity to an antibody. For example, the entity attached to the antibody can be used for detection, purification, and/or isolation purposes. For example, antibody labeling can comprise linking the antibody candidate with a label to produce a labelled antibody candidate; incubating the labeled antibody candidate with a population of cells, wherein the labeled antibody candidate binds to a target on the surface of the cells to produce an antibody-target conjugate; isolating the antibody-target conjugate from the population of cells (for example, bu cell lysis); and identifying and/or validating the target.

In embodiments, the antibody candidate can be linked to a label. The term “label” or “antibody label” can refer to an entity attached for the purposes of identifying, detecting, purifying, and/or isolating. Antibody labels will be known to the skilled artisan, and include a trifunctional crosslinker comprising biotin, a sulfhydryl group and a aldehyde-reactive aminooxy group linked by LC-SPDP or PEG4-SPDP, HRP, or a trifunctional crosslinker (TriCEPS).

In embodiments, the antibody candidate can be linked to a lable with a cleavable linker. The term “cleavable linker” can refer to a bioconjugation linker which can connect two or more molecules together and can be cleaved under certain conditions. Non-limiting examples of cleavable linkers include disulfide linkages, pyrophosphate diester linkages, and biotin linkages.

Aspects of the invention are also drawn to compositions and methods for identifying a target or antibody target. For example, the target can be a disease-specific target, a cancer-specific target, and/or a therapeutic target.

In embodiments, the method comprises embodiments described herein. For example, embodiments can comprise subjecting an input display library to affinity selection to produce an output library, wherein affinity selection comprises live cell panning; analyzing the output library to identify one or more antibody candidates; and identifying the target of the one or more antibody candidates, thereby identifying a target, an antibody candidate, or both.

For example, the term “target” or “antibody target” can refer to refer to an object or entity whose detection or modulation is desired. Non-limiting examples of antibody targets comprise HER2, EPHA2, ITGA3, ITGA6, BCAM, ICAM1, CADM1, MME, ANPEP, or ENG.

In embodiments, the target can comprise a disease-specific target, a cancer-specific target, and/or a therapeutic target. In embodiments, the terms target and biomarker can be used interchangeably. A “disease-specific target” or a “disease target” can refer to molecule (e.g., protein, nucleic acid, or otherwise) that is associated with any anatomical abnormality or impairment of the normal function of an organism (e.g. a human) or any of its parts. The disease can be caused by environmental factors, infective agents, genetic disease or any combination thereof and can include cancer. In embodiments, for example, the disease-specific target can be on the surface of a cell, such as a cancer cell.

A “cancer-specific target” or “cancer target” can be expressed or synthesized in cancer cells, tissues and/or tumors. For example, a cancer target can include, but are not limited to, enzymes and proteins (including peptides, for example) such as cell surface receptors; nucleic acids; lipids and phospholipids.

A “therapeutic target” can refer to any environment or molecule (such as a gene or a protein) that is instrumental to a disease process, though not necessarily directly involved, that can be targeted by a therapeutic agent to regulate that environment's or molecule's activity for therapeutic purposes.

Aspects described herein can be used for generating a cell surface map. “Mapping” can refer to a process of spatially determining a physical, electrical, electromagnetic, chemical, biochemical and/or thermal property of an object or surface. In embodiments, the surface can be the surface of a cell (e.g., cell surface mapping). Various biomolecules (e.g., sugars, complex sugars, receptors, transmembrane proteins, and the like) exist on the cell surface, and many are unique to cells. In embodiments, the cell surface map can comprise mapping of the surface of a cancer cell, or the mapping of the surface of a normal cell.

Aspects of the invention are also drawn towards methods for producing antibodies described herein. For example, methods for producing antibodies are known to the skilled artisan.

### Kits of the Invention

Any of the, antibodies, compositions or assays described herein can be comprised in a kit.

Some components of the kits can be packaged either in aqueous media or in lyophilized form. The container means of the kits can include at least one vial, test tube, flask, bottle, syringe or other container means, into which a component can be placed, and suitably aliquoted. Where there is more than one component in the kit, the kit also can contain a second, third or other additional container into which the additional components can be separately placed. However, various combinations of components can be comprised in a vial. The kits of the invention also can include a means for containing the components in close confinement for commercial sale. Such containers can include injection or blow molded plastic containers into which the desired vials are retained.

When the components of the kit are provided in one and/or more liquid solutions, the liquid solution is an aqueous solution, with a sterile aqueous solution being useful. In some cases, the container means can itself be a syringe, pipette, and/or other such like apparatus, from which the formulation can be applied to an infected area of the body, injected into an animal, and/or even applied to and/or mixed with the other components of the kit.

However, the components of the kit can be provided as dried powder(s). When reagents and/or components are provided as a dry powder, the powder can be reconstituted by the addition of a suitable solvent. For example, the solvent can also be provided in another container means. The kits can also comprise a second container means for containing a sterile, pharmaceutically acceptable buffer and/or another diluent.

In embodiments of the invention, antibodies that are to be used for antibody-based therapy are provided in a kit, and in some cases the antibodies can be the sole component of the kit. The kit can comprise reagents and materials to make the desired antibody. In embodiments, the reagents and materials include primers for amplifying desired sequences, nucleotides, suitable buffers or buffer reagents, salt, and so forth, and in some cases the reagents include vectors and/or DNA that encodes a CAR as described herein and/or regulatory elements therefor.

In embodiments, there are one or more apparatuses in the kit suitable for extracting one or more samples from an individual. The apparatus can be a syringe, scalpel, and so forth.

In embodiments of the invention, the kit, in addition to cell therapy embodiments, also includes a second cancer therapy, such as chemotherapy, hormone therapy, and/or immunotherapy, for example. The kit(s) can be tailored to a particular cancer for an individual and comprise respective second cancer therapies for the individual.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

PhASTdiscovery-A platform for the phenotypic antibody and simultaneous target discovery

Described herein is a high throughput platform for the simultaneous discovery of therapeutic antibodies and associated targets based on their phenotypic binding profiles. It is a multistep process that allows for the unbiased discovery of hundreds of antibody/target pairs selective to a cancer specific surface with limited or no binding to the surface of unrelated cell types in a single round of screening. As first step, an input library (in our case a VHH phage display library derived from alpacas that were immunized with plasma membrane protein, but any other antibody format can be used instead) is enriched for cancer specific binders, while depleted of binders to healthy cells. Output libraries of both, cancer specific and healthy cell lines, are characterized by NGS. Differential analysis of each sequence results in predictive binding profiles which is used as basis for selection. Candidate sequences are then expressed as Fc-fusion proteins and validated for their binding pattern in a high throughput flow cytometry based assay. The confirmed antibodies are then matched to their targets using a proteomic based protocol.

### Target Discovery:

The power of conventional gene/protein expression analysis which is used to identify new targets is limited in that it does not take into account the physiological state for surface proteins. Therefore, targets that are uniformly expressed but adopt cancer specific conformations or modifications or which surface exposure is regulated differentially will be missed in such analysis. By contrast, embodiments described herein are built on screening live cells, thus ensuring that we capture the true cancer specific surfaceome. In addition, conventional target discovery requires validation to ensure physiological relevance on cancer cells prior to initiating the antibody discovery process. Given our live cell and NGS based approach, we are unbiasedly selecting cancer specific antibody/target pairs in physiologically relevant conditions based on their binding phenotype, eliminating the need for extensive validation.

### Antibody Discovery:

Conventional antibody discovery is a time consuming, one target at a time approach. Our platform is based on pooled screening and thus allows the identification of hundreds of antibody/target pairs in a single round of screening, vastly accelerating the antibody discovery process.

### NGS Based Candidate Selection:

To eliminate non-specific binders, conventional phage display selection requires 4-6 rounds of selection introducing undesired antibody independent biases. In our platform the selection is based on NGS and differential analysis of antibody sequences in cancer cells versus healthy cells, allowing computational elimination of non-specific binders, reducing the number of required selection rounds to 1-2, reducing biases. In addition, our NGS based phenotypic selection pipeline allows the selection of follow up candidates based on predicted binding profiles, rather than pure sequence abundance which is the basis of selection in conventional methods. Clustering of highly homologous sequences ensures screening the same antibody only once, instead of 100-1000 times with classic colony-picking methods, further streamlining the process.

The platform allows for the rapid discovery of new cancer specific targets and therapeutic antibodies for cancer treatment. Depending on their targets and the properties of the discovered antibodies they can be used as scFvs, reformatted as T-cell engagers, used as antibody-drug conjugates, expressed on CAR-T cells etc. The associated identification of cancer specific targets and their expression profiles can inform on additional indications and combination therapies.

### Example 2

### Discovery of a Single Domain Antibody Leads to the Identification of BCAM as Therapeutic Target for High Grade Ovarian Cancer

Using our PhASTdiscovery platform we have discovered a number of single domain antibodies specific to a variety of targets within protein classes of receptor tyrosine kinases, adhesion/migration, proteases, and angiogenesis regulating proteins. As a representative example, we are highlighting a single domain antibody (6N2_22) that specifically binds to the extracellular domain of BCAM, a cell surface receptor for Laminin 5, that is involved in cell adhesion and migration. BCAM is highly overexpressed in a subset of ovarian cell lines, with enrichment in high-grade serous ovarian cancer cell lines. Analysis of Cancer Genome Atlas data revealed that BCAM is also highly overexpressed in a subset of patients with ovarian cancer. By contrast, BCAM expression is low in most normal tissues, with highest levels detected in thyroid gland and kidney. These expression characteristics make it an attractive target in ovarian cancer, in particular for high-grade serous carcinomas, the most common and aggressive subtype. The antibody, 6N2_22, binds recombinant BCAM with an affinity of about 4 nM, and an affinity of 7 nM on live cells, its binding is independent of BCAMs glycosylation status and its receptor occupancy. From a functional perspective, the antibody is able to potently induce antigen dependent cellular cytotoxicity (ADCC), the main mode of action of therapeutic antibodies.

High-grade serous carcinoma (HGSC) is the most common and lethal ovarian cancer subtype, with the vast majority of women diagnosed at an advanced stage of disease. The current standard treatment is surgical debulking combined with chemotherapy. While standard therapy induces an initial response, tumors ultimately recur, and 70% of patients die within 5 years of diagnosis. To achieve better outcomes, new therapeutic targets are needed. The discovery that BCAM is highly overexpressed in a subset of ovarian cancer patients makes it an attractive new therapeutic target. Given that our antibody shows high specificity, high affinity, and potent ADCC activity, it possesses some key characteristics required for the development of an therapeutic antibody targeting BCAM overexpressing tumors.

Our single domain antibodies can be developed into a therapeutic antibody in a variety of different formats and strategies. For example, it can be used as a single domain antibody (sdAb), within T-cell redirecting molecules, or in the context of targeted cell therapy approaches (eg.CAR-T) for the treatment of a subset of ovarian tumors. It could also be used for targeted radiotherapy. BCAM is also be highly expressed in KRAS mutant metastatic tumors as well as in a subset of prostate tumors. Hence our antibody could be an effective therapeutic against these cancers as well.

### Example 3

PhAST discovery: A Platform for the Rapid and Simultaneous Discovery of Cell Surface Targets and Therapeutic Antibodies

### Abstract

Although molecularly targeted antibody therapies have been used successfully in treatment of cancer, the identification of cancer specific targets has remained a bottleneck in development of new therapeutics. To overcome this challenge, we developed a high throughput platform for the simultaneous discovery of therapeutic antibodies and associated targets based on phenotypic binding profiles. This multistep process allows for the unbiased discovery of hundreds of antibody/target pairs selective for particular cancers through a single round of screening using genomic, mass spectrometry and flow cytometry. Applying this platform to ovarian cancer as a representative example, we identified a set of unique Her2 specific VHHs with biochemical and therapeutic properties similar to clinically used antibodies. Furthermore, we discovered an antibody specific for BCAM as a therapeutic target in high grade serous ovarian cancers (HGSOC).

### Introduction

Several therapeutic antibodies are approved for the treatment of specific cancers. Some of these antibodies target proteins essential for the malignant phenotype while others target proteins expressed primarily on tumor cells. More recently, antibodies that modulate immune activity have shown sometimes dramatic responses in subsets of patients. There are many efforts to identify new antigens for antibody-based therapies but these efforts require prior knowledge of targets or arduous validation schemes.

Since antibodies cannot naturally penetrate the cell membrane, antibody based therapeutics rely on their reactivity to cell surface proteins. Accordingly, currently available antibody therapeutics mostly target surface proteins that are involved in tumor growth (eg EGFR) or that are overexpressed in a cancer specific manner. One major challenge in the development of clinically effective biologics has been off-tumor cytotoxicity, mostly driven by on-target effects due to non-cancer specific expression of the target. Other challenges include immune-escape through target downregulation as well as target heterogeneity within the tumor. To overcome these obstacles the discovery of alternative cell/tumor type specific targets with high, and homogeneous expression is essential.

The target selection for the development of antibody directed therapies has so far been guided mostly by exploring expression databases for cancer or tissue specific cell surface markers, and by hypothesis-driven approaches including genome wide genetic screens exploring cancer vulnerabilities. While these strategies have led to the approval of several promising therapies, they have several important limitations as they lack information on the targets' phenotypic expression pattern. For example, the surface exposer of many receptors, transporters, and channels is tightly regulated and can vary widely depending on the microenvironmental context. Accordingly, the tumor microenvironment can alter the surface abundance of proteins without detectable differences in gene expression profiles. Proteins regulated in this manner are thus missed as highly tumor specific targets by conventional gene expression analysis. Similarly, many surface proteins have been shown to be posttranslationally modified, or to be expressed in cancer specific protein complexes which might affect their target conformation, rendering them cancer specific targets despite uniform expression.

Once a candidate target is identified, conventionally, target specific antibodies are generated using hybridoma technologies, B-cell cloning, or synthetic display approaches. Screening for specificity can be based on binding to purified recombinant proteins. Therefore, upon identification of some candidates, antibody specificity has to be validated rigorously in physiologically relevant settings before moving forward with antibody development. The process from target candidate nomination through antibody generation is a one target at a time approach, it is time consuming, labor intense, and expensive, without a guarantee that the discovered antibodies indeed possess the ability to bind to a native or cancer specific state of the target.

With these shortcomings in mind we here describe the development of a platform that selects antibodies based on desired cell surface binding patterns on live cells followed by identification of their targets. This approach allows the simultaneous discovery of hundreds of target-antibody pairs specific to the native or cancer specific state in a single round of screening. Applying this platform to ovarian cancer as a representative example we identified a set of Her2 specific VHH antibodies (nanobodies) with comparable properties to antibodies currently in clinical use. Furthermore we demonstrate that the platform can be used to identify highly potent antibodies against previously underappreciated targets in high grade ovarian cancer.

### Results

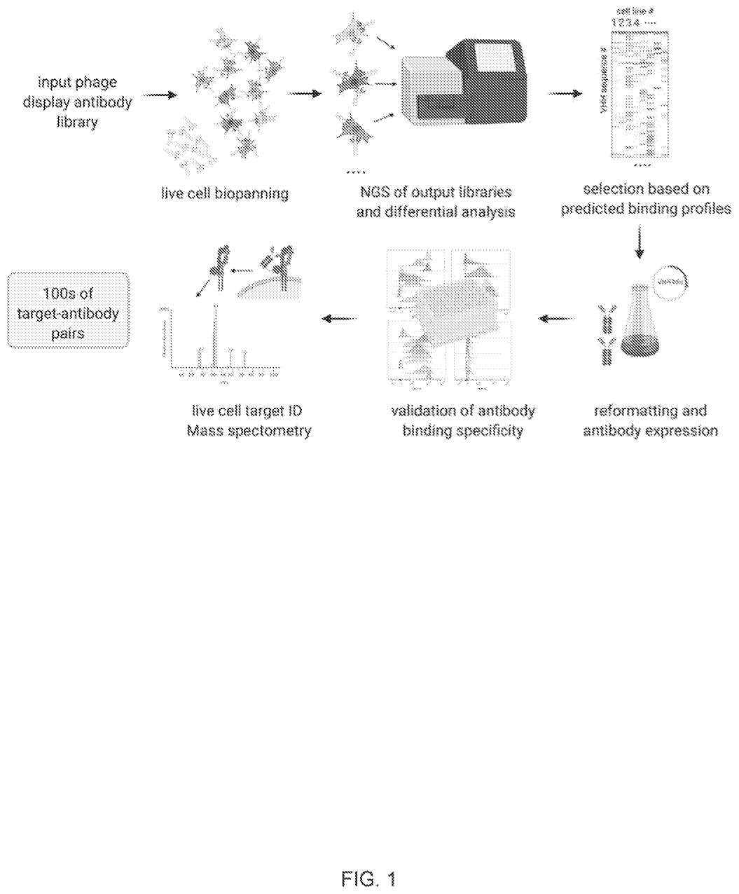

To identify antibodies with specific cell surface binding phenotypes, we developed PhASTdiscovery, a platform that is based on large scale antibody selection and screening of a phage display library in live cells. Antibodies with desired binding properties are then matched to their targets using a proteomic approach. The individual steps of the platform are summarized in FIG. 1.

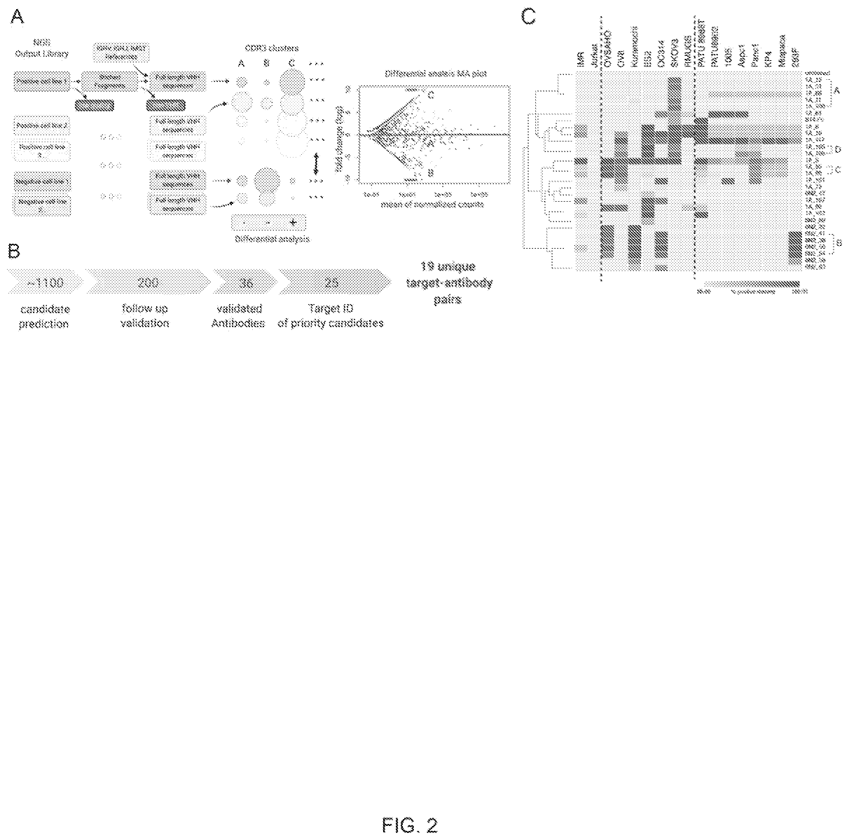

As a representative example, we applied our platform to discover antibodies specific for binding to ovarian cancer cell lines with no binding to lymphocytes, and limited binding to fibroblasts and non-ovarian cancer cells. First, to deplete our VHH-bacteriophage display library of lymphocyte specific VHH, we performed a negative selection using primary PBMCs, followed by enrichment for ovarian cell specific binders using a pool of 6 ovarian cancer cell lines. After library amplification and an additional negative selection step the depleted library was subjected to biopanning against each ovarian line (positive cell lines), and each negative cell line (PBMCs, an immortalized fibroblast cell line, and a pancreatic cell line) individually. The output libraries were characterized by NGS, and following some quality control and normalization steps, individual CDR3s from each cell line were clustered based on homology and subjected to differential analysis (FIG. 2, Panel A).

Around 1100 sequences showed enrichment in at least one ovarian cell line over the negative samples (FIG. 2, Panel B). Of these we randomly selected 200 candidates for follow up binding analysis. Sequences were synthesized, fused to human IgG1-Fc, expressed in the Expi293 expression system before validating their predicted binding profiles in a FACS based multiplex binding assay. Of the 200 antibodies tested, only one showed binding to the negative Jurkat lymphocyte cell line, while 36 antibodies showed specific binding to at least one ovarian cell line, with a subset weakly also binding to fibroblasts and the pancreatic cell line (FIG. 2, Panel C). The remaining antibodies showed only weak or no binding to any cell line tested, a subset of which could be explained by low antibody expression. To further validate the selectivity of the specific binders to ovarian cell lines we tested their binding to a panel of pancreatic and fibroblast cell lines. As illustrated in FIG. 2, Panel C, most antibodies showed no or weak binding to most cell lines while some were less cell line specific.

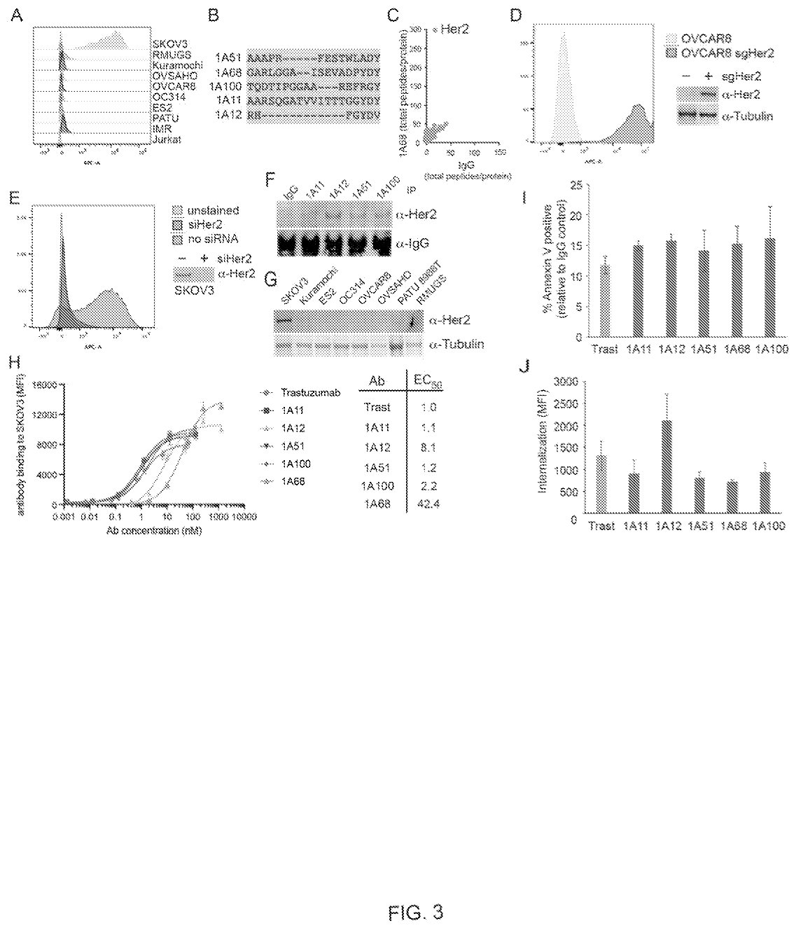

We prioritized antibodies with weak or no cross reactivity to fibroblasts and pancreatic lines for follow up target identification. One cluster of 5 antibodies (highlighted as cluster A in FIG. 2 Panel C) showed particularly high specificity as it selectively bound to only one ovarian cancer cell line (SKOV3) (FIG. 3 Panel A). Comparison of their CDR3 regions confirmed that these antibodies were indeed distinct molecules (FIG. 3 Panel B). Using a biotin transfer based crosslinking approach, we identified the target of these antibodies as Her2 (FIG. 3 Panel C). CRISPRa induced expression of Her2 in OVCAR8 cells, a cell line the antibodies didn't bind to, resulted in strong antibody binding (FIG. 3 Panel D), while knockdown of Her2 in SKOV3 cells lead to loss of binding (FIG. 3 Panel E), validating Her2 as the target of cluster A antibodies. In addition, antibodies were able to immunoprecipitate Her2 from SKOV3 whole cell extracts, further confirming Her2 as their target (FIG. 3 Panel F).

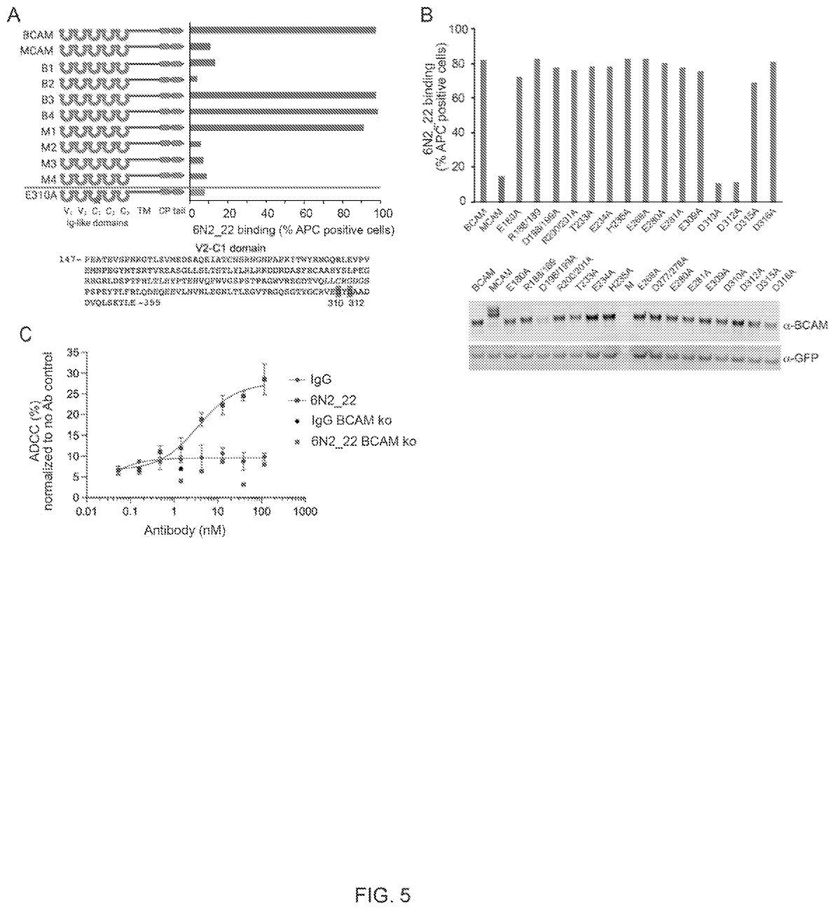

Immunoblot analysis with an established Her2 antibody showed that protein expression of Her2 was high in SKOV3 cells and weak or undetectable in the other ovarian cell lines we analyzed (FIG. 3 Panel G). In agreement, analysis of Her2 gene expression across CCLE cell lines showed high expression of Her2 in SKOV3 cells comparable to high expressing breast cancer cell lines, while expression in other ovarian cell lines was considerably lower (FIG. 5 Panel A), correlating well with the selectivity of cluster A antibodies to Her2.

Trastuzumab, a humanized IgG1 monoclonal antibody that targets the extracellular domain of Her2, is FDA approved for the treatment of Her2-overexpressing breast cancers and metastatic gastric cancers. To validate that these new Her2-specific antibodies compare to Trastuzumab, we first tested their affinity for binding to SKOV3 cells. As shown in FIG. 3 Panel H, 4 out of 5 of our antibodies had a low nanomolar affinity between 1-10 nM, comparable to the 1 nM affinity of Trastuzumab. Only 1A68 had a somewhat lower affinity of 42.2 nM. Next, we validated that the antibodies have distinct or overlapping epitopes to that of Trastuzumab and Pertuzumab, another clinically used anti-Her2 antibody. The competition experiment shown in FIG. 5 Panel C demonstrates that the binding of Trastuzumab was unaffected by preincubation with saturating amounts of any of the Cluster A antibodies. A similar experiment with Pertuzumab showed that 1A51 partially inhibited Pertuzumab binding, indicating that these two antibodies can have partially overlapping epitopes. Together these experiments demonstrate that cluster A antibodies are distinct from Trastuzumab, and with the exception of 1A51 also from Pertuzumab, with regard to their epitope binding sites.

One mode of action of Trastuzumab is the induction of ADCC. In agreement with published literature, Trastuzumab induced potent ADCC in a FACS based assay in which PBMCs were used as effector cells and SKOV3 as target cells (FIG. 3 Panel I). Interestingly, all of the cluster A antibodies induced ADCC to comparable levels as Trastuzumab. Together, these results demonstrate that our platform is not only able to identify cancer relevant targets, but also that the discovered antibodies have properties that are comparable to antibodies currently in clinical use. For example, antibodies discovered by the platform described herein can have different epitopes to the known antibodies.

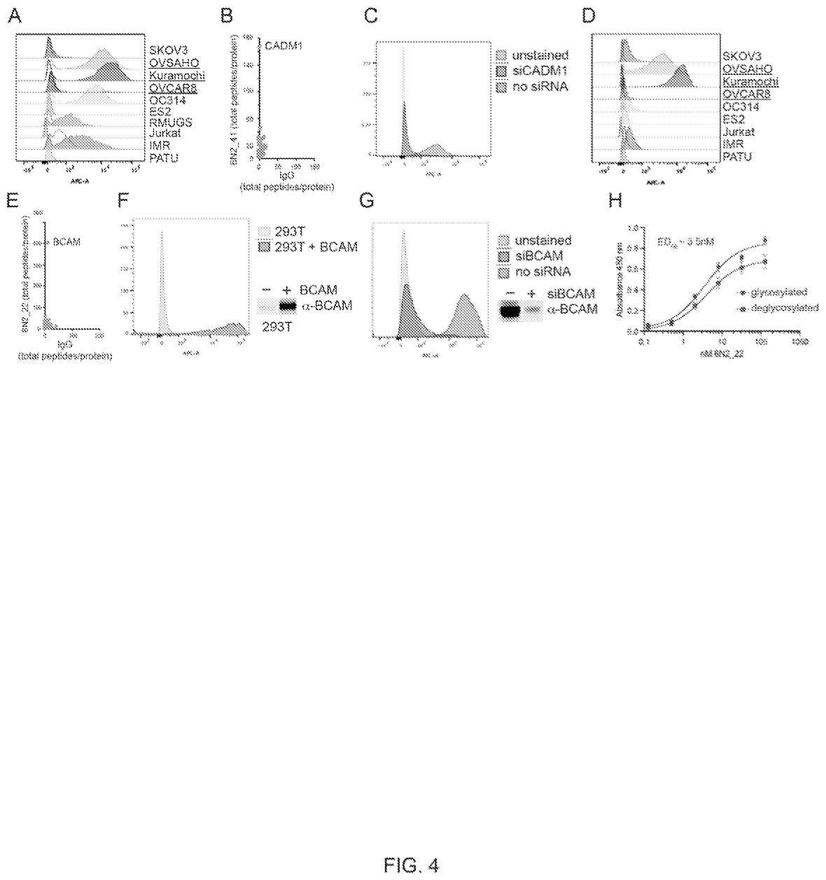

High-grade serous carcinoma (HGSOC) is the most common and lethal subtype of ovarian cancers, with the vast majority of women diagnosed at an advanced stage of disease. The current standard treatment is surgical debulking combined with chemotherapy. While standard therapy induces an initial response, tumors ultimately recur, and 70% of patients die within 5 years of diagnosis. To achieve better outcomes, new therapeutic targets are needed. One of the antibodies we discovered, 6N2_22, showed remarkable specificity to two HGSC cell lines (Kuramochi and OVSAHO), without binding to any other cell line we tested (FIG. 4 Panel A). Proteomics analysis indicated BCAM as its target (FIG. 4 Panel B). Transient transfection of a human BCAM expression vector into 293T cells resulted in robust binding of 6N2 22 to BCAM overexpressing cells by facs while no binding was detected in control transfected cells (FIG. 4 Panel C). Conversely, silencing of BCAM in the Kuramochi cell line lead to a loss of 6N2_22 binding (FIG. 4 Panel D).

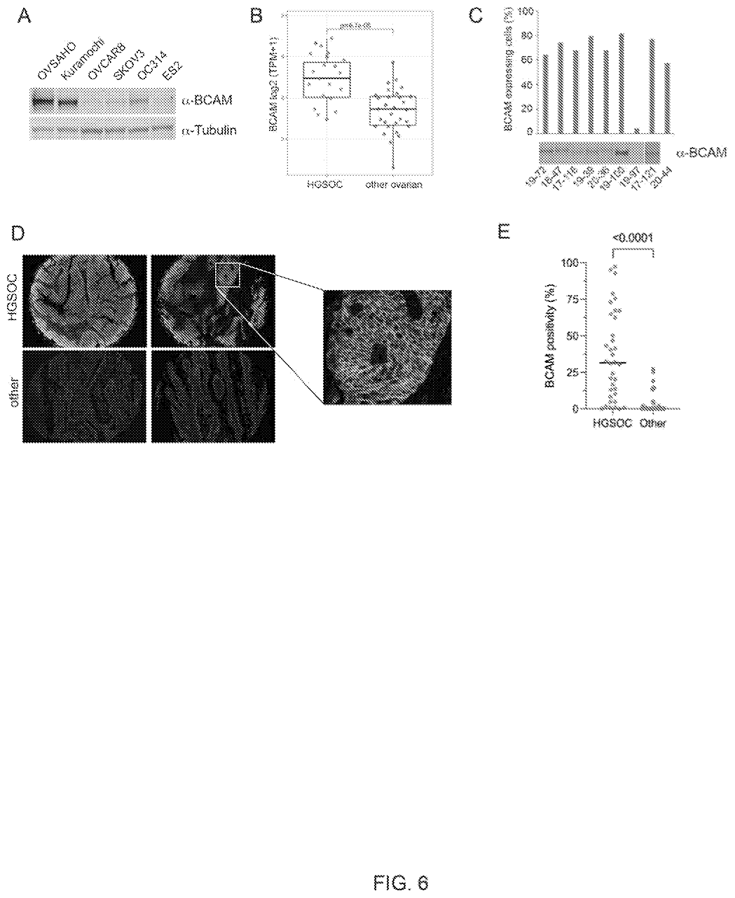

Western blot analysis with a commercially available BCAM antibody showed high protein expression in Kuramochi and OVSAHO cell lines, while expression was considerably weaker in the other ovarian cell lines analyzed, correlating well with the 6N2_22 cell line binding pattern (FIG. 4 Panel E). In agreement, gene expression data derived from CCLE showed Kuramochi and OVSAHO cell lines among the highest BCAM expressing ovarian cell lines (FIG. 4 Panel F). Given the selectivity of our antibody to two high grade cell lines we wanted validate that high BCAM expression is enriched in HGSOC over other ovarian subtypes. Indeed, comparing BCAM expression across CCLE ovarian cell lines showed highly significant enrichment for high BCAM expression in HGSOC cell lines. Analysis of TCGA data across cancer types reveals high BCAM expression on various cancers, with serous ovarian adenocarcinomas as highest expressing cancer (FIG. 6 Panel A). Importantly, expression data across healthy tissue show low BCAM levels across most tissues, with elevated expression in Kidney and Thyroid (FIG. 6 Panel B). Together these data indicate BCAM as a therapeutic target against a subset of cancers, in particular for BCAM-overexpressing HGSOC.

To further characterize the antibody, we first measured its binding affinity in live cells, as well as to recombinant BCAM by ELISA. On Kuramochi cells 6N2_22 showed an affinity of ˜7 nM, and an affinity of 3.5 nM to recombinant BCAM (FIG. 4 Panel G and FIG. 6 Panel C). As BCAM is known to be heavily glycosylated we set out to test if 6N2_22 binding depends on BCAMs glycosylation status. Deglycosylation with PNGase resulted in a ˜20 kDa shift in BCAM migration on a Coomassie gel indicating successful deglycosylation (FIG. 6 Panel D). ELISA showed that the binding affinity of 6N2_22 was unaffected by BCAMs glycosylation status, indicating that the antibody recognizes BCAM irrespective of glycosylation (FIG. 4 Panel G).

As ADCC is a major mode of action in targeted antibody therapies, we validated the ability of 6N2_22 to induce ADCC in a reporter-based assay on Kuramochi cells. As shown in FIG. 4 Panel H, 6N2_22 potently induced ADCC in a dose dependent manner while S14, a non-specific control VHH-Fc antibody did not have an effect. Together these data demonstrate that we discovered a therapeutic antibody and indicates BCAM as a target in treatment of HGSOC.

### Discussion

For the development of an effective and save therapy identification of physiologically relevant cancer specific surface targets is prerequisite, as is the discovery of specific and potent antibodies to target them. Although much progress has been made in the antibody therapeutics field, both target nomination and subsequent antibody discovery regiments have been a bottleneck in the development of targeted cancer therapeutics, in part due to the requirement for intensive research to validate the cancer relevance of the target, low throughput of the antibody discovery process, and associated high costs. Our platform overcomes several of these challenges. First, PhASTdiscovery is a high-throughput approach to identify hundreds of antibody-target pairs in a single round of screening. Our live cell-based screening approach ensures that discovered antibodies indeed bind to physiologically relevant states of the target, and possess the desired phenotypic binding characteristics, reducing the need for target validation. Second, our binding profile predicting selection method streamlines screening by selecting candidates by desired binding specificity while cutting down on screening of highly homologous molecules by hundred—to thousand-fold, increasing both, throughput, and cost effectiveness. Third, most antibodies we discovered not only show high binding selectivity, but they are also expressed at high levels, have low nanomolar affinity without the need for further affinity maturation, and can mediate ADCC, indicating that they are developable as therapeutics. Together, our platform can identify large sets of targets in an unbiased way in their true cancer specific state while simultaneously discovering potent antibodies against them in as little as two months, greatly accelerating the process.

Anti-Her2 antibodies have been successfully used in treatment of Her2-overexpressing breast and metastatic gastric cancers. The discovery of a set of Her2 antibodies in the context of ovarian cell lines indicates it might also be a good target in a subset of ovarian cancers. Indeed, analysis of CCLE data shows high expression of Her2 in some ovarian cancer cell lines of which a small subset is highly Her2 dependent. Furthermore, HER2 overexpression/amplification has been reported in ovarian cancer, especially in clear cell and mucinous tumors. Monotherapies using Trastuzumab or Pertuzumab showed limited clinical benefits, while combination with chemotherapy had somewhat better outcomes. In analogy to breast cancer were antibody combination therapies or use of Her2 targeted drug conjugated antibodies led to vastly improved responses similar approaches can be used for ovarian cancer treatment.

The discovery of an antibody with binding specificity to HGSOC, the most common and aggressive form of ovarian cancer, led to the identification of BCAM as a therapeutic target. BCAM, first shown to be highly expressed on sickle red blood cells, is overexpressed in a number of tumors, notably, highest in HGSOC, while its expression appears relatively low in normal tissues, with moderate expression in the kidney and the thyroid (FIG. 6). Immunohistochemistry staining of tumors confirms high expression in about 35-40% of primary HGSOC tumors (own data and/or reference), and it has been reported to be overexpressed on metastasis of colon and breast cancers, further indicating BCAM as therapeutic anti-cancer target.

BCAM is a transmembrane glycoprotein with 5 immunoglobulin-like domains that acts as a receptor for Laminin a5. Their interaction was demonstrated to promote adhesion and migration of carcinoma cells. Accordingly, inhibition of BCAM-LAMA interaction has an inhibitory effect on migration. Our 6N2_22 antibody doesn't affect BCAMs ability to bind to LAMA5 and it doesn't have an apparent effect on cell adhesion. A previous study described an a-BCAM antibody-drug conjugate that induced cancer cell killing, indicating the antibodies can induce receptor internalization. We did not see any evidence that 6N2_22 triggers BCAM internalization in 2 cell lines tested, but it showed potent ADCC activity underscoring its use as therapeutic antibody in the VHH-hIgG1-Fc format. That said, the superior properties of VHHs (nanobodies) such as small size, high stability, strong antigen-binding affinity, water solubility, and high modularity also make them well suited for development of antibody therapeutics, such as bi- or multi-specific T-cell engagers.

The ability of our platform to screen VHHs for specific binding profiles in high throughput opens up the door for the identification of highly selective target combinations useful for the development of the next generation of combinatorial therapies. The simultaneous discovery of VHHs specific to these targets will make the engineering of these innovative therapeutics relatively easy and time efficient.

### Example 4

### NGS Analysis

Illumina paired-end 2×250 bp sequencing was performed on targeted VHH sequences. Trimmomatic (version 0.38) was first used to remove fragments with low base calling quality (average Phred score <30) and clip Illumina adapter sequences from all reads [1]. Reads were additionally cropped at 225 bp to remove low quality positions. Quality passing paired reads were merged using FLASh (version 1.2.11) with a fragment length and standard deviation set to 375 bp and 35 bp, respectively [6].

Merged reads were filtered to only those which appeared to be valid VHH sequences based on heavy chain structure. Reference sequences for the camelid heavy chain framework regions (FR) were obtained from IGHV and IGHJ alleles of the closely related Vicugna pacos in the IMGT/V-QUEST reference directory set (release 201908-4) [3]. Position weight matrices (PWMs) were constructed for each of the four FRs based on the reference alleles. A sequence was determined to be valid if all FRs were matched with scores >60\% of the maximum PWM score. FRs were also required to be in frame, on the same strand, and checked for certain conserved heavy chain amino acids (cysteine at 23, tryptophan at 41, hydrophobic amino acid at 89, and cysteine at 104). Full length VHH sequences were trimmed and translated to amino acid sequences.

Amino acid (AA) sequences for the complementary determining region 3 (CDR3) were extracted from reads based on the previously matched FR3 and FR4 positions. CDR3 sequences shorter than 2 AAs were dropped. Unique CDR3 sequences were clustered across all samples using CD-HIT (version 4.8.1) [2,4]. CDR3 sequences were clustered if sequences had the same length and had similarity above 0.6 for shorter sequences (<10 AAs) or 0.7 for longer sequences (<10 AAs). CDR3 sequences were sorted by total fragment counts prior to clustering with CD-HIT. Clustering was performed jointly across all samples.

For each CDR3 cluster, we counted the number of fragments matching a CDR3 sequence in the cluster for each sample. The matrix of sample fragment counts across CDR3 clusters was next used for differential analysis. CDR3 clusters differentially present across positive and negative selection samples were identified using DESeq2 [5]. Testing was performed with outlier imputation disabled as samples within each group were heterogeneous. The default Cook's distance filtering and independent filtering procedures were also disabled while testing with DESeq2.

References Cited in this Example:
[1] Bolger, A. M., Lohse, M., and Usadel, B. Trimmomatic: a flexible trimmer for illumina sequence data. Bioinformatics 30, 15 (2014), 2114-2120.
[2] Fu, L., Niu, B., Zhu, Z., Wu, S., and Li, W. Cd-hit: accelerated for clustering the next-generation sequencing data. Bioinformatics 28, 23 (2012), 3150-3152.
[3] Lefranc, M.-P., and Lefranc, G. The immunoglobulin factsbook. Academic Press, 2001.
[4] Li, W., and Godzik, A. Cd-hit: a fast program for clustering and comparing large sets of protein or nucleotide sequences. Bioinformatics 22, 13 (2006), 1658-1659.
[5] Love, M. I., Huber, W., and Anders, S. Moderated estimation of fold change and dispersion for RNA-seq data with DESeq2. Genome Biology 15, 12 (2014), 550.
[6] Magoč, T., and Salzberg, S. L. FLASH: fast length adjustment of short reads to improve genome assemblies. Bioinformatics 27, 21 (2011), 2957-2963.

### Example 5

Discovery of antibodies and cognate surface targets for ovarian cancer by surface profiling

### Abstract

Although antibodies targeting specific tumor-expressed antigens are the standard of care for some cancers, the identification of cancer-specific targets amenable to antibody binding has remained a bottleneck in development of new therapeutics. To overcome this challenge, we developed a high throughput platform that allows for the unbiased, simultaneous discovery of antibodies and targets based on phenotypic binding profiles. Applying this platform to ovarian cancer, we identified a wide diversity of cancer targets including receptor tyrosine kinases, adhesion and migration proteins, proteases and proteins regulating angiogenesis in a single round of screening using genomics, flow cytometry, and mass spectrometry. In particular, we identified BCAM as a promising candidate for targeted therapy in high grade serous ovarian cancers. More generally, this approach provides a rapid and flexible framework to identify cancer targets and antibodies.

### Significance Statement

Target selection for the development of antibody directed therapies is commonly driven by a specific hypothesis or based on expression profile analysis, which require laboriosu experimental validation. We developed a pooled screening platform for the unbiased, high throughput identification of multiple cancer specific targets in a single screen. Applying this technology resulted in the identification of multiple new therapeutic target candidates in ovarian cancer. In addition to the selection of cell-type specific targets, the platform simultaneously allows the discovery of antibodies with therapeutic potential, thereby bypassing the need for lengthy antibody discovery campaigns.

### Introduction

Several therapeutic antibodies are approved for the treatment of specific cancers. Some of these antibodies target proteins essential for the malignant phenotype, such as HER2; while others target proteins aberrantly expressed by tumor cells, such as VEGF. More recently, antibodies, such as those that target PD1, that modulate immune activity have exhibited dramatic responses in subsets of patients (1, 2). Most efforts to identify new antigens for antibody-based therapies require prior knowledge of targets or arduous validation schemes.

Since antibodies cannot easily penetrate the cell membrane, antibody-based therapeutics rely on their reactivity to cell surface proteins. One major challenge in the development of clinically effective biologics has been off-tumor cytotoxicity, mostly driven by on-target effects mediated by expression of the antibody target on non-malignant tissues (2). Other challenges include immune escape through target downregulation as well as target heterogeneity within the tumor (2). To overcome these obstacles, methods to facilitate the discovery of alternative cell/tumor type specific targets with high expression in malignant tissue is essential.

The target selection for the development of antibody directed therapies has primarily been guided by exploring expression databases for potential cancer or tissue specific cell surface markers, and by hypothesis driven approaches based on the study of specific oncogenes. While these strategies have led to the approval of several promising therapies, they have several important limitations. First, the measurement of mRNA as readout for expression has historically been done on bulk samples resulting in loss of information on the cellular distribution of expression within the tumor. Second, gene expression does not always correlate well with protein expression due to translational control mechanisms. Third, gene expression data generally does not provide critical information about the surface localization of many receptors, transporters, and channels, which can be tightly regulated and vary widely depending on the microenvironmental context. Accordingly, the tumor microenvironment can alter the surface abundance of proteins without detectable differences in gene expression profiles (3, 4), which would then be missed by conventional gene expression analysis. Similarly, many surface proteins have been shown to be posttranslationally modified, or to be expressed in cancer specific protein complexes which might affect their conformation, rendering them cancer specific targets despite uniform expression. Given these shortcomings, targets predicted by gene expression approaches require in depth experimental follow up validation, which can be costly and time consuming.

To address these challenges, we developed the Phenotypic Antibody and Simultaneoius Target (PhAST)-discovery platform, an approach utilizing a bacteriophage display-based VHH library to select for antibodies that bind with desired cell surface binding specificity on live cells followed by mass spectrometric identification of the antibody target. This approach allows the discovery of multiple antibody-target pairs specific to the native or cancer specific state in a single round of screening. Applying this platform, we identified a set of new therapeutic target candidates in ovarian cancer which we chose because of the high mortality rate and lack of therapies beyond standard chemotherapy and surgery.

### Results

To identify targets with cell surface expression phenotypes specific for ovarian cancer, we performed a phage display antibody library screen on live cells. We chose an array of ovarian cancer cell lines across multiple histologies to try to identify not just ovarian cancer-specific, but also possibly subtype specific targets (5). Antibodies with desired binding properties were then matched to their targets using a proteomic approach. The individual steps of the approach are summarized in FIG. 1.

Our goal was to identify antibodies that exhibited specific binding to ovarian cancer cell lines but failed to bind to lymphocytes, fibroblasts and non-ovarian cancer cells. We used a commercially available VHH-bacteriophage display library that was generated from 10 naïve lamas (Abcore) and a library constructed after immunization of 2 lamas with whole cell plasma-membrane preparations (Prosci). To deplete the VHH libraries of lymphocyte specific VHHs, we performed a negative selection using primary peripheral blood mononuclear cells (PBMCs), followed by enrichment of the unbound fraction for ovarian cell specific binders using a pool of 6 ovarian cancer cell lines (positive cell lines). After rigorous washing, phages were eluted by low pH treatment followed by bacterial amplification. The amplified output library was further depleted of unwanted VHHs by a second round of negative selection on PBMCs. Unbound phages were then subjected to biopanning against each ovarian line, and each negative cell line (PBMCs, an immortalized fibroblast cell line, and a pancreatic cell line) individually. The output libraries were characterized by massively parallel sequencing and compared against reported camelid V-gene and J-gene alleles in the IMGT/GENE-DB. We excluded sequences that were dissimilar to reported alleles or that showed alternations to the expected conserved amino acids. Extracted full-length VHH sequences were translated and clustered across cell lines based on the amino acid sequence similarity of the CDR3s. To select sequences that were specifically enriched in the cell lines of interest, we performed differential analysis between positive and negative cell lines across this set of CDR3 clusters (FIG. 2A).

1032 clusters showed enrichment in at least one ovarian cell line over the negative samples (FIG. 2B). For follow up binding analysis, we selected 200 sequences that were enriched in at least one HGSOC ovarian line or that showed high selectivity to one specific cell type. These sequences were synthesized, fused to human IgG1-Fc, expressed in the Expi293 expression system before validating their predicted binding profiles in a FACS based multiplex binding assay. Of the 200 antibody supernatants tested, only one showed binding to the negative Jurkat lymphocyte cell line (data not shown), while 36 antibodies showed specific binding to at least one ovarian cell line, with a subset weakly also binding to fibroblasts and a pancreatic cell line (FIG. 2C). The remaining antibodies exhibited only weak or absence of binding to any cell line tested, a subset of which could be explained by low antibody abundance in the supernatant as suggested by expression analysis of antibody supernatants by SDS-PAGE Coomassie staining (data not shown). To further evaluate the selectivity of the specific binders to ovarian cell lines, we tested binding to an additional panel of pancreatic and fibroblast cell lines. As summarized in FIG. 2C, the majority of antibodies showed binding to only a small subset of cell lines while a small subset bound to a much broader range of cell lines. Four clusters of antibodies shared similar binding patterns (clusters A-D highlighted in FIG. 2C), and other individual antibodies showed distinct binding profiles.

To identify the targets of these antibodies, we prioritized antibodies within the 4 clusters as well as ones with weak or no cross reactivity to fibroblasts. We used an in vivo biotin transfer based crosslinking approach (6). Specifically, we incubated an antibody labelled with a trifunctional aminooxy-sulfhydryl-biotin (ASB) crosslinker with oxidized live cells to induce formation of crosslinks with aldehyde-containing glycans on the antibody bound cell surface protein. Subsequent reduction of the disulfide bond triggered biotin transfer from the antibody to the surface protein. Upon cell lysis, biotinylated proteins were enriched using streptavidin beads followed by mass spectrometry. For data analysis, we quantified relative peptide enrichment against similarly labelled IgG or unrelated antibody controls. Using this method, we identified 10 targets for 19 antibodies. A summary of the antibody-target pairs is listed in Table 1. The targets belong to a diverse set of protein classes, including two receptor tyrosine kinases, five adhesion molecules, two proteases, and one protein reported to regulate angiogenesis.

Cluster A showed particularly high specificity as it selectively bound to only one ovarian cancer cell line (SKOV3) (FIG. 3A). This observation is particularly interesting as SKOV3 is a p53 wildtype cell line with an uncertain histology differing from most of the other lines in our panel (5). Comparison of their CDR3 regions confirmed that these antibodies were indeed distinct molecules (FIG. 3B). Using our proteomics approach, we identified the target of these antibodies as Her2 (FIG. 3C). To confirm the mass spectrometry results, we drove Her2 expression using CRISPRa in OVCAR8, a cell line that this antibody failed to bind. Overexpression of Her2 (FIG. 3D, right panel) resulted in strong antibody binding (FIG. 3D, left panel), while we failed to detect binding in the parental OVCAR8 dCas9 cell line. Conversely siRNA mediated knockdown of Her2 in SKOV3 cells lead to loss of binding (FIG. 3E) comparable to a no antibody control while strong binding was apparent in non-targeting siRNA control transfected cells. Next, we tested if the antibodies immunoprecipitated Her2 from SKOV3 whole cell extracts. We found that three of the four antibodies immunoprecipitated Her2 as assessed by immunoblotting immune complexes using a commercially available anti-Her2 antibody as probe (FIG. 3F). Together, these observations validate Her2 as the target of cluster A antibodies.

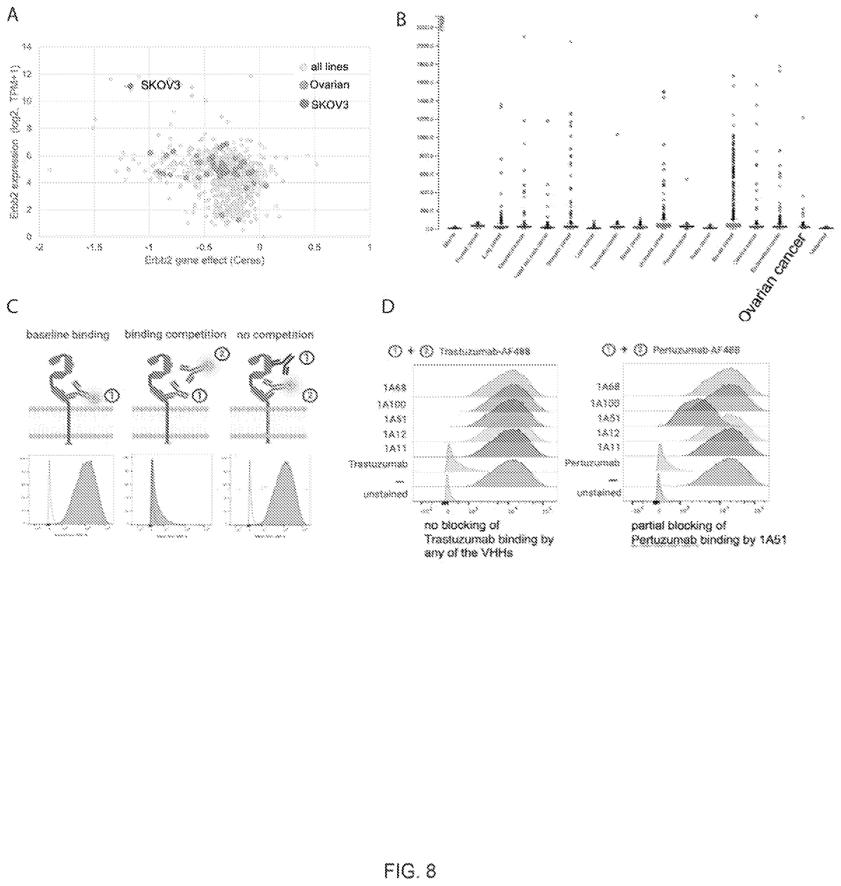

Immunoblot analysis with an established Her2 antibody showed that protein expression of Her2 was high in SKOV3 cells and weak or undetectable in the other ovarian cell lines we analyzed (FIG. 3G). In agreement, analysis of Her2 gene expression across Cancer Cell Line Encyclopedia (CCLE) cell lines (7) showed high expression of Her2 in SKOV3 cells comparable to high expressing breast cancer cell lines, while expression in other ovarian cell lines was considerably lower (FIG. 8A), correlating well with the selectivity of cluster A antibodies to Her2. Interestingly, in the Dependency Map portal (DepMap), an initiative to systematically identify cancer cell line vulnerabilities (8), the SKOV3 cell line exhibited high Her2 dependency, further confirming our findings.

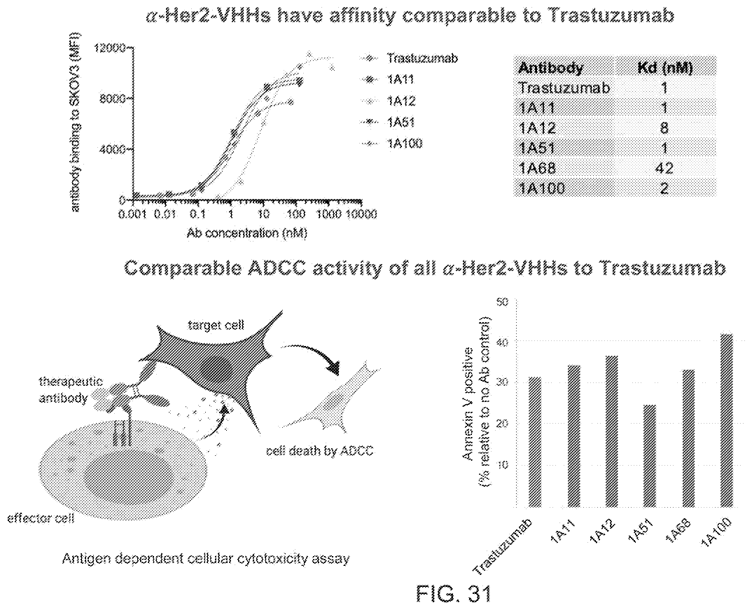

Her2 is a well-known target in the subset of breast cancers that harbor Her2 amplifications as well as metastatic uterine serous and gastric cancer that overexpress Her2 (9). One mode of action of Her2 targeting therapeutic antibodies is their ability to induce antibody dependent cellular cytotoxicity (ADCC) (10, 11). Accordingly, we tested whether Trastuzumab, an anti-Her2 humanized monoclonal antibody in clinical use, and the cluster A chimeric single domain antibodies induced ADCC in the context of ovarian cancer cell lines. The affinity to the target and target density on the cell surface are important factors necessary for ADCC (12). Therefore, we first compared the affinity of Trastuzumab and the cluster A antibodies for binding to SKOV3 cells. As shown in FIG. 3H, 4 out of 5 of the antibodies had a low nanomolar affinity between 1-10 nM, comparable to Trastuzumab (InM). Only 1 A68 had a somewhat lower affinity of 42.2 nM. Epitope binning experiments demonstrated that all antibodies had distinct epitopes from Trastuzumab, Pertuzumab and from each other (FIG. 8C, 8D, and data not shown). In a FACS-based assay in which PBMCs were used as effector cells and SKOV3 as target cells, Trastuzumab induced potent ADCC (FIG. 31). We found that all of the cluster A antibodies induced ADCC to comparable levels as

Trastuzumab when used in saturating concentrations. To further characterize the antibodies, we tested their internalization properties by measuring the induction of fluorescent signal that is triggered by uptake of pHrodo iFL labelled antibodies into low-pH endosomes. As shown in FIG. 3J, we detected internalization of all antibodies. Together, these observations suggest that in addition to breast, uterine serous, and gastric cancers, Her2 is an attractive target in Her2 overexpressing ovarian cancers, and that the antibodies we discovered in our screen have features comparable to antibodies in clinical use.

Several antibodies showed intriguing specificity to HGSOC cell lines. Of these, cluster B antibodies showed high binding specificity to two HGSOC cell lines (OVSAHO and Kuramochi) and one clear cell ovarian cell line (OC314) and exhibited low or no binding to control cells (FIG. 2C and FIG. 4A). Using the in vivo crosslinking-mass spectrometry method, we identified the target of these antibodies as CADM1 (FIG. 4B). To validate these observations, we knocked down CADM1 in Kuramochi cells and tested antibody binding by flow cytometry. As shown in FIG. 4C, while strong binding of 6N2_38 was detected in control transfected cells, binding was abolished in siRNA transfected cells, confirming the specificity of cluster B antibodies to CADM1.

In addition, we discovered 6N2_22, an antibody which showed remarkable specificity to the two HGSOC cell lines Kuramochi and OVSAHO, but did not bind to any other cell line we tested (FIG. 4D). Using our proteomics approach, we identified BCAM, a transmembrane glycoprotein that acts as a receptor for Laminin a5 (LAMA5) as the target of 6N2_22 (FIG. 4E). When we ectopically expressed human BCAM in 293T cells, we found robust binding of 6N2_22 to BCAM overexpressing cells, while no binding was detected in control transfected cells (FIG. 4F). Conversely, silencing of BCAM in the Kuramochi cell line led to loss of 6N2_22 binding (FIG. 4G), while control siRNA transfection did not affect antibody binding.

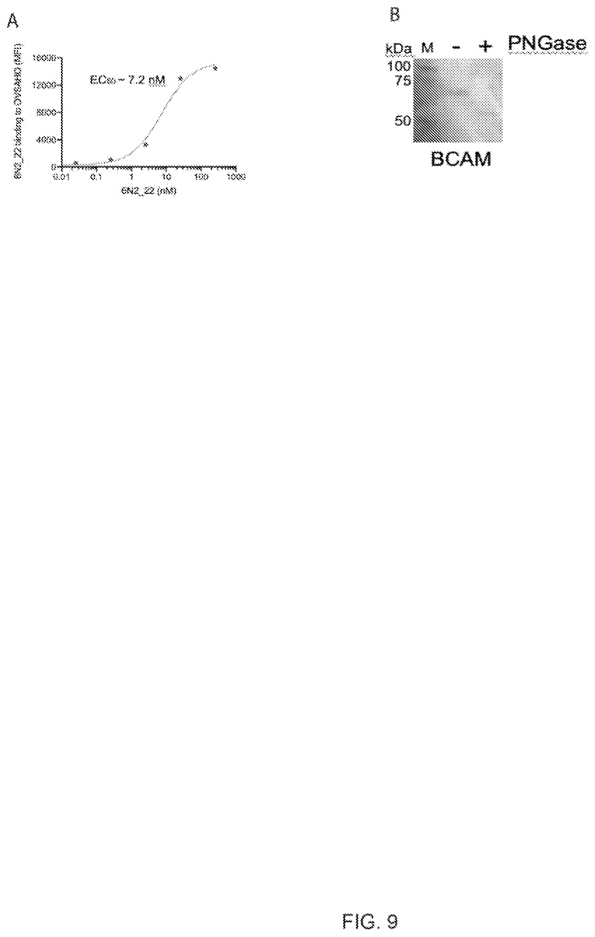

To further confirm the specificity of 6N2_22 to BCAM, we measured its binding affinity to recombinant BCAM by ELISA. 6N2_22 showed an affinity of 3.5 nM to recombinant BCAM, compared to an affinity of ˜7 nM on Kuramochi cells (FIG. 4H and FIG. 9A). Since BCAM is known to be heavily glycosylated (13), we tested whether 6N2_22 binding depends on BCAMs glycosylation status. Deglycosylation with PNGase resulted in a ˜20 kDa shift in BCAM migration on a Coomassie gel indicating successful deglycosylation (FIG. 9B). ELISA showed that the binding affinity of 6N2_22 was unaffected by BCAMs glycosylation status, indicating that the antibody recognizes BCAM irrespective of glycosylation (FIG. 4H).

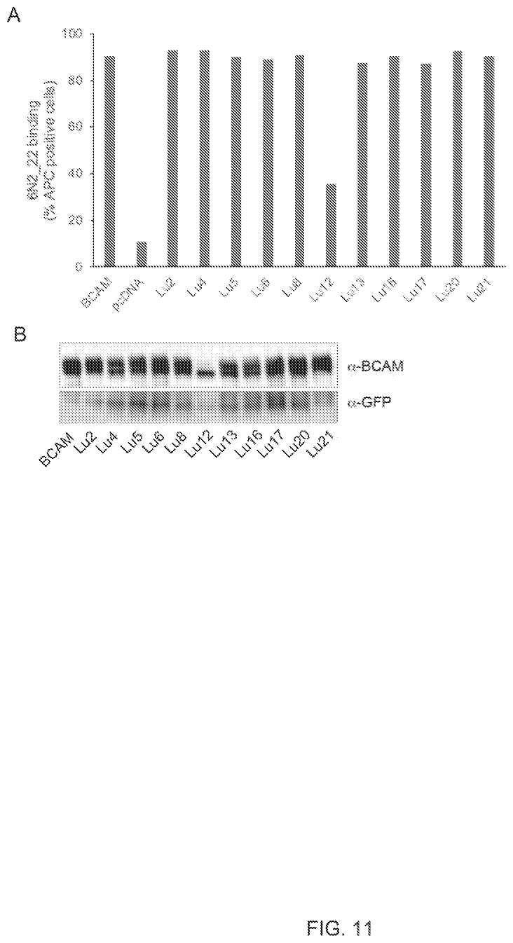

To further characterize the 6N2_22 antibody, we mapped the epitope for 6N2 22. BCAM belongs to the immunoglobulin superfamily (IgSF), and the extracellular region is composed of five immunoglobulin like domains (V1-2, C1-3) (14, 15). To narrow down the region necessary for antibody binding, we constructed chimeras between BCAM and MCAM, a closely related protein with similar Ig-like domain architecture that 6N2 22 does not bind to (16, 17) (FIG. 5A). Chimeras were tested for antibody binding by flow cytometry of 293T cells transiently transfected with the respective constructs. As expected, the antibody did not bind to MCAM, and swapping domains V1 and V2 to those of BCAM had no effect on the ability of the antibody to bind (FIG. 5A). The additional exchange of domain C1 however resulted in antibody binding comparable to that of full length BCAM, indicating that the epitope is located within domains V1, V2 and C1. Conversely, swapping BCAMs V1 domain with V1 of MCAM resulted in binding comparable to full length BCAM, suggesting that this region is dispensable for binding. BCAM binding was lost when V2 was replaced with the respective MCAM domain. Further replacement failed to restore the ability of these antibodies to bind BCAM. Together these studies demonstrate that the epitope is located on V2 and C1 of BCAM. To further map the amino acids involved in binding, we performed mutagenesis within this region, replacing structure predicted surface exposed charged residues with alanine (14, 15). Expression of all constructs were comparable (FIG. 5B, lower panel). Although most mutations had no effect on binding, mutation of aspartic acids 310 and 312 to alanine both abolished 6N2_22 binding to BCAM, indicating that these residues are an essential part of the binding interface (FIG. 5B, upper panel). Several polymorphisms in the extracellular domain of BCAM have been reported (REF). We therefore tested 6N2_22 binding to the most common polymorphismsm and found that 6N2_22 binding to all mutants was comparable to wild-type BCAM, except for Lu12 which was poorly expressed (FIG. 11).

We then assessed whether 6N2_22 mediates killing of BCAM overexpressing cells. Specifically, we performed an ADCC assay, using PBMCs as effectors and CSFE labelled Kuramochi cells as target cells. Upon incubation with 6N2_22 or control antibody, cells were stained with Annexin V-488 and analyzed by flow cytometry. As shown in FIG. 5C, 6N2_22 potently induced ADCC of Kuramochi cells in a dose dependent manner. The activity was dependent on BCAM expression as the antibody lost its ability to induce ADCC of BCAM KO cells.

To assess BCAM as a potential target for ovarian cancer, we first performed immunoblot analysis with a commercially available BCAM antibody in ovarian cancer cell lines and patient-derived organoids. As shown in FIG. 6A, high protein expression was detected in Kuramochi and OVSAHO cell lines, while expression was considerably weaker in the other ovarian cell lines analyzed, correlating well with the 6N2_22 cell line binding pattern. In agreement, gene expression data derived from CCLE showed Kuramochi and OVSAHO cell lines among the highest BCAM expressing ovarian cell lines (FIG. 6B). Given the selectivity of the antibody to two high grade cell lines, we tested whether high BCAM expression is enriched in HGSOC over other ovarian subtypes. Indeed, comparing BCAM expression across CCLE ovarian cell lines showed highly significant enrichment for high BCAM expression in HGSOC cell lines. We next evaluated patient-derived ovarian cancer derived-organoids. As shown in FIG. 6C, 8 of the 9 organoids analyzed showed strong 6N2_22 staining in at least 65% of cells. Together, these observations demonstrate that BCAM is preferentially expressed in HGSOC cell lines and on a large fraction of organoids derived from ovarian cancer patients.

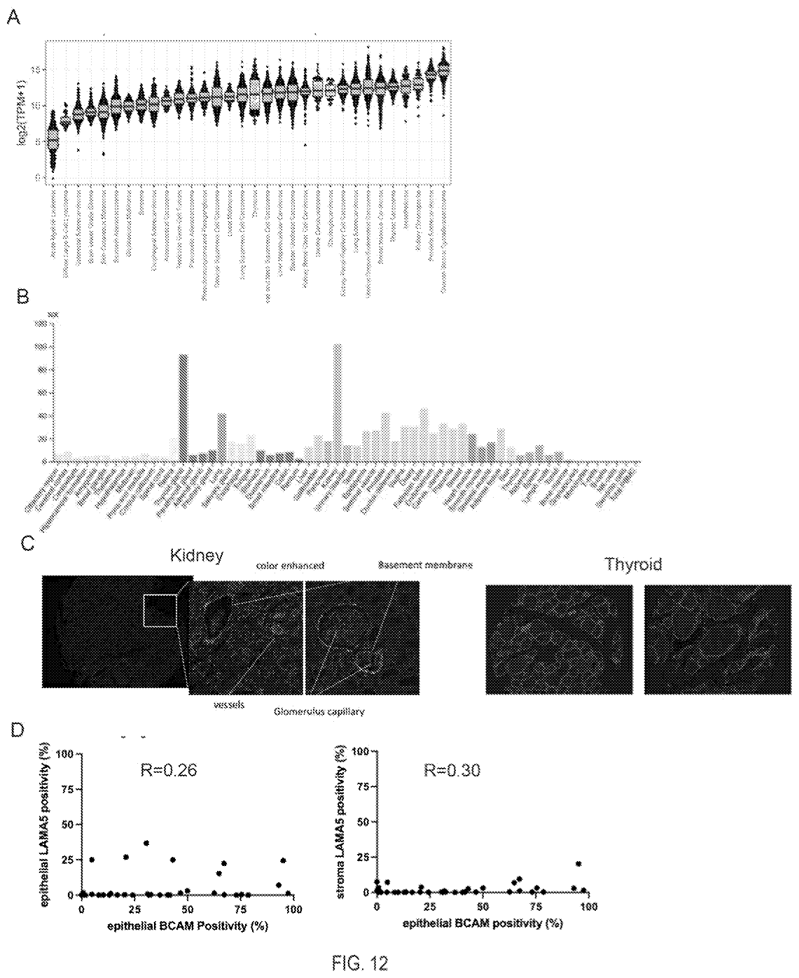

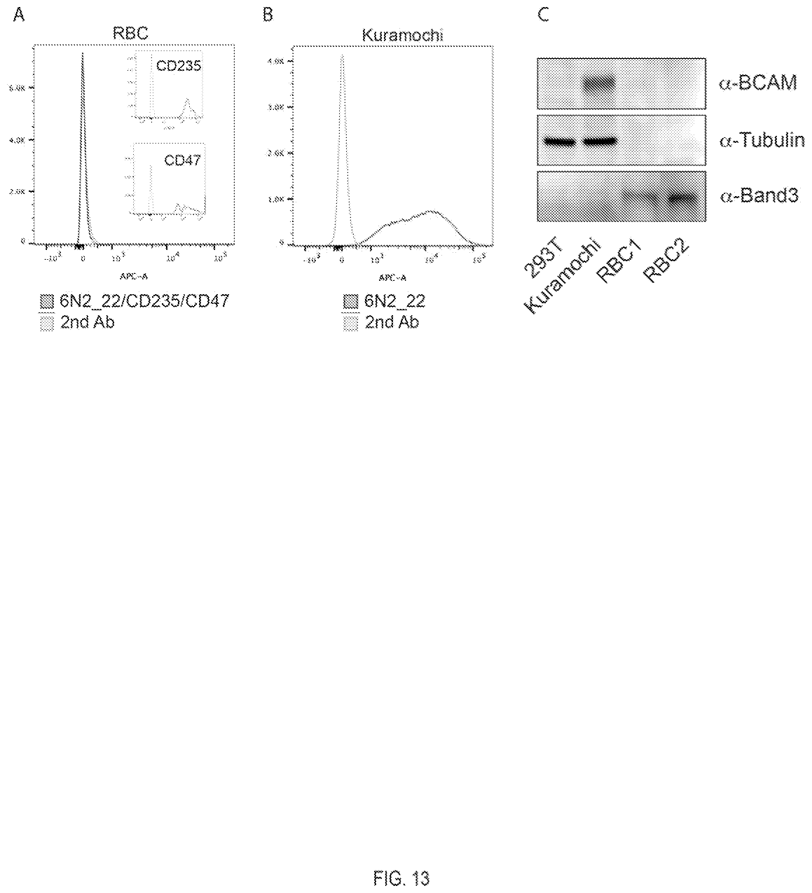

To examine BCAM levels on primary tumors, we analyzed ovarian tissue microarrays for BCAM. The microarray included 36 HGSOC cores and 33 cores from other ovarian cancer subtypes. Strong staining was detected on the surface of HGSOC tumor cells while no or weak BCAM expression was observed on adjacent stromal cells or tumor cores from other tumor subtypes (FIG. 6D). The cell staining pattern suggested that BCAM is expressed on the cell surface. When we compared the percentage of tumor cells expressing BCAM (positivity score) between HGSOC and other cancer subtypes, we found a highly significant enrichment for BCAM expression on HGSOC (FIG. 6E). Interestingly when we co-stained these samples with laminin5a (LAMA5), BCAMs primary ligand (18, 19), we found no correlation between BCAM and Laminin 5 expression neither on tumor cells nor stroma (FIG. 12C). Of note, BCAM expression was weak or absent in healthy ovarian, kidney and thyroid tissues (FIG. 12D). BCAM has been reported to be expressed on red blood cells (RBCs), in particular RBCs in patients with Sickle cell anemia (20). To assess the level of BCAM expression on healthy RBCs we performed flow cytometry binding studies with 6N2_22 on whole blood. As illustrated in FIG. 13, we did not detect appreciable binding of 6N2_22 to RBCs, while expression of the RBC marker CD235 and of CD47, a surface protein well known to be expressed at high levels on RBC were readily detectable. We obtained similar results with a commercial BCAM antibody and confirmed the low levels of expression of BCAM on RBCs by immunoblotting (FIG. 13 right panel). Together these data suggest BCAM as an attractive therapeutic target in a subset of HGSOC.

### DISCUSSION

Although antibodies are a well-established therapeutic modality, target nomination remains a bottleneck in the development of targeted cancer therapeutics, in part due to the requirement for intensive research to identify potential candidates that then need to undergo rigorous validation to confirm their suitability as cancer targets. Using a phenotypic library screening approach, we report a platform that overcomes some of these challenges and identified numerous physiologically relevant ovarian cancer specific surface targets in an unbiased manner. Selecting for antibodies with desired cell line binding profiles allowed us to biochemically identify several highly cell type specific targets bypassing the need for extensive target validation.

In this study we used single domain antibody libraries, but the platform is highly versatile and with some adjustments can readily be adopted to other formats, such as scFv-phage display libraries. We used two antibody library sources, a library derived from 2 llamas immunized with ovarian cancer membrane preparations, and a naïve VHH library. Although both libraries resulted in the identification of a number of surface targets, the identity of the targets differed. The low diversity (˜10\hat{ }4) immunized library likely shows a bias towards highly expressed and highly immunogenic targets (such as Her2), while the more diverse naïve library (10\hat{ }7) has the potential to identify targets with varying expression patterns, and might thus be better suited for this approach. Although we performed follow up studies on the limited number of 200 candidates, we anticipate that deeper candidate mining and the use of more diverse libraries would lead to the identification of many additional targets.

While the target identification method we applied proved to be successful for about 75% of antibodies, we were unfortunately unable to identify the target of a few antibodies with interesting binding patterns, including one HGSOC specific antibody cluster (Cluster C). Alternative genome-based approaches such as CRISPR knockout or CRISPR activation screens could be considered for these challenging to identify antibody targets.

Anti-Her2 antibodies have been successfully used in treatment of Her2-overexpressing breast, uterine serous, and metastatic gastric cancers (9, 21). The discovery of a set of Her2 antibodies in the context of ovarian cell lines suggests Her2 should be evaluated as a target in a subset of ovarian cancers. Indeed, analysis of CCLE data shows high expression of Her2 in some ovarian cancer cell lines of which a small subset is highly Her2 dependent; for example, the ovarian cell line SKOV3 shows levels of Her2 overexpression and dependency similar to the SKBR3 breast cancer cell line (FIG. 8A). Furthermore, HER2 overexpression/amplification has been reported in ovarian cancer, especially in clear cell and mucinous tumors (22). Her2 targeting has been explored in a limited number of clinical trials in ovarian cancer using Trastuzumab or Pertuzumab. These trials showed limited clinical benefit in ovarian cancer patients, particularly when compared to combination chemotherapy (23-25). However, in breast cancer, single agent Her2 therapy showed a similar limited response. Combining anti-Her2 therapy with chemotherapy or as Her2 conjugated antibodies led to vastly improved responses, suggesting that a similar approach might be worth considering for future ovarian cancer trials.

HGSOC is the most common and lethal subtype of ovarian cancers, with the vast majority of women diagnosed at an advanced stage of disease. The current standard treatment is surgical debulking combined with chemotherapy. While standard therapy induces an initial response, tumors ultimately recur, and 70% of patients die within 5 years of diagnosis (26). To achieve better outcomes, new therapeutic targets are needed. Our screen led to the identification of CADM1 and BCAM, two adhesion proteins that are highly expressed on the surface of HGSOC cell lines. Previous studies have demonstrated that CADM1 can act as tumor suppressor and is frequently inactivated by promoter hypermethylation in many solid tumors, including pancreatic, lung, melanoma, esophageal, and cervical cancer (27-32). However, CADM1 has also been reported to be overexpressed and pro-tumorigenic in T-cell leukemia, lymphoma, and small cell lung cancer (33, 34). Ectopic expression of CADM1 has been suggested to inhibit cell proliferation and migration in an endometrial ovarian cancer cell line model (35). The high endogenous expression of CADM1 in the two HGSOC cell lines in this study does not appear to support these findings and suggests a more complex, possibly context dependent function. The physiological roles of CADM1 in ovarian cancer will need further investigation to determine its suitability for targeted therapy of HGSOC.

BCAM, first shown to be highly expressed on sickle red blood cells (36, 37), is overexpressed in a number of tumors (35, 36), notably highest in HGSOC, while its expression appears relatively low in normal tissues, with moderate expression in the kidney and the thyroid (FIG. 11A,B). We found that BCAM shows high expression in about 35-40% of primary HGSOC tumors and low to undetectable levels in kidney and thyroid (FIG. 11C). BCAM has previously been suggested as ovarian specific target (38). We found that BCAM expression is very low or undetectable on the surface of healthy RBCs, alleviating potential off-tumor toxicity concens. A recurrent BCAM-AKT2 fusion has also been described in some HGSOC (39). While we did not find evidence for this fusion in our cell line and organoid models, targeting the BCAM extracellular domain would allow the elimination of BCAM expressing cells irrespective of their fusion status.

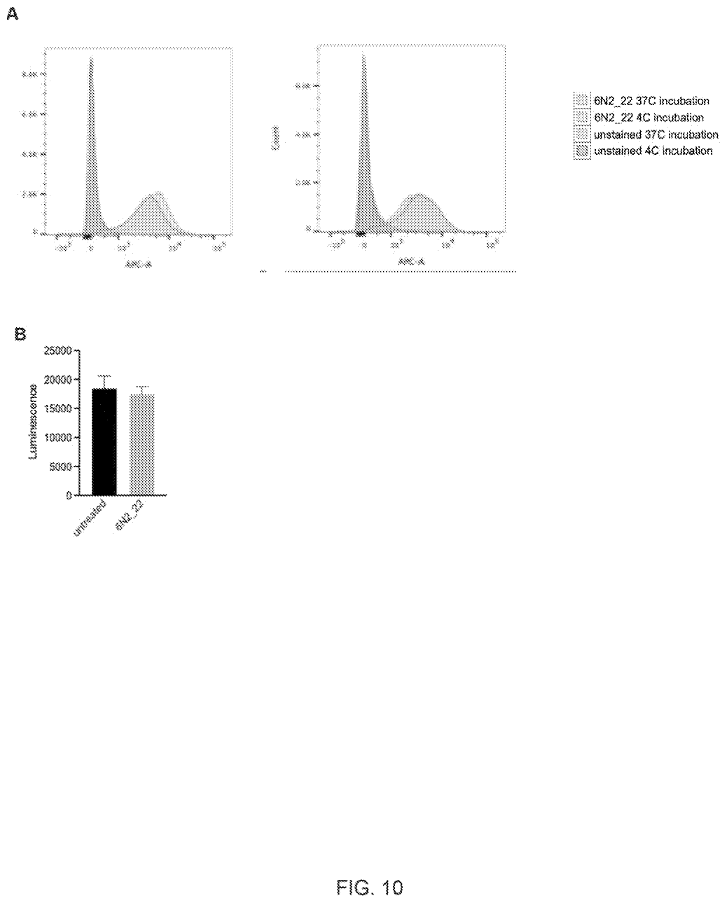

BCAM is a transmembrane glycoprotein with 5 immunoglobulin-like domains that acts as a receptor for Laminin a5 (LAMA5) (18, 19). Their interaction was demonstrated to promote adhesion and migration of carcinoma cells (18, 19). Accordingly, inhibition of BCAM-LAMA interaction has been demonstrated to have an inhibitory effect on migration (40, 41). Surprisingly, we no correlation between Laminin 5 and BCAM expression in HGSOC in tissue microarrays (FIG. 11D), raising the question as to whether laminin 5 is primary ligand for BCAM in ovarian cancer. In agreement with this, although our mutagenesis data suggest that the 6N2_22 epitope at least partially overlaps with the LAMA5 binding region, it does not have an apparent effect on cell adhesion (FIG. 10B). A previous study described an α-BCAM antibody-drug conjugate that induced cancer cell killing (42). We did not observe evidence that 6N2_22 triggers BCAM internalization in 2 cell lines tested (FIG. 10A), but it showed potent ADCC activity in vitro and in vivo, underscoring its potential use as therapeutic antibody in the VHH-hIgG1-Fc format.

Together, using an unbiased approach focused on identifying antibodies with specific binding patterns we identified a number of novel and surprising surface proteins as candidates for targeted therapy against subsets of ovarian cancer. Applying similar screens to other cancers, with a focus on individual cancer subtypes, or in the context of different microenvironmental conditions will likely lead to the discovery of many new highly specific targets that will accelerate the design of innovative new single or combination cancer therapies.

### Materials and Methods

### Cell Culture and Cell Line Generation

293T, A549, IMR, JIMTI, KP4, MIAPACA, PANC1, PATU8902, and PATU 8988T cell lines we cultured in DMEM media (Life Technologies). The JHOC5 cell line was cultured in DMEMF12 (Life Technologies). RMUGS cells were cultured in HAM's F12 (Fischer Scientific). SKBR3, SKOV3, and HT29 lines were cultured in McCoy's 5A media (Life Technologies). ASPC1, BXPC3, ES2, HCC1395, HCC202, Jurkat, Kuramochi, OC314, OVCAR8, OVSAHO, and PANC1005 lines were all cultured in RPMI (Life Technologies). All cells were grown at 37° C. and 5% CO2 and supplemented with 10% fetal bovine serum (FBS) and 1% penicillin streptomycin (Life Technologies). Organoid cultures were grown as previously described (43).

### Expression Vectors and Cloning

For expression of VHH-hFc chimeric antibodies pcDNA3 was modified to carry the IgGKSP (METDTLLLWVLLLWVPGSTG (SEQ ID NO: 176)) signal peptide for antibody secretion and human IgG1-Fc by Gibson cloning. VHH sequences were synthesized and cloned in frame into the modified vector using Agel/EcoRI restriction sites (Genscript). Vectors for CRISPR ko and CRISPRa have been obtained from the Genomics Perturbations Platform at the Broad Institute of Harvard and MIT (Cambridge, MA). ORF expression vectors for human and mouse BCAM, MCAM and Her2 were obtained from Origene and sequence verified. Chimeras and point mutants were generated by overlapping PCR and Gibson cloning into EcoRI/Xhol cut pcDNA3.

### Transfections and Lentiviral Transduction

For overexpression experiments, 293T cells were transfected using Lipofectamine P3000 according to manufacturer instructions. Cells were analyzed by western blotting or Flow cytometry 2-3 days post transfection. For siRNA transfection, lipofectamine RNAiMAX (Life Technologies) was used according to manufacturer instructions. Typically 10 UM siRNA was transfected and knockdown validated by immunoblot 2-3 days post transfection.

For lentiviral production, virus was produced by cotransfecting 293T cells with the lentiviral vector, D8.9 packaging construct, and VSV-G using Lipofectamine P3000 reagent (Life Technologies) according to manufacturer protocol. Media was changed the following day and virus harvested 2 days post transfection. After filtration through a 0.45 nM syringe filter (Fisher Scientific) cell lines were infected in the presence of polybrene (Santa Cruz). Media was changed 24h post infection and selection with puromycin (Fisher Scientific) or blasticidin (Fisher Scientific) was started 2 days post infection.

### Immunoblotting

Confluent plates of cells were harvested, washed with PBS, and lysed with cold RIPA buffer (Sigma Aldrich) with protease inhibitor and phosphatase inhibitor tablets (Sigma Aldrich). Lysates were cleared by centrifugation and protein quantified using the Thermo Fisher BCA Protein Assay protocol. Equal amounts of proteins were prepared in SDS loading buffer supplemented with β-mercaptoethanol, boiled at 95° C. to denature proteins, loaded onto precast 4-12% Bis-Tris gels (Life Technologies), and subjected to electrophoresis at 100 V. They were then transferred to PVDF membranes (Life Technologies) with the iBlot2 Transfer System for 7 min or 1 hr wet transfer at 100V. Membranes were blocked in Intercept Blocking Buffer (LICOR Biosciences) followed by incubation with indicated primary and IRDye-labelled secondary antibodies (LICOR Biosciences). Bands were visualized with the Odyssey® Imaging Systems. Primary antibodies used were as follows: α-Her-2 (2242, Cell Signaling), α-BCAM (AF148, R&D), α-α-Tubulin ( ) α-Myc-tag (05-419, Upstate).

### Membrane Preparation of Immunization

Membrane preparations were prepared as described in (44). Briefly, 1×10\hat{ }7 of each ovarian cancer cell line (SKOV3, OVCAR8, Kuramochi, OVSAHO, ES2, OC314, RMUGS) were resuspend in 5 ml cold 25 mM Tris-HCl, pH 7.4, 320 mM sucrose, protease inhibitor, lyze by sonication followed by spinning at 1000g for 12 min 4C to remove unbroken cell nuclei and cell debris. The supernatant was centrifuged at 40.000 rpm for 30 min, the pellet was resuspended in 50 mM Tris-HCl; pH 7.4 and again centrifuged at 40.000 rpm for 20 min. The pellet was resuspended in 1 ml 0.02M bicarbonate buffer (pH 9.6) and passed through 27G needle to homogenize membrane fraction. Equal amounts of membrane fractions from each cell line were pooled and used for immunization. Llama immunizaitons and library preparation was performed by Prosci.

### Biopanning and NGS

For negative selection, PBMCs were isolated from blood collars by Ficol gradient centrifugation. The phage display library was incubated with PBMCs for 1h on ice. After centrifugation, the supernatant was transferred to the harvested positive cell lines and incubated for 2-4h gentle mixing. Cells were washed with PBS/5% BSA/0.5% Tween followed by elution of bound phages with 0.1M Glycin-HCl pH 2.6 and neutralization with Tris-base. Output library was rescued in TG1 cells and amplified. For the second round of negative selection the new sub-library was incubated with PBMCs followed by incubation with fibroblasts. Supernatant was added to individual positive cell lines and incubated for 2-4h, followed by washing and elution in Glycin-HCl. Eluted phages were rescued in TG1 cells by culturing O/N at 30C in presence of Ampicillin and glucose. For NGS phagemids from each output library were isolated using a plasmid midiprep kit (Qiagen) followed by restriction digest with Agel/Sfil to isolate VHH fragments. Illumina paired-end 2×250 bp sequencing was performed on targeted VHH sequences.

### NGS Analysis

Trimmomatic (version 0.38) was first used to remove fragments with low base calling quality (average Phred score <30) and clip Illumina adapter sequences from all reads (45). Reads were additionally cropped at 225 bp to remove low quality positions. Quality passing paired reads were merged using FLASh (version 1.2.11) with expected fragment length and standard deviation set to 375 bp and 35 bp, respectively (46). Merged reads were filtered to only those which appeared to be valid VHH sequences based on expected heavy chain structure. Reference sequences for the camelid heavy chain framework regions (FR) were obtained from IGHV and IGHJ alleles of the closely related Vicugna pacos in the IMGT/V-QUEST reference directory set (release 201908-4) (47). Position weight matrices (PWMs) were constructed for each of the four FRs based on the reference alleles. Full length VHH sequences were trimmed and translated to amino acid sequences. Amino acid (AA) sequences for the complementary determining region 3 (CDR3) were extracted from reads based on the previously matched FR3 and FR4 positions. CDR3 sequences shorter than 2 AAs were dropped. Unique CDR3 sequences were clustered across all samples using CD-HIT (version 4.8.1) (48, 49). CDR3 sequences were sorted by total fragment counts prior to clustering with CD-HIT. For each CDR3 cluster, we counted the number of fragments matching a CDR3 sequence in the cluster for each sample. The matrix of sample fragment counts across CDR3 clusters was next used for differential analysis (50). Selection of sequences for follow up was based on overlap between replicates, the number of cell lines we identified them with priority given to sequences found in at least 2 ovarian cell lines while absend in PBMCs, and the highest single cell line hits.

### Target Identification

Target identification was essentially done as described (6). Briefly, to prepare the ASB crosslinked antibody 100 μg of purified antibody were incubated with PEG4-SPDP (Thermo Fischer Scientific) at room temperature for 1h followed by quenched with glycine (Santa Cruz Biotechnology). The antibody was then incubated overnight with 60 μg reduced ASB in 1 mM EDTA (Life Technologies). Antibodies were buffer exchanged with PBS in Amicon filters (Thermo Fisher Scientific). To confirm crosslinking, 1 μg of sample was run on a gel and Coomassie stained in parallel with unlabeled purified antibody. Successful crosslinking indicated an upshifted band in the labelled sample. Approximately 10⁸ cells were harvested and suspended in 2 mM sodium meta-periodate (Sigma-Aldrich) in PBS pH 6.5, followed by 4° C. incubation. Cells were incubated with 100 μg of ASB-labelled antibody followed by addition of 10 mM p-phenylenediamine (Sigma-Aldrich) to catalyze crosslinking. After washing cells were flash frozen in a dry ice and ethanol bath, and stored at −80° C. The cell pellet was lysed in 2% sodium dodecyl sulfate (Sigma-Aldrich) with protease inhibitor (Sigma-Aldrich), benzonase (Santa Cruz Biotechnology) and cell clumps were dissociated by passing through a syringe needle (Sigma-Aldrich 22 gauge, L 1 in). 50 mM DTT (Sigma-Aldrich) was added to cleared lysates and boiled to cleave biotin crosslinks. Cooled samples were treated with 375 mM IAA (Sigma-Aldrich) in 50 mM ammonium bicarbonate (Westnet Inc) in the dark, and subsequently quenched with 200 mM DTT. Biotinylated proteins were isolated from sample by incubating with Streptavidin magnetic beads (Life Technologies) followed by multiple washes with 0.5% SDS, 2M urea (Life Technologies), and 50 mM AMBIC. The samples was finally resuspended in 50 mM AMBIC and stored at 4° C. until mass spec analysis. Mass spec analysis was performed after on bead Trypsin digestion on a LTQ Orbitrap Velos Pro ion-trap mass spectrometer (Thermo Fisher) at the Harvard Medical Schools Taplin Core facility as described previously (51, 52)

### Antibody Expression

Expi293F cells (Life Technologies) were grown with Expi293 Expression Medium (Fisher Scientific) at 120 rpm at 37° C. with >80% relative humidity and 8% CO2. Cells at a density between 3-5×10⁶ viable cells/ml were transfected with 2.5 μg of desired antibody expression plasmid with ExpiFectamine 293 transfection kit reagent (Life Technologies) according to manufacture instructions. Cell supernatants containing the antibodies were harvested 3 days later by centrifugation. Expression of antibodies was verified by SDS-PAGE/Coomassie staining.

### Antibody Purification

For antibody purification crude antibody supernatant was incubated with Protein A Plus agarose beads (Pierce) for 2h followed by washing with PBS. Beads were collected by centrifugation at 1,000 rpm and antibodies eluted in 4× bead volumes of Elution Buffer at pH2.0 (Pierce). The eluate was neutralized with 3M Tris-Base. Antibodies were run on SDS-PAGE to confirm size and purity.

### Flow Cytometry

For antibody binding studies, cells were washed with PBS and harvested using CellStripper (Thermo Fisher). Antibody staining was performed in PBS/5% BSA with indicated amounts of primary antibody. Additional staining with corresponding fluorescently labelled secondary antibody was performed when primary antibody was not directly conjugated. All wash steps were performed in PBS/5% BSA. To assess cell viability cells were stained with Fixable Viability Dye eFluor 780 (Biolegend). For the multiplexed screening assay each cell lines were labelled individually with different concentrations of CellTrace CSFE and/or CellTraceViolet. Briefly, 1.2×10⁶ cells per cell line were labelled with Violet and CFSE dye combinations (CellTrace Violet 40 μM, Violet 5 μM, CFSE 10 μM, and CFSE 1 μM) for 30 minutes. The reaction was quenched with FBS. For antibody screening, labelled cell lines were mixed and aliquoted to 3×10⁵ total cells/well. All antibody staining reactions were performed on ice, protected from light, and wash steps are performed with PBS/5% BSA. Cells were incubated with 20 μg/mL antibody in 100 μL PBS/5% BSA for 30 minutes, followed by incubation with APC conjugated anti-human IgG Fc (BioLegend) at 5 μg/mL in 100 μL PBS/5% BSA for 30 minutes. Fixable Viability Dye eFluor 780 (eBioscience) at 1000× dilution was added for 15 minutes. For antibody competition assays antibodies were labelled with Alexa Fluor 488 according to manufactures instructions (Thermo Fisher). For staining 3×10⁵ cells were aliquoted into each well. For blocking, unlabeled antibodies are added to the samples at saturating amounts (20 μg/ml) for 30 minutes on ice. Control cells were incubated with no antibody or an unrelated antibody. After washing, 488-labeled antibodies were added for 30 minutes to blocked and control samples. Upon washing cells were resuspended in PBS/5% BSA for flow analysis. Facs analysis was performed on the BD Fortessa. All data was analyzed using FlowJo software. Commercial primary antibodies used were as follows: α-hERBB2-APC (FAB1129A, R&D), α-human-APC (HP6017, Biolegend), α-human-488 (A10631, Invitrogen); Trastuzumab and Pertuzumab were purchased from the DANA-Farber Cancer Institute Pharmacy.

### ADCC Assay

Effector PBMCs were isolated from buffy coats using Percoll (Sigma) density gradient centrifugation and stimulated with 100 ng/ml IL-2 overnight. Target cells were stained with CellTrace Violet (Life Technologies) according to manufactures instructions. 10⁴ Violet-stained cells were seeded into round bottom 96-well plates in RPMI/5% human AB serum (Sigma). Indicated amounts of antibodies were incubated at 37° C./5% CO2 for 30 minutes. 2.5×10⁵ peripheral blood mononuclear cells (PBMCs) were added and incubated at 37° C./5% CO2 for 4 hours. The media was replaced with 1:20 Annexin V-488 (Life Technologies) diluted in Annexin V buffer (Life Technologies) and incubated at room temperature for 30 minutes. The samples were adjusted to 200 μL before being assessed on BD Fortessa II Cytometer and analyzed on FlowJo™

### BCAM ELISA

20 μg/ml recombinant BCAM (Sino) was coated onto 96-well high-attachment plates and incubated at 4° C. O/N. The plate was washed two times with PBS (Thermo Fisher) and blocked with PBS/10% bovine serum albumin (BSA) for 1 hour at room temperature. Primary antibodies in PBS/10% BSA were added in 1:4 dilutions from 10 μg/ml to 0.01 μg/ml and incubated for 1 hour at room temperature. The plate was washed three times with PBS/0.1% Tween before adding the secondary anti-human-HRP (Cell Technologies) in a 1:1000 ratio diluted in PBS/10% BSA and incubated for 30 minutes at room temperature. The samples were washed three times with PBS/0.1% Tween, and TMB substrate (Pierce) was added to the wells for 15 minutes at room temperature. 2M Sulfuric acid was added directly to the TMB substrate to stop the reaction. The absorbance was recorded at 450 nm on SpectraMax M5E (Molecular Devices).

### Immunohistochemistry

Tissue microarrays (BC11012b, OV812) were purchased from US Biomax. Fluorescence Immunohistochemistry staining was performed on 5-μm sections of the TMAs for detection of BCAM on the automated Leica Bond RX system, with α-BCAM antibody PA5-50376 (Thermo Fisher), at dilution of 1:50 for 15 minutes, a tyramide conjugated FITC fluorophore and a DAPI counterstain. For spectral imaging & analysis the stained slides were scanned on a Vectra 3 imaging system (Akoya Bio) and analyzed using Halo (Indica Labs). We run an algorithm learning tool utilizing the Halo training for the epithelial cells and stroma regions, and subsequently completed cell segmentation. The reported expression intensity for a cell is the average intensity of the total pixel values in the antigen presented subcomponent of the cell. The threshold for BCAM was set based on the staining intensity normalized by the background slide. Cells with the intensity above the setting threshold were defined as positive.

### REFERENCES

1. S. Bagchi, R. Yuan, E. G. Engleman, Immune Checkpoint Inhibitors for the Treatment of Cancer: Clinical Impact and Mechanisms of Response and Resistance. Annu Rev Pathol 16, 223-249 (2021).
2. A. D. Waldman, J. M. Fritz, M. J. Lenardo, A guide to cancer immunotherapy: from T cell basic science to clinical practice. Nat Rev Immunol 20, 651-668 (2020).
3. Y. Liu, A. Beyer, R. Aebersold, On the Dependency of Cellular Protein Levels on mRNA Abundance. Cell 165, 535-550 (2016).
4. D. Wang et al., A deep proteome and transcriptome abundance atlas of 29 healthy human tissues. Mol Syst Biol 15, e8503 (2019).
5. S. Domcke, R. Sinha, D. A. Levine, C. Sander, N. Schultz, Evaluating cell lines as tumour models by comparison of genomic profiles. Nat Commun 4, 2126 (2013).
6. T. L. Tremblay, J. J. Hill, Biotin-transfer from a trifunctional crosslinker for identification of cell surface receptors of soluble protein ligands. Sci Rep 7, 46574 (2017).
7. M. Ghandi et al., Next-generation characterization of the Cancer Cell Line Encyclopedia. Nature 569, 503-508 (2019).
8. A. Tsherniak et al., Defining a Cancer Dependency Map. Cell 170, 564-576 e516 (2017).
9. A. N. Fader et al., Randomized Phase II Trial of Carboplatin-Paclitaxel Versus Carboplatin-Paclitaxel-Trastuzumab in Uterine Serous Carcinomas That Overexpress Human Epidermal Growth Factor Receptor 2/neu. J Clin Oncol 36, 2044-2051 (2018).
10. G. Valabrega, F. Montemurro, M. Aglietta, Trastuzumab: mechanism of action, resistance and future perspectives in HER2-overexpressing breast cancer. Ann Oncol 18, 977-984 (2007).
11. T. Vu, F. X. Claret, Trastuzumab: updated mechanisms of action and resistance in breast cancer. Front Oncol 2, 62 (2012).
12. M. P. Velders et al., The impact of antigen density and antibody affinity on antibody-dependent cellular cytotoxicity: relevance for immunotherapy of carcinomas. Br J Cancer 78, 478-483 (1998).
13. C. E. Eyler, M. J. Telen, The Lutheran glycoprotein: a multifunctional adhesion receptor. Transfusion 46, 668-677 (2006).
14. N. M. Burton, R. L. Brady, Molecular structure of the extracellular region of Lutheran blood group glycoprotein and location of the laminin binding site. Blood Cells Mol Dis 40, 446-448 (2008).
15. T. J. Mankelow et al., The Laminin 511/521-binding site on the Lutheran blood group glycoprotein is located at the flexible junction of Ig domains 2 and 3. Blood 110, 3398-3406 (2007).
16. M. A. Bowen et al., Cloning, mapping, and characterization of activated leukocyte-cell adhesion molecule (ALCAM), a CD6 ligand. J Exp Med 181, 2213-2220 (1995).
17. J. P. Johnson, U. Rothbacher, C. Sers, The progression associated antigen MUC18: a unique member of the immunoglobulin supergene family. Melanoma Res 3, 337-340 (1993).
18. W. El Nemer et al., The Lutheran blood group glycoproteins, the erythroid receptors for laminin, are adhesion molecules. J Biol Chem 273, 16686-16693 (1998).
19. M. Udani et al., Basal cell adhesion molecule/lutheran protein. The receptor critical for sickle cell adhesion to laminin. J Clin Invest 101, 2550-2558 (1998).
20. S. F. Parsons et al., The Lutheran blood group glycoprotein, another member of the immunoglobulin superfamily, is widely expressed in human tissues and is developmentally regulated in human liver. Proc Natl Acad Sci USA 92, 5496-5500 (1995).
21. D. Y. Oh, Y. J. Bang, HER2-targeted therapies—a role beyond breast cancer. Nat Rev Clin Oncol 17, 33-48 (2020).
22. M. Fujimura et al., HER2 is frequently over-expressed in ovarian clear cell adenocarcinoma: possible novel treatment modality using recombinant monoclonal antibody against HER2, trastuzumab. Jpn J Cancer Res 93, 1250-1257 (2002).
23. M. A. Bookman, K. M. Darcy, D. Clarke-Pearson, R. A. Boothby, I. R.

Horowitz, Evaluation of monoclonal humanized anti-HER2 antibody, trastuzumab, in patients with recurrent or refractory ovarian or primary peritoneal carcinoma with overexpression of HER2: a phase II trial of the Gynecologic Oncology Group. J Clin Oncol 21, 283-290 (2003).
24. S. Makhija et al., Clinical activity of gemcitabine plus pertuzumab in platinum-resistant ovarian cancer, fallopian tube cancer, or primary peritoneal cancer. J Clin Oncol 28, 1215-1223 (2010).
25. A. H. Sims et al., Defining the molecular response to trastuzumab, pertuzumab and combination therapy in ovarian cancer. Br J Cancer 106, 1779-1789 (2012).
26. B. M. Reid, J. B. Permuth, T. A. Sellers, Epidemiology of ovarian cancer: a review. Cancer Biol Med 14, 9-32 (2017).
27. Y. Chen et al., Lost expression of cell adhesion molecule 1 is associated with bladder cancer progression and recurrence and its overexpression inhibited tumor cell malignant behaviors. Oncol Lett 17, 2047-2056 (2019).
28. L. M. De Strooper et al., CADM1, MAL and miR124-2 methylation analysis in cervical scrapes to detect cervical and endometrial cancer. J Clin Pathol 67, 1067-1071 (2014).
29. T. Fukami et al., Promoter methylation of the TSLC1 gene in advanced lung tumors and various cancer cell lines. Int J Cancer 107, 53-59 (2003).
30. R. M. Overmeer et al., Association between dense CADM1 promoter methylation and reduced protein expression in high-grade CIN and cervical SCC. J Pathol 215, 388-397 (2008).
31. L. van der Weyden et al., Increased tumorigenesis associated with loss of the tumor suppressor gene Cadm1. Mol Cancer 11, 29 (2012).
32. Y. You et al., TSLC1 gene silencing in cutaneous melanoma. Melanoma Res 20, 179-183 (2010).
33. H. Sasaki et al., Overexpression of a cell adhesion molecule, TSLC1, as a possible molecular marker for acute-type adult T-cell leukemia. Blood 105, 1204-1213 (2005).
34. S. Kikuchi et al., Expression of a splicing variant of the CADM1 specific to small cell lung cancer. Cancer Sci 103, 1051-1057 (2012).
35. X. Si et al., CADM1 inhibits ovarian cancer cell proliferation and migration by potentially regulating the PI3K/Akt/mTOR pathway. Biomed Pharmacother 123, 109717 (2020).
36. Y. Kikkawa, J. H. Miner, Review: Lutheran/B-CAM: a laminin receptor on red blood cells and in various tissues. Connect Tissue Res 46, 193-199 (2005).
37. V. Chaar et al., Aggregation of mononuclear and red blood cells through an {alpha} 4 {beta} 1-Lu/basal cell adhesion molecule interaction in sickle cell disease. Haematologica 95, 1841-1848 (2010).
38. P. Garinchesa, M. Sanzmoncasi, I. Campbell, W. Rettig, Non-polarized expression of Basal-cell adhesion molecule B-cam in epithelial ovarian cancers. Int J Oncol 5, 1261-1266 (1994).
39. K. Kannan et al., Recurrent BCAM-AKT2 fusion gene leads to a constitutively activated AKT2 fusion kinase in high-grade serous ovarian carcinoma. Proc Natl Acad Sci USA 112, E1272-1277 (2015).
40. A. Guadall et al., Dimerization and phosphorylation of Lutheran/basal cell adhesion molecule are critical for its function in cell migration on laminin. J Biol Chem 294, 14911-14921 (2019).
41. Y. Kikkawa et al., The lutheran/basal cell adhesion molecule promotes tumor cell migration by modulating integrin-mediated cell attachment to laminin-511 protein. J Biol Chem 288, 30990-31001 (2013).
42. Y. Kikkawa et al., Internalization of CD239 highly expressed in breast cancer cells: a potential antigen for antibody-drug conjugates. Sci Rep 8, 6612 (2018).
43. J. G. Doench et al., Optimized sgRNA design to maximize activity and minimize off-target effects of CRISPR-Cas9. Nat Biotechnol 34, 184-191 (2016).
44. M. K. Tur, M. Huhn, S. Sasse, A. Engert, S. Barth, Selection of scFv phages on intact cells under low pH conditions leads to a significant loss of insert-free phages. Biotechniques 30, 404-408, 410, 412-403 (2001).
45. A. M. Bolger, M. Lohse, B. Usadel, Trimmomatic: a flexible trimmer for Illumina sequence data. Bioinformatics 30, 2114-2120 (2014).
46. T. Magoč, S. L. Salzberg, FLASH: fast length adjustment of short reads to improve genome assemblies. Bioinformatics 27, 2957-2963 (2011).
47. M.-P. Lefranc, G. r. Lefranc, The immunoglobulin factsbook, Factsbook series (Academic Press, San Diego, 2001), pp. xiv, 457 pages.
48. L. Fu, B. Niu, Z. Zhu, S. Wu, W. Li, CD-HIT: accelerated for clustering the next-generation sequencing data. Bioinformatics 28, 3150-3152 (2012).
49. W. Li, A. Godzik, Cd-hit: a fast program for clustering and comparing large sets of protein or nucleotide sequences. Bioinformatics 22, 1658-1659 (2006).
50. M. I. Love, W. Huber, S. Anders, Moderated estimation of fold change and dispersion for RNA-seq data with DESeq2. Genome Biol 15, 550 (2014).
51. J. Peng, S. P. Gygi, Proteomics: the move to mixtures. J Mass Spectrom 36, 1083-1091 (2001).
52. J. K. Eng, A. L. McCormack, J. R. Yates, An approach to correlate tandem mass spectral data of peptides with amino acid sequences in a protein database. J Am Soc Mass Spectrom 5, 976-989 (1994).

### Example 6

FASTdiscovery platform—Flexible Antibody and Simiultaneous Target discovery

PhASTdiscovery platform—Phenotypic Antibody and Simulataneous Target discovery

A platform for the rapid discovery of cell surface tagest with simultaneous selection of antibodies

### Discovery of Multiple Ovarian Specific Antibody-Target Pairs in a Single Round of Screening

Identification of antibody-target pairs with specific binding characteristics in ovarian cancer using a panel of ovarian cell lines as model system
Considerations for the platform development
   Antibody format
      conventional IgGs, scFv, VHHs
   Display technology/Type of library
      mammalian, yeast, bacterial
   Selection strategy
      colony picking, computational guided selection
   Screening technology
      live cell binding assays, ELISAs, HTP/multiplexed
   Target ID methodology
      CRISPRa, CRISPRko, ORFmicroarrays, Biochemical/Mass-spec

### Basal Cell Adhesion Molecule/DC239

Biological Function
   Transmembrane glycoprotein with 5 immunoglobulin-like domains
   Laminin α5 (LAMA5) receptor
   Its intracellular region carries SH3 binding motif (likely mediating signaling)
   Without wishing to be bound by theory, plays a role in development
   Mediates adhesion and migration
   People with BCAM null phenotype don't show any clinical conditions in physiological setting
   Null mice are healthy, show kidney and intestinal abnormalities without functional defects
Pathology
   Overexpressed in sickle red blood cells
   Mediates abnormal adhesion of sickle red blood cells to vascular wall
   Highly overexpressed in ovarian and endometrial cancers
   Overexpressed in metastasis of several cancers (including colon, breast)
   BCAM/LAMA5 act at the tumor: TME interface
   Promotes migration of carcinoma cells
   Blocking BCAM/LAMA5 interaction inhibits migration
Library:
   Use primary tumors, such as patient tumor samples, for library generation (need to eliminate immune cells)
   Use organoids for library generation (early passage-cleaner system than primary tumors, more material)
Panning:
   Using patient derived organoids-healthy controls also available; tumor material
Screening:
   1. validation:
      Using organoids
      Circulating tumor cells
      Large panel of cell lines (across cancer types)
         to determine specificity and validate indications beyond ovarian cancer
         develop a surface target map (explore multiplex options, such as SNP for barcoding, DNA labelling of antibodies,
         to have high-throughput multiplexed binding assay (cell line+antibody multiplexed/readout through NGS, and, optionally, combined with RNAseq to infer functionality-REAP seq/CITE-seq)
   2. Functional screen of validated antibodies:
      Link binding profile to functionality of antibody (to identify “surprising” biologies; helps prioritization of candidates)
      assays-ADCC, cell death, proliferation, adhesion
Target ID:
   Of antibodies with validated binding profile of interest, preference given functional antibodies
      Comparison of the PhASTdiscovery Platform with Conventional Target-Antibody Discovery Workflows
High Throughput Approach-discovery of hundreds of target-antibody pairs in just 2-3 months (instead of years)
Identification of Targets in the relevant context-Discovery of antibodies specific for physiologically relevant target states
High Versatility-Platform permits discovery of antibodies and targets in specific settings:
   Antibody target specificity
      Tumor vs normal
      Cancer subtype
      WT vs. mutant genes
      Metastatic vs. primary
      In presence/absensce of microenvironmental factors (e.g. GF, cytokines, INF, ECM)
      Surface properties of target: internalized vs. constitutive surface expression
   Tunable affinity: high vs moderate/low
   Adoptable to diverse antibody formats

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1-7. (canceled)

8. A method for the sequential selection and identification of an anti-cancer antibody and its target, the method comprising:
subjecting a phage display input library to affinity selection to produce an output library, wherein affinity selection comprises at least one panning step with a live cell sample positive for a biomarker and at least one panning step with a live cell sample negative for a biomarker;
selecting from the output library at least one antibody candidate based on sequence analysis, binding profiles, or both;
synthesizing, manufacturing, or isolating the selected antibody candidate;
optionally, validating the antibody candidate by flow cytometry; and
identifying the antibody candidate's target by mass spectrometry.

9. The method of claim 8, further comprising obtaining the input library.

10. The method of claim 8, further comprising producing the antibody candidate.

11. The method of claim 10, wherein producing comprises cloning or synthesizing, reformatting, and expressing the antibody candidate.

12. The method of claim 8, further comprising analyzing the output library to identify the antibody candidate.

13. (canceled)

14. The method of claim 8, wherein the input library comprises phage display, mammalian display, yeast display, bacterial display, ribosome display, or B-cells.

15. The method of claim 8, wherein the input library comprises a naïve library, a synthetic library, a library generated after immunization of animals, or a combination thereof.

16-19. (canceled)

20. The method of claim 8, wherein the sample negative for a biomarker and/or the sample positive for a biomarker comprises a diseased state, a non-diseased state, and/or a combination thereof.

2. (canceled)

21-25. (canceled)

26. The method of claim 12, wherein analyzing comprises sequencing, computational pre-processing, and computational guided selection.

27-29. (canceled)

30. The method of claim 11, wherein the antibody candidate comprises a full-length antibody, a fusion protein, or an antibody fragment.

31. The method of claim 30, wherein the antibody fragment comprises IgG, VH, Fab, scFv-Fc, diabody, scFv-CH3, scFab, scFv-zipper, scFv, or VHH.

32. The method of claim 8, wherein validating comprises an immunoassay, a live cell binding assay, high throughput cell line multiplexing through fluorescent barcoding, plate based binding assays, high content analysis, or any combination thereof.

33. The method of claim 32, wherein the immunoassay comprises flow cytometry, enzyme-linked immunosorbent assay (ELISA), plate based fluorescence binding assays, immunohistochemistry/fluorescent imaging, western blotting.

34. The method of claim 33, wherein flow cytometry comprises fluorescence-activated cell sorting (FACS).

35. (canceled)

36. The method of claim 8, wherein the identifying comprises immunoprecipitation, affinity purification, protein microarray, or genetic approaches.

37. The method of claim 36, wherein immunoprecipitation or affinity purification comprises:
linking an antibody with a label to produce a labeled antibody;
incubating the labeled antibody with a population of cells, wherein the labeled antibody binds to a target on the surface of the cells to produce an antibody-target conjugate;
isolating the antibody-target conjugate from the population of cells; and
identifying the target.

38. (canceled)

39. The method of claim 37, wherein the immunoprecipitation or affinity purification comprises antibody crosslinking.

40-42. (canceled)

43. The method of claim 8, wherein mass spectrometry analysis comprises LC-MS/MS, MALDI-TOF MS, ESI, or label free analysis based on MS signal intensity.

44-45. (canceled)

46. A method for generating a cancer cell surface map, the method comprising the method of claim 8, thereby producing the cancer cell surface map.

47. A method for identifying a therapeutic target, comprising:
subjecting an input display library to affinity selection to produce an output library, wherein affinity selection comprises live cell panning;
analyzing the output library to identify one or more antibodies; and
identifying the target of the one or more antibodies, thereby identifying a therapeutic target and therapeutic antibody.

48-49. (canceled)
